SEQUENCE LISTING (435 sequences)

SEQ ID NO: 1 - protein, 572 aa, Homo sapiens
  REGION 1..572: note = misc_feature - Full-length Human GPNMB Amino Acid Sequence
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 2 - protein, 498 aa, Homo sapiens
  REGION 1..498: note = misc_feature - Soluble Human GPNMB Amino Acid Sequence
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMAN

SEQ ID NO: 3 - protein, 515 aa, Macaca fascicularis
  REGION 1..515: note = misc_feature - Soluble Cynomolgus GPNMB Amino Acid Sequence
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFVVNLVFPRCQKEDANGNIVYEKNCRNEAGLSADPYVYNWTAWSEDSDGENGTGQSHHNVFPDGKPFPHHPGWRRWNFVYVFHTLGQYFQKLGRCSVRVSVNTANVTLGPQLMEVTVYRRHGRAYVPIAQVKDVYVVTDQIPVFVTMSQKNDRNSSDETFLKDLPITFDVLIHDPSHFLNYSTINYKWSFGDNTGLFVSINHTVNHTYVLNGTFSLNLTVKAAAPGPCPPPPPPPPRPPKPTPSLGPAGDNPLGLSEIPDENCQINRYGHFQATITIVEGILEVNIIQTTDVLMLVPGPDSSLIDFVVTCQGSIPTEVCTIISDPTCEITQNAVCSPVDVDEMCLLTVRRAFNGSGTYCVNLTLGDDTSLALTSTLISVPDRDPASSLRMANSGGGGLNDIFEAQKIEWHEHHHHHHHHH

SEQ ID NO: 4 - protein, 502 aa, Mus musculus
  REGION 1..502: note = misc_feature - Soluble Mouse GPNMB Amino Acid Sequence
MESLCGVLGFLLLAAGLPLQAAKRFRDVLGHEQYPDHMREHNQLRGWSSDENEWDEHLYPVWRRGDGRWKDSWEGGRVQAVLTSDSPALVGSNITFVVNLVFPRCQKEDANGNIVYEKNCRNDLGLTSDLHVYNWTAGADDGDWEDGTSRSQHLRFPDRRPFPRPHGWKKWSFVYVFHTLGQYFQKLGRCSARVSINTVNLTAGPQVMEVTVFRRYGRAYIPISKVKDVYVITDQIPVFVTMSQKNDRNLSDEIFLRDLPIVFDVLIHDPSHFLNDSAISYKWNFGDNTGLFVSNNHTLNHTYVLNGTFNLNLTVQTAVPGPCPPPSPSTPPPPSTPPSPPPSPLPTLSTPSPSLMPTGYKSMELSDISNENCRINRYGYFRATITIVEGILEVSIMQIADVPMPTPQPANSLMDFTVTCKGATPMEACTIISDPTCQIAQNRVCSPVAVDGLCLLSVRRAFNGSGTYCVNFTLGDDASLALTSTLISIPGKDPDSPLRAVN

SEQ ID NO: 5 - protein, 5 aa, synthetic construct
  REGION 1..5: note = Synthetic
NYCIH

SEQ ID NO: 6 - protein, 17 aa, synthetic construct
  REGION 1..17: note = Synthetic
YINPSTGYTEYNQKFKD

SEQ ID NO: 7 - protein, 8 aa, synthetic construct
  REGION 1..8: note = Synthetic
GRYDEFTY

SEQ ID NO: 8 - protein, 5 aa, synthetic construct
  REGION 1..5: note = Synthetic
NYYMY

SEQ ID NO: 9 - protein, 17 aa, synthetic construct
  REGION 1..17: note = Synthetic
EINPRNGGTNFNEKFKS

SEQ ID NO: 10 - protein, 4 aa, synthetic construct
  REGION 1..4: note = Synthetic
GLAY

SEQ ID NO: 11 - protein, 5 aa, synthetic construct
  REGION 1..5: note = Synthetic
NYYMY

SEQ ID NO: 12 - protein, 17 aa, synthetic construct
  REGION 1..17: note = Synthetic
EINTRNGGTNFNEKFKS

SEQ ID NO: 13 - protein, 4 aa, synthetic construct
  REGION 1..4: note = Synthetic
GFAY

SEQ ID NO: 14 - protein, 5 aa, synthetic construct
  REGION 1..5: note = Synthetic
SYYMY

SEQ ID NO: 15 - protein, 17 aa, synthetic construct
  REGION 1..17: note = Synthetic
EINPSNGGTYFNEKFKS

SEQ ID NO: 16 - protein, 7 aa, synthetic construct
  REGION 1..7: note = Synthetic
GGTYYAY

SEQ ID NO: 17 - protein, 5 aa, synthetic construct
  REGION 1..5: note = Synthetic
SYWLH

SEQ ID NO: 18 - protein, 17 aa, synthetic construct
  REGION 1..17: note = Synthetic
EINPSDGRTNYIEKVKT

SEQ ID NO: 19 - protein, 10 aa, synthetic construct
  REGION 1..10: note = Synthetic
SGGNYAWFAY

SEQ ID NO: 20 - protein, 5 aa, synthetic construct
  REGION 1..5: note = Synthetic
SYYIY

SEQ ID NO: 21 - protein, 17 aa, synthetic construct
  REGION 1..17: note = Synthetic
EINPSNGGTYFNEKFKN

SEQ ID NO: 22 - protein, 7 aa, synthetic construct
  REGION 1..7: note = Synthetic
GGNYYAY

SEQ ID NO: 23 - protein, 5 aa, synthetic construct
  REGION 1..5: note = Synthetic
SYWMH

SEQ ID NO: 24 - protein, 17 aa, synthetic construct
  REGION 1..17: note = Synthetic
EINSSNGRTNYNEKFKT

SEQ ID NO: 25 - protein, 16 aa, synthetic construct
  REGION 1..16: note = Synthetic
LGYYSGSPYPYYAMDC

SEQ ID NO: 26 - protein, 5 aa, synthetic construct
  REGION 1..5: note = Synthetic
SYWIT

SEQ ID NO: 27 - protein, 17 aa, synthetic construct
  REGION 1..17: note = Synthetic
DIFPGSGSTNYHEKFKS

SEQ ID NO: 28 - protein, 10 aa, synthetic construct
  REGION 1..10: note = Synthetic
GYFGSSFSDY

SEQ ID NO: 29 - protein, 5 aa, synthetic construct
  REGION 1..5: note = Synthetic
SYWIT

SEQ ID NO: 30 - protein, 17 aa, synthetic construct
  REGION 1..17: note = Synthetic
DIHPGSGRSNYNEKFKN

SEQ ID NO: 31 - protein, 6 aa, synthetic construct
  REGION 1..6: note = Synthetic
DYLFAY

SEQ ID NO: 32 - protein, 5 aa, synthetic construct
  REGION 1..5: note = Synthetic
GYYMN

SEQ ID NO: 33 - protein, 17 aa, synthetic construct
  REGION 1..17: note = Synthetic
EINPGSGITTYNQKFKA

SEQ ID NO: 34 - protein, 7 aa, synthetic construct
  REGION 1..7: note = Synthetic
PASWFAY

SEQ ID NO: 35 - protein, 5 aa, synthetic construct
  REGION 1..5: note = Synthetic
GYYMN

SEQ ID NO: 36 - protein, 17 aa, synthetic construct
  REGION 1..17: note = Synthetic
EINPSTGITTYNQKFKA

SEQ ID NO: 37 - protein, 7 aa, synthetic construct
  REGION 1..7: note = Synthetic
PASWFDY

SEQ ID NO: 38 - protein, 5 aa, synthetic construct
  REGION 1..5: note = Synthetic
GYYMN

SEQ ID NO: 39 - protein, 17 aa, synthetic construct
  REGION 1..17: note = Synthetic
EINPNTGFTTYNQNFKA

SEQ ID NO: 40 - protein, 7 aa, synthetic construct
  REGION 1..7: note = Synthetic
PSPSFDY

SEQ ID NO: 41 - protein, 5 aa, synthetic construct
  REGION 1..5: note = Synthetic
GYYMN

SEQ ID NO: 42 - protein, 17 aa, synthetic construct
  REGION 1..17: note = Synthetic
EINPNTGGTTYNQKFKA

SEQ ID NO: 43 - protein, 7 aa, synthetic construct
  REGION 1..7: note = Synthetic
PASWFDY

SEQ ID NO: 44 - protein, 5 aa, synthetic construct
  REGION 1..5: note = Synthetic
NYWMD

SEQ ID NO: 45 - protein, 17 aa, synthetic construct
  REGION 1..17: note = Synthetic
EINTDGSKTNYAPSIKD

SEQ ID NO: 46 - protein, 4 aa, synthetic construct
  REGION 1..4: note = Synthetic
GPGA

SEQ ID NO: 47 - protein, 5 aa, synthetic construct
  REGION 1..5: note = Synthetic
DYWMG

SEQ ID NO: 48 - protein, 17 aa, synthetic construct
  REGION 1..17: note = Synthetic
EINKDSRTINYTPFLKD

SEQ ID NO: 49 - protein, 8 aa, synthetic construct
  REGION 1..8: note = Synthetic
EGRVPFDY

SEQ ID NO: 50 - protein, 5 aa, synthetic construct
  REGION 1..5: note = Synthetic
DYYMT

SEQ ID NO: 51 - protein, 17 aa, synthetic construct
  REGION 1..17: note = Synthetic
SINHDGGGTYYRDSVKG

SEQ ID NO: 52 - protein, 5 aa, synthetic construct
  REGION 1..5: note = Synthetic
RAGWH

SEQ ID NO: 53 - protein, 6 aa, synthetic construct
  REGION 1..6: note = Synthetic
SKSGMA

SEQ ID NO: 54 - protein, 17 aa, synthetic construct
  REGION 1..17: note = Synthetic
SISNGGGDTYYRDSVKG

SEQ ID NO: 55 - protein, 12 aa, synthetic construct
  REGION 1..12: note = Synthetic
HRDYGYDAYFDY

SEQ ID NO: 56 - protein, 5 aa, synthetic construct
  REGION 1..5: note = Synthetic
SYWIH

SEQ ID NO: 57 - protein, 17 aa, synthetic construct
  REGION 1..17: note = Synthetic
VIDPSDSYTNYNQQFNV

SEQ ID NO: 58 - protein, 12 aa, synthetic construct
  REGION 1..12: note = Synthetic
GLITAVNWYFDV

SEQ ID NO: 59 - protein, 5 aa, synthetic construct
  REGION 1..5: note = Synthetic
SYWMN

SEQ ID NO: 60 - protein, 17 aa, synthetic construct
  REGION 1..17: note = Synthetic
QIYPGDGDTNDNGKFEG

SEQ ID NO: 61 - protein, 11 aa, synthetic construct
  REGION 1..11: note = Synthetic
SPLHLRGSMDY

SEQ ID NO: 62 - protein, 5 aa, synthetic construct
  REGION 1..5: note = Synthetic
SYAMS

SEQ ID NO: 63 - protein, 17 aa, synthetic construct
  REGION 1..17: note = Synthetic
YISSGGDYIYYADTVKG

SEQ ID NO: 64 - protein, 16 aa, synthetic construct
  REGION 1..16: note = Synthetic
DRYYDYDVRDYYTMDY

SEQ ID NO: 65 - protein, 5 aa, synthetic construct
  REGION 1..5: note = Synthetic
DYYMN

SEQ ID NO: 66 - protein, 17 aa, synthetic construct
  REGION 1..17: note = Synthetic
DINPYHGGSTYNQKIKV

SEQ ID NO: 67 - protein, 7 aa, synthetic construct
  REGION 1..7: note = Synthetic
NWPYFDY

SEQ ID NO: 68 - protein, 5 aa, synthetic construct
  REGION 1..5: note = Synthetic
SFWMN

SEQ ID NO: 69 - protein, 17 aa, synthetic construct
  REGION 1..17: note = Synthetic
RIYPGKGDTLYNGKFKD

SEQ ID NO: 70 - protein, 12 aa, synthetic construct
  REGION 1..12: note = Synthetic
SIYYYDYWYFDV

SEQ ID NO: 71 - protein, 7 aa, synthetic construct
  REGION 1..7: note = Synthetic
TGNYRWS

SEQ ID NO: 72 - protein, 16 aa, synthetic construct
  REGION 1..16: note = Synthetic
YIYYSGTITYNPSLTS

SEQ ID NO: 73 - protein, 9 aa, synthetic construct
  REGION 1..9: note = Synthetic
DLTHWYFDV

SEQ ID NO: 74 - protein, 7 aa, synthetic construct
  REGION 1..7: note = Synthetic
TGNYRWS

SEQ ID NO: 75 - protein, 16 aa, synthetic construct
  REGION 1..16: note = Synthetic
YIYYSGTITYNPSLTS

SEQ ID NO: 76 - protein, 9 aa, synthetic construct
  REGION 1..9: note = Synthetic
DLRHWYFDV

SEQ ID NO: 77 - protein, 5 aa, synthetic construct
  REGION 1..5: note = Synthetic
SYWMN

SEQ ID NO: 78 - protein, 17 aa, synthetic construct
  REGION 1..17: note = Synthetic
QIYPGDGDTNDNGKFEG

SEQ ID NO: 79 - protein, 11 aa, synthetic construct
  REGION 1..11: note = Synthetic
SPLHLRGSMDY

SEQ ID NO: 80-82 - skipped (3 entries)

SEQ ID NO: 83 - protein, 15 aa, synthetic construct
  REGION 1..15: note = Synthetic
RASKSVSTSTYSYMH

SEQ ID NO: 84 - protein, 7 aa, synthetic construct
  REGION 1..7: note = Synthetic
LASNLES

SEQ ID NO: 85 - protein, 9 aa, synthetic construct
  REGION 1..9: note = Synthetic
QHSRELPPT

SEQ ID NO: 86 - protein, 16 aa, synthetic construct
  REGION 1..16: note = Synthetic
RSSQSLVHSDGNTYLH

SEQ ID NO: 87 - protein, 7 aa, synthetic construct
  REGION 1..7: note = Synthetic
KVSTRFS

SEQ ID NO: 88 - protein, 9 aa, synthetic construct
  REGION 1..9: note = Synthetic
SQSTHVPWT

SEQ ID NO: 89 - protein, 11 aa, synthetic construct
  REGION 1..11: note = Synthetic
RASENIYSYLV

SEQ ID NO: 90 - protein, 7 aa, synthetic construct
  REGION 1..7: note = Synthetic
NAKTLAE

SEQ ID NO: 91 - protein, 9 aa, synthetic construct
  REGION 1..9: note = Synthetic
QHHYGTPFT

SEQ ID NO: 92 - protein, 11 aa, synthetic construct
  REGION 1..11: note = Synthetic
RASQEMSGHLS

SEQ ID NO: 93 - protein, 7 aa, synthetic construct
  REGION 1..7: note = Synthetic
AASTLDS

SEQ ID NO: 94 - protein, 9 aa, synthetic construct
  REGION 1..9: note = Synthetic
LQYASYPYT

SEQ ID NO: 95 - protein, 11 aa, synthetic construct
  REGION 1..11: note = Synthetic
KASQDVSPSVA

SEQ ID NO: 96 - protein, 7 aa, synthetic construct
  REGION 1..7: note = Synthetic
SASYRYT

SEQ ID NO: 97 - protein, 9 aa, synthetic construct
  REGION 1..9: note = Synthetic
QQHYSIPLT

SEQ ID NO: 98 - protein, 11 aa, synthetic construct
  REGION 1..11: note = Synthetic
RASQEISGHLG

SEQ ID NO: 99 - protein, 7 aa, synthetic construct
  REGION 1..7: note = Synthetic
AALTLDS

SEQ ID NO: 100 - protein, 9 aa, synthetic construct
  REGION 1..9: note = Synthetic
LQYASYPYT

SEQ ID NO: 101 - protein, 11 aa, synthetic construct
  REGION 1..11: note = Synthetic
RASENIYSNLA

SEQ ID NO: 102 - protein, 7 aa, synthetic construct
  REGION 1..7: note = Synthetic
AATNLAD

SEQ ID NO: 103 - protein, 9 aa, synthetic construct
  REGION 1..9: note = Synthetic
QHFWNTPPT

SEQ ID NO: 104 - protein, 11 aa, synthetic construct
  REGION 1..11: note = Synthetic
RTSQEISGYLS

SEQ ID NO: 105 - protein, 7 aa, synthetic construct
  REGION 1..7: note = Synthetic
STSTLDS

SEQ ID NO: 106 - protein, 9 aa, synthetic construct
  REGION 1..9: note = Synthetic
LQYASSPPT

SEQ ID NO: 107 - protein, 11 aa, synthetic construct
  REGION 1..11: note = Synthetic
RASQEISGYLS

SEQ ID NO: 108 - protein, 7 aa, synthetic construct
  REGION 1..7: note = Synthetic
STSTLDS

SEQ ID NO: 109 - protein, 9 aa, synthetic construct
  REGION 1..9: note = Synthetic
LQYTSSPPT

SEQ ID NO: 110 - protein, 11 aa, synthetic construct
  REGION 1..11: note = Synthetic
RASQEISGHLS

SEQ ID NO: 111 - protein, 7 aa, synthetic construct
  REGION 1..7: note = Synthetic
AASTLDS

SEQ ID NO: 112 - protein, 9 aa, synthetic construct
  REGION 1..9: note = Synthetic
LQYASYPYT

SEQ ID NO: 113 - protein, 11 aa, synthetic construct
  REGION 1..11: note = Synthetic
RASQEISGHLS

SEQ ID NO: 114 - protein, 7 aa, synthetic construct
  REGION 1..7: note = Synthetic
AASTLDS

SEQ ID NO: 115 - protein, 9 aa, synthetic construct
  REGION 1..9: note = Synthetic
LQYASYPYT

SEQ ID NO: 116 - protein, 11 aa, synthetic construct
  REGION 1..11: note = Synthetic
RASQEISGHLS

SEQ ID NO: 117 - protein, 7 aa, synthetic construct
  REGION 1..7: note = Synthetic
AASTLDS

SEQ ID NO: 118 - protein, 9 aa, synthetic construct
  REGION 1..9: note = Synthetic
LQYASYPYT

SEQ ID NO: 119 - protein, 11 aa, synthetic construct
  REGION 1..11: note = Synthetic
RASQEISGHLS

SEQ ID NO: 120 - protein, 7 aa, synthetic construct
  REGION 1..7: note = Synthetic
AASTLDS

SEQ ID NO: 121 - protein, 9 aa, synthetic construct
  REGION 1..9: note = Synthetic
LQYASYPYT

SEQ ID NO: 122 - protein, 16 aa, synthetic construct
  REGION 1..16: note = Synthetic
KSSQSLKFSDGKTYLN

SEQ ID NO: 123 - protein, 7 aa, synthetic construct
  REGION 1..7: note = Synthetic
QVSKLDS

SEQ ID NO: 124 - protein, 9 aa, synthetic construct
  REGION 1..9: note = Synthetic
CQGSYSPWT

SEQ ID NO: 125 - protein, 11 aa, synthetic construct
  REGION 1..11: note = Synthetic
RASQDVGSYVN

SEQ ID NO: 126 - protein, 7 aa, synthetic construct
  REGION 1..7: note = Synthetic
RATNLAD

SEQ ID NO: 127 - protein, 9 aa, synthetic construct
  REGION 1..9: note = Synthetic
QQYAEYPLT

SEQ ID NO: 128 - protein, 11 aa, synthetic construct
  REGION 1..11: note = Synthetic
LASEDIYNYLT

SEQ ID NO: 129 - protein, 7 aa, synthetic construct
  REGION 1..7: note = Synthetic
SASGLQD

SEQ ID NO: 130 - protein, 9 aa, synthetic construct
  REGION 1..9: note = Synthetic
LQDSEYPLT

SEQ ID NO: 131 - protein, 11 aa, synthetic construct
  REGION 1..11: note = Synthetic
LASEDIYSSLA

SEQ ID NO: 132 - protein, 7 aa, synthetic construct
  REGION 1..7: note = Synthetic
DASSLQD

SEQ ID NO: 133 - protein, 9 aa, synthetic construct
  REGION 1..9: note = Synthetic
QQHHDYPYT

SEQ ID NO: 134 - protein, 11 aa, synthetic construct
  REGION 1..11: note = Synthetic
RASENIYSYLT

SEQ ID NO: 135 - protein, 7 aa, synthetic construct
  REGION 1..7: note = Synthetic
NAKTLAE

SEQ ID NO: 136 - protein, 9 aa, synthetic construct
  REGION 1..9: note = Synthetic
QHHYGTPLT

SEQ ID NO: 137 - protein, 11 aa, synthetic construct
  REGION 1..11: note = Synthetic
KASQSVSNDVA

SEQ ID NO: 138 - protein, 7 aa, synthetic construct
  REGION 1..7: note = Synthetic
SASNRYT

SEQ ID NO: 139 - protein, 9 aa, synthetic construct
  REGION 1..9: note = Synthetic
QQNYYSPFT

SEQ ID NO: 140 - protein, 16 aa, synthetic construct
  REGION 1..16: note = Synthetic
RSSQSLVHSNGNTYLH

SEQ ID NO: 141 - protein, 7 aa, synthetic construct
  REGION 1..7: note = Synthetic
RVSNRFS

SEQ ID NO: 142 - protein, 9 aa, synthetic construct
  REGION 1..9: note = Synthetic
SQTTHVPRT

SEQ ID NO: 143 - protein, 11 aa, synthetic construct
  REGION 1..11: note = Synthetic
RASENIYSFLV

SEQ ID NO: 144 - protein, 7 aa, synthetic construct
  REGION 1..7: note = Synthetic
NAKTLAE

SEQ ID NO: 145 - protein, 10 aa, synthetic construct
  REGION 1..10: note = Synthetic
QHHFGTPPYT

SEQ ID NO: 146 - protein, 11 aa, synthetic construct
  REGION 1..11: note = Synthetic
KASQDVNSAIA

SEQ ID NO: 147 - protein, 7 aa, synthetic construct
  REGION 1..7: note = Synthetic
SASYRFI

SEQ ID NO: 148 - protein, 9 aa, synthetic construct
  REGION 1..9: note = Synthetic
QQHYTTPLT

SEQ ID NO: 149 - protein, 15 aa, synthetic construct
  REGION 1..15: note = Synthetic
RASESVDYYGIGFMN

SEQ ID NO: 150 - protein, 7 aa, synthetic construct
  REGION 1..7: note = Synthetic
AASNQGS

SEQ ID NO: 151 - protein, 9 aa, synthetic construct
  REGION 1..9: note = Synthetic
QQSKEVPPT

SEQ ID NO: 152 - protein, 15 aa, synthetic construct
  REGION 1..15: note = Synthetic
RASENIDYYGVSFMN

SEQ ID NO: 153 - protein, 7 aa, synthetic construct
  REGION 1..7: note = Synthetic
AASNQGS

SEQ ID NO: 154 - protein, 9 aa, synthetic construct
  REGION 1..9: note = Synthetic
QQSQEVPPT

SEQ ID NO: 155 - protein, 11 aa, synthetic construct
  REGION 1..11: note = Synthetic
KASQSVSNDVA

SEQ ID NO: 156 - protein, 7 aa, synthetic construct
  REGION 1..7: note = Synthetic
SASNRYT

SEQ ID NO: 157 - protein, 9 aa, synthetic construct
  REGION 1..9: note = Synthetic
QQNYYSPFT

SEQ ID NO: 158-160 - skipped (3 entries)

SEQ ID NO: 161 - protein, 117 aa, synthetic construct
  REGION 1..117: note = Synthetic
QVQLQQSGAELAKPGASVKLSCKASGYNFTNYCIHWVKQRPGQGLEWIGYINPSTGYTEYNQKFKDKATLTADKSSTTAYMQLSSLTSEDSAVYYCARGRYDEFTYWGQGTLVTVSA

SEQ ID NO: 162 - protein, 111 aa, synthetic construct
  REGION 1..111: note = Synthetic
DIVLTQSPASLAVSLGQRATISCRASKSVSTSTYSYMHWYQQKPGQPPKLLIYLASNLESGVPARFSGSGSGTDFTLNIHPVEEEDAATYYCQHSRELPPTFGGGTKLEIK

SEQ ID NO: 163 - protein, 113 aa, synthetic construct
  REGION 1..113: note = Synthetic
QVQLQQSGPELVKPGASVKLSCKASGYTFTNYYMYWVKQRPGQGLEWIGEINPRNGGTNFNEKFKSKATLTVDKSSSTAYMQLSSLTFEDSAVYYCTTGLAYWGQGTLVTVSA

SEQ ID NO: 164 - protein, 112 aa, synthetic construct
  REGION 1..112: note = Synthetic
DVVMTQTPLSLPVSLGDQASISCRSSQSLVHSDGNTYLHWYLQKPGQSPKLLIYKVSTRFSGVPDRFSGSGSGTDFTLKISRVEAEDLGVYFCSQSTHVPWTFGGGTKLEIK

SEQ ID NO: 165 - protein, 113 aa, synthetic construct
  REGION 1..113: note = Synthetic
QVQLQQSGAELVKPGASVKLSCKASGYTFTNYYMYWVKQRPGQGLEWIGEINTRNGGTNFNEKFKSKATLTVDKSSSTAYMQLSSLTSEDSAVYYCTTGFAYWGQGTLVTVSA

SEQ ID NO: 166 - protein, 107 aa, synthetic construct
  REGION 1..107: note = Synthetic
DIQMTQSPASLSASVGETVTITCRASENIYSYLVWYQQNQGKSPQLLVYNAKTLAEGVPSRFSGSGSGTQFSLKINSLQPEDFGTYYCQHHYGTPFTFGSGTKLEIK

SEQ ID NO: 167 - protein, 116 aa, synthetic construct
  REGION 1..116: note = Synthetic
QVQLQQSGAEQVKPGASVKLSCKASGYTFTSYYMYWVKQRPGQGLEWIGEINPSNGGTYFNEKFKSKATLTVDKSSSTAYMHLSSLTSEDSAVYYCTRGGTYYAYWGQGTLVTVSA

SEQ ID NO: 168 - protein, 107 aa, synthetic construct
  REGION 1..107: note = Synthetic
DIQMTQSPSSLSASLGERVSLTCRASQEMSGHLSWLQQKPDGTIKRLIYAASTLDSGVPKRFSGSRSGSDYSLTISSLESEDFADYYCLQYASYPYTFGGGTKLEIK

SEQ ID NO: 169 - protein, 119 aa, synthetic construct
  REGION 1..119: note = Synthetic
QVQLQQPGAELVKPGASVKLSCRASGYTFTSYWLHWVKQRPGQGLEWIGEINPSDGRTNYIEKVKTKATLTVDKSSSTAYMQLSSLTSEDSAVYYCARSGGNYAWFAYWGQGTLVTVSA

SEQ ID NO: 170 - protein, 107 aa, synthetic construct
  REGION 1..107: note = Synthetic
DIVMTQSHKFMSTSVGDRVSITCKASQDVSPSVAWYQQRPGQSPKLLIYSASYRYTGVPDRFTGSGSGTDFTFTISSVQAEDLAVYYCQQHYSIPLTFGAGTKLELK

SEQ ID NO: 171 - protein, 116 aa, synthetic construct
  REGION 1..116: note = Synthetic
QVQLQQSGAELVKPGASVKLSCKASGYTFTSYYIYWVKQRPGQGLEWIGEINPSNGGTYFNEKFKNKATLTVDKSSSTAYMQLSSLTSEDSAVYYCTRGGNYYAYWGQGTLVTVSA

SEQ ID NO: 172 - protein, 107 aa, synthetic construct
  REGION 1..107: note = Synthetic
DIQMTQSPSSLSASLGERVSLTCRASQEISGHLGWLQQKPDGTIKRLIYAALTLDSGVPKRFSGSRSGSDYSLTISSLESEDFADYYCLQYASYPYTFGGGTKLEIK

SEQ ID NO: 173 - protein, 125 aa, synthetic construct
  REGION 1..125: note = Synthetic
QVQLQQPGAELVKPGASVKLSCKASGYTFTSYWMHWVKQRPGQGLEWIGEINSSNGRTNYNEKFKTKATLTVDRSSSTAYMQLSSLTSEDSAVYYCARLGYYSGSPYPYYAMDCWGQGTSVTVSS

SEQ ID NO: 174 - protein, 107 aa, synthetic construct
  REGION 1..107: note = Synthetic
DIQMTQSPASLSVSVGETVTITCRASENIYSNLAWYQQKQGKSPQLLVYAATNLADGVPSRFSGSGSGTQYSLKINSLQSEDFGSYYCQHFWNTPPTFGGGTKLEIK

SEQ ID NO: 175 - protein, 119 aa, synthetic construct
  REGION 1..119: note = Synthetic
QVQLQQPGAERVKPGASVKMSCKASGYTFTSYWITWVKQRPGQGLEWIGDIFPGSGSTNYHEKFKSKATLTVDTSSSTAYMQLSSLTSEDSAVYYCSRGYFGSSFSDYWGQGTSVTVSS

SEQ ID NO: 176 - protein, 107 aa, synthetic construct
  REGION 1..107: note = Synthetic
DIQMTQSPSSLSASLGERVSLTCRTSQEISGYLSWLQQKPDGTIKRLIYSTSTLDSGVPKRFSGSRSGSDYSLTISSLESEDFADYYCLQYASSPPTFGSGTKLEIK

SEQ ID NO: 177 - protein, 115 aa, synthetic construct
  REGION 1..115: note = Synthetic
QVQLQQPGAELVKPGASVKMSCKASGYTFTSYWITWVKQRPGQGLEWIGDIHPGSGRSNYNEKFKNKATLNVDTSSSTVYMQLSSLTSEDSAVYYCARDYLFAYWGQGTLVTVSA

SEQ ID NO: 178 - protein, 107 aa, synthetic construct
  REGION 1..107: note = Synthetic
DIQMTQSPSSLSASLGERVSLTCRASQEISGYLSWLQQKPDGTIKRLIYSTSTLDSGVPKRFSGSRSGSDYSLTISSLESEDFADYYCLQYTSSPPTFGGGTKLEIK

SEQ ID NO: 179 - protein, 116 aa, synthetic construct
  REGION 1..116: note = Synthetic
EVQLQQSGPELVKPGASVKISCKASGYSFTGYYMNWVKQSPEKSLEWIGEINPGSGITTYNQKFKAKATLTVDKSSSTAYLQLKSLTSEDSAVYYCARPASWFAYWGQGTLVTVSA

SEQ ID NO: 180 - protein, 107 aa, synthetic construct
  REGION 1..107: note = Synthetic
DIQMTQSPSSLSASLGERVSFICRASQEISGHLSWLQQKPDGTIKRLIYAASTLDSGVPKRFSGSRSGSDYSLTISSLESEDFADYYCLQYASYPYTFGGGTKLEIK

SEQ ID NO: 181 - protein, 116 aa, synthetic construct
  REGION 1..116: note = Synthetic
EVQLQQSGPELVKPGASVKISCKASGYSFTGYYMNWVRQSPEKSLEWIGEINPSTGITTYNQKFKAKATLTVDKSSNTAFMQLKSLTSEDSAVYYCARPASWFDYWGQGTLVTVSA

SEQ ID NO: 182 - protein, 107 aa, synthetic construct
  REGION 1..107: note = Synthetic
DIQMTQSPSSLSASLGERVSLTCRASQEISGHLSWLQQKPDGTIKRLIYAASTLDSGVPKRFSGSRSGSDYSLTISSLESEDFADYYCLQYASYPYTFGGGTKLEIK

SEQ ID NO: 183 - protein, 116 aa, synthetic construct
  REGION 1..116: note = Synthetic
EVQLQQSGPELVNPGASVRISCKASGYSFTGYYMNWVKQSPEKSLEWIGEINPNTGFTTYNQNFKAKATLTVDKSSSTAYIQLKSLTSEDSAVYYCTRPSPSFDYWGQGTTLTVSS

SEQ ID NO: 184 - protein, 107 aa, synthetic construct
  REGION 1..107: note = Synthetic
DIQMTQSPSSLSASLGERGSLTCRASQEISGHLSWLQQKPDGTIKRLIYAASTLDSGVPKRFSGSRSGSDYSLTISSLESEDFADYYCLQYASYPYTFGGGTKLEIK

SEQ ID NO: 185 - protein, 116 aa, synthetic construct
  REGION 1..116: note = Synthetic
EVQLQQSGPELVKPGASVKISCKASGYSFTGYYMNWVKQSPEKNLEWIGEINPNTGGTTYNQKFKAKATLTVDKSSTTAYMQLKSLTSEDSAVYYCTRPASWFDYWGQGTLVTVSA

SEQ ID NO: 186 - protein, 107 aa, synthetic construct
  REGION 1..107: note = Synthetic
DIQMTQSPSSLSASLGERVSLTCRASQEISGHLSWLQQKPDGTIKRLIYAASTLDSGVPKRFSGRRSGSDYSLTISSLESEDFADYYCLQYASYPYTFGGGTKLAIK

SEQ ID NO: 187 - protein, 113 aa, synthetic construct
  REGION 1..113: note = Synthetic
EVQLVESGGSLVQPGGSLKLSCVASGYTFSNYWMDWVRQTPGKSLEWIGEINTDGSKTNYAPSIKDRFTISRDNAKSTLYLQMSNVKADDTAIYYCTRGPGAWGQGTSVTVSS

SEQ ID NO: 188 - protein, 112 aa, synthetic construct
  REGION 1..112: note = Synthetic
DIVMTQTPLSLSVAIGQSASISCKSSQSLKFSDGKTYLNWVFQSPGQSPKRLIYQVSKLDSGVPDRFSGTGSETDFILKISRVEAEDLGVYYCCQGSYSPWTIGGGTKLELK

SEQ ID NO: 189 - protein, 117 aa, synthetic construct
  REGION 1..117: note = Synthetic
EVKLVESGGGLVQPGRSLKLSCAASGFNFYDYWMGWVRQAPGKGLEWIGEINKDSRTINYTPFLKDRFTISRDNAQNTLYLQMSKLGSEDTAIYYCAREGRVPFDYWGQGVMVTVSS

SEQ ID NO: 190 - protein, 107 aa, synthetic construct
  REGION 1..107: note = Synthetic
DIQMTQSPSSMSVSLGDTVTIPCRASQDVGSYVNWFQQKPGKSPRHMIYRATNLADGVPSRFSGSRSGADYSLTISSLESEDVADYHCQQYAEYPLTFGSGTKLEIK

SEQ ID NO: 191 - protein, 114 aa, synthetic construct
  REGION 1..114: note = Synthetic
EVQLVESGGGSVRPGRSLKLSCAAAGFTFSDYYMTWVRQAPTKGLEWVASINHDGGGTYYRDSVKGRFTISRDNARSSLYLQMDSLRSEDTGTYYCTTRAGWHWGQGVMVTVSS

SEQ ID NO: 192 - protein, 107 aa, synthetic construct
  REGION 1..107: note = Synthetic
DIQMTQSPASLSASLGETVSIECLASEDIYNYLTWYQQKPGKSPQVLIYSASGLQDGVPSRFSGSGSGRQYSLKINGLESEDAATYFCLQDSEYPLTFGSGTKLEIK

SEQ ID NO: 193 - protein, 122 aa, synthetic construct
  REGION 1..122: note = Synthetic
EVQLVESGGGLVQPGRSLKLSCAASGFTFSSKSGMAWVRQAPTKGLEWVASISNGGGDTYYRDSVKGRFTISRDNAKNTQYLQMDSLRPEDTATYYCARHRDYGYDAYFDYWGQGVMVTVSS

SEQ ID NO: 194 - protein, 107 aa, synthetic construct
  REGION 1..107: note = Synthetic
DIQMTQSPASLSASLGETVTIECLASEDIYSSLAWYQQKPGKSPQLLIYDASSLQDGVPSRFSGSESGTQYSLEINSLQSEDAATYFCQQHHDYPYTFGAGTKLELK

SEQ ID NO: 195 - protein, 121 aa, synthetic construct
  REGION 1..121: note = Synthetic
QVQLQQPGAELVRPGTSVRLSCKASGYTFTSYWIHWVKQRPGRGLEWIGVIDPSDSYTNYNQQFNVKATLTVDTSSSAAYMQLSSLTSEDSAVYYCARGLITAVNWYFDVWGTGTTVTVSS

SEQ ID NO: 196 - protein, 107 aa, synthetic construct
  REGION 1..107: note = Synthetic
DIQMTQSPASLSTSVGEIVTITCRASENIYSYLTWYQQKQGKSPQLLVYNAKTLAEGVPSRFSGSGSGTQFSLKINSLQPEDFGTYYCQHHYGTPLTFGSGTKLEIK

SEQ ID NO: 197 - protein, 120 aa, synthetic construct
  REGION 1..120: note = Synthetic
QLQLQQSGTEVVKPGASVKIPCKASGYAFSSYWMNWVKQRPGKGLEWIGQIYPGDGDTNDNGKFEGKATLTVDKSSNTAYIQLSSLTSEDSAVYFCARSPLHLRGSMDYWGQGTSVTVSS

SEQ ID NO: 198 - protein, 107 aa, synthetic construct
  REGION 1..107: note = Synthetic
SIVMTQTPKFLLVSAGDRVTITCKASQSVSNDVAWFQQKPGQSPELLIYSASNRYTGVPDRFTASGYGTDFTFTISAVQAEDLAVYFCQQNYYSPFTFGGGTRLEIK

SEQ ID NO: 199 - protein, 125 aa, synthetic construct
  REGION 1..125: note = Synthetic
DVKLVESGEDLVKPGGSLKLSCAASGFTFSSYAMSWVRQTPEKRLEWVAYISSGGDYIYYADTVKGRFTISRDNARNTLYLQMSSLKSEDTAMYYCTRDRYYDYDVRDYYTMDYWGQGTSVTVSS

SEQ ID NO: 200 - protein, 112 aa, synthetic construct
  REGION 1..112: note = Synthetic
DVMMTQTPLSLPVSLGDQASISCRSSQSLVHSNGNTYLHWFLQKPGQSPKLLIFRVSNRFSGVPDRFSGRGSGTDFTLKISRVEAEDLGVYFCSQTTHVPRTFGGGTTLEIK

SEQ ID NO: 201 - protein, 116 aa, synthetic construct
  REGION 1..116: note = Synthetic
EVQLQQSGPELVKPGASVKISCKASGYTFTDYYMNWVRQSHGKSLEWIGDINPYHGGSTYNQKIKVKATLTVDKSSSTAYMELRSLTSEDSAVYYCARNWPYFDYWGQGTTLTVSS

SEQ ID NO: 202 - protein, 108 aa, synthetic construct
  REGION 1..108: note = Synthetic
DIQMTQSPASLSASVGETVTITCRASENIYSFLVWYQQKQGKSPQLLVYNAKTLAEGVPSRFSGSGSGTQFSLKISSLQPEDFGSYYCQHHFGTPPYTFGGGTKLEIK

SEQ ID NO: 203 - protein, 121 aa, synthetic construct
  REGION 1..121: note = Synthetic
QVQLQQSGPELVKPGASVKLSCRASGYGFSSFWMNWVKQRPGKGLEWIGRIYPGKGDTLYNGKFKDKATLTADESSSTAYMQLSSLTSEDSAVYFCARSIYYYDYWYFDVWDTGTTVTVSS

SEQ ID NO: 204 - protein, 107 aa, synthetic construct
  REGION 1..107: note = Synthetic
DIVMTQSHKFMSTSVGDRVNITCKASQDVNSAIAWYQQKPGQSPKLLIYSASYRFIGVPDRFTGSGSGTGFTFTISSVQAEDLAVYYCQQHYTTPLTFGAGTKLELK

SEQ ID NO: 205 - protein, 119 aa, synthetic construct
  REGION 1..119: note = Synthetic
DVQLQESGPGLVKPSQTVFLTCTVTGISITTGNYRWSWIRQFPGNKLEWIGYIYYSGTITYNPSLTSRITITRDTPKNEFFLEMNSLTAEDTATYYCARDLTHWYFDVWGTGTTVTVSS

SEQ ID NO: 206 - protein, 111 aa, synthetic construct
  REGION 1..111: note = Synthetic
DIVLTQSPASLAVSLGQRATISCRASESVDYYGIGFMNWFQQKPGQPPKLLIYAASNQGSGVPARFSGSGSGTDFSLNIHPMEEDDTAMYFCQQSKEVPPTFGGGTKLEIK

SEQ ID NO: 207 - protein, 119 aa, synthetic construct
  REGION 1..119: note = Synthetic
DVQLQESGPGLVKPSQTVFLTCTVTGISITTGNYRWSWIRQFPGNKLEWIGYIYYSGTITYNPSLTSRTTITRDTPKNQFFLEMSSLTAEDTATYFCARDLRHWYFDVWGTGTTVTVSS

SEQ ID NO: 208 - protein, 111 aa, synthetic construct
  REGION 1..111: note = Synthetic
DIVLTQSPASLAVSLGQRATISCRASENIDYYGVSFMNWFQQKPGQPPKLLIYAASNQGSGVPARFSGSGSGTDFSLNIHPLEEDDTAMYFCQQSQEVPPTFGGGTKLEIK

SEQ ID NO: 209 - protein, 120 aa, synthetic construct; identical to SEQ ID NO: 197
  REGION 1..120: note = Synthetic

SEQ ID NO: 210 - protein, 107 aa, synthetic construct
  REGION 1..107: note = Synthetic
SIVMTQTPKFLLVSAGDRVTITCKASQSVSNDVAWFQQKPGQSPELLIYSASNRYTGVPDRFTGSGYGTDFTFTISTVQAEDLAVYFCQQNYYSPFTFGGGTRLEIK

SEQ ID NO: 211-212 - skipped (2 entries)

SEQ ID NO: 213 - protein, 330 aa, synthetic construct [sequence omitted]

SEQ ID NO: 214 - protein, 329 aa, synthetic construct
  REGION 1..329: note = Synthetic: huIgG1 - WT without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 215 - protein, 330 aa, synthetic construct
  REGION 1..330: note = Synthetic: huIgG1 - K322A with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 329 aa, synthetic construct
  REGION 1..329: note = Synthetic: huIgG1 - K322A without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 217 - protein, 330 aa, synthetic construct
  REGION 1..330: note = Synthetic: huIgG1 - LALAPS (L234A; L235A, P331S) with C-terminallysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 329 aa, synthetic construct
  REGION 1..329: note = Synthetic: huIgG1 - LALAPS (L234A; L235A, P331S) withoutC-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 330 aa, synthetic construct
  REGION 1..330: note = Synthetic: huIgG1 - PS (P331S) with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 329 aa, synthetic construct
  REGION 1..329: note = Synthetic: huIgG1 - PS (P331S) without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 221 - protein, 330 aa, synthetic construct
  REGION 1..330: note = Synthetic: huIgG1 - PSEG (P331S; E430G) with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 222 - protein, 329 aa, synthetic construct
  REGION 1..329: note = Synthetic: huIgG1 - PSEG (P331S; E430G) without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 223 - protein, 330 aa, synthetic construct
  REGION 1..330: note = Synthetic: huIgG1 - NSLF (N325S; L328F) with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 329 aa, synthetic construct
  REGION 1..329: note = Synthetic: huIgG1 - NSLF (N325S; L328F) without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 225 - protein, 330 aa, synthetic construct
  REGION 1..330: note = Synthetic: huIgG1 - SELF (S267E; L328F) with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 329 aa, synthetic construct
  REGION 1..329: note = Synthetic: huIgG1 - SELF (S267E; L328F) without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 227 - protein, 330 aa, synthetic construct
  REGION 1..330: note = Synthetic: huIgG1 - LS with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 228 - protein, 329 aa, synthetic construct
  REGION 1..329: note = Synthetic: huIgG1 - LS without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 229 - protein, 326 aa, synthetic construct
  REGION 1..326: note = Synthetic: huIgG2 - WT with C-terminal lysine
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 230 - protein, 325 aa, synthetic construct
  REGION 1..325: note = Synthetic: huIgG2 - WT without C-terminal lysine
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 231 - protein, 327 aa, synthetic construct
  REGION 1..327: note = Synthetic: huIgG4 - WT with C-terminal lysine
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 232 - protein, 326 aa, synthetic construct
  REGION 1..326: note = Synthetic: huIgG4 - WT without C-terminal lysine
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 233 - protein, 107 aa, Homo sapiens [sequence omitted]

SEQ ID NO: 234 - protein, 119 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSS

SEQ ID NO: 235 - protein, 119 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSS

SEQ ID NO: 236 - protein, 119 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSS

SEQ ID NO: 237 - protein, 119 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSS

SEQ ID NO: 238 - protein, 119 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSS

SEQ ID NO: 239 - protein, 119 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSS

SEQ ID NO: 240 - protein, 119 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSS

SEQ ID NO: 241 - protein, 111 aa, synthetic construct
DIVMTQSPDSLAVSLGERATINCKSSESVDYYGIGFLNWYQQKPGQPPKLLIYAASTQGSGVPDRFSGSGSGTDFTLTISSLQAEDVAVYYCQQSKEVPPTFGGGTKVEIK

SEQ ID NO: 242 - protein, 111 aa, synthetic construct
DIVMTQSPDSLAVSLGERATINCKSSRSVDYYGIGFLNWYQQKPGQPPKLLIYATSTQGSGVPDRFSGSGSGTDFTLTISSLQAEDVAVYYCQQQKEVPPTFGGGTKVEIK

SEQ ID NO: 243 - protein, 111 aa, synthetic construct
DIVMTQSPDSLAVSLGERATINCKSSRSVDYYGIGFVNWYQQKPGQPPKLLIYATSIQGSGVPDRFSGSGSGTDFTLTISSLQAEDVAVYYCQQQKEVPPTFGGGTKVEIK

SEQ ID NO: 244 - protein, 111 aa, synthetic construct
DIVMTQSPDSLAVSLGERATINCKSSRSVDYFGIGFLNWYQQKPGQPPKLLIYATSHQGSGVPDRFSGSGSGTDFTLTISSLQAEDVAVYYCQQQKEVPPTFGGGTKVEIK

SEQ ID NO: 245 - protein, 111 aa, synthetic construct
DIVMTQSPDSLAVSLGERATINCKSSRSVDYYGIGFLNWYQQKPGQPPKLLIYATSKQGSGVPDRFSGSGSGTDFTLTISSLQAEDVAVYYCQQQKEVPPTFGGGTKVEIK

SEQ ID NO: 246 - protein, 111 aa, synthetic construct
DIVMTQSPDSLAVSLGERATINCKSSRSVDYYGIGFLNWYQQKPGQPPKLLIYATSHQGSGVPDRFSGSGSGTDFTLTISSLQAEDVAVYYCQQQKHVPPTFGGGTKVEIK

SEQ ID NO: 247 - protein, 111 aa, synthetic construct
DIVMTQSPDSLAVSLGERATINCKSSRSVDYYGIGFLNWYQQKPGQPPKLLIYATSHQGSGVPDRFSGSGSGTDFTLTISSLQAEDVAVYYCQQQKEVPPTFGGGTKVEIK

SEQ ID NO: 248 - protein, 111 aa, synthetic construct
DIVMTQSPDSLAVSLGERATINCKSSRSVDYYGIGFLNWYQQKPGQPPKLLIYATSHQGSGVPDRFSGSGSGTDFTLTISSLQAEDVAVYYCQQQKERPPTFGGGTKVEIK

SEQ ID NO: 249 - protein, 107 aa, synthetic construct
DIVMTQSPDSLAVSLGERATINCKASQDVSPSLAWYQQKPGQPPKLLIYSASYRYTGVPDRFSGSGSGTDFTLTISSLQAEDVAVYYCQQHYSIPLTFGGGTKVEIK

SEQ ID NO: 250 - protein, 107 aa, synthetic construct
DIVMTQSPDSLAVSLGERATINCKTSQDVSPSLAWYQQKPGQPPKLLIYSASKRYTGVPDRFSGSGSGTDFTLTISSLQAEDVAVYYCQQHYSIPLQFGGGTKVEIK

SEQ ID NO: 251 - protein, 107 aa, synthetic construct
DIVMTQSPDSLAVSLGERATINCKTSQDVSPSLAWYQQKPGQPPKLLIYSASKRYTGVPDRFSGSGSGTDFTLTISSLQAEDVAVYYCQQHYSIPLTFGGGTKVEIK

SEQ ID NO: 252 - protein, 107 aa, synthetic construct
DIVMTQSPDSLAVSLGERATINCKTSQDVSPSLAWYQQKPGQPPKLLIYSARYRYTGVPDRFSGSGSGTDFTLTISSLQAEDVAVYYCQQHYSIPLQFGGGTKVEIK

SEQ ID NO: 253 - protein, 107 aa, synthetic construct
DIVMTQSPDSLAVSLGERATINCKASQDVSPSLAWYQQKPGQPPKLLIYRASYRYTGVPDRFSGSGSGTDFTLTISSLQAEDVAVYYCQQHYSIPLTFGGGTKVEIK

SEQ ID NO: 254 - protein, 107 aa, synthetic construct
DIVMTQSPDSLAVSLGERATINCKTSQDVSPSLAWYQQKPGQPPKLLIYSGSYRYTGVPDRFSGSGSGTDFTLTISSLQAEDVAVYYCQQHYSIPLTFGGGTKVEIK

SEQ ID NO: 255 - protein, 16 aa, synthetic construct
YIYYSGTITYNPSLKS

SEQ ID NO: 256 - protein, 16 aa, synthetic construct
YIYYSGTIIYNPSLKS

SEQ ID NO: 257 - protein, 16 aa, synthetic construct
YIDYSGTITYNPSLKS

SEQ ID NO: 258 - protein, 16 aa, synthetic construct
YIDYSGTITYNPSLKQ

SEQ ID NO: 259 - protein, 17 aa, synthetic construct
EINPSDGRTNYAQKFQG

SEQ ID NO: 260 - protein, 17 aa, synthetic construct
EINPYDQRTNYAQKFQG

SEQ ID NO: 261 - protein, 9 aa, synthetic construct
DLTHWYYDV

SEQ ID NO: 262 - protein, 10 aa, synthetic construct
STGNYAWFAY

SEQ ID NO: 263 - protein, 15 aa, synthetic construct
KSSESVDYYGIGFLN

SEQ ID NO: 264 - protein, 15 aa, synthetic construct
KSSRSVDYYGIGFLN

SEQ ID NO: 265 - protein, 15 aa, synthetic construct
KSSRSVDYYGIGFVN

SEQ ID NO: 266 - protein, 15 aa, synthetic construct
KSSRSVDYFGIGFLN

SEQ ID NO: 267 - protein, 11 aa, synthetic construct
KASQDVSPSLA

SEQ ID NO: 268 - protein, 11 aa, synthetic construct
KTSQDVSPSLA

SEQ ID NO: 269 - protein, 7 aa, synthetic construct
AASTQGS

SEQ ID NO: 270 - protein, 7 aa, synthetic construct
ATSTQGS

SEQ ID NO: 271 - protein, 7 aa, synthetic construct
ATSIQGS

SEQ ID NO: 272 - protein, 7 aa, synthetic construct
ATSHQGS

SEQ ID NO: 273 - protein, 7 aa, synthetic construct
ATSKQGS

SEQ ID NO: 274 - protein, 7 aa, synthetic construct
SASKRYT

SEQ ID NO: 275 - protein, 7 aa, synthetic construct
SARYRYT

SEQ ID NO: 276 - protein, 7 aa, synthetic construct
RASYRYT

SEQ ID NO: 277 - protein, 7 aa, synthetic construct
SGSYRYT

SEQ ID NO: 278 - protein, 9 aa, synthetic construct
QQQKEVPPT

SEQ ID NO: 279 - protein, 9 aa, synthetic construct
QQQKHVPPT

SEQ ID NO: 280 - protein, 9 aa, synthetic construct
QQQKERPPT

SEQ ID NO: 281 - protein, 9 aa, synthetic construct
QQHYSIPLQ

SEQ ID NO: 282 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 283 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 284 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 285 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 286 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 288 - protein, 328 aa, synthetic construct
  VARIANT 229: note = Any amino acid
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVXYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 289 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 290 - protein, 449 aa, Homo sapiens
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 291 - protein, 448 aa, Homo sapiens
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 292 - protein, 449 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 448 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 294 - protein, 449 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 295 - protein, 448 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 296 - protein, 449 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 297 - protein, 448 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 298 - protein, 449 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 448 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 300 - protein, 449 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 301 - protein, 448 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 302 - protein, 449 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - protein, 448 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 304 - protein, 446 aa, Homo sapiens
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 305 - protein, 445 aa, Homo sapiens
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 306 - protein, 446 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 307 - protein, 445 aa, synthetic construct
QVQLQESGPGLVKPSETLSLTCTVSGISITTGNYRWSWIRQPPGKGLEWIGYIYYSGTITYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 308 - protein, 449 aa, Homo sapiens
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 448 aa, Homo sapiens
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 310 - protein, 449 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 448 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 312 - protein, 449 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 448 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 314 - protein, 449 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 448 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 316 - protein, 449 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 448 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 318 - protein, 449 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 319 - protein, 448 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 320 - protein, 449 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 448 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 322 - protein, 446 aa, Homo sapiens
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 323 - protein, 445 aa, Homo sapiens
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 324 - protein, 446 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 325 - protein, 445 aa, synthetic construct
QVQLQESGPGLVKPSETLSLTCTVSGISITTGNYRWSWIRQPPGKGLEWIGYIYYSGTIIYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 326 - protein, 449 aa, Homo sapiens
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 448 aa, Homo sapiens
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 328 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 330 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 332 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 334 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 336 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 337 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 338 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 340 - protein, 446 aa, Homo sapiens
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 341 - protein, 445 aa, Homo sapiens
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 342 - protein, 446 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 343 - protein, 445 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 344 - protein, 449 aa, Homo sapiens
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 448 aa, Homo sapiens
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 346 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 348 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 350 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 352 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 354 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 355 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 356 - protein, 449 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 448 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 358 - protein, 446 aa, Homo sapiens
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 445 aa, Homo sapiens
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 360 - protein, 446 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 361 - protein, 445 aa, synthetic construct
QVQLQESGPGLVKPSETLSLTCTVSGISITTGNYRWSWIRQPPGKGLEWIGYIDYSGTITYNPSLKSRVTITRDTSKNQFSLELSSVTAADTAVYYCARDLTHWYYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 362 - protein, 449 aa, Homo sapiens
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 363 - protein, 448 aa, Homo sapiens
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 364 - protein, 449 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 448 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 366 - protein, 449 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 448 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 368 - protein, 449 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - protein, 448 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 370 - protein, 449 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 448 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 372 - protein, 449 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 373 - protein, 448 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 374 - protein, 449 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 448 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 376 - protein, 446 aa, Homo sapiens
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 377 - protein, 445 aa, Homo sapiens
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 378 - protein, 446 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 379 - protein, 445 aa, synthetic construct
QVQLQESGPGLVKPSETLSLTCTVSGISITTGNYRWSWIRQPPGKGLEWIGYIDYSGTITYNPSLKQRVTITRDTSKNQFSLELSSVTAADTAVYYCARDLTHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 380 - protein, 449 aa, Homo sapiens
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 381 - protein, 448 aa, Homo sapiens
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 382 - protein, 449 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - protein, 448 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 384 - protein, 449 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - protein, 448 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 386 - protein, 449 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 387 - protein, 448 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 388 - protein, 449 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 389 - protein, 448 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 390 - protein, 449 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 391 - protein, 448 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 392 - protein, 449 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 393 - protein, 448 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 394 - protein, 446 aa, Homo sapiens
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 395 - protein, 445 aa, Homo sapiens
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 396 - protein, 446 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 397 - protein, 445 aa, synthetic construct
QVQLVQSGAEVKKPGASVKVSCKASGYTFTSYWMHWVRQAPGQGLEWMGEINPSDGRTNYAQKFQGRVTMTVDKSTSTVYMELSSLRSEDTAVYYCARSGGNYAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 398 - protein, 449 aa, Homo sapiens
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 399 - protein, 448 aa, Homo sapiens
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 400 - protein, 449 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 401 - protein, 448 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 402 - protein, 449 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 403 - protein, 448 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 404 - protein, 449 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 405 - protein, 448 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 406 - protein, 449 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 407 - protein, 448 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 408 - protein, 449 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 409 - protein, 448 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 410 - protein, 449 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 411 - protein, 448 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 412 - protein, 446 aa, Homo sapiens
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 413 - protein, 445 aa, Homo sapiens
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 414 - protein, 446 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 415 - protein, 445 aa, synthetic construct
QVQLVQSGAEVKKPGASVKVSCKASGYTFTSYWMHWVRQAPGQGLEWMGEINPYDQRTNYAQKFQGRVTMTVDKSTSTVYMELSSLRSEDTAVYYCARSTGNYAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 416 - protein, 218 aa, synthetic construct
DIVMTQSPDSLAVSLGERATINCKSSESVDYYGIGFLNWYQQKPGQPPKLLIYAASTQGSGVPDRFSGSGSGTDFTLTISSLQAEDVAVYYCQQSKEVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 417 - protein, 218 aa, synthetic construct
DIVMTQSPDSLAVSLGERATINCKSSRSVDYYGIGFLNWYQQKPGQPPKLLIYATSTQGSGVPDRFSGSGSGTDFTLTISSLQAEDVAVYYCQQQKEVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 418 - protein, 218 aa, synthetic construct
DIVMTQSPDSLAVSLGERATINCKSSRSVDYYGIGFVNWYQQKPGQPPKLLIYATSIQGSGVPDRFSGSGSGTDFTLTISSLQAEDVAVYYCQQQKEVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 419 - protein, 218 aa, synthetic construct; identical to SEQ ID NO: 418

SEQ ID NO: 420 - protein, 218 aa, synthetic construct
DIVMTQSPDSLAVSLGERATINCKSSRSVDYFGIGFLNWYQQKPGQPPKLLIYATSHQGSGVPDRFSGSGSGTDFTLTISSLQAEDVAVYYCQQQKEVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 421 - protein, 218 aa, synthetic construct
DIVMTQSPDSLAVSLGERATINCKSSRSVDYYGIGFLNWYQQKPGQPPKLLIYATSKQGSGVPDRFSGSGSGTDFTLTISSLQAEDVAVYYCQQQKEVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 422 - protein, 218 aa, synthetic construct; identical to SEQ ID NO: 421

SEQ ID NO: 423 - protein, 218 aa, synthetic construct
DIVMTQSPDSLAVSLGERATINCKSSRSVDYYGIGFLNWYQQKPGQPPKLLIYATSHQGSGVPDRFSGSGSGTDFTLTISSLQAEDVAVYYCQQQKHVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 424 - protein, 218 aa, synthetic construct
DIVMTQSPDSLAVSLGERATINCKSSRSVDYYGIGFLNWYQQKPGQPPKLLIYATSHQGSGVPDRFSGSGSGTDFTLTISSLQAEDVAVYYCQQQKEVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 425 - protein, 218 aa, synthetic construct; identical to SEQ ID NO: 424

SEQ ID NO: 426 - protein, 218 aa, synthetic construct
DIVMTQSPDSLAVSLGERATINCKSSRSVDYYGIGFLNWYQQKPGQPPKLLIYATSHQGSGVPDRFSGSGSGTDFTLTISSLQAEDVAVYYCQQQKERPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 427 - protein, 214 aa, synthetic construct
DIVMTQSPDSLAVSLGERATINCKASQDVSPSLAWYQQKPGQPPKLLIYSASYRYTGVPDRFSGSGSGTDFTLTISSLQAEDVAVYYCQQHYSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 428 - protein, 214 aa, synthetic construct
DIVMTQSPDSLAVSLGERATINCKTSQDVSPSLAWYQQKPGQPPKLLIYSASKRYTGVPDRFSGSGSGTDFTLTISSLQAEDVAVYYCQQHYSIPLQFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 429 - protein, 214 aa, synthetic construct
DIVMTQSPDSLAVSLGERATINCKTSQDVSPSLAWYQQKPGQPPKLLIYSASKRYTGVPDRFSGSGSGTDFTLTISSLQAEDVAVYYCQQHYSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 430 - protein, 214 aa, synthetic construct
DIVMTQSPDSLAVSLGERATINCKTSQDVSPSLAWYQQKPGQPPKLLIYSARYRYTGVPDRFSGSGSGTDFTLTISSLQAEDVAVYYCQQHYSIPLQFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 431 - protein, 214 aa, synthetic construct
DIVMTQSPDSLAVSLGERATINCKASQDVSPSLAWYQQKPGQPPKLLIYRASYRYTGVPDRFSGSGSGTDFTLTISSLQAEDVAVYYCQQHYSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 432 - protein, 214 aa, synthetic construct
DIVMTQSPDSLAVSLGERATINCKTSQDVSPSLAWYQQKPGQPPKLLIYSGSYRYTGVPDRFSGSGSGTDFTLTISSLQAEDVAVYYCQQHYSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 433 - protein, 214 aa, synthetic construct; identical to SEQ ID NO: 427

SEQ ID NO: 434 - protein, 573 aa, Macaca fascicularis
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFVVNLVFPRCQKEDANGNIVYEKNCRNEAGLSADPYVYNWTAWSEDSDGENGTGQSHHNVFPDGKPFPHHPGWRRWNFVYVFHTLGQYFQKLGRCSVRVSVNTANVTLGPQLMEVTVYRRHGRAYVPIAQVKDVYVVTDQIPVFVTMSQKNDRNSSDETFLKDLPITFDVLIHDPSHFLNYSTINYKWSFGDNTGLFVSINHTVNHTYVLNGTFSLNLTVKAAAPGPCPPPPPPPPRPPKPTPSLATTLKSCYSNTPGPAGDNPLGLSEIPDENCQINRYGHFQATITIVEGILEVNIIQTTDVLMLVPGPDSSLIDFVVTCQGSIPTEVCTIISDPTCEITQNAVCSPVDVDEMCLLTVRRAFNGSGTYCVNLTLGDDTSLALTSTLISVPDRDPASSLRMANTALISVGCLAIFVTVISLLVYKKHKKYKPIENSPANVVGSKGLSAFLNRAKAVFFPGNQEKDPLLKNQEFKGVS

SEQ ID NO: 435 - protein, 574 aa, Mus musculus
MESLCGVLGFLLLAAGLPLQAAKRFRDVLGHEQYPDHMREHNQLRGWSSDENEWDEHLYPVWRRGDGRWKDSWEGGRVQAVLTSDSPALVGSNITFVVNLVFPRCQKEDANGNIVYEKNCRNDLGLTSDLHVYNWTAGADDGDWEDGTSRSQHLRFPDRRPFPRPHGWKKWSFVYVFHTLGQYFQKLGRCSARVSINTVNLTAGPQVMEVTVFRRYGRAYIPISKVKDVYVITDQIPVFVTMSQKNDRNLSDEIFLRDLPIVFDVLIHDPSHFLNDSAISYKWNFGDNTGLFVSNNHTLNHTYVLNGTFNLNLTVQTAVPGPCPPPSPSTPPPPSTPPSPPPSPLPTLSTPSPSLMPTGYKSMELSDISNENCRINRYGYFRATITIVEGILEVSIMQIADVPMPTPQPANSLMDFTVTCKGATPMEACTIISDPTCQIAQNRVCSPVAVDGLCLLSVRRAFNGSGTYCVNFTLGDDASLALTSTLISIPGKDPDSPLRAVNGVLISIGCLAVLVTMVTILLYKKHKAYKPIGNCPRNTVKGKGLSVLLSHAKAPFFRGDQEKDPLLQDKPRTL

(19) United States
(12) Patent Application Publication
(10) Pub. No.: US 2025/0270343 A1
(43) Pub. Date: Aug. 28, 2025

(54) ANTI-GPNMB ANTIBODIES AND METHODS OF USE THEREOF

(71) Applicant: ALECTOR LLC, South San Francisco, CA (US)

(72) Inventors: Andrew PINCETIC, San Francisco, CA (US); Angie Grace YEE, San Francisco, CA (US); Marjorie BATEMAN, Alameda, CA (US); Marina ROELL, Concord, CA (US); Wei-Hsien HO, Belmont, CA (US); Meer Kamal MUSTAFA, Pacifica, CA (US); Daniel P. BERMINGHAM, San Francisco, CA (US); Christopher James WEDELES, Pacifica, CA (US)

(21) Appl. No.: 19/039,395

(22) Filed: Jan. 28, 2025

Related U.S. Application Data
(63) Continuation of application No. PCT/US2023/071195, filed on Jul. 28, 2023.
(60) Provisional application No. 63/514,318, filed on Jul. 18, 2023.
(60) Provisional application No. 63/369,890, filed on Jul. 29, 2022.

(51) Int. Cl. C07K 16/30; A61P 35/00

(52) U.S. Cl. CPC C07K 16/30; A61P 35/00; C07K 2317/24; C07K 2317/33; C07K 2317/76; C07K 2317/92

(57) ABSTRACT

The present disclosure is generally directed to compositions that include monovalent antibodies, e.g., monoclonal monovalent antibodies that specifically bind a GPNMB polypeptide, e.g., a mammalian GPNMB polypeptide or human GPNMB polypeptide, and use of such compositions in treating an individual in need thereof.

### SEQUENCE LISTING

The present application is being filed with a Sequence Listing which has been submitted electronically in XML format and is hereby incorporated by reference in its entirety. The Sequence Listing is provided as a file entitled “01209-0014-00PCT_ST26.xml”, created on Jul. 20, 2023, and is 524,000 bytes in size.

### FIELD OF THE PRESENT DISCLOSURE

The present disclosure relates to anti-GPNMB antibodies and uses (e.g., therapeutic uses) of such antibodies.

### BACKGROUND OF THE PRESENT DISCLOSURE

Glycoprotein nonmetastatic melanoma protein B (GPNMB), also known as osteoactivin (rat ortholog), dendritic cell-heparin integrin ligand (DC-HIL, mouse ortholog), or hematopoietic growth factor inducible neurokinin-1 type (HGFIN), is a type 1 transmembrane protein.

Increased expression of GPNMB is associated with genome wide significant risk for Parkinson's disease (Murthy et al, 2017, Neurogenetics, 18:121-133). rs199347 SNP is associated with increased expression of GPNMB in brain. GPNMB protein expression has been linked to ALS and to certain lysosomal storage disorders, including Gaucher's disease and Neimann-Pick type C disease (Tanaka et al, 2012, Sci Rep, 2:537; Kramer et al, 2016, FEBS Open Bio, 6:902-913; Marques et al, 2016, PLoS One 11:e0147208).

Additionally, GPNMB is upregulated in various cancers, including glioblastoma multiform, melanomas, and breast cancer (Tse et al, 2006, Clinical Cancer Res, 12:1373-1382; Kuan et al, 2006, Clinical Cancer Res, 12:1970-1982; Rose et al, 2007, Molecular Cancer Res, 5:1001-1014; Zhou et al, 2012, Neoplasma, 59:105; Taya and Hammes, 2018, Steroids, 133:102-107).

Anti-GPNMB antibodies have been previously described. See, e.g., WO2006/071441, WO2007/053718, US2007/0190575, US2013/0156784, U.S. Pat. No. 7,115,265, WO2008/133641, WO2010/135547, WO2016/145022, WO2017/046061, WO2018/217945, and WO2019/137138.

There is a need for novel therapeutic anti-GPNMB antibodies having antagonistic activity that are effective at treating various diseases, disorders, and conditions associated with GPNMB, such as cancer, neurodegenerative disorders, including Parkinson's disease, and lysosomal storage disorders.

All references cited herein, including patent applications and publications, are hereby incorporated by reference in their entirety.

### SUMMARY OF THE PRESENT DISCLOSURE

The present disclosure is generally directed to anti-GPNMB antibodies and methods of using such antibodies.

The present disclosure meets a need for novel therapeutic anti-GPNMB antibodies having antagonistic activity that are effective at treating conditions such as cancer, neurodegenerative disorders, and lysosomal storage disorders. Exemplary embodiments of the disclosure include, inter alia, an isolated anti-Glycoprotein nonmetastatic melanoma protein B (GPNMB) antibody, wherein the anti-GPNMB antibody antagonizes GPNMB activity. In some embodiments, the anti-GPNMB antibody: modulates expression of activation surface markers on myeloid cells, increases cell surface expression of PD-L1 in human macrophages, increases cell surface expression of CD40 in human macrophages, increases cell surface expression of CD80 in human macrophages, modulates lysosome function in myeloid cells, increases glucocerebrosidase activity in human macrophages, decreases cell surface expression of GPNMB in human macrophages, changes interferon pathway gene expression patterns in human macrophages, reduces tumor volume in a murine tumor model, such as in an MC38 model, reduces tumor growth rate in a murine tumor model, such as in an MC38 model, increases levels of IL-12p40 in serum, increases levels of CCL5 in serum, is a GPNMB ligand blocking antibody, is a non-blocking antibody, antagonizes GPNMB activity independent of ligand blocking activity, antagonizes GPNMB activity in vivo, binds to GPNMB expressed on a cell surface the antibody promotes macrophage activation, overcomes a decrease in glucocerebrosidase activity associated with reduced progranulin levels, decreases GPNMB expression levels in cells, optionally in macrophages, decreases LAMP2 expression levels in cells, optionally in macrophages, monocytes, or neutrophils, inhibits or reduces inflammasome activation; inhibits IL-1β expression or release, reduces neural inflammation, reduces expression of C1q, GFAP, IBA1, and CTSD associated with neural inflammation, and/or reduces lysosomal stress.

In some cases, the anti-GPNMB antibody competes with one or more antibodies selected from GPN-01, GPN-03, GPN-06, GPN-07, GPN-08, GPN-09, GPN-11, GPN-22, GPN-24, GPN-25, GPN-26, GPN-30, GPN-31, GPN-33, GPN-34, GPN-35, GPN-37, GPN-38, GPN-41, GPN-42, GPN-43, GPN-48, GPN-52, GPN-61, GPN-65, GPN-81, GPN-82, GPN-83, GPN-84, GPN-85, GPN-86, GPN-87, GPN-88, GPN-89, GPN-90, GPN-91, GPN-92, GPN-93, GPN-94, GPN-95, GPN-96, GPN-97, GPN-98, and any combination thereof for binding to GPNMB. In some cases, the anti-GPNMB antibody comprises a light chain variable domain and a heavy chain variable domain, wherein the light chain variable domain, the heavy chain variable domain, or both comprise at least one, at least two, at least three, at least four, at least five, or six HVRs selected from HVR-L1, HVR-L2, HVR-L3, HVR-H1, HVR-H2, and HVR-H3 of an antibody selected from: GPN-01, GPN-03, GPN-06, GPN-07, GPN-08, GPN-09, GPN-11, GPN-22, GPN-24, GPN-25, GPN-26, GPN-30, GPN-31, GPN-33, GPN-34, GPN-35, GPN-37, GPN-38, GPN-41, GPN-42, GPN-43, GPN-48, GPN-52, GPN-61, GPN-65, GPN-81, GPN-82, GPN-83, GPN-84, GPN-85, GPN-86, GPN-87, GPN-88, GPN-89, GPN-90, GPN-91, GPN-92, GPN-93, GPN-94, GPN-95, GPN-96, GPN-97, and GPN-98. In some cases: (a) the HVR-H1 comprises an amino acid sequence selected from the group consisting of SEQ ID NOs: 5, 8, 11, 14, 17, 20, 23, 26, 29, 32, 35, 38, 41, 44, 47, 50, 53, 56, 59, 62, 65, 68, 71, 74, and 77; (b) the HVR-H2 comprises an amino acid sequence selected from the group consisting of SEQ ID NOs: 6, 9, 12, 15, 18, 21, 24, 27, 30, 33, 36, 39, 42, 45, 48, 51, 54, 57, 60, 63, 66, 69, 72, 75, 78, 255, 256, 257, 258, 259, and 260; (c) the HVR-H3 comprises an amino acid sequence selected from the group consisting of SEQ ID NOs: 7, 10, 13, 16, 19, 22, 25, 28, 31, 34, 37, 40, 43, 46, 49, 52, 55, 58, 61, 64, 67, 70, 73, 76, 79, 261, and 262; (d) the HVR-L1 comprises an amino acid sequence selected from the group consisting of SEQ ID NOs: 83, 86, 89, 92, 95, 98, 101, 104, 107, 110, 113, 116, 119, 122, 125, 128, 131, 134, 137, 140, 143, 146, 149, 152, 155, 263, 264, 265, 266, 267, and 268; (e) the HVR-L2 comprises an amino acid sequence selected from the group consisting of SEQ ID NOs: 84, 87, 90, 93, 96, 99, 102, 105, 108, 111, 114, 117, 120, 123, 126, 129, 132, 135, 138, 141, 144, 147, 150, 153, 156, 269, 270, 271, 272, 273, 274, 275, 276, and 277; and/or (f) the HVR-L3 comprises an amino acid sequence selected from the group consisting of SEQ ID NOs:: 85, 88, 91, 94, 97, 100, 103, 106, 109, 112, 115, 118, 121, 124, 127, 130, 133, 136, 139, 142, 145, 148, 151, 154, 157, 278, 279, 280, and 281. In some cases: the HVR-H1 comprises the amino acid sequence of SEQ ID NO:5, the HVR-H2 comprises the amino acid sequence of SEQ ID NO:6, the HVR-H3 comprises the amino acid sequence of SEQ ID NO:7, the HVR-L1 comprises the amino acid sequence of SEQ ID NO:83, the HVR-L2 comprises the amino acid sequence of SEQ ID NO:84, and the HVR-L3 comprises the amino acid sequence of SEQ ID NO:85; or the HVR-H1 comprises the amino acid sequence of SEQ ID NO:8, the HVR-H2 comprises the amino acid sequence of SEQ ID NO:9, the HVR-H3 comprises the amino acid sequence of SEQ ID NO:10, the HVR-L1 comprises the amino acid sequence of SEQ ID NO:86, the HVR-L2 comprises the amino acid sequence of SEQ ID NO:87, and the HVR-L3 comprises the amino acid sequence of SEQ ID NO:88; or the HVR-H1 comprises the amino acid sequence of SEQ ID NO:11, the HVR-H2 comprises the amino acid sequence of SEQ ID NO:12, the HVR-H3 comprises the amino acid sequence of SEQ ID NO:13, the HVR-L1 comprises the amino acid sequence of SEQ ID NO:89, the HVR-L2 comprises the amino acid sequence of SEQ ID NO:90, and the HVR-L3 comprises the amino acid sequence of SEQ ID NO:91; or the HVR-H1 comprises the amino acid sequence of SEQ ID NO:14, the HVR-H2 comprises the amino acid sequence of SEQ ID NO:15, the HVR-H3 comprises the amino acid sequence of SEQ ID NO:16, the HVR-L1 comprises the amino acid sequence of SEQ ID NO:92, the HVR-L2 comprises the amino acid sequence of SEQ ID NO:93, and the HVR-L3 comprises the amino acid sequence of SEQ ID NO:94; or the HVR-H1 comprises the amino acid sequence of SEQ ID NO:17, the HVR-H2 comprises the amino acid sequence of SEQ ID NO:18, the HVR-H3 comprises the amino acid sequence of SEQ ID NO:19, the HVR-L1 comprises the amino acid sequence of SEQ ID NO:95, the HVR-L2 comprises the amino acid sequence of SEQ ID NO:96, and the HVR-L3 comprises the amino acid sequence of SEQ ID NO:97; or the HVR-H1 comprises the amino acid sequence of SEQ ID NO:20, the HVR-H2 comprises the amino acid sequence of SEQ ID NO:21, the HVR-H3 comprises the amino acid sequence of SEQ ID NO:22, the HVR-L1 comprises the amino acid sequence of SEQ ID NO:98, the HVR-L2 comprises the amino acid sequence of SEQ ID NO:99, and the HVR-L3 comprises the amino acid sequence of SEQ ID NO:100; or the HVR-H1 comprises the amino acid sequence of SEQ ID NO:23, the HVR-H2 comprises the amino acid sequence of SEQ ID NO:24, the HVR-H3 comprises the amino acid sequence of SEQ ID NO:25, the HVR-L1 comprises the amino acid sequence of SEQ ID NO:101, the HVR-L2 comprises the amino acid sequence of SEQ ID NO:102, and the HVR-L3 comprises the amino acid sequence of SEQ ID NO:103; or the HVR-H1 comprises the amino acid sequence of SEQ ID NO:26, the HVR-H2 comprises the amino acid sequence of SEQ ID NO:27, the HVR-H3 comprises the amino acid sequence of SEQ ID NO:28, the HVR-L1 comprises the amino acid sequence of SEQ ID NO:104, the HVR-L2 comprises the amino acid sequence of SEQ ID NO:105, and the HVR-L3 comprises the amino acid sequence of SEQ ID NO:106; or the HVR-H1 comprises the amino acid sequence of SEQ ID NO:29, the HVR-H2 comprises the amino acid sequence of SEQ ID NO:30, the HVR-H3 comprises the amino acid sequence of SEQ ID NO:31, the HVR-L1 comprises the amino acid sequence of SEQ ID NO:107, the HVR-L2 comprises the amino acid sequence of SEQ ID NO:108, and the HVR-L3 comprises the amino acid sequence of SEQ ID NO:109; or the HVR-H1 comprises the amino acid sequence of SEQ ID NO:32, the HVR-H2 comprises the amino acid sequence of SEQ ID NO:33, the HVR-H3 comprises the amino acid sequence of SEQ ID NO:34, the HVR-L1 comprises the amino acid sequence of SEQ ID NO:110, the HVR-L2 comprises the amino acid sequence of SEQ ID NO:111, and the HVR-L3 comprises the amino acid sequence of SEQ ID NO:112; or the HVR-H1 comprises the amino acid sequence of SEQ ID NO:35, the HVR-H2 comprises the amino acid sequence of SEQ ID NO:36, the HVR-H3 comprises the amino acid sequence of SEQ ID NO:37, the HVR-L1 comprises the amino acid sequence of SEQ ID NO:113, the HVR-L2 comprises the amino acid sequence of SEQ ID NO:114, and the HVR-L3 comprises the amino acid sequence of SEQ ID NO:115; or the HVR-H1 comprises the amino acid sequence of SEQ ID NO:38, the HVR-H2 comprises the amino acid sequence of SEQ ID NO:39, the HVR-H3 comprises the amino acid sequence of SEQ ID NO:40, the HVR-L1 comprises the amino acid sequence of SEQ ID NO:116, the HVR-L2 comprises the amino acid sequence of SEQ ID NO:117, and the HVR-L3 comprises the amino acid sequence of SEQ ID NO:118; or the HVR-H1 comprises the amino acid sequence of SEQ ID NO:41, the HVR-H2 comprises the amino acid sequence of SEQ ID NO:42, the HVR-H3 comprises the amino acid sequence of SEQ ID NO:43, the HVR-L1 comprises the amino acid sequence of SEQ ID NO:119, the HVR-L2 comprises the amino acid sequence of SEQ ID NO:120, and the HVR-L3 comprises the amino acid sequence of SEQ ID NO:121; or the HVR-H1 comprises the amino acid sequence of SEQ ID NO:44, the HVR-H2 comprises the amino acid sequence of SEQ ID NO:45, the HVR-H3 comprises the amino acid sequence of SEQ ID NO:46, the HVR-L1 comprises the amino acid sequence of SEQ ID NO:122, the HVR-L2 comprises the amino acid sequence of SEQ ID NO:123, and the HVR-L3 comprises the amino acid sequence of SEQ ID NO:124; or the HVR-H1 comprises the amino acid sequence of SEQ ID NO:47, the HVR-H2 comprises the amino acid sequence of SEQ ID NO:48, the HVR-H3 comprises the amino acid sequence of SEQ ID NO:49, the HVR-L1 comprises the amino acid sequence of SEQ ID NO:125, the HVR-L2 comprises the amino acid sequence of SEQ ID NO:126, and the HVR-L3 comprises the amino acid sequence of SEQ ID NO:127; or the HVR-H1 comprises the amino acid sequence of SEQ ID NO:50, the HVR-H2 comprises the amino acid sequence of SEQ ID NO:51, the HVR-H3 comprises the amino acid sequence of SEQ ID NO:52, the HVR-L1 comprises the amino acid sequence of SEQ ID NO:128, the HVR-L2 comprises the amino acid sequence of SEQ ID NO:129, and the HVR-L3 comprises the amino acid sequence of SEQ ID NO:130; or the HVR-H1 comprises the amino acid sequence of SEQ ID NO:53, the HVR-H2 comprises the amino acid sequence of SEQ ID NO:54, the HVR-H3 comprises the amino acid sequence of SEQ ID NO:55, the HVR-L1 comprises the amino acid sequence of SEQ ID NO:131, the HVR-L2 comprises the amino acid sequence of SEQ ID NO:132, and the HVR-L3 comprises the amino acid sequence of SEQ ID NO:133; or the HVR-H1 comprises the amino acid sequence of SEQ ID NO:56, the HVR-H2 comprises the amino acid sequence of SEQ ID NO:57, the HVR-H3 comprises the amino acid sequence of SEQ ID NO:58, the HVR-L1 comprises the amino acid sequence of SEQ ID NO:134, the HVR-L2 comprises the amino acid sequence of SEQ ID NO:135, and the HVR-L3 comprises the amino acid sequence of SEQ ID NO:136; or the HVR-H1 comprises the amino acid sequence of SEQ ID NO:59, the HVR-H2 comprises the amino acid sequence of SEQ ID NO:60, the HVR-H3 comprises the amino acid sequence of SEQ ID NO:61, the HVR-L1 comprises the amino acid sequence of SEQ ID NO:137, the HVR-L2 comprises the amino acid sequence of SEQ ID NO:138, and the HVR-L3 comprises the amino acid sequence of SEQ ID NO:139; or the HVR-H1 comprises the amino acid sequence of SEQ ID NO:62, the HVR-H2 comprises the amino acid sequence of SEQ ID NO:63, the HVR-H3 comprises the amino acid sequence of SEQ ID NO:64, the HVR-L1 comprises the amino acid sequence of SEQ ID NO:140, the HVR-L2 comprises the amino acid sequence of SEQ ID NO:141, and the HVR-L3 comprises the amino acid sequence of SEQ ID NO:142; or the HVR-H1 comprises the amino acid sequence of SEQ ID NO:65, the HVR-H2 comprises the amino acid sequence of SEQ ID NO:66, the HVR-H3 comprises the amino acid sequence of SEQ ID NO:67, the HVR-L1 comprises the amino acid sequence of SEQ ID NO:143, the HVR-L2 comprises the amino acid sequence of SEQ ID NO:144, and the HVR-L3 comprises the amino acid sequence of SEQ ID NO:145; or the HVR-H1 comprises the amino acid sequence of SEQ ID NO:68, the HVR-H2 comprises the amino acid sequence of SEQ ID NO:69, the HVR-H3 comprises the amino acid sequence of SEQ ID NO:70, the HVR-L1 comprises the amino acid sequence of SEQ ID NO:146, the HVR-L2 comprises the amino acid sequence of SEQ ID NO:147, and the HVR-L3 comprises the amino acid sequence of SEQ ID NO:148; or the HVR-H1 comprises the amino acid sequence of SEQ ID NO:71, the HVR-H2 comprises the amino acid sequence of SEQ ID NO:72, the HVR-H3 comprises the amino acid sequence of SEQ ID NO:73, the HVR-L1 comprises the amino acid sequence of SEQ ID NO:149, the HVR-L2 comprises the amino acid sequence of SEQ ID NO:150, and the HVR-L3 comprises the amino acid sequence of SEQ ID NO:151; or the HVR-H1 comprises the amino acid sequence of SEQ ID NO:74, the HVR-H2 comprises the amino acid sequence of SEQ ID NO:75, the HVR-H3 comprises the amino acid sequence of SEQ ID NO:76, the HVR-L1 comprises the amino acid sequence of SEQ ID NO:152, the HVR-L2 comprises the amino acid sequence of SEQ ID NO:153, and the HVR-L3 comprises the amino acid sequence of SEQ ID NO:154; or the HVR-H1 comprises the amino acid sequence of SEQ ID NO:77, the HVR-H2 comprises the amino acid sequence of SEQ ID NO:78, the HVR-H3 comprises the amino acid sequence of SEQ ID NO:79, the HVR-L1 comprises the amino acid sequence of SEQ ID NO:155, the HVR-L2 comprises the amino acid sequence of SEQ ID NO:156, and the HVR-L3 comprises the amino acid sequence of SEQ ID NO:157 or the HVR-H1 comprises the amino acid sequence of SEQ ID NO:71, the HVR-H2 comprises the amino acid sequence of SEQ ID NO:255, the HVR-H3 comprises the amino acid sequence of SEQ ID NO:73, the HVR-L1 comprises the amino acid sequence of SEQ ID NO:263, the HVR-L2 comprises the amino acid sequence of SEQ ID NO:269, and the HVR-L3 comprises the amino acid sequence of SEQ ID NO:151; or the HVR-H1 comprises the amino acid sequence of SEQ ID NO:71, the HVR-H2 comprises the amino acid sequence of SEQ ID NO:256, the HVR-H3 comprises the amino acid sequence of SEQ ID NO:73, the HVR-L1 comprises the amino acid sequence of SEQ ID NO:264, the HVR-L2 comprises the amino acid sequence of SEQ ID NO:270, and the HVR-L3 comprises the amino acid sequence of SEQ ID NO:278; or the HVR-H1 comprises the amino acid sequence of SEQ ID NO:71, the HVR-H2 comprises the amino acid sequence of SEQ ID NO:257, the HVR-H3 comprises the amino acid sequence of SEQ ID NO:73, the HVR-L1 comprises the amino acid sequence of SEQ ID NO:265, the HVR-L2 comprises the amino acid sequence of SEQ ID NO:271, and the HVR-L3 comprises the amino acid sequence of SEQ ID NO:278; or the HVR-H1 comprises the amino acid sequence of SEQ ID NO:71, the HVR-H2 comprises the amino acid sequence of SEQ ID NO:257, the HVR-H3 comprises the amino acid sequence of SEQ ID NO:261, the HVR-L1 comprises the amino acid sequence of SEQ ID NO:265, the HVR-L2 comprises the amino acid sequence of SEQ ID NO:271, and the HVR-L3 comprises the amino acid sequence of SEQ ID NO:278; or the HVR-H1 comprises the amino acid sequence of SEQ ID NO:71, the HVR-H2 comprises the amino acid sequence of SEQ ID NO:258, the HVR-H3 comprises the amino acid sequence of SEQ ID NO:73, the HVR-L1 comprises the amino acid sequence of SEQ ID NO:266, the HVR-L2 comprises the amino acid sequence of SEQ ID NO:272, and the HVR-L3 comprises the amino acid sequence of SEQ ID NO:278; or the HVR-H1 comprises the amino acid sequence of SEQ ID NO:71, the HVR-H2 comprises the amino acid sequence of SEQ ID NO:257, the HVR-H3 comprises the amino acid sequence of SEQ ID NO:73, the HVR-L1 comprises the amino acid sequence of SEQ ID NO:264, the HVR-L2 comprises the amino acid sequence of SEQ ID NO:273, and the HVR-L3 comprises the amino acid sequence of SEQ ID NO:278; or the HVR-H1 comprises the amino acid sequence of SEQ ID NO:71, the HVR-H2 comprises the amino acid sequence of SEQ ID NO:257, the HVR-H3 comprises the amino acid sequence of SEQ ID NO:261, the HVR-L1 comprises the amino acid sequence of SEQ ID NO:264, the HVR-L2 comprises the amino acid sequence of SEQ ID NO:273, and the HVR-L3 comprises the amino acid sequence of SEQ ID NO:278; or the HVR-H1 comprises the amino acid sequence of SEQ ID NO:71, the HVR-H2 comprises the amino acid sequence of SEQ ID NO:257, the HVR-H3 comprises the amino acid sequence of SEQ ID NO:73, the HVR-L1 comprises the amino acid sequence of SEQ ID NO:264, the HVR-L2 comprises the amino acid sequence of SEQ ID NO:272, and the HVR-L3 comprises the amino acid sequence of SEQ ID NO:279; or the HVR-H1 comprises the amino acid sequence of SEQ ID NO:71, the HVR-H2 comprises the amino acid sequence of SEQ ID NO:257, the HVR-H3 comprises the amino acid sequence of SEQ ID NO:261, the HVR-L1 comprises the amino acid sequence of SEQ ID NO:264, the HVR-L2 comprises the amino acid sequence of SEQ ID NO:272, and the HVR-L3 comprises the amino acid sequence of SEQ ID NO:278; or the HVR-H1 comprises the amino acid sequence of SEQ ID NO:71, the HVR-H2 comprises the amino acid sequence of SEQ ID NO:258, the HVR-H3 comprises the amino acid sequence of SEQ ID NO:73, the HVR-L1 comprises the amino acid sequence of SEQ ID NO:264, the HVR-L2 comprises the amino acid sequence of SEQ ID NO:272, and the HVR-L3 comprises the amino acid sequence of SEQ ID NO:278; or the HVR-H1 comprises the amino acid sequence of SEQ ID NO:71, the HVR-H2 comprises the amino acid sequence of SEQ ID NO:257, the HVR-H3 comprises the amino acid sequence of SEQ ID NO:73, the HVR-L1 comprises the amino acid sequence of SEQ ID NO:264, the HVR-L2 comprises the amino acid sequence of SEQ ID NO:272, and the HVR-L3 comprises the amino acid sequence of SEQ ID NO:280; or the HVR-H1 comprises the amino acid sequence of SEQ ID NO:23, the HVR-H2 comprises the amino acid sequence of SEQ ID NO:259, the HVR-H3 comprises the amino acid sequence of SEQ ID NO:19, the HVR-L1 comprises the amino acid sequence of SEQ ID NO:267, the HVR-L2 comprises the amino acid sequence of SEQ ID NO:96, and the HVR-L3 comprises the amino acid sequence of SEQ ID NO:97; or the HVR-H1 comprises the amino acid sequence of SEQ ID NO:23, the HVR-H2 comprises the amino acid sequence of SEQ ID NO:260, the HVR-H3 comprises the amino acid sequence of SEQ ID NO:262, the HVR-L1 comprises the amino acid sequence of SEQ ID NO:268, the HVR-L2 comprises the amino acid sequence of SEQ ID NO:274, and the HVR-L3 comprises the amino acid sequence of SEQ ID NO:281; or the HVR-H1 comprises the amino acid sequence of SEQ ID NO:23, the HVR-H2 comprises the amino acid sequence of SEQ ID NO:260, the HVR-H3 comprises the amino acid sequence of SEQ ID NO:262, the HVR-L1 comprises the amino acid sequence of SEQ ID NO:268, the HVR-L2 comprises the amino acid sequence of SEQ ID NO:274, and the HVR-L3 comprises the amino acid sequence of SEQ ID NO:97; or the HVR-H1 comprises the amino acid sequence of SEQ ID NO:23, the HVR-H2 comprises the amino acid sequence of SEQ ID NO:260, the HVR-H3 comprises the amino acid sequence of SEQ ID NO:262, the HVR-L1 comprises the amino acid sequence of SEQ ID NO:268, the HVR-L2 comprises the amino acid sequence of SEQ ID NO:275, and the HVR-L3 comprises the amino acid sequence of SEQ ID NO:281; or the HVR-H1 comprises the amino acid sequence of SEQ ID NO:23, the HVR-H2 comprises the amino acid sequence of SEQ ID NO:260, the HVR-H3 comprises the amino acid sequence of SEQ ID NO:262, the HVR-L1 comprises the amino acid sequence of SEQ ID NO:267, the HVR-L2 comprises the amino acid sequence of SEQ ID NO:276, and the HVR-L3 comprises the amino acid sequence of SEQ ID NO:97; or the HVR-H1 comprises the amino acid sequence of SEQ ID NO:23, the HVR-H2 comprises the amino acid sequence of SEQ ID NO:260, the HVR-H3 comprises the amino acid sequence of SEQ ID NO:262, the HVR-L1 comprises the amino acid sequence of SEQ ID NO:268, the HVR-L2 comprises the amino acid sequence of SEQ ID NO:277, and the HVR-L3 comprises the amino acid sequence of SEQ ID NO:97; or the HVR-H1 comprises the amino acid sequence of SEQ ID NO:23, the HVR-H2 comprises the amino acid sequence of SEQ ID NO:260, the HVR-H3 comprises the amino acid sequence of SEQ ID NO:262, the HVR-L1 comprises the amino acid sequence of SEQ ID NO:267, the HVR-L2 comprises the amino acid sequence of SEQ ID NO:96, and the HVR-L3 comprises the amino acid sequence of SEQ ID NO:97.

The present disclosure also relates to an isolated anti-Glycoprotein nonmetastatic melanoma protein B (GPNMB) antibody, wherein the anti-GPNMB antibody comprises a heavy chain variable region (VH) and a light chain variable region (VL), wherein the heavy chain variable region (VH) comprises: an HVR-H1 comprising an amino acid sequence chosen from any one of SEQ ID NOs: 5, 8, 11, 14, 17, 20, 23, 26, 29, 32, 35, 38, 41, 44, 47, 50, 53, 56, 59, 62, 65, 68, 71, 74, and 77; an HVR-H2 comprising an amino acid sequence chosen from any one of SEQ ID NOs: 6, 9, 12, 15, 18, 21, 24, 27, 30, 33, 36, 39, 42, 45, 48, 51, 54, 57, 60, 63, 66, 69, 72, 75, 78, 255, 256, 257, 258, 259, and 260; and an HVR-H3 comprising an amino acid sequence chosen from any one of SEQ ID NOs: 7, 10, 13, 16, 19, 22, 25, 28, 31, 34, 37, 40, 43, 46, 49, 52, 55, 58, 61, 64, 67, 70, 73, 76, 79, 261, and 262. In some cases, the light chain variable region (VL) comprises: an HVR-L1 comprising an amino acid sequence chosen from any one of SEQ ID NOs: 83, 86, 89, 92, 95, 98, 101, 104, 107, 110, 113, 116, 119, 122, 125, 128, 131, 134, 137, 140, 143, 146, 149, 152, 155, 263, 264, 265, 266, 267, and 268; an HVR-L2 comprising an amino acid sequence chosen from any one of SEQ ID NOs: 84, 87, 90, 93, 96, 99, 102, 105, 108, 111, 114, 117, 120, 123, 126, 129, 132, 135, 138, 141, 144, 147, 150, 153, 156, 269, 270, 271, 272, 273, 274, 275, 276, and 277; and an HVR-L3 comprising an amino acid sequence chosen from any one of SEQ ID NOs: 85, 88, 91, 94, 97, 100, 103, 106, 109, 112, 115, 118, 121, 124, 127, 130, 133, 136, 139, 142, 145, 148, 151, 154, 157, 278, 279, 280, and 281. In some cases, the antibody comprises a VH that is at least 90%, at least 95%, at least 97%, or at least 99% identical to an amino acid sequence selected from any one of SEQ ID NOs: 161, 163, 165, 167, 169, 171, 173, 175, 177, 179, 181, 183, 185, 187, 189, 191, 193, 195, 197, 199, 201, 203, 205, 207, 209, 234, 235, 236, 237, 238, 239, and 240, and/or the antibody comprises a VL that is at least 90%, at least 95%, at least 97%, or at least 99% identical to an amino acid sequence selected from any one of SEQ ID NOs: 162, 164, 166, 168, 170, 172, 174, 176, 178, 180, 182, 184, 186, 188, 190, 192, 194, 196, 198, 200, 202, 204, 206, 208, 210, 241, 242, 243, 244, 245, 246, 247, 248, 249, 250, 251, 252, 253, and 254. In some cases, the VH comprises 1, 2, 3, 4, 5, 6, 7, 8, 9, or 10 amino acid substitutions, insertions, and/or deletions compared to an amino acid sequence selected from any one of SEQ ID NOs: 161, 163, 165, 167, 169, 171, 173, 175, 177, 179, 181, 183, 185, 187, 189, 191, 193, 195, 197, 199, 201, 203, 205, 207, 209, 234, 235, 236, 237, 238, 239, and 240, and/or the VL comprises 1, 2, 3, 4, 5, 6, 7, 8, 9, or 10 amino acid substitutions, insertions, and/or deletions compared to an amino acid sequence selected from any one of SEQ ID NOs: 162, 164, 166, 168, 170, 172, 174, 176, 178, 180, 182, 184, 186, 188, 190, 192, 194, 196, 198, 200, 202, 204, 206, 208, 210, 241, 242, 243, 244, 245, 246, 247, 248, 249, 250, 251, 252, 253, and 254. In some cases, the antibody comprises a VH comprising an amino acid sequence selected from any one of SEQ ID NOs: 161, 163, 165, 167, 169, 171, 173, 175, 177, 179, 181, 183, 185, 187, 189, 191, 193, 195, 197, 199, 201, 203, 205, 207, 209, 234, 235, 236, 237, 238, 239, and 240, and/or a VL comprising an amino acid sequence selected from any one of SEQ ID NOs: 162, 164, 166, 168, 170, 172, 174, 176, 178, 180, 182, 184, 186, 188, 190, 192, 194, 196, 198, 200, 202, 204, 206, 208, 210, 241, 242, 243, 244, 245, 246, 247, 248, 249, 250, 251, 252, 253, and 254.

The disclosure further relates to an isolated anti-Glycoprotein nonmetastatic melanoma protein B (GPNMB) antibody, wherein the anti-GPNMB antibody comprises: a VH comprising an HVR-H1 comprising the amino acid sequence of SEQ ID NO: 5, an HVR-H2 comprising the amino acid sequence of SEQ ID NO: 6, and an HVR-H3 comprising the amino acid sequence of SEQ ID NO: 7; and a VL comprising an HVR-L1 comprising the amino acid sequence of SEQ ID NO: 83, an HVR-L2 comprising the amino acid sequence of SEQ ID NO: 84, and an HVR-L3 comprising the amino acid sequence of SEQ ID NO: 85; or a VH comprising an HVR-H1 comprising the amino acid sequence of SEQ ID NO: 8, an HVR-H2 comprising the amino acid sequence of SEQ ID NO: 9, and an HVR-H3 comprising the amino acid sequence of SEQ ID NO: 10; and a VL comprising an HVR-L1 comprising the amino acid sequence of SEQ ID NO: 86, an HVR-L2 comprising the amino acid sequence of SEQ ID NO: 87, and an HVR-L3 comprising the amino acid sequence of SEQ ID NO: 88; or a VH comprising an HVR-H1 comprising the amino acid sequence of SEQ ID NO: 11, an HVR-H2 comprising the amino acid sequence of SEQ ID NO: 12, and an HVR-H3 comprising the amino acid sequence of SEQ ID NO: 13; and a VL comprising an HVR-L1 comprising the amino acid sequence of SEQ ID NO: 89, an HVR-L2 comprising the amino acid sequence of SEQ ID NO: 90, and an HVR-L3 comprising the amino acid sequence of SEQ ID NO: 91; or a VH comprising an HVR-H1 comprising the amino acid sequence of SEQ ID NO: 14, an HVR-H2 comprising the amino acid sequence of SEQ ID NO: 15, and an HVR-H3 comprising the amino acid sequence of SEQ ID NO: 16; and a VL comprising an HVR-L1 comprising the amino acid sequence of SEQ ID NO: 92, an HVR-L2 comprising the amino acid sequence of SEQ ID NO: 93, and an HVR-L3 comprising the amino acid sequence of SEQ ID NO: 94; or a VH comprising an HVR-H1 comprising the amino acid sequence of SEQ ID NO: 17, an HVR-H2 comprising the amino acid sequence of SEQ ID NO: 18, and an HVR-H3 comprising the amino acid sequence of SEQ ID NO: 19; and a VL comprising an HVR-L1 comprising the amino acid sequence of SEQ ID NO: 95, an HVR-L2 comprising the amino acid sequence of SEQ ID NO: 96, and an HVR-L3 comprising the amino acid sequence of SEQ ID NO: 97; or a VH comprising an HVR-H1 comprising the amino acid sequence of SEQ ID NO: 20, an HVR-H2 comprising the amino acid sequence of SEQ ID NO: 21, and an HVR-H3 comprising the amino acid sequence of SEQ ID NO: 22; and a VL comprising an HVR-L1 comprising the amino acid sequence of SEQ ID NO: 98, an HVR-L2 comprising the amino acid sequence of SEQ ID NO: 99, and an HVR-L3 comprising the amino acid sequence of SEQ ID NO: 100; or a VH comprising an HVR-H1 comprising the amino acid sequence of SEQ ID NO: 23, an HVR-H2 comprising the amino acid sequence of SEQ ID NO: 24, and an HVR-H3 comprising the amino acid sequence of SEQ ID NO: 25; and a VL comprising an HVR-L1 comprising the amino acid sequence of SEQ ID NO: 101, an HVR-L2 comprising the amino acid sequence of SEQ ID NO: 102, and an HVR-L3 comprising the amino acid sequence of SEQ ID NO: 103; or a VH comprising an HVR-H1 comprising the amino acid sequence of SEQ ID NO: 26, an HVR-H2 comprising the amino acid sequence of SEQ ID NO: 27, and an HVR-H3 comprising the amino acid sequence of SEQ ID NO: 28; and a VL comprising an HVR-L1 comprising the amino acid sequence of SEQ ID NO: 104, an HVR-L2 comprising the amino acid sequence of SEQ ID NO: 105, and an HVR-L3 comprising the amino acid sequence of SEQ ID NO: 106; or a VH comprising an HVR-H1 comprising the amino acid sequence of SEQ ID NO: 29, an HVR-H2 comprising the amino acid sequence of SEQ ID NO: 30, and an HVR-H3 comprising the amino acid sequence of SEQ ID NO: 31; and a VL comprising an HVR-L1 comprising the amino acid sequence of SEQ ID NO: 107, an HVR-L2 comprising the amino acid sequence of SEQ ID NO: 108, and an HVR-L3 comprising the amino acid sequence of SEQ ID NO: 109; or a VH comprising an HVR-H1 comprising the amino acid sequence of SEQ ID NO: 32, an HVR-H2 comprising the amino acid sequence of SEQ ID NO: 33, and an HVR-H3 comprising the amino acid sequence of SEQ ID NO: 34; and a VL comprising an HVR-L1 comprising the amino acid sequence of SEQ ID NO: 110, an HVR-L2 comprising the amino acid sequence of SEQ ID NO: 111, and an HVR-L3 comprising the amino acid sequence of SEQ ID NO: 112; or a VH comprising an HVR-H1 comprising the amino acid sequence of SEQ ID NO: 35, an HVR-H2 comprising the amino acid sequence of SEQ ID NO: 36, and an HVR-H3 comprising the amino acid sequence of SEQ ID NO: 37; and a VL comprising an HVR-L1 comprising the amino acid sequence of SEQ ID NO: 113, an HVR-L2 comprising the amino acid sequence of SEQ ID NO: 114, and an HVR-L3 comprising the amino acid sequence of SEQ ID NO: 115; or a VH comprising an HVR-H1 comprising the amino acid sequence of SEQ ID NO: 38, an HVR-H2 comprising the amino acid sequence of SEQ ID NO: 39, and an HVR-H3 comprising the amino acid sequence of SEQ ID NO: 40; and a VL comprising an HVR-L1 comprising the amino acid sequence of SEQ ID NO: 116, an HVR-L2 comprising the amino acid sequence of SEQ ID NO: 117, and an HVR-L3 comprising the amino acid sequence of SEQ ID NO: 118; or a VH comprising an HVR-H1 comprising the amino acid sequence of SEQ ID NO: 41, an HVR-H2 comprising the amino acid sequence of SEQ ID NO: 42, and an HVR-H3 comprising the amino acid sequence of SEQ ID NO: 43; and a VL comprising an HVR-L1 comprising the amino acid sequence of SEQ ID NO: 119, an HVR-L2 comprising the amino acid sequence of SEQ ID NO: 120, and an HVR-L3 comprising the amino acid sequence of SEQ ID NO: 121; or a VH comprising an HVR-H1 comprising the amino acid sequence of SEQ ID NO: 44, an HVR-H2 comprising the amino acid sequence of SEQ ID NO: 45, and an HVR-H3 comprising the amino acid sequence of SEQ ID NO: 46; and a VL comprising an HVR-L1 comprising the amino acid sequence of SEQ ID NO: 122, an HVR-L2 comprising the amino acid sequence of SEQ ID NO: 123, and an HVR-L3 comprising the amino acid sequence of SEQ ID NO: 124; or a VH comprising an HVR-H1 comprising the amino acid sequence of SEQ ID NO: 47, an HVR-H2 comprising the amino acid sequence of SEQ ID NO: 48, and an HVR-H3 comprising the amino acid sequence of SEQ ID NO: 49; and a VL comprising an HVR-L1 comprising the amino acid sequence of SEQ ID NO: 125, an HVR-L2 comprising the amino acid sequence of SEQ ID NO: 126, and an HVR-L3 comprising the amino acid sequence of SEQ ID NO: 127; or a VH comprising an HVR-H1 comprising the amino acid sequence of SEQ ID NO: 50, an HVR-H2 comprising the amino acid sequence of SEQ ID NO: 51, and an HVR-H3 comprising the amino acid sequence of SEQ ID NO: 52; and a VL comprising an HVR-L1 comprising the amino acid sequence of SEQ ID NO: 128, an HVR-L2 comprising the amino acid sequence of SEQ ID NO: 129, and an HVR-L3 comprising the amino acid sequence of SEQ ID NO: 130; or a VH comprising an HVR-H1 comprising the amino acid sequence of SEQ ID NO: 53, an HVR-H2 comprising the amino acid sequence of SEQ ID NO: 54, and an HVR-H3 comprising the amino acid sequence of SEQ ID NO: 55; and a VL comprising an HVR-L1 comprising the amino acid sequence of SEQ ID NO: 131, an HVR-L2 comprising the amino acid sequence of SEQ ID NO: 132, and an HVR-L3 comprising the amino acid sequence of SEQ ID NO: 133; or a VH comprising an HVR-H1 comprising the amino acid sequence of SEQ ID NO: 56, an HVR-H2 comprising the amino acid sequence of SEQ ID NO: 57, and an HVR-H3 comprising the amino acid sequence of SEQ ID NO: 58; and a VL comprising an HVR-L1 comprising the amino acid sequence of SEQ ID NO: 134, an HVR-L2 comprising the amino acid sequence of SEQ ID NO: 135, and an HVR-L3 comprising the amino acid sequence of SEQ ID NO: 136; or a VH comprising an HVR-H1 comprising the amino acid sequence of SEQ ID NO: 59, an HVR-H2 comprising the amino acid sequence of SEQ ID NO: 60, and an HVR-H3 comprising the amino acid sequence of SEQ ID NO: 61; and a VL comprising an HVR-L1 comprising the amino acid sequence of SEQ ID NO: 137, an HVR-L2 comprising the amino acid sequence of SEQ ID NO: 138, and an HVR-L3 comprising the amino acid sequence of SEQ ID NO: 139; or a VH comprising an HVR-H1 comprising the amino acid sequence of SEQ ID NO: 62, an HVR-H2 comprising the amino acid sequence of SEQ ID NO: 63, and an HVR-H3 comprising the amino acid sequence of SEQ ID NO: 64; and a VL comprising an HVR-L1 comprising the amino acid sequence of SEQ ID NO: 140, an HVR-L2 comprising the amino acid sequence of SEQ ID NO: 141, and an HVR-L3 comprising the amino acid sequence of SEQ ID NO: 142; or a VH comprising an HVR-H1 comprising the amino acid sequence of SEQ ID NO: 65, an HVR-H2 comprising the amino acid sequence of SEQ ID NO: 66, and an HVR-H3 comprising the amino acid sequence of SEQ ID NO: 67; and a VL comprising an HVR-L1 comprising the amino acid sequence of SEQ ID NO: 143, an HVR-L2 comprising the amino acid sequence of SEQ ID NO: 144, and an HVR-L3 comprising the amino acid sequence of SEQ ID NO: 145; or a VH comprising an HVR-H1 comprising the amino acid sequence of SEQ ID NO: 68, an HVR-H2 comprising the amino acid sequence of SEQ ID NO: 69, and an HVR-H3 comprising the amino acid sequence of SEQ ID NO: 70; and a VL comprising an HVR-L1 comprising the amino acid sequence of SEQ ID NO: 146, an HVR-L2 comprising the amino acid sequence of SEQ ID NO: 147, and an HVR-L3 comprising the amino acid sequence of SEQ ID NO: 148; or a VH comprising an HVR-H1 comprising the amino acid sequence of SEQ ID NO: 71, an HVR-H2 comprising the amino acid sequence of SEQ ID NO: 72, and an HVR-H3 comprising the amino acid sequence of SEQ ID NO: 73; and a VL comprising an HVR-L1 comprising the amino acid sequence of SEQ ID NO: 149, an HVR-L2 comprising the amino acid sequence of SEQ ID NO: 150, and an HVR-L3 comprising the amino acid sequence of SEQ ID NO: 151; or a VH comprising an HVR-H1 comprising the amino acid sequence of SEQ ID NO: 74, an HVR-H2 comprising the amino acid sequence of SEQ ID NO: 75, and an HVR-H3 comprising the amino acid sequence of SEQ ID NO: 76; and a VL comprising an HVR-L1 comprising the amino acid sequence of SEQ ID NO: 152, an HVR-L2 comprising the amino acid sequence of SEQ ID NO: 153, and an HVR-L3 comprising the amino acid sequence of SEQ ID NO: 154; or a VH comprising an HVR-H1 comprising the amino acid sequence of SEQ ID NO: 77, an HVR-H2 comprising the amino acid sequence of SEQ ID NO: 78, and an HVR-H3 comprising the amino acid sequence of SEQ ID NO: 79; and a VL comprising an HVR-L1 comprising the amino acid sequence of SEQ ID NO: 155, an HVR-L2 comprising the amino acid sequence of SEQ ID NO: 156, and an HVR-L3 comprising the amino acid sequence of SEQ ID NO: 157; or a VH comprising an HVR-H1 comprising the amino acid sequence of SEQ ID NO:71, an HVR-H2 comprising the amino acid sequence of SEQ ID NO:255, an HVR-H3 comprising the amino acid sequence of SEQ ID NO:73, a VL comprising an HVR-L1 comprising the amino acid sequence of SEQ ID NO:263, an HVR-L2 comprising the amino acid sequence of SEQ ID NO:269, and an HVR-L3 comprising the amino acid sequence of SEQ ID NO:151; or a VH comprising an HVR-H1 comprising amino acid sequence of SEQ ID NO:71, an HVR-H2 comprising the amino acid sequence of SEQ ID NO:256, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:270, and an HVR-L3 comprising the amino acid sequence of SEQ ID NO:278; or a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:73, a VL comprising an HVR-L1 comprising the amino acid sequence of SEQ ID NO:265, an HVR-L2 comprising the amino acid sequence of SEQ ID NO:271, and an HVR-L3 comprising the amino acid sequence of SEQ ID NO:278; or a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:261, a VL comprising an HVR-L1 comprising the amino acid sequence of SEQ ID NO:265, an HVR-L2 comprising the amino acid sequence of SEQ ID NO:271, and an HVR-L3 comprising the amino acid sequence of SEQ ID NO:278; or a VH comprising an HVR-H1 comprising the amino acid sequence of SEQ ID NO:71, an HVR-H2 comprising the amino acid sequence of SEQ ID NO:258, an HVR-H3 comprising the amino acid sequence of SEQ ID NO:73, a VL comprising an HVR-L1 comprising the amino acid sequence of SEQ ID NO:266, an HVR-L2 comprising the amino acid sequence of SEQ ID NO:272, and an HVR-L3 comprising the amino acid sequence of SEQ ID NO:278; or a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:273, and an HVR-L3 comprising the amino acid sequence of SEQ ID NO:278; or a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:261, a VL comprising an HVR-L1 comprising the amino acid sequence of SEQ ID NO:264, an HVR-L2 comprising the amino acid sequence of SEQ ID NO:273, and an HVR-L3 comprising the amino acid sequence of SEQ ID NO:278; or a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:272, and an HVR-L3 comprising the amino acid sequence of SEQ ID NO:279; or a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:261, a VL comprising an HVR-L1 comprising the amino acid sequence of SEQ ID NO:264, an HVR-L2 comprising the amino acid sequence of SEQ ID NO:272, and an HVR-L3 comprising the amino acid sequence of SEQ ID NO:278; or a VH comprising an HVR-H1 comprising the amino acid sequence of SEQ ID NO:71, an HVR-H2 comprising the amino acid sequence of SEQ ID NO:258, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:272, and an HVR-L3 comprising the amino acid sequence of SEQ ID NO:278; or a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:272, and an HVR-L3 comprising the amino acid sequence of SEQ ID NO:280; or a VH comprising an HVR-H1 comprising the amino acid sequence of SEQ ID NO:23, an HVR-H2 comprising the amino acid sequence of SEQ ID NO:259, an HVR-H3 comprising the amino acid sequence of SEQ ID NO: 19, a VL comprising an HVR-L1 comprising the amino acid sequence of SEQ ID NO:267, an HVR-L2 comprising the amino acid sequence of SEQ ID NO:96, and an HVR-L3 comprising the amino acid sequence of SEQ ID NO:97; or a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:268, an HVR-L2 comprising the amino acid sequence of SEQ ID NO:274, and an HVR-L3 comprising the amino acid sequence of SEQ ID NO:281; or a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:268, an HVR-L2 comprising the amino acid sequence of SEQ ID NO:274, and an HVR-L3 comprising the amino acid sequence of SEQ ID NO:97; or a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:268, an HVR-L2 comprising the amino acid sequence of SEQ ID NO:275, and an HVR-L3 comprising the amino acid sequence of SEQ ID NO:281; or a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:267, an HVR-L2 comprising the amino acid sequence of SEQ ID NO:276, and an HVR-L3 comprising the amino acid sequence of SEQ ID NO:97; or a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:268, an HVR-L2 comprising the amino acid sequence of SEQ ID NO:277, and an HVR-L3 comprising the amino acid sequence of SEQ ID NO:97; or a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:267, an HVR-L2 comprising the amino acid sequence of SEQ ID NO:96, and an HVR-L3 comprising the amino acid sequence of SEQ ID NO:97.

In some such cases, the antibody comprises: a VH that is at least 90%, at least 95%, at least 97%, or at least 99% identical to the amino acid sequence of SEQ ID NO:161; a VH that is at least 90%, at least 95%, at least 97%, or at least 99% identical to the amino acid sequence of SEQ ID NO:163; a VH that is at least 90%, at least 95%, at least 97%, or at least 99% identical to the amino acid sequence of SEQ ID NO:165; a VH that is at least 90%, at least 95%, at least 97%, or at least 99% identical to the amino acid sequence of SEQ ID NO:167; a VH that is at least 90%, at least 95%, at least 97%, or at least 99% identical to the amino acid sequence of SEQ ID NO:169; a VH that is at least 90%, at least 95%, at least 97%, or at least 99% identical to the amino acid sequence of SEQ ID NO:171; a VH that is at least 90%, at least 95%, at least 97%, or at least 99% identical to the amino acid sequence of SEQ ID NO:173; s a VH that is at least 90%, at least 95%, at least 97%, or at least 99% identical to the amino acid sequence of SEQ ID NO:175; a VH that is at least 90%, at least 95%, at least 97%, or at least 99% identical to the amino acid sequence of SEQ ID NO:177; a VH that is at least 90%, at least 95%, at least 97%, or at least 99% identical to the amino acid sequence of SEQ ID NO:179; a VH that is at least 90%, at least 95%, at least 97%, or at least 99% identical to the amino acid sequence of SEQ ID NO:181; a VH that is at least 90%, at least 95%, at least 97%, or at least 99% identical to the amino acid sequence of SEQ ID NO:183; a VH that is at least 90%, at least 95%, at least 97%, or at least 99% identical to the amino acid sequence of SEQ ID NO:185; a VH that is at least 90%, at least 95%, at least 97%, or at least 99% identical to the amino acid sequence of SEQ ID NO:187; a VH that is at least 90%, at least 95%, at least 97%, or at least 99% identical to the amino acid sequence of SEQ ID NO:189; a VH that is at least 90%, at least 95%, at least 97%, or at least 99% identical to the amino acid sequence of SEQ ID NO:191; a VH that is at least 90%, at least 95%, at least 97%, or at least 99% identical to the amino acid sequence of SEQ ID NO:193; a VH that is at least 90%, at least 95%, at least 97%, or at least 99% identical to the amino acid sequence of SEQ ID NO:195; a VH that is at least 90%, at least 95%, at least 97%, or at least 99% identical to the amino acid sequence of SEQ ID NO:197; a VH that is at least 90%, at least 95%, at least 97%, or at least 99% identical to the amino acid sequence of SEQ ID NO:199; a VH that is at least 90%, at least 95%, at least 97%, or at least 99% identical to the amino acid sequence of SEQ ID NO:201; a VH that is at least 90%, at least 95%, at least 97%, or at least 99% identical to the amino acid sequence of SEQ ID NO:203; a VH that is at least 90%, at least 95%, at least 97%, or at least 99% identical to the amino acid sequence of SEQ ID NO:205; a VH that is at least 90%, at least 95%, at least 97%, or at least 99% identical to the amino acid sequence of SEQ ID NO:207; a VH that is at least 90%, at least 95%, at least 97%, or at least 99% identical to the amino acid sequence of SEQ ID NO:209; a VH that is at least 90%, at least 95%, at least 97%, or at least 99% identical to the amino acid sequence of SEQ ID NO:234; a VH that is at least 90%, at least 95%, at least 97%, or at least 99% identical to the amino acid sequence of SEQ ID NO:235; a VH that is at least 90%, at least 95%, at least 97%, or at least 99% identical to the amino acid sequence of SEQ ID NO:236; a VH that is at least 90%, at least 95%, at least 97%, or at least 99% identical to the amino acid sequence of SEQ ID NO:237; a VH that is at least 90%, at least 95%, at least 97%, or at least 99% identical to the amino acid sequence of SEQ ID NO:238; a VH that is at least 90%, at least 95%, at least 97%, or at least 99% identical to the amino acid sequence of SEQ ID NO:236; a VH that is at least 90%, at least 95%, at least 97%, or at least 99% identical to the amino acid sequence of SEQ ID NO:237; s a VH that is at least 90%, at least 95%, at least 97%, or at least 99% identical to the amino acid sequence of SEQ ID NO:236; a VH that is at least 90%, at least 95%, at least 97%, or at least 99% identical to the amino acid sequence of SEQ ID NO:237; a VH that is at least 90%, at least 95%, at least 97%, or at least 99% identical to the amino acid sequence of SEQ ID NO:238; a VH that is at least 90%, at least 95%, at least 97%, or at least 99% identical to the amino acid sequence of SEQ ID NO:236; a VH that is at least 90%, at least 95%, at least 97%, or at least 99% identical to the amino acid sequence of SEQ ID NO:239; a VH that is at least 90%, at least 95%, at least 97%, or at least 99% identical to the amino acid sequence of SEQ ID NO:240; a VH that is at least 90%, at least 95%, at least 97%, or at least 99% identical to the amino acid sequence of SEQ ID NO:240; a VH that is at least 90%, at least 95%, at least 97%, or at least 99% identical to the amino acid sequence of SEQ ID NO:240; a VH that is at least 90%, at least 95%, at least 97%, or at least 99% identical to the amino acid sequence of SEQ ID NO:240; a VH that is at least 90%, at least 95%, at least 97%, or at least 99% identical to the amino acid sequence of SEQ ID NO:240; or a VH that is at least 90%, at least 95%, at least 97%, or at least 99% identical to the amino acid sequence of SEQ ID NO:240. In some cases, the antibody further comprises a VL that is at least 90%, at least 95%, at least 97%, or at least 99% identical to the amino acid sequence of SEQ ID NO: 162; a VL that is at least 90%, at least 95%, at least 97%, or at least 99% identical to the amino acid sequence of SEQ ID NO: 164; a VL that is at least 90%, at least 95%, at least 97%, or at least 99% identical to the amino acid sequence of SEQ ID NO: 166; a VL that is at least 90%, at least 95%, at least 97%, or at least 99% identical to the amino acid sequence of SEQ ID NO: 168; a VL that is at least 90%, at least 95%, at least 97%, or at least 99% identical to the amino acid sequence of SEQ ID NO: 170; a VL that is at least 90%, at least 95%, at least 97%, or at least 99% identical to the amino acid sequence of SEQ ID NO: 172; a VL that is at least 90%, at least 95%, at least 97%, or at least 99% identical to the amino acid sequence of SEQ ID NO: 174; a VL that is at least 90%, at least 95%, at least 97%, or at least 99% identical to the amino acid sequence of SEQ ID NO: 176; a VL that is at least 90%, at least 95%, at least 97%, or at least 99% identical to the amino acid sequence of SEQ ID NO: 178; a VL that is at least 90%, at least 95%, at least 97%, or at least 99% identical to the amino acid sequence of SEQ ID NO: 180; a VL that is at least 90%, at least 95%, at least 97%, or at least 99% identical to the amino acid sequence of SEQ ID NO: 182; a VL that is at least 90%, at least 95%, at least 97%, or at least 99% identical to the amino acid sequence of SEQ ID NO: 184; a VL that is at least 90%, at least 95%, at least 97%, or at least 99% identical to the amino acid sequence of SEQ ID NO: 186; a VL that is at least 90%, at least 95%, at least 97%, or at least 99% identical to the amino acid sequence of SEQ ID NO: 188; a VL that is at least 90%, at least 95%, at least 97%, or at least 99% identical to the amino acid sequence of SEQ ID NO: 190; a VL that is at least 90%, at least 95%, at least 97%, or at least 99% identical to the amino acid sequence of SEQ ID NO: 192; a VL that is at least 90%, at least 95%, at least 97%, or at least 99% identical to the amino acid sequence of SEQ ID NO: 194; a VL that is at least 90%, at least 95%, at least 97%, or at least 99% identical to the amino acid sequence of SEQ ID NO: 196; a VL that is at least 90%, at least 95%, at least 97%, or at least 99% identical to the amino acid sequence of SEQ ID NO: 198; a VL that is at least 90%, at least 95%, at least 97%, or at least 99% identical to the amino acid sequence of SEQ ID NO: 200; a VL that is at least 90%, at least 95%, at least 97%, or at least 99% identical to the amino acid sequence of SEQ ID NO: 202; a VL that is at least 90%, at least 95%, at least 97%, or at least 99% identical to the amino acid sequence of SEQ ID NO: 204; a VL that is at least 90%, at least 95%, at least 97%, or at least 99% identical to the amino acid sequence of SEQ ID NO: 206; a VL that is at least 90%, at least 95%, at least 97%, or at least 99% identical to the amino acid sequence of SEQ ID NO: 208; a VL that is at least 90%, at least 95%, at least 97%, or at least 99% identical to the amino acid sequence of SEQ ID NO: 210; a VL that is at least 90%, at least 95%, at least 97%, or at least 99% identical to the amino acid sequence of SEQ ID NO: 241; a VL that is at least 90%, at least 95%, at least 97%, or at least 99% identical to the amino acid sequence of SEQ ID NO: 242; a VL that is at least 90%, at least 95%, at least 97%, or at least 99% identical to the amino acid sequence of SEQ ID NO: 243; a VL that is at least 90%, at least 95%, at least 97%, or at least 99% identical to the amino acid sequence of SEQ ID NO: 243; a VL that is at least 90%, at least 95%, at least 97%, or at least 99% identical to the amino acid sequence of SEQ ID NO: 244; a VL that is at least 90%, at least 95%, at least 97%, or at least 99% identical to the amino acid sequence of SEQ ID NO: 245; a VL that is at least 90%, at least 95%, at least 97%, or at least 99% identical to the amino acid sequence of SEQ ID NO: 245; a VL that is at least 90%, at least 95%, at least 97%, or at least 99% identical to the amino acid sequence of SEQ ID NO: 246; a VL that is at least 90%, at least 95%, at least 97%, or at least 99% identical to the amino acid sequence of SEQ ID NO: 247; a VL that is at least 90%, at least 95%, at least 97%, or at least 99% identical to the amino acid sequence of SEQ ID NO: 247; a VL that is at least 90%, at least 95%, at least 97%, or at least 99% identical to the amino acid sequence of SEQ ID NO: 248; a VL that is at least 90%, at least 95%, at least 97%, or at least 99% identical to the amino acid sequence of SEQ ID NO: 249; a VL that is at least 90%, at least 95%, at least 97%, or at least 99% identical to the amino acid sequence of SEQ ID NO: 250; a VL that is at least 90%, at least 95%, at least 97%, or at least 99% identical to the amino acid sequence of SEQ ID NO: 251; a VL that is at least 90%, at least 95%, at least 97%, or at least 99% identical to the amino acid sequence of SEQ ID NO: 252; a VL that is at least 90%, at least 95%, at least 97%, or at least 99% identical to the amino acid sequence of SEQ ID NO: 253; a VL that is at least 90%, at least 95%, at least 97%, or at least 99% identical to the amino acid sequence of SEQ ID NO: 254; or a VL that is at least 90%, at least 95%, at least 97%, or at least 99% identical to the amino acid sequence of SEQ ID NO: 249. In yet further cases, the antibody comprises: a VH comprising the amino acid sequence of SEQ ID NO: 161; a VH comprising the amino acid sequence of SEQ ID NO:163; a VH comprising the amino acid sequence of SEQ ID NO:165; a VH comprising the amino acid sequence of SEQ ID NO:167; a VH comprising the amino acid sequence of SEQ ID NO:169; a VH comprising the amino acid sequence of SEQ ID NO:171; a VH comprising the amino acid sequence of SEQ ID NO:173; a VH comprising the amino acid sequence of SEQ ID NO:175; a VH comprising the amino acid sequence of SEQ ID NO:177; a VH comprising the amino acid sequence of SEQ ID NO:179; a VH comprising the amino acid sequence of SEQ ID NO:181; a VH comprising the amino acid sequence of SEQ ID NO:183; a VH comprising the amino acid sequence of SEQ ID NO:185; a VH comprising the amino acid sequence of SEQ ID NO:187; a VH comprising the amino acid sequence of SEQ ID NO:189; a VH comprising the amino acid sequence of SEQ ID NO:191; a VH comprising the amino acid sequence of SEQ ID NO:193; a VH comprising the amino acid sequence of SEQ ID NO:195; a VH comprising the amino acid sequence of SEQ ID NO:197; a VH comprising the amino acid sequence of SEQ ID NO:199; a VH comprising the amino acid sequence of SEQ ID NO:201; a VH comprising the amino acid sequence of SEQ ID NO:203; a VH comprising the amino acid sequence of SEQ ID NO:205; a VH comprising the amino acid sequence of SEQ ID NO:207; a VH comprising the amino acid sequence of SEQ ID NO:209; a VH comprising the amino acid sequence of SEQ ID NO:234; a VH comprising the amino acid sequence of SEQ ID NO:235; a VH comprising the amino acid sequence of SEQ ID NO:236; a VH comprising the amino acid sequence of SEQ ID NO:237; a VH comprising the amino acid sequence of SEQ ID NO:238; a VH comprising the amino acid sequence of SEQ ID NO:236; a VH comprising the amino acid sequence of SEQ ID NO:237; a VH comprising the amino acid sequence of SEQ ID NO:236; a VH comprising the amino acid sequence of SEQ ID NO:237; a VH comprising the amino acid sequence of SEQ ID NO:238; a VH comprising the amino acid sequence of SEQ ID NO:236; a VH comprising the amino acid sequence of SEQ ID NO:239; a VH comprising the amino acid sequence of SEQ ID NO:240; a VH comprising the amino acid sequence of SEQ ID NO:240; a VH comprising the amino acid sequence of SEQ ID NO:240; a VH comprising the amino acid sequence of SEQ ID NO:240; a VH comprising the amino acid sequence of SEQ ID NO:240; or a VH comprising the amino acid sequence of SEQ ID NO:240. And in yet further cases, the antibody comprises: a VL comprising the amino acid sequence of SEQ ID NO: 162; a VL comprising the amino acid sequence of SEQ ID NO: 164; a VL comprising the amino acid sequence of SEQ ID NO: 166; a VL comprising the amino acid sequence of SEQ ID NO: 168; a VL comprising the amino acid sequence of SEQ ID NO: 170; a VL comprising the amino acid sequence of SEQ ID NO: 172; a VL comprising the amino acid sequence of SEQ ID NO: 174; a VL comprising the amino acid sequence of SEQ ID NO: 176; a VL comprising the amino acid sequence of SEQ ID NO: 178; a VL comprising the amino acid sequence of SEQ ID NO: 180; a VL comprising the amino acid sequence of SEQ ID NO: 182; a VL comprising the amino acid sequence of SEQ ID NO: 184; a VL comprising the amino acid sequence of SEQ ID NO: 186; a VL comprising the amino acid sequence of SEQ ID NO: 188; a VL comprising the amino acid sequence of SEQ ID NO: 190; a VL comprising the amino acid sequence of SEQ ID NO: 192; a VL comprising the amino acid sequence of SEQ ID NO: 194; a VL comprising the amino acid sequence of SEQ ID NO: 196; a VL comprising the amino acid sequence of SEQ ID NO: 198; a VL comprising the amino acid sequence of SEQ ID NO: 200; a VL comprising the amino acid sequence of SEQ ID NO: 202; a VL comprising the amino acid sequence of SEQ ID NO: 204; a VL comprising the amino acid sequence of SEQ ID NO: 206; a VL comprising the amino acid sequence of SEQ ID NO: 208; a VL comprising the amino acid sequence of SEQ ID NO: 210; a VL comprising the amino acid sequence of SEQ ID NO: 241; a VL comprising the amino acid sequence of SEQ ID NO: 242; a VL comprising the amino acid sequence of SEQ ID NO: 243; a VL comprising the amino acid sequence of SEQ ID NO: 243; a VL comprising the amino acid sequence of SEQ ID NO: 244; a VL comprising the amino acid sequence of SEQ ID NO: 245; a VL comprising the amino acid sequence of SEQ ID NO: 246; a VL comprising the amino acid sequence of SEQ ID NO: 247; a VL comprising the amino acid sequence of SEQ ID NO: 247; a VL comprising the amino acid sequence of SEQ ID NO: 248; a VL comprising the amino acid sequence of SEQ ID NO: 249; a VL comprising the amino acid sequence of SEQ ID NO: 250; a VL comprising the amino acid sequence of SEQ ID NO: 251; a VL comprising the amino acid sequence of SEQ ID NO: 252; a VL comprising the amino acid sequence of SEQ ID NO: 253; a VL comprising the amino acid sequence of SEQ ID NO: 254; or a VL comprising the amino acid sequence of SEQ ID NO: 249. In some embodiments, the antibody comprises a VH comprising the amino acid sequence of SEQ ID NO: 161 and a VL comprising the amino acid sequence of SEQ ID NO: 162 (as shown in Table 1); a VH comprising the amino acid sequence of SEQ ID NO: 163 and a VL comprising the amino acid sequence of SEQ ID NO: 164 (as shown in Table 1); a VH comprising the amino acid sequence of SEQ ID NO: 165 and a VL comprising the amino acid sequence of SEQ ID NO: 166 (as shown in Table 1); a VH comprising the amino acid sequence of SEQ ID NO: 167 and a VL comprising the amino acid sequence of SEQ ID NO: 168 (as shown in Table 1); a VH comprising the amino acid sequence of SEQ ID NO: 169 and a VL comprising the amino acid sequence of SEQ ID NO: 170 (as shown in Table 1); a VH comprising the amino acid sequence of SEQ ID NO: 171 and a VL comprising the amino acid sequence of SEQ ID NO: 172 (as shown in Table 1); a VH comprising the amino acid sequence of SEQ ID NO: 173 and a VL comprising the amino acid sequence of SEQ ID NO: 174 (as shown in Table 1); a VH comprising the amino acid sequence of SEQ ID NO: 175 and a VL comprising the amino acid sequence of SEQ ID NO: 176 (as shown in Table 1); a VH comprising the amino acid sequence of SEQ ID NO: 177 and a VL comprising the amino acid sequence of SEQ ID NO: 178 (as shown in Table 1); a VH comprising the amino acid sequence of SEQ ID NO: 179 and a VL comprising the amino acid sequence of SEQ ID NO: 180 (as shown in Table 1); a VH comprising the amino acid sequence of SEQ ID NO: 181 and a VL comprising the amino acid sequence of SEQ ID NO: 182 (as shown in Table 1); a VH comprising the amino acid sequence of SEQ ID NO: 183 and a VL comprising the amino acid sequence of SEQ ID NO: 184 (as shown in Table 1); a VH comprising the amino acid sequence of SEQ ID NO: 185 and a VL comprising the amino acid sequence of SEQ ID NO: 186 (as shown in Table 1); a VH comprising the amino acid sequence of SEQ ID NO: 187 and a VL comprising the amino acid sequence of SEQ ID NO: 188 (as shown in Table 1); a VH comprising the amino acid sequence of SEQ ID NO: 189 and a VL comprising the amino acid sequence of SEQ ID NO: 190 (as shown in Table 1); a VH comprising the amino acid sequence of SEQ ID NO: 191 and a VL comprising the amino acid sequence of SEQ ID NO: 192 (as shown in Table 1); a VH comprising the amino acid sequence of SEQ ID NO: 193 and a VL comprising the amino acid sequence of SEQ ID NO: 194 (as shown in Table 1); a VH comprising the amino acid sequence of SEQ ID NO: 195 and a VL comprising the amino acid sequence of SEQ ID NO: 196 (as shown in Table 1); a VH comprising the amino acid sequence of SEQ ID NO: 197 and a VL comprising the amino acid sequence of SEQ ID NO: 198 (as shown in Table 1); a VH comprising the amino acid sequence of SEQ ID NO: 199 and a VL comprising the amino acid sequence of SEQ ID NO: 200 (as shown in Table 1); a VH comprising the amino acid sequence of SEQ ID NO: 201 and a VL comprising the amino acid sequence of SEQ ID NO: 202 (as shown in Table 1); a VH comprising the amino acid sequence of SEQ ID NO: 203 and a VL comprising the amino acid sequence of SEQ ID NO: 204 (as shown in Table 1); a VH comprising the amino acid sequence of SEQ ID NO: 205 and a VL comprising the amino acid sequence of SEQ ID NO: 206 (as shown in Table 1); a VH comprising the amino acid sequence of SEQ ID NO: 207 and a VL comprising the amino acid sequence of SEQ ID NO: 208 (as shown in Table 1); a VH comprising the amino acid sequence of SEQ ID NO: 209 and a VL comprising the amino acid sequence of SEQ ID NO: 210 (as shown in Table 1); a VH comprising the amino acid sequence of SEQ ID NO: 234 and a VL comprising the amino acid sequence of SEQ ID NO: 241 (as shown in Table 15); a VH comprising the amino acid sequence of SEQ ID NO: 235 and a VL comprising the amino acid sequence of SEQ ID NO: 242 (as shown in Table 15); a VH comprising the amino acid sequence of SEQ ID NO: 236 and a VL comprising the amino acid sequence of SEQ ID NO: 243 (as shown in Table 15); a VH comprising the amino acid sequence of SEQ ID NO: 237 and a VL comprising the amino acid sequence of SEQ ID NO: 243 (as shown in Table 15); a VH comprising the amino acid sequence of SEQ ID NO: 238 and a VL comprising the amino acid sequence of SEQ ID NO: 244 (as shown in Table 15); a VH comprising the amino acid sequence of SEQ ID NO: 236 and a VL comprising the amino acid sequence of SEQ ID NO: 245 (as shown in Table 15); a VH comprising the amino acid sequence of SEQ ID NO: 237 and a VL comprising the amino acid sequence of SEQ ID NO: 245 (as shown in Table 15); a VH comprising the amino acid sequence of SEQ ID NO: 236 and a VL comprising the amino acid sequence of SEQ ID NO: 246 (as shown in Table 15); a VH comprising the amino acid sequence of SEQ ID NO: 237 and a VL comprising the amino acid sequence of SEQ ID NO: 247 (as shown in Table 15); a VH comprising the amino acid sequence of SEQ ID NO: 238 and a VL comprising the amino acid sequence of SEQ ID NO: 247 (as shown in Table 15); a VH comprising the amino acid sequence of SEQ ID NO: 236 and a VL comprising the amino acid sequence of SEQ ID NO: 248 (as shown in Table 15); a VH comprising the amino acid sequence of SEQ ID NO: 239 and a VL comprising the amino acid sequence of SEQ ID NO: 249 (as shown in Table 15); a VH comprising the amino acid sequence of SEQ ID NO: 240 and a VL comprising the amino acid sequence of SEQ ID NO: 250 (as shown in Table 15); a VH comprising the amino acid sequence of SEQ ID NO: 240 and a VL comprising the amino acid sequence of SEQ ID NO: 251 (as shown in Table 15); a VH comprising the amino acid sequence of SEQ ID NO: 240 and a VL comprising the amino acid sequence of SEQ ID NO: 252 (as shown in Table 15); a VH comprising the amino acid sequence of SEQ ID NO: 240 and a VL comprising the amino acid sequence of SEQ ID NO: 253 (as shown in Table 15); a VH comprising the amino acid sequence of SEQ ID NO: 240 and a VL comprising the amino acid sequence of SEQ ID NO: 254 (as shown in Table 15); or a VH comprising the amino acid sequence of SEQ ID NO: 240 and a VL comprising the amino acid sequence of SEQ ID NO: 249 (as shown in Table 15).

In some embodiments, the antibody comprises a heavy chain that is at least 90%, at least 95%, at least 97%, or at least 99% identical to the amino acid sequence of SEQ ID NO:290; a heavy chain that is at least 90%, at least 95%, at least 97%, or at least 99% identical to the amino acid sequence of SEQ ID NO:291; a heavy chain that is at least 90%, at least 95%, at least 97%, or at least 99% identical to the amino acid sequence of SEQ ID NO:292; a heavy chain that is at least 90%, at least 95%, at least 97%, or at least 99% identical to the amino acid sequence of SEQ ID NO:293; a heavy chain that is at least 90%, at least 95%, at least 97%, or at least 99% identical to the amino acid sequence of SEQ ID NO:294; a heavy chain that is at least 90%, at least 95%, at least 97%, or at least 99% identical to the amino acid sequence of SEQ ID NO:295; a heavy chain that is at least 90%, at least 95%, at least 97%, or at least 99% identical to the amino acid sequence of SEQ ID NO:296; a heavy chain that is at least 90%, at least 95%, at least 97%, or at least 99% identical to the amino acid sequence of SEQ ID NO:297; a heavy chain that is at least 90%, at least 95%, at least 97%, or at least 99% identical to the amino acid sequence of SEQ ID NO:298; a heavy chain that is at least 90%, at least 95%, at least 97%, or at least 99% identical to the amino acid sequence of SEQ ID NO:299; a heavy chain that is at least 90%, at least 95%, at least 97%, or at least 99% identical to the amino acid sequence of SEQ ID NO:300; a heavy chain that is at least 90%, at least 95%, at least 97%, or at least 99% identical to the amino acid sequence of SEQ ID NO:301; a heavy chain that is at least 90%, at least 95%, at least 97%, or at least 99% identical to the amino acid sequence of SEQ ID NO:302; a heavy chain that is at least 90%, at least 95%, at least 97%, or at least 99% identical to the amino acid sequence of SEQ ID NO:303; a heavy chain that is at least 90%, at least 95%, at least 97%, or at least 99% identical to the amino acid sequence of SEQ ID NO:304; a heavy chain that is at least 90%, at least 95%, at least 97%, or at least 99% identical to the amino acid sequence of SEQ ID NO:305; a heavy chain that is at least 90%, at least 95%, at least 97%, or at least 99% identical to the amino acid sequence of SEQ ID NO:306; a heavy chain that is at least 90%, at least 95%, at least 97%, or at least 99% identical to the amino acid sequence of SEQ ID NO:307; a heavy chain that is at least 90%, at least 95%, at least 97%, or at least 99% identical to the amino acid sequence of SEQ ID NO:308; a heavy chain that is at least 90%, at least 95%, at least 97%, or at least 99% identical to the amino acid sequence of SEQ ID NO:309; a heavy chain that is at least 90%, at least 95%, at least 97%, or at least 99% identical to the amino acid sequence of SEQ ID NO:310; a heavy chain that is at least 90%, at least 95%, at least 97%, or at least 99% identical to the amino acid sequence of SEQ ID NO:311; a heavy chain that is at least 90%, at least 95%, at least 97%, or at least 99% identical to the amino acid sequence of SEQ ID NO:312; a heavy chain that is at least 90%, at least 95%, at least 97%, or at least 99% identical to the amino acid sequence of SEQ ID NO:313; a heavy chain that is at least 90%, at least 95%, at least 97%, or at least 99% identical to the amino acid sequence of SEQ ID NO:314; a heavy chain that is at least 90%, at least 95%, at least 97%, or at least 99% identical to the amino acid sequence of SEQ ID NO:315; a heavy chain that is at least 90%, at least 95%, at least 97%, or at least 99% identical to the amino acid sequence of SEQ ID NO:316; a heavy chain that is at least 90%, at least 95%, at least 97%, or at least 99% identical to the amino acid sequence of SEQ ID NO:317; a heavy chain that is at least 90%, at least 95%, at least 97%, or at least 99% identical to the amino acid sequence of SEQ ID NO:318; a heavy chain that is at least 90%, at least 95%, at least 97%, or at least 99% identical to the amino acid sequence of SEQ ID NO:319; a heavy chain that is at least 90%, at least 95%, at least 97%, or at least 99% identical to the amino acid sequence of SEQ ID NO:320; a heavy chain that is at least 90%, at least 95%, at least 97%, or at least 99% identical to the amino acid sequence of SEQ ID NO:321; a heavy chain that is at least 90%, at least 95%, at least 97%, or at least 99% identical to the amino acid sequence of SEQ ID NO:322; a heavy chain that is at least 90%, at least 95%, at least 97%, or at least 99% identical to the amino acid sequence of SEQ ID NO:323; a heavy chain that is at least 90%, at least 95%, at least 97%, or at least 99% identical to the amino acid sequence of SEQ ID NO:324; a heavy chain that is at least 90%, at least 95%, at least 97%, or at least 99% identical to the amino acid sequence of SEQ ID NO:325; a heavy chain that is at least 90%, at least 95%, at least 97%, or at least 99% identical to the amino acid sequence of SEQ ID NO:326; a heavy chain that is at least 90%, at least 95%, at least 97%, or at least 99% identical to the amino acid sequence of SEQ ID NO:327; a heavy chain that is at least 90%, at least 95%, at least 97%, or at least 99% identical to the amino acid sequence of SEQ ID NO:328; a heavy chain that is at least 90%, at least 95%, at least 97%, or at least 99% identical to the amino acid sequence of SEQ ID NO:329; a heavy chain that is at least 90%, at least 95%, at least 97%, or at least 99% identical to the amino acid sequence of SEQ ID NO:330; a heavy chain that is at least 90%, at least 95%, at least 97%, or at least 99% identical to the amino acid sequence of SEQ ID NO:331; a heavy chain that is at least 90%, at least 95%, at least 97%, or at least 99% identical to the amino acid sequence of SEQ ID NO:332; a heavy chain that is at least 90%, at least 95%, at least 97%, or at least 99% identical to the amino acid sequence of SEQ ID NO:333; a heavy chain that is at least 90%, at least 95%, at least 97%, or at least 99% identical to the amino acid sequence of SEQ ID NO:334; a heavy chain that is at least 90%, at least 95%, at least 97%, or at least 99% identical to the amino acid sequence of SEQ ID NO:335; a heavy chain that is at least 90%, at least 95%, at least 97%, or at least 99% identical to the amino acid sequence of SEQ ID NO:336; a heavy chain that is at least 90%, at least 95%, at least 97%, or at least 99% identical to the amino acid sequence of SEQ ID NO:337; a heavy chain that is at least 90%, at least 95%, at least 97%, or at least 99% identical to the amino acid sequence of SEQ ID NO:338; a heavy chain that is at least 90%, at least 95%, at least 97%, or at least 99% identical to the amino acid sequence of SEQ ID NO:339; a heavy chain that is at least 90%, at least 95%, at least 97%, or at least 99% identical to the amino acid sequence of SEQ ID NO:340; a heavy chain that is at least 90%, at least 95%, at least 97%, or at least 99% identical to the amino acid sequence of SEQ ID NO:341; a heavy chain that is at least 90%, at least 95%, at least 97%, or at least 99% identical to the amino acid sequence of SEQ ID NO:342; a heavy chain that is at least 90%, at least 95%, at least 97%, or at least 99% identical to the amino acid sequence of SEQ ID NO:343; a heavy chain that is at least 90%, at least 95%, at least 97%, or at least 99% identical to the amino acid sequence of SEQ ID NO:344; a heavy chain that is at least 90%, at least 95%, at least 97%, or at least 99% identical to the amino acid sequence of SEQ ID NO:345; a heavy chain that is at least 90%, at least 95%, at least 97%, or at least 99% identical to the amino acid sequence of SEQ ID NO:346; a heavy chain that is at least 90%, at least 95%, at least 97%, or at least 99% identical to the amino acid sequence of SEQ ID NO:347; a heavy chain that is at least 90%, at least 95%, at least 97%, or at least 99% identical to the amino acid sequence of SEQ ID NO:348; a heavy chain that is at least 90%, at least 95%, at least 97%, or at least 99% identical to the amino acid sequence of SEQ ID NO:349; a heavy chain that is at least 90%, at least 95%, at least 97%, or at least 99% identical to the amino acid sequence of SEQ ID NO:350; a heavy chain that is at least 90%, at least 95%, at least 97%, or at least 99% identical to the amino acid sequence of SEQ ID NO:351; a heavy chain that is at least 90%, at least 95%, at least 97%, or at least 99% identical to the amino acid sequence of SEQ ID NO:352; a heavy chain that is at least 90%, at least 95%, at least 97%, or at least 99% identical to the amino acid sequence of SEQ ID NO:353; a heavy chain that is at least 90%, at least 95%, at least 97%, or at least 99% identical to the amino acid sequence of SEQ ID NO:354; a heavy chain that is at least 90%, at least 95%, at least 97%, or at least 99% identical to the amino acid sequence of SEQ ID NO:355; a heavy chain that is at least 90%, at least 95%, at least 97%, or at least 99% identical to the amino acid sequence of SEQ ID NO:356; a heavy chain that is at least 90%, at least 95%, at least 97%, or at least 99% identical to the amino acid sequence of SEQ ID NO:357; a heavy chain that is at least 90%, at least 95%, at least 97%, or at least 99% identical to the amino acid sequence of SEQ ID NO:358; a heavy chain that is at least 90%, at least 95%, at least 97%, or at least 99% identical to the amino acid sequence of SEQ ID NO:359; a heavy chain that is at least 90%, at least 95%, at least 97%, or at least 99% identical to the amino acid sequence of SEQ ID NO:360; a heavy chain that is at least 90%, at least 95%, at least 97%, or at least 99% identical to the amino acid sequence of SEQ ID NO:361; a heavy chain that is at least 90%, at least 95%, at least 97%, or at least 99% identical to the amino acid sequence of SEQ ID NO:237; a heavy chain that is at least 90%, at least 95%, at least 97%, or at least 99% identical to the amino acid sequence of SEQ ID NO:362; a heavy chain that is at least 90%, at least 95%, at least 97%, or at least 99% identical to the amino acid sequence of SEQ ID NO:363; a heavy chain that is at least 90%, at least 95%, at least 97%, or at least 99% identical to the amino acid sequence of SEQ ID NO:364; a heavy chain that is at least 90%, at least 95%, at least 97%, or at least 99% identical to the amino acid sequence of SEQ ID NO:365; a heavy chain that is at least 90%, at least 95%, at least 97%, or at least 99% identical to the amino acid sequence of SEQ ID NO:366; a heavy chain that is at least 90%, at least 95%, at least 97%, or at least 99% identical to the amino acid sequence of SEQ ID NO:367; a heavy chain that is at least 90%, at least 95%, at least 97%, or at least 99% identical to the amino acid sequence of SEQ ID NO:368; a heavy chain that is at least 90%, at least 95%, at least 97%, or at least 99% identical to the amino acid sequence of SEQ ID NO:369; a heavy chain that is at least 90%, at least 95%, at least 97%, or at least 99% identical to the amino acid sequence of SEQ ID NO:370; a heavy chain that is at least 90%, at least 95%, at least 97%, or at least 99% identical to the amino acid sequence of SEQ ID NO:371; a heavy chain that is at least 90%, at least 95%, at least 97%, or at least 99% identical to the amino acid sequence of SEQ ID NO:372; a heavy chain that is at least 90%, at least 95%, at least 97%, or at least 99% identical to the amino acid sequence of SEQ ID NO:373; a heavy chain that is at least 90%, at least 95%, at least 97%, or at least 99% identical to the amino acid sequence of SEQ ID NO:374; a heavy chain that is at least 90%, at least 95%, at least 97%, or at least 99% identical to the amino acid sequence of SEQ ID NO:375; a heavy chain that is at least 90%, at least 95%, at least 97%, or at least 99% identical to the amino acid sequence of SEQ ID NO:376; a heavy chain that is at least 90%, at least 95%, at least 97%, or at least 99% identical to the amino acid sequence of SEQ ID NO:377; a heavy chain that is at least 90%, at least 95%, at least 97%, or at least 99% identical to the amino acid sequence of SEQ ID NO:378; a heavy chain that is at least 90%, at least 95%, at least 97%, or at least 99% identical to the amino acid sequence of SEQ ID NO:379; a heavy chain that is at least 90%, at least 95%, at least 97%, or at least 99% identical to the amino acid sequence of SEQ ID NO:380; a heavy chain that is at least 90%, at least 95%, at least 97%, or at least 99% identical to the amino acid sequence of SEQ ID NO:381; comprises a heavy chain that is at least 90%, at least 95%, at least 97%, or at least 99% identical to the amino acid sequence of SEQ ID NO:382; a heavy chain that is at least 90%, at least 95%, at least 97%, or at least 99% identical to the amino acid sequence of SEQ ID NO:383; a heavy chain that is at least 90%, at least 95%, at least 97%, or at least 99% identical to the amino acid sequence of SEQ ID NO:384; a heavy chain that is at least 90%, at least 95%, at least 97%, or at least 99% identical to the amino acid sequence of SEQ ID NO:385; a heavy chain that is at least 90%, at least 95%, at least 97%, or at least 99% identical to the amino acid sequence of SEQ ID NO:386; a heavy chain that is at least 90%, at least 95%, at least 97%, or at least 99% identical to the amino acid sequence of SEQ ID NO:387; a heavy chain that is at least 90%, at least 95%, at least 97%, or at least 99% identical to the amino acid sequence of SEQ ID NO:388; a heavy chain that is at least 90%, at least 95%, at least 97%, or at least 99% identical to the amino acid sequence of SEQ ID NO:389; a heavy chain that is at least 90%, at least 95%, at least 97%, or at least 99% identical to the amino acid sequence of SEQ ID NO:390; a heavy chain that is at least 90%, at least 95%, at least 97%, or at least 99% identical to the amino acid sequence of SEQ ID NO:391; a heavy chain that is at least 90%, at least 95%, at least 97%, or at least 99% identical to the amino acid sequence of SEQ ID NO:392; a heavy chain that is at least 90%, at least 95%, at least 97%, or at least 99% identical to the amino acid sequence of SEQ ID NO:393; a heavy chain that is at least 90%, at least 95%, at least 97%, or at least 99% identical to the amino acid sequence of SEQ ID NO:394; a heavy chain that is at least 90%, at least 95%, at least 97%, or at least 99% identical to the amino acid sequence of SEQ ID NO:395; a heavy chain that is at least 90%, at least 95%, at least 97%, or at least 99% identical to the amino acid sequence of SEQ ID NO:396; a heavy chain that is at least 90%, at least 95%, at least 97%, or at least 99% identical to the amino acid sequence of SEQ ID NO:397; a heavy chain that is at least 90%, at least 95%, at least 97%, or at least 99% identical to the amino acid sequence of SEQ ID NO:398; a heavy chain that is at least 90%, at least 95%, at least 97%, or at least 99% identical to the amino acid sequence of SEQ ID NO:399; a heavy chain that is at least 90%, at least 95%, at least 97%, or at least 99% identical to the amino acid sequence of SEQ ID NO:400; comprises a heavy chain that is at least 90%, at least 95%, at least 97%, or at least 99% identical to the amino acid sequence of SEQ ID NO:401; a heavy chain that is at least 90%, at least 95%, at least 97%, or at least 99% identical to the amino acid sequence of SEQ ID NO:402; a heavy chain that is at least 90%, at least 95%, at least 97%, or at least 99% identical to the amino acid sequence of SEQ ID NO:403; a heavy chain that is at least 90%, at least 95%, at least 97%, or at least 99% identical to the amino acid sequence of SEQ ID NO:404; a heavy chain that is at least 90%, at least 95%, at least 97%, or at least 99% identical to the amino acid sequence of SEQ ID NO:405; a heavy chain that is at least 90%, at least 95%, at least 97%, or at least 99% identical to the amino acid sequence of SEQ ID NO:406; a heavy chain that is at least 90%, at least 95%, at least 97%, or at least 99% identical to the amino acid sequence of SEQ ID NO:407; a heavy chain that is at least 90%, at least 95%, at least 97%, or at least 99% identical to the amino acid sequence of SEQ ID NO:408; a heavy chain that is at least 90%, at least 95%, at least 97%, or at least 99% identical to the amino acid sequence of SEQ ID NO:409; a heavy chain that is at least 90%, at least 95%, at least 97%, or at least 99% identical to the amino acid sequence of SEQ ID NO:410; a heavy chain that is at least 90%, at least 95%, at least 97%, or at least 99% identical to the amino acid sequence of SEQ ID NO:411; a heavy chain that is at least 90%, at least 95%, at least 97%, or at least 99% identical to the amino acid sequence of SEQ ID NO:412; a heavy chain that is at least 90%, at least 95%, at least 97%, or at least 99% identical to the amino acid sequence of SEQ ID NO:413; a heavy chain that is at least 90%, at least 95%, at least 97%, or at least 99% identical to the amino acid sequence of SEQ ID NO:414; or a heavy chain that is at least 90%, at least 95%, at least 97%, or at least 99% identical to the amino acid sequence of SEQ ID NO:415. In some embodiments, the antibody comprises a light chain that is at least 90%, at least 95%, at least 97%, or at least 99% identical to the amino acid sequence of SEQ ID NO:416; a light chain that is at least 90%, at least 95%, at least 97%, or at least 99% identical to the amino acid sequence of SEQ ID NO:417; a light chain that is at least 90%, at least 95%, at least 97%, or at least 99% identical to the amino acid sequence of SEQ ID NO:418; a light chain that is at least 90%, at least 95%, at least 97%, or at least 99% identical to the amino acid sequence of SEQ ID NO:419; a light chain that is at least 90%, at least 95%, at least 97%, or at least 99% identical to the amino acid sequence of SEQ ID NO:420; a light chain that is at least 90%, at least 95%, at least 97%, or at least 99% identical to the amino acid sequence of SEQ ID NO:421; a light chain that is at least 90%, at least 95%, at least 97%, or at least 99% identical to the amino acid sequence of SEQ ID NO:422; a light chain that is at least 90%, at least 95%, at least 97%, or at least 99% identical to the amino acid sequence of SEQ ID NO:423; a light chain that is at least 90%, at least 95%, at least 97%, or at least 99% identical to the amino acid sequence of SEQ ID NO:424; a light chain that is at least 90%, at least 95%, at least 97%, or at least 99% identical to the amino acid sequence of SEQ ID NO:425; a light chain that is at least 90%, at least 95%, at least 97%, or at least 99% identical to the amino acid sequence of SEQ ID NO:426; a light chain that is at least 90%, at least 95%, at least 97%, or at least 99% identical to the amino acid sequence of SEQ ID NO:427; a light chain that is at least 90%, at least 95%, at least 97%, or at least 99% identical to the amino acid sequence of SEQ ID NO:428; a light chain that is at least 90%, at least 95%, at least 97%, or at least 99% identical to the amino acid sequence of SEQ ID NO:429; a light chain that is at least 90%, at least 95%, at least 97%, or at least 99% identical to the amino acid sequence of SEQ ID NO:430; a light chain that is at least 90%, at least 95%, at least 97%, or at least 99% identical to the amino acid sequence of SEQ ID NO:431; a light chain that is at least 90%, at least 95%, at least 97%, or at least 99% identical to the amino acid sequence of SEQ ID NO:432; or a light chain that is at least 90%, at least 95%, at least 97%, or at least 99% identical to the amino acid sequence of SEQ ID NO:433. In some embodiments, the antibody comprises a heavy chain that is at least 90%, at least 95%, at least 97%, or at least 99% identical to the amino acid sequence of SEQ ID NO:290 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:291 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:292 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:293 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:294 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:295 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:296 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:297 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:298 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:299 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:300 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:301 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:302 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:303 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:304 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:305 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:306 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:307 and a light chain that is at least 90%, at least 95%, at least 97%, or at least 99% identical to the amino acid sequence of SEQ ID NO:416; a heavy chain that is at least 90%, at least 95%, at least 97%, or at least 99% identical to the amino acid sequence of SEQ ID NO:308 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:309 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:310 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:311 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:312 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:313 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:314 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:315 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:316 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:317 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:318 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:319 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:320 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:321 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:322 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:323 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:324 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:325 and a light chain that is at least 90%, at least 95%, at least 97%, or at least 99% identical to the amino acid sequence of SEQ ID NO:417; a heavy chain that is at least 90%, at least 95%, at least 97%, or at least 99% identical to the amino acid sequence of SEQ ID NO:326 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:327 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:328 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:329 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:330 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:331 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:332 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:333 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:334 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:335 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:336 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:337 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:338 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:339 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:340 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:341 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:342 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:343 and a light chain that is at least 90%, at least 95%, at least 97%, or at least 99% identical to the amino acid sequence of SEQ ID NO:418; a heavy chain that is at least 90%, at least 95%, at least 97%, or at least 99% identical to the amino acid sequence of SEQ ID NO:344 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:345 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:346 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:347 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:348 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:349 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:350 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:351 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:352 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:353 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:354 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:355 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:356 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:357 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:358 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:359 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:360 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:361 and a light chain that is at least 90%, at least 95%, at least 97%, or at least 99% identical to the amino acid sequence of SEQ ID NO:419; a heavy chain that is at least 90%, at least 95%, at least 97%, or at least 99% identical to the amino acid sequence of SEQ ID NO:362 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:363 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:364 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:365 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:366 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:367 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:368 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:369 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:370 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:371 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:372 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:373 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:374 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:375 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:376 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:377 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:378 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:379 and a light chain that is at least 90%, at least 95%, at least 97%, or at least 99% identical to the amino acid sequence of SEQ ID NO:420; a heavy chain that is at least 90%, at least 95%, at least 97%, or at least 99% identical to the amino acid sequence of SEQ ID NO:326 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:327 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:328 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:329 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:330 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:331 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:332 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:333 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:334 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:335 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:336 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:337 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:338 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:339 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:340 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:341 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:342 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:343 and a light chain that is at least 90%, at least 95%, at least 97%, or at least 99% identical to the amino acid sequence of SEQ ID NO:421; a heavy chain that is at least 90%, at least 95%, at least 97%, or at least 99% identical to the amino acid sequence of SEQ ID NO:344 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:345 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:346 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:347 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:348 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:349 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:350 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:351 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:352 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:353 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:354 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:355 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:356 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:357 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:358 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:359 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:360 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:361 and a light chain that is at least 90%, at least 95%, at least 97%, or at least 99% identical to the amino acid sequence of SEQ ID NO:422; a heavy chain that is at least 90%, at least 95%, at least 97%, or at least 99% identical to the amino acid sequence of SEQ ID NO:326 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:327 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:328 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:329 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:330 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:331 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:332 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:333 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:334 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:335 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:336 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:337 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:338 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:339 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:340 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:341 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:342 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:343 and a light chain that is at least 90%, at least 95%, at least 97%, or at least 99% identical to the amino acid sequence of SEQ ID NO:423; a heavy chain that is at least 90%, at least 95%, at least 97%, or at least 99% identical to the amino acid sequence of SEQ ID NO:344 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:345 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:346 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:347 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:348 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:349 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:350 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:351 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:352 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:353 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:354 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:355 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:356 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:357 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:358 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:359 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:360 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:361 and a light chain that is at least 90%, at least 95%, at least 97%, or at least 99% identical to the amino acid sequence of SEQ ID NO:424; a heavy chain that is at least 90%, at least 95%, at least 97%, or at least 99% identical to the amino acid sequence of SEQ ID NO:362 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:363 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:364 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:365 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:366 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:367 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:368 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:369 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:370 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:371 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:372 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:373 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:374 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:375 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:376 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:377 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:378 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:379 and a light chain that is at least 90%, at least 95%, at least 97%, or at least 99% identical to the amino acid sequence of SEQ ID NO:425; a heavy chain that is at least 90%, at least 95%, at least 97%, or at least 99% identical to the amino acid sequence of SEQ ID NO:326 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:327 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:328 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:329 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:330 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:331 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:332 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:333 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:334 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:335 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:336 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:337 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:338 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:339 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:340 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:341 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:342 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:343 and a light chain that is at least 90%, at least 95%, at least 97%, or at least 99% identical to the amino acid sequence of SEQ ID NO:426; a heavy chain that is at least 90%, at least 95%, at least 97%, or at least 99% identical to the amino acid sequence of SEQ ID NO:380 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:381 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:382 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:383 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:384 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:385 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:386 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:387 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:388 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:389 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:390 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:391 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:392 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:393 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:394 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:395 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:396 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:397 and a light chain that is at least 90%, at least 95%, at least 97%, or at least 99% identical to the amino acid sequence of SEQ ID NO:427; a heavy chain that is at least 90%, at least 95%, at least 97%, or at least 99% identical to the amino acid sequence of SEQ ID NO:398 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:399 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:400 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:401 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:402 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:403 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:404 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:405 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:406 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:407 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:408 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:409 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:410 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:411 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:412 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:413 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:414 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:415 and a light chain that is at least 90%, at least 95%, at least 97%, or at least 99% identical to the amino acid sequence of SEQ ID NO:428; a heavy chain that is at least 90%, at least 95%, at least 97%, or at least 99% identical to the amino acid sequence of SEQ ID NO:398 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:399 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:400 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:401 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:402 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:403 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:404 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:405 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:406 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:407 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:408 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:409 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:410 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:411 and a light chain that is at least 90%, at least 95%, at least 97%, or at least 99% identical to the amino acid sequence of SEQ ID NO:429; a heavy chain that is at least 90%, at least 95%, at least 97%, or at least 99% identical to the amino acid sequence of SEQ ID NO:398 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:399 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:400 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:401 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:402 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:403 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:404 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:405 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:406 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:407 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:408 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:409 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:410 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:411 and a light chain that is at least 90%, at least 95%, at least 97%, or at least 99% identical to the amino acid sequence of SEQ ID NO:430; a heavy chain that is at least 90%, at least 95%, at least 97%, or at least 99% identical to the amino acid sequence of SEQ ID NO:398 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:399 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:400 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:401 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:402 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:403 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:404 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:405 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:406 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:407 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:408 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:409 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:410 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:411 and a light chain that is at least 90%, at least 95%, at least 97%, or at least 99% identical to the amino acid sequence of SEQ ID NO:431; a heavy chain that is at least 90%, at least 95%, at least 97%, or at least 99% identical to the amino acid sequence of SEQ ID NO:398 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:399 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:400 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:401 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:402 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:403 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:404 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:405 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:406 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:407 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:408 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:409 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:410 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:411 and a light chain that is at least 90%, at least 95%, at least 97%, or at least 99% identical to the amino acid sequence of SEQ ID NO:432; a heavy chain that is at least 90%, at least 95%, at least 97%, or at least 99% identical to the amino acid sequence of SEQ ID NO:398 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:399 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:400 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:401 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:402 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:403 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:404 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:405 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:406 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:407 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:408 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:409 and a light chain that is at least 90%, at least 95%, at least 97%, or at least 99% identical to the amino acid sequence of SEQ ID NO:433; a heavy chain that is at least 90%, at least 95%, at least 97%, or at least 99% identical to the amino acid sequence of SEQ ID NO:410 and a light chain that is at least 90%, at least 95%, at least 97%, or at least 99% identical to the amino acid sequence of SEQ ID NO:433; or a heavy chain that is at least 90%, at least 95%, at least 97%, or at least 99% identical to the amino acid sequence of SEQ ID NO:411 and a light chain that is at least 90%, at least 95%, at least 97%, or at least 99% identical to the amino acid sequence of SEQ ID NO:433.

In some embodiments the disclosure herein relates to an anti-Glycoprotein nonmetastatic melanoma protein B (GPNMB) antibody comprising a heavy chain comprising the amino acid sequence of SEQ ID NO:290 or SEQ ID NO:291 and a light chain comprising the amino acid sequence of SEQ ID NO:416; a heavy chain comprising the amino acid sequence of SEQ ID NO:292 or SEQ ID NO:293 and a light chain comprising the amino acid sequence of SEQ ID NO:416; a heavy chain comprising the amino acid sequence of SEQ ID NO:294 or SEQ ID NO:295 and a light chain comprising the amino acid sequence of SEQ ID NO:416; a heavy chain comprising the amino acid sequence of SEQ ID NO:296 or SEQ ID NO:297 and a light chain comprising the amino acid sequence of SEQ ID NO:416; a heavy chain comprising the amino acid sequence of SEQ ID NO:298 or SEQ ID NO:299 and a light chain comprising the amino acid sequence of SEQ ID NO:416; a heavy chain comprising the amino acid sequence of SEQ ID NO:300 SEQ ID NO:301 and a light chain comprising the amino acid sequence of SEQ ID NO:416; a heavy chain comprising the amino acid sequence of SEQ ID NO:302 or SEQ ID NO:303 and a light chain comprising the amino acid sequence of SEQ ID NO:416; a heavy chain comprising the amino acid sequence of SEQ ID NO:304 SEQ ID NO:305 and a light chain comprising the amino acid sequence of SEQ ID NO:416; a heavy chain comprising the amino acid sequence of SEQ ID NO:306 or SEQ ID NO:307 and a light chain comprising the amino acid sequence of SEQ ID NO:416; a heavy chain comprising the amino acid sequence of SEQ ID NO:308 or SEQ ID NO:309 and a light chain comprising the amino acid sequence of SEQ ID NO:417; a heavy chain comprising the amino acid sequence of SEQ ID NO:310 or SEQ ID NO:311 and a light chain comprising the amino acid sequence of SEQ ID NO:417; a heavy chain comprising the amino acid sequence of SEQ ID NO:312 or SEQ ID NO:313 and a light chain comprising the amino acid sequence of SEQ ID NO:417; a heavy chain comprising the amino acid sequence of SEQ ID NO:314 or SEQ ID NO:315 and a light chain comprising the amino acid sequence of SEQ ID NO:417; a heavy chain comprising the amino acid sequence of SEQ ID NO:316 or SEQ ID NO:317 and a light chain comprising the amino acid sequence of SEQ ID NO:417; a heavy chain comprising the amino acid sequence of SEQ ID NO:318 or SEQ ID NO:319 and a light chain comprising the amino acid sequence of SEQ ID NO:417; a heavy chain comprising the amino acid sequence of SEQ ID NO:320 or SEQ ID NO:321 and a light chain comprising the amino acid sequence of SEQ ID NO:417; a heavy chain comprising the amino acid sequence of SEQ ID NO:322 or SEQ ID NO:323 and a light chain comprising the amino acid sequence of SEQ ID NO:417; a heavy chain comprising the amino acid sequence of SEQ ID NO:324 or SEQ ID NO:325 and a light chain comprising the amino acid sequence of SEQ ID NO:417; a heavy chain comprising the amino acid sequence of SEQ ID NO:326 or SEQ ID NO:327 and a light chain comprising the amino acid sequence of SEQ ID NO:418; a heavy chain comprising the amino acid sequence of SEQ ID NO:328 or SEQ ID NO:329 and a light chain comprising the amino acid sequence of SEQ ID NO:418; a heavy chain comprising the amino acid sequence of SEQ ID NO:330 or SEQ ID NO:331 and a light chain comprising the amino acid sequence of SEQ ID NO:418; a heavy chain comprising the amino acid sequence of SEQ ID NO:332 or SEQ ID NO:333 and a light chain comprising the amino acid sequence of SEQ ID NO:418; a heavy chain comprising the amino acid sequence of SEQ ID NO:334 or SEQ ID NO:335 and a light chain comprising the amino acid sequence of SEQ ID NO:418; a heavy chain comprising the amino acid sequence of SEQ ID NO:336 or SEQ ID NO:337 and a light chain comprising the amino acid sequence of SEQ ID NO:418; a heavy chain comprising the amino acid sequence of SEQ ID NO:338 or SEQ ID NO:339 and a light chain comprising the amino acid sequence of SEQ ID NO:418; a heavy chain comprising the amino acid sequence of SEQ ID NO:340 or SEQ ID NO:341 and a light chain comprising the amino acid sequence of SEQ ID NO:418; a heavy chain comprising the amino acid sequence of SEQ ID NO:342 or SEQ ID NO:343 and a light chain comprising the amino acid sequence of SEQ ID NO:418; a heavy chain comprising the amino acid sequence of SEQ ID NO:344 or SEQ ID NO:345 and a light chain comprising the amino acid sequence of SEQ ID NO:419; a heavy chain comprising the amino acid sequence of SEQ ID NO:346 or SEQ ID NO:347 and a light chain comprising the amino acid sequence of SEQ ID NO:419; a heavy chain comprising the amino acid sequence of SEQ ID NO:348 or SEQ ID NO:349 and a light chain comprising the amino acid sequence of SEQ ID NO:419; a heavy chain comprising the amino acid sequence of SEQ ID NO:350 or SEQ ID NO:351 and a light chain comprising the amino acid sequence of SEQ ID NO:419; a heavy chain comprising amino acid sequence of SEQ ID NO:352 or SEQ ID NO:353 and a light chain comprising the amino acid sequence of SEQ ID NO:419; a heavy chain comprising the amino acid sequence of SEQ ID NO:354 or SEQ ID NO:355 and a light chain comprising the amino acid sequence of SEQ ID NO:419; a heavy chain comprising the amino acid sequence of SEQ ID NO:356 or SEQ ID NO:357 and a light chain comprising the amino acid sequence of SEQ ID NO:419; a heavy chain comprising the amino acid sequence of SEQ ID NO:358 or SEQ ID NO:359 and a light chain comprising the amino acid sequence of SEQ ID NO:419; a heavy chain comprising the amino acid sequence of SEQ ID NO:360 or SEQ ID NO:361 and a light chain comprising the amino acid sequence of SEQ ID NO:419; a heavy chain comprising the amino acid sequence of SEQ ID NO:362 or SEQ ID NO:363 and a light chain comprising the amino acid sequence of SEQ ID NO:420; a heavy chain comprising the amino acid sequence of SEQ ID NO:364 or SEQ ID NO:365 and a light chain comprising the amino acid sequence of SEQ ID NO:420; a heavy chain comprising the amino acid sequence of SEQ ID NO:366 or SEQ ID NO:367 and a light chain comprising the amino acid sequence of SEQ ID NO:420; a heavy chain comprising the amino acid sequence of SEQ ID NO:368 or SEQ ID NO:369 and a light chain comprising the amino acid sequence of SEQ ID NO:420; a heavy chain comprising the amino acid sequence of SEQ ID NO:370 or SEQ ID NO:371 and a light chain comprising the amino acid sequence of SEQ ID NO:420; a heavy chain comprising the amino acid sequence of SEQ ID NO:372 or SEQ ID NO:373 and a light chain comprising the amino acid sequence of SEQ ID NO:420; a heavy chain comprising the amino acid sequence of SEQ ID NO:374 or SEQ ID NO:375 and a light chain comprising the amino acid sequence of SEQ ID NO:420; a heavy chain comprising the amino acid sequence of SEQ ID NO:376 or SEQ ID NO:377 and a light chain comprising the amino acid sequence of SEQ ID NO:420; a heavy chain comprising the amino acid sequence of SEQ ID NO:378 or SEQ ID NO:379 and a light chain comprising the amino acid sequence of SEQ ID NO:420; a heavy chain comprising the amino acid sequence of SEQ ID NO:326 or SEQ ID NO:327 and a light chain comprising the amino acid sequence of SEQ ID NO:421; a heavy chain comprising the amino acid sequence of SEQ ID NO:328 or SEQ ID NO:329 and a light chain comprising the amino acid sequence of SEQ ID NO:421; a heavy chain comprising the amino acid sequence of SEQ ID NO:330 or SEQ ID NO:331 and a light chain comprising the amino acid sequence of SEQ ID NO:421; a heavy chain comprising the amino acid sequence of SEQ ID NO:332 or SEQ ID NO:333 and a light chain comprising the amino acid sequence of SEQ ID NO:421; a heavy chain comprising the amino acid sequence of SEQ ID NO:334 or SEQ ID NO:335 and a light chain comprising the amino acid sequence of SEQ ID NO:421; a heavy chain comprising the amino acid sequence of SEQ ID NO:336 or SEQ ID NO:337 and a light chain comprising the amino acid sequence of SEQ ID NO:421; a heavy chain comprising the amino acid sequence of SEQ ID NO:338 or SEQ ID NO:339 and a light chain comprising the amino acid sequence of SEQ ID NO:421; a heavy chain comprising the amino acid sequence of SEQ ID NO:340 or SEQ ID NO:341 and a light chain comprising the amino acid sequence of SEQ ID NO:421; a heavy chain comprising the amino acid sequence of SEQ ID NO:342 or SEQ ID NO:343 and a light chain comprising the amino acid sequence of SEQ ID NO:421; a heavy chain comprising the amino acid sequence of SEQ ID NO:344 or SEQ ID NO:345 and a light chain comprising the amino acid sequence of SEQ ID NO:422; a heavy chain comprising the amino acid sequence of SEQ ID NO:346 or SEQ ID NO:347 and a light chain comprising the amino acid sequence of SEQ ID NO:422; a heavy chain comprising the amino acid sequence of SEQ ID NO:348 or SEQ ID NO:349 and a light chain comprising the amino acid sequence of SEQ ID NO:422; a heavy chain comprising the amino acid sequence of SEQ ID NO:350 or SEQ ID NO:351 and a light chain comprising the amino acid sequence of SEQ ID NO:422; a heavy chain comprising the amino acid sequence of SEQ ID NO:352 or SEQ ID NO:353 and a light chain comprising the amino acid sequence of SEQ ID NO:422; a heavy chain comprising the amino acid sequence of SEQ ID NO:354 or SEQ ID NO:355 and a light chain comprising the amino acid sequence of SEQ ID NO:422; a heavy chain comprising the amino acid sequence of SEQ ID NO:356 or SEQ ID NO:357 and a light chain comprising the amino acid sequence of SEQ ID NO:422; a heavy chain comprising the amino acid sequence of SEQ ID NO:358 or SEQ ID NO:359 and a light chain comprising the amino acid sequence of SEQ ID NO:422; a heavy chain comprising the amino acid sequence of SEQ ID NO:360 or SEQ ID NO:361 and a light chain comprising the amino acid sequence of SEQ ID NO:422; a heavy chain comprising the amino acid sequence of SEQ ID NO:326 or SEQ ID NO:327 and a light chain comprising the amino acid sequence of SEQ ID NO:423; a heavy chain comprising the amino acid sequence of SEQ ID NO:328 or SEQ ID NO:329 and a light chain comprising the amino acid sequence of SEQ ID NO:423; a heavy chain comprising the amino acid sequence of SEQ ID NO:330 or SEQ ID NO:331 and a light chain comprising the amino acid sequence of SEQ ID NO:423; a heavy chain comprising the amino acid sequence of SEQ ID NO:332 or SEQ ID NO:333 and a light chain comprising the amino acid sequence of SEQ ID NO:423; a heavy chain comprising the amino acid sequence of SEQ ID NO:334 or SEQ ID NO:335 and a light chain comprising the amino acid sequence of SEQ ID NO:423; a heavy chain comprising the amino acid sequence of SEQ ID NO:336 or SEQ ID NO:337 and a light chain comprising the amino acid sequence of SEQ ID NO:423; a heavy chain comprising the amino acid sequence of SEQ ID NO:338 or SEQ ID NO:339 and a light chain comprising the amino acid sequence of SEQ ID NO:423; a heavy chain comprising the amino acid sequence of SEQ ID NO:340 or SEQ ID NO:341 and a light chain comprising the amino acid sequence of SEQ ID NO:423; a heavy chain comprising the amino acid sequence of SEQ ID NO:342 or SEQ ID NO:343 and a light chain comprising the amino acid sequence of SEQ ID NO:423; a heavy chain comprising the amino acid sequence of SEQ ID NO:344 or SEQ ID NO:345 and a light chain comprising the amino acid sequence of SEQ ID NO:424; a heavy chain comprising the amino acid sequence of SEQ ID NO:346 or SEQ ID NO:347 and a light chain comprising the amino acid sequence of SEQ ID NO:424; a heavy chain comprising the amino acid sequence of SEQ ID NO:348 or SEQ ID NO:349 and a light chain comprising the amino acid sequence of SEQ ID NO:419; a heavy chain comprising the amino acid sequence of SEQ ID NO:350 or SEQ ID NO:351 and a light chain comprising the amino acid sequence of SEQ ID NO:424; a heavy chain comprising amino acid sequence of SEQ ID NO:352 or SEQ ID NO:353 and a light chain comprising the amino acid sequence of SEQ ID NO:424; a heavy chain comprising the amino acid sequence of SEQ ID NO:354 or SEQ ID NO:355 and a light chain comprising the amino acid sequence of SEQ ID NO:424; a heavy chain comprising the amino acid sequence of SEQ ID NO:356 or SEQ ID NO:357 and a light chain comprising the amino acid sequence of SEQ ID NO:424; a heavy chain comprising the amino acid sequence of SEQ ID NO:358 or SEQ ID NO:359 and a light chain comprising the amino acid sequence of SEQ ID NO:424; a heavy chain comprising the amino acid sequence of SEQ ID NO:360 or SEQ ID NO:361 and a light chain comprising the amino acid sequence of SEQ ID NO:424; a heavy chain comprising the amino acid sequence of SEQ ID NO:362 or SEQ ID NO:363 and a light chain comprising the amino acid sequence of SEQ ID NO:425; a heavy chain comprising the amino acid sequence of SEQ ID NO:364 or SEQ ID NO:365 and a light chain comprising the amino acid sequence of SEQ ID NO:425; a heavy chain comprising the amino acid sequence of SEQ ID NO:366 or SEQ ID NO:367 and a light chain comprising the amino acid sequence of SEQ ID NO:425; a heavy chain comprising the amino acid sequence of SEQ ID NO:368 or SEQ ID NO:369 and a light chain comprising the amino acid sequence of SEQ ID NO:425; a heavy chain comprising the amino acid sequence of SEQ ID NO:370 or SEQ ID NO:371 and a light chain comprising the amino acid sequence of SEQ ID NO:425; a heavy chain comprising the amino acid sequence of SEQ ID NO:372 or SEQ ID NO:373 and a light chain comprising the amino acid sequence of SEQ ID NO:425; a heavy chain comprising the amino acid sequence of SEQ ID NO:374 or SEQ ID NO:375 and a light chain comprising the amino acid sequence of SEQ ID NO:425; a heavy chain comprising the amino acid sequence of SEQ ID NO:376 or SEQ ID NO:377 and a light chain comprising the amino acid sequence of SEQ ID NO:425; a heavy chain comprising the amino acid sequence of SEQ ID NO:378 or SEQ ID NO:379 and a light chain comprising the amino acid sequence of SEQ ID NO:425; a heavy chain comprising the amino acid sequence of SEQ ID NO:326 or SEQ ID NO:327 and a light chain comprising the amino acid sequence of SEQ ID NO:426; a heavy chain comprising the amino acid sequence of SEQ ID NO:328 or SEQ ID NO:329 and a light chain comprising the amino acid sequence of SEQ ID NO:426; a heavy chain comprising the amino acid sequence of SEQ ID NO:330 or SEQ ID NO:331 and a light chain comprising the amino acid sequence of SEQ ID NO:426; a heavy chain comprising the amino acid sequence of SEQ ID NO:332 or SEQ ID NO:333 and a light chain comprising the amino acid sequence of SEQ ID NO:426; a heavy chain comprising the amino acid sequence of SEQ ID NO:334 or SEQ ID NO:335 and a light chain comprising the amino acid sequence of SEQ ID NO:426; a heavy chain comprising the amino acid sequence of SEQ ID NO:336 or SEQ ID NO:337 and a light chain comprising the amino acid sequence of SEQ ID NO:426; a heavy chain comprising the amino acid sequence of SEQ ID NO:338 or SEQ ID NO:339 and a light chain comprising the amino acid sequence of SEQ ID NO:426; a heavy chain comprising the amino acid sequence of SEQ ID NO:340 or SEQ ID NO:341 and a light chain comprising the amino acid sequence of SEQ ID NO:426; a heavy chain comprising the amino acid sequence of SEQ ID NO:342 or SEQ ID NO:343 and a light chain comprising the amino acid sequence of SEQ ID NO:426; a heavy chain comprising the amino acid sequence of SEQ ID NO:380 or SEQ ID NO:381 and a light chain comprising the amino acid sequence of SEQ ID NO:427; a heavy chain comprising the amino acid sequence of SEQ ID NO:382 or SEQ ID NO:383 and a light chain comprising the amino acid sequence of SEQ ID NO:427; a heavy chain comprising the amino acid sequence of SEQ ID NO:384 or SEQ ID NO:385 and a light chain comprising the amino acid sequence of SEQ ID NO:427; a heavy chain comprising the amino acid sequence of SEQ ID NO:386 or SEQ ID NO:387 and a light chain comprising the amino acid sequence of SEQ ID NO:427; a heavy chain comprising the amino acid sequence of SEQ ID NO:388 or SEQ ID NO:389 and a light chain comprising the amino acid sequence of SEQ ID NO:427; a heavy chain comprising the amino acid sequence of SEQ ID NO:390 or SEQ ID NO:391 and a light chain comprising the amino acid sequence of SEQ ID NO:427; a heavy chain comprising the amino acid sequence of SEQ ID NO:392 or SEQ ID NO:393 and a light chain comprising the amino acid sequence of SEQ ID NO:427; a heavy chain comprising the amino acid sequence of SEQ ID NO:394 or SEQ ID NO:395 and a light chain comprising the amino acid sequence of SEQ ID NO:427; a heavy chain comprising the amino acid sequence of SEQ ID NO:396 or SEQ ID NO:397 and a light chain comprising the amino acid sequence of SEQ ID NO:427; a heavy chain comprising the amino acid sequence of SEQ ID NO:398 or SEQ ID NO:399 and a light chain comprising the amino acid sequence of SEQ ID NO:428; a heavy chain comprising the amino acid sequence of SEQ ID NO:400 or SEQ ID NO:401 and a light chain comprising the amino acid sequence of SEQ ID NO:428; a heavy chain comprising the amino acid sequence of SEQ ID NO:402 or SEQ ID NO:403 and a light chain comprising the amino acid sequence of SEQ ID NO:428; a heavy chain comprising the amino acid sequence of SEQ ID NO:404 or SEQ ID NO:405 and a light chain comprising the amino acid sequence of SEQ ID NO:428; a heavy chain comprising the amino acid sequence of SEQ ID NO:406 or SEQ ID NO:407 and a light chain comprising the amino acid sequence of SEQ ID NO:428; a heavy chain comprising the amino acid sequence of SEQ ID NO:408 or SEQ ID NO:409 and a light chain comprising the amino acid sequence of SEQ ID NO:428; a heavy chain comprising the amino acid sequence of SEQ ID NO:410 or SEQ ID NO:411 and a light chain comprising the amino acid sequence of SEQ ID NO:428; a heavy chain comprising the amino acid sequence of SEQ ID NO:412 or SEQ ID NO:413 and a light chain comprising the amino acid sequence of SEQ ID NO:428; a heavy chain comprising the amino acid sequence of SEQ ID NO:414 or SEQ ID NO:415 and a light chain comprising the amino acid sequence of SEQ ID NO:428; a heavy chain comprising the amino acid sequence of SEQ ID NO:398 or SEQ ID NO:399 and a light chain comprising the amino acid sequence of SEQ ID NO:429; a heavy chain comprising the amino acid sequence of SEQ ID NO:400 or SEQ ID NO:401 and a light chain comprising the amino acid sequence of SEQ ID NO:429; a heavy chain comprising the amino acid sequence of SEQ ID NO:402 or SEQ ID NO:403 and a light chain comprising the amino acid sequence of SEQ ID NO:429; a heavy chain comprising the amino acid sequence of SEQ ID NO:404 or SEQ ID NO:405 and a light chain comprising the amino acid sequence of SEQ ID NO:429; a heavy chain comprising the amino acid sequence of SEQ ID NO:406 or SEQ ID NO:407 and a light chain comprising the amino acid sequence of SEQ ID NO:429; a heavy chain comprising the amino acid sequence of SEQ ID NO:408 or SEQ ID NO:409 and a light chain comprising the amino acid sequence of SEQ ID NO:429; a heavy chain comprising the amino acid sequence of SEQ ID NO:410 or SEQ ID NO:411 and a light chain comprising the amino acid sequence of SEQ ID NO:429; a heavy chain comprising the amino acid sequence of SEQ ID NO:412 or SEQ ID NO:413 and a light chain comprising the amino acid sequence of SEQ ID NO:429; a heavy chain comprising the amino acid sequence of SEQ ID NO:414 or SEQ ID NO:415 and a light chain comprising the amino acid sequence of SEQ ID NO:429; a heavy chain comprising the amino acid sequence of SEQ ID NO:398 or SEQ ID NO:399 and a light chain comprising the amino acid sequence of SEQ ID NO:430; a heavy chain comprising the amino acid sequence of SEQ ID NO:400 or SEQ ID NO:401 and a light chain comprising the amino acid sequence of SEQ ID NO:430; a heavy chain comprising the amino acid sequence of SEQ ID NO:402 or SEQ ID NO:403 and a light chain comprising the amino acid sequence of SEQ ID NO:430; a heavy chain comprising the amino acid sequence of SEQ ID NO:404 or SEQ ID NO:405 and a light chain comprising the amino acid sequence of SEQ ID NO:430; a heavy chain comprising the amino acid sequence of SEQ ID NO:406 or SEQ ID NO:407 and a light chain comprising the amino acid sequence of SEQ ID NO:430; a heavy chain comprising the amino acid sequence of SEQ ID NO:408 or SEQ ID NO:409 and a light chain comprising the amino acid sequence of SEQ ID NO:430; a heavy chain comprising the amino acid sequence of SEQ ID NO:410 or SEQ ID NO:411 and a light chain comprising the amino acid sequence of SEQ ID NO:430; a heavy chain comprising the amino acid sequence of SEQ ID NO:412 or SEQ ID NO:413 and a light chain comprising the amino acid sequence of SEQ ID NO:430; a heavy chain comprising the amino acid sequence of SEQ ID NO:414 or SEQ ID NO:415 and a light chain comprising the amino acid sequence of SEQ ID NO:430; a heavy chain comprising the amino acid sequence of SEQ ID NO:398 or SEQ ID NO:399 and a light chain comprising the amino acid sequence of SEQ ID NO:431; a heavy chain comprising the amino acid sequence of SEQ ID NO:400 or SEQ ID NO:401 and a light chain comprising the amino acid sequence of SEQ ID NO:431; a heavy chain comprising the amino acid sequence of SEQ ID NO:402 or SEQ ID NO:403 and a light chain comprising the amino acid sequence of SEQ ID NO:431; a heavy chain comprising the amino acid sequence of SEQ ID NO:404 or SEQ ID NO:405 and a light chain comprising the amino acid sequence of SEQ ID NO:431; a heavy chain comprising the amino acid sequence of SEQ ID NO:406 or SEQ ID NO:407 and a light chain comprising the amino acid sequence of SEQ ID NO:431; a heavy chain comprising the amino acid sequence of SEQ ID NO:408 or SEQ ID NO:409 and a light chain comprising the amino acid sequence of SEQ ID NO:431; a heavy chain comprising the amino acid sequence of SEQ ID NO:410 or SEQ ID NO:411 and a light chain comprising the amino acid sequence of SEQ ID NO:431; a heavy chain comprising the amino acid sequence of SEQ ID NO:412 or SEQ ID NO:413 and a light chain comprising the amino acid sequence of SEQ ID NO:431; a heavy chain comprising the amino acid sequence of SEQ ID NO:414 or SEQ ID NO:415 and a light chain comprising the amino acid sequence of SEQ ID NO:431; a heavy chain comprising the amino acid sequence of SEQ ID NO:398 or SEQ ID NO:399 and a light chain comprising the amino acid sequence of SEQ ID NO:432; a heavy chain comprising the amino acid sequence of SEQ ID NO:400 or SEQ ID NO:401 and a light chain comprising the amino acid sequence of SEQ ID NO:432; a heavy chain comprising the amino acid sequence of SEQ ID NO:402 or SEQ ID NO:403 and a light chain comprising the amino acid sequence of SEQ ID NO:432; a heavy chain comprising the amino acid sequence of SEQ ID NO:404 or SEQ ID NO:405 and a light chain comprising the amino acid sequence of SEQ ID NO:432; a heavy chain comprising the amino acid sequence of SEQ ID NO:406 or SEQ ID NO:407 and a light chain comprising the amino acid sequence of SEQ ID NO:432; a heavy chain comprising the amino acid sequence of SEQ ID NO:408 or SEQ ID NO:409 and a light chain comprising the amino acid sequence of SEQ ID NO:432; a heavy chain comprising the amino acid sequence of SEQ ID NO:410 or SEQ ID NO:411 and a light chain comprising the amino acid sequence of SEQ ID NO:432; a heavy chain comprising the amino acid sequence of SEQ ID NO:412 or SEQ ID NO:413 and a light chain comprising the amino acid sequence of SEQ ID NO:432; a heavy chain comprising the amino acid sequence of SEQ ID NO:414 or SEQ ID NO:415 and a light chain comprising the amino acid sequence of SEQ ID NO:432; a heavy chain comprising the amino acid sequence of SEQ ID NO:398 or SEQ ID NO:399 and a light chain comprising the amino acid sequence of SEQ ID NO:433; a heavy chain comprising the amino acid sequence of SEQ ID NO:400 or SEQ ID NO:401 and a light chain comprising the amino acid sequence of SEQ ID NO:433; a heavy chain comprising the amino acid sequence of SEQ ID NO:402 or SEQ ID NO:403 and a light chain comprising the amino acid sequence of SEQ ID NO:433; a heavy chain comprising the amino acid sequence of SEQ ID NO:404 or SEQ ID NO:405 and a light chain comprising the amino acid sequence of SEQ ID NO:433; a heavy chain comprising the amino acid sequence of SEQ ID NO:406 or SEQ ID NO:407 and a light chain comprising the amino acid sequence of SEQ ID NO:433; a heavy chain comprising the amino acid sequence of SEQ ID NO:408 or SEQ ID NO:409 and a light chain comprising the amino acid sequence of SEQ ID NO:433; a heavy chain comprising the amino acid sequence of SEQ ID NO:410 or SEQ ID NO:411 and a light chain comprising the amino acid sequence of SEQ ID NO:433; a heavy chain comprising the amino acid sequence of SEQ ID NO:412 or SEQ ID NO:413 and a light chain comprising the amino acid sequence of SEQ ID NO:433; or a heavy chain comprising the amino acid sequence of SEQ ID NO:414 or SEQ ID NO:415 and a light chain comprising the amino acid sequence of SEQ ID NO:433.

In yet further embodiments, the disclosure herein relates to an anti-GPNMB antibody comprising a VH comprising HVR-H1, HVR-H2, and HVR-H3 and a VL comprising HVR-L1, HVR-L2, and HVR-L3 of any one of antibodies GPN-01, GPN-03, GPN-06, GPN-07, GPN-08, GPN-09, GPN-11, GPN-22, GPN-24, GPN-25, GPN-26, GPN-30, GPN-31, GPN-33, GPN-34, GPN-35, GPN-37, GPN-38, GPN-41, GPN-42, GPN-43, GPN-48, GPN-52, GPN-61, GPN-65, GPN-81, GPN-82, GPN-83, GPN-84, GPN-85, GPN-86, GPN-87, GPN-88, GPN-89, GPN-90, GPN-91, GPN-92, GPN-93, GPN-94, GPN-95, GPN-96, GPN-97, and GPN-98. In some cases, the antibody comprises a VH and/or a VL at least 90%, at least 95%, at least 97%, or at least 99% identical to those of any one of antibodies GPN-01, GPN-03, GPN-06, GPN-07, GPN-08, GPN-09, GPN-11, GPN-22, GPN-24, GPN-25, GPN-26, GPN-30, GPN-31, GPN-33, GPN-34, GPN-35, GPN-37, GPN-38, GPN-41, GPN-42, GPN-43, GPN-48, GPN-52, GPN-61, GPN-65, GPN-81, GPN-82, GPN-83, GPN-84, GPN-85, GPN-86, GPN-87, GPN-88, GPN-89, GPN-90, GPN-91, GPN-92, GPN-93, GPN-94, GPN-95, GPN-96, GPN-97, and GPN-98. In some cases, the antibody comprises the VH and/or the VL of any one of antibodies GPN-01, GPN-03, GPN-06, GPN-07, GPN-08, GPN-09, GPN-11, GPN-22, GPN-24, GPN-25, GPN-26, GPN-30, GPN-31, GPN-33, GPN-34, GPN-35, GPN-37, GPN-38, GPN-41, GPN-42, GPN-43, GPN-48, GPN-52, GPN-61, GPN-65, GPN-81, GPN-82, GPN-83, GPN-84, GPN-85, GPN-86, GPN-87, GPN-88, GPN-89, GPN-90, GPN-91, GPN-92, GPN-93, GPN-94, GPN-95, GPN-96, GPN-97, and GPN-98. In some cases, the antibody comprises: a VH and VL, wherein the VH comprises the HVR-H1, HVR-H2, and HVR-H3 of the antibody GPN-01 (as shown in Table 2) and the VL comprises the HVR-L1, HVR-L2, and HVR-L3 of the antibody GPN-01 (as shown in Table 3); or wherein the VH comprises the HVR-H1, HVR-H2, and HVR-H3 of the antibody GPN-03 (as shown in Table 2) and the VL comprises the HVR-L1, HVR-L2, and HVR-L3 of the antibody GPN-03 (as shown in Table 3); or wherein the VH comprises the HVR-H1, HVR-H2, and HVR-H3 of the antibody GPN-06 (as shown in Table 2) and the VL comprises the HVR-L1, HVR-L2, and HVR-L3 of the antibody GPN-06 (as shown in Table 3); or wherein the VH comprises the HVR-H1, HVR-H2, and HVR-H3 of the antibody GPN-07 (as shown in Table 2) and the VL comprises the HVR-L1, HVR-L2, and HVR-L3 of the antibody GPN-07 (as shown in Table 3); or wherein the VH comprises the HVR-H1, HVR-H2, and HVR-H3 of the antibody GPN-08 (as shown in Table 2) and the VL comprises the HVR-L1, HVR-L2, and HVR-L3 of the antibody GPN-08 (as shown in Table 3); or wherein the VH comprises the HVR-H1, HVR-H2, and HVR-H3 of the antibody GPN-09 (as shown in Table 2) and the VL comprises the HVR-L1, HVR-L2, and HVR-L3 of the antibody GPN-09 (as shown in Table 3); or wherein the VH comprises the HVR-H1, HVR-H2, and HVR-H3 of the antibody GPN-11 (as shown in Table 2) and the VL comprises the HVR-L1, HVR-L2, and HVR-L3 of the antibody GPN-11 (as shown in Table 3); or wherein the VH comprises the HVR-H1, HVR-H2, and HVR-H3 of the antibody GPN-22 (as shown in Table 2) and the VL comprises the HVR-L1, HVR-L2, and HVR-L3 of the antibody GPN-22 (as shown in Table 3); or wherein the VH comprises the HVR-H1, HVR-H2, and HVR-H3 of the antibody GPN-24 (as shown in Table 2) and the VL comprises the HVR-L1, HVR-L2, and HVR-L3 of the antibody GPN-24 (as shown in Table 3); or wherein the VH comprises the HVR-H1, HVR-H2, and HVR-H3 of the antibody GPN-25 (as shown in Table 2) and the VL comprises the HVR-L1, HVR-L2, and HVR-L3 of the antibody GPN-25 (as shown in Table 3); or wherein the VH comprises the HVR-H1, HVR-H2, and HVR-H3 of the antibody GPN-26 (as shown in Table 2) and the VL comprises the HVR-L1, HVR-L2, and HVR-L3 of the antibody GPN-26 (as shown in Table 3); or wherein the VH comprises the HVR-H1, HVR-H2, and HVR-H3 of the antibody GPN-30 (as shown in Table 2) and the VL comprises the HVR-L1, HVR-L2, and HVR-L3 of the antibody GPN-30 (as shown in Table 3); or wherein the VH comprises the HVR-H1, HVR-H2, and HVR-H3 of the antibody GPN-31 (as shown in Table 2) and the VL comprises the HVR-L1, HVR-L2, and HVR-L3 of the antibody GPN-31 (as shown in Table 3); or wherein the VH comprises the HVR-H1, HVR-H2, and HVR-H3 of the antibody GPN-33 (as shown in Table 2) and the VL comprises the HVR-L1, HVR-L2, and HVR-L3 of the antibody GPN-33 (as shown in Table 3); or wherein the VH comprises the HVR-H1, HVR-H2, and HVR-H3 of the antibody GPN-34 (as shown in Table 2) and the VL comprises the HVR-L1, HVR-L2, and HVR-L3 of the antibody GPN-34 (as shown in Table 3); or wherein the VH comprises the HVR-H1, HVR-H2, and HVR-H3 of the antibody GPN-35 (as shown in Table 2) and the VL comprises the HVR-L1, HVR-L2, and HVR-L3 of the antibody GPN-35 (as shown in Table 3); or wherein the VH comprises the HVR-H1, HVR-H2, and HVR-H3 of the antibody GPN-37 (as shown in Table 2) and the VL comprises the HVR-L1, HVR-L2, and HVR-L3 of the antibody GPN-37 (as shown in Table 3); or wherein the VH comprises the HVR-H1, HVR-H2, and HVR-H3 of the antibody GPN-38 (as shown in Table 2) and the VL comprises the HVR-L1, HVR-L2, and HVR-L3 of the antibody GPN-38 (as shown in Table 3); or wherein the VH comprises the HVR-H1, HVR-H2, and HVR-H3 of the antibody GPN-41 (as shown in Table 2) and the VL comprises the HVR-L1, HVR-L2, and HVR-L3 of the antibody GPN-41 (as shown in Table 3); or wherein the VH comprises the HVR-H1, HVR-H2, and HVR-H3 of the antibody GPN-42 (as shown in Table 2) and the VL comprises the HVR-L1, HVR-L2, and HVR-L3 of the antibody GPN-42 (as shown in Table 3); or wherein the VH comprises the HVR-H1, HVR-H2, and HVR-H3 of the antibody GPN-43 (as shown in Table 2) and the VL comprises the HVR-L1, HVR-L2, and HVR-L3 of the antibody GPN-43 (as shown in Table 3); or wherein the VH comprises the HVR-H1, HVR-H2, and HVR-H3 of the antibody GPN-48 (as shown in Table 2) and the VL comprises the HVR-L1, HVR-L2, and HVR-L3 of the antibody GPN-48 (as shown in Table 3); or wherein the VH comprises the HVR-H1, HVR-H2, and HVR-H3 of the antibody GPN-52 (as shown in Table 2) and the VL comprises the HVR-L1, HVR-L2, and HVR-L3 of the antibody GPN-52 (as shown in Table 3); or wherein the VH comprises the HVR-H1, HVR-H2, and HVR-H3 of the antibody GPN-61 (as shown in Table 2) and the VL comprises the HVR-L1, HVR-L2, and HVR-L3 of the antibody GPN-61 (as shown in Table 3); or wherein the VH comprises the HVR-H1, HVR-H2, and HVR-H3 of the antibody GPN-65 (as shown in Table 2) and the VL comprises the HVR-L1, HVR-L2, and HVR-L3 of the antibody GPN-65 (as shown in Table 3); or wherein the VH comprises the HVR-H1, HVR-H2, and HVR-H3 of the antibody GPN-81 (as shown in Table 16) and the VL comprises the HVR-L1, HVR-L2, and HVR-L3 of the antibody GPN-81 (as shown in Table 17); or wherein the VH comprises the HVR-H1, HVR-H2, and HVR-H3 of the antibody GPN-82 (as shown in Table 16) and the VL comprises the HVR-L1, HVR-L2, and HVR-L3 of the antibody GPN-82 (as shown in Table 17); or wherein the VH comprises the HVR-H1, HVR-H2, and HVR-H3 of the antibody GPN-83 (as shown in Table 16) and the VL comprises the HVR-L1, HVR-L2, and HVR-L3 of the antibody GPN-83 (as shown in Table 17); or wherein the VH comprises the HVR-H1, HVR-H2, and HVR-H3 of the antibody GPN-84 (as shown in Table 16) and the VL comprises the HVR-L1, HVR-L2, and HVR-L3 of the antibody GPN-84 (as shown in Table 17); or wherein the VH comprises the HVR-H1, HVR-H2, and HVR-H3 of the antibody GPN-85 (as shown in Table 16) and the VL comprises the HVR-L1, HVR-L2, and HVR-L3 of the antibody GPN-85 (as shown in Table 17); or wherein the VH comprises the HVR-H1, HVR-H2, and HVR-H3 of the antibody GPN-86 (as shown in Table 16) and the VL comprises the HVR-L1, HVR-L2, and HVR-L3 of the antibody GPN-86 (as shown in Table 17); or wherein the VH comprises the HVR-H1, HVR-H2, and HVR-H3 of the antibody GPN-87 (as shown in Table 16) and the VL comprises the HVR-L1, HVR-L2, and HVR-L3 of the antibody GPN-87 (as shown in Table 17); or wherein the VH comprises the HVR-H1, HVR-H2, and HVR-H3 of the antibody GPN-88 (as shown in Table 16) and the VL comprises the HVR-L1, HVR-L2, and HVR-L3 of the antibody GPN-88 (as shown in Table 17); or wherein the VH comprises the HVR-H1, HVR-H2, and HVR-H3 of the antibody GPN-89 (as shown in Table 16) and the VL comprises the HVR-L1, HVR-L2, and HVR-L3 of the antibody GPN-89 (as shown in Table 17); or wherein the VH comprises the HVR-H1, HVR-H2, and HVR-H3 of the antibody GPN-90 (as shown in Table 16) and the VL comprises the HVR-L1, HVR-L2, and HVR-L3 of the antibody GPN-90 (as shown in Table 17); or wherein the VH comprises the HVR-H1, HVR-H2, and HVR-H3 of the antibody GPN-91 (as shown in Table 16) and the VL comprises the HVR-L1, HVR-L2, and HVR-L3 of the antibody GPN-91 (as shown in Table 17); or wherein the VH comprises the HVR-H1, HVR-H2, and HVR-H3 of the antibody GPN-92 (as shown in Table 16) and the VL comprises the HVR-L1, HVR-L2, and HVR-L3 of the antibody GPN-92 (as shown in Table 17); or wherein the VH comprises the HVR-H1, HVR-H2, and HVR-H3 of the antibody GPN-93 (as shown in Table 16) and the VL comprises the HVR-L1, HVR-L2, and HVR-L3 of the antibody GPN-93 (as shown in Table 17); or wherein the VH comprises the HVR-H1, HVR-H2, and HVR-H3 of the antibody GPN-94 (as shown in Table 16) and the VL comprises the HVR-L1, HVR-L2, and HVR-L3 of the antibody GPN-94 (as shown in Table 17); or wherein the VH comprises the HVR-H1, HVR-H2, and HVR-H3 of the antibody GPN-95 (as shown in Table 16) and the VL comprises the HVR-L1, HVR-L2, and HVR-L3 of the antibody GPN-95 (as shown in Table 17); or wherein the VH comprises the HVR-H1, HVR-H2, and HVR-H3 of the antibody GPN-96 (as shown in Table 16) and the VL comprises the HVR-L1, HVR-L2, and HVR-L3 of the antibody GPN-96 (as shown in Table 17); or wherein the VH comprises the HVR-H1, HVR-H2, and HVR-H3 of the antibody GPN-97 (as shown in Table 16) and the VL comprises the HVR-L1, HVR-L2, and HVR-L3 of the antibody GPN-97 (as shown in Table 17); or wherein the VH comprises the HVR-H1, HVR-H2, and HVR-H3 of the antibody GPN-98 (as shown in Table 16) and the VL comprises the HVR-L1, HVR-L2, and HVR-L3 of the antibody GPN-98 (as shown in Table 17).

In some cases, an antibody herein antagonizes GPNMB activity. In some embodiments, the anti-GPNMB antibody: modulates expression of activation surface markers on myeloid cells, increases cell surface expression of PD-L1 in human macrophages, increases cell surface expression of CD40 in human macrophages, increases cell surface expression of CD80 in human macrophages, modulates lysosome function in myeloid cells, increases glucocerebrosidase activity in human macrophages, decreases cell surface expression of GPNMB in human macrophages, changes interferon pathway gene expression patterns in human macrophages, reduces tumor volume in a murine tumor model, such as in an MC38 model, reduces tumor growth rate in a murine tumor model, such as in an MC38 model, increases levels of IL-12p40 in serum, increases levels of CCL5 in serum, is a GPNMB ligand blocking antibody, is a non-blocking antibody, antagonizes GPNMB activity independent of ligand blocking activity, antagonizes GPNMB activity in vivo, binds to GPNMB expressed on a cell surface, overcomes a decrease in glucocerebrosidase activity associated with reduced progranulin levels, decreases GPNMB expression levels in cells, optionally in macrophages, decreases LAMP2 expression levels in cells, optionally in macrophages, monocytes, or neutrophils, inhibits or reduces inflammasome activation; inhibits IL-1β expression or release, reduces neural inflammation, reduces expression of C1q, GFAP, IBA1, and CTSD associated with neural inflammation, and/or reduces lysosomal stress.

In some embodiments, the antibody binds human GPNMB, binds mouse GPNMB, binds cynomolgus GMNMB, binds both human and mouse GPNMB, or binds human, mouse, and cynomolgus GPNMB. In some embodiments, the antibody binds human GPNMB with an affinity of about 0.4 nM to about 120 nM, of about 0.3 nM to about 5 nM, of about 0.4 nM to about 1.04 nM, of about 0.14 to about 0.65 nM and bind mouse GPNMB with an affinity of about 0.18 nM to about 0.44 nM.

In some cases, the antibody is a monoclonal antibody, humanized antibody, antigen binding fragment, such as an Fab, Fab′, Fab′-SH, F(ab′)2, Fv, or scFv fragment, or is a bispecific or multispecific antibody. In some cases, the antibody of the IgG class, the IgM class, or the IgA class, such as a human IgG1, IgG2, IgG3, or IgG4 isotype or of a mouse IgG1 or IgG2 isotype. In some cases, the antibody binds to an inhibitory Fc receptor, such as to an inhibitory Fc-gamma receptor IIB (FcgRIIB). In some cases, the antibody decreases cellular levels of FcgRIIB. In some cases, the antibody has a human or mouse IgG1 isotype and comprises one or more amino acid substitutions in the Fc region at an amino acid residue selected from the group consisting of N297A, D265A, D270A, L234A, L235A, G237A, P238D, L328E, E233D, G237D, H268D, P271G, A330R, C226S, C229S, E233P, L234V, L234F, L235E, P331S, P331G, S267E, L328F, A330L, M252Y, S254T, T256E, N297Q, P238S, P238A, A327Q, A327G, P329A, P329S, P329G, K322A, N325S, T394D, A330S, E430G, E430S, E430F, E430T, E345K, E345Q, E345R, E345Y, S440Y, S440W, and any combination thereof, wherein the numbering of the residues is according to EU numbering. In some cases, the antibody has a human or mouse IgG2 isotype and comprises one or more amino acid substitutions in the Fc region at an amino acid residue selected from the group consisting of A330S, C127S, C214S, C219S, C220S, E345K, E345Q, E345R, E345Y, E430F, E430G, E430S, E430T, G237A, H268Q, L328F, M252Y, P331S, S254T, S267E, S440W, S440Y, T256E, V234A, V309L, and any combination thereof, wherein the numbering of the residues is according to EU numbering. In some cases, the antibody has a human or mouse IgG4 isotype and comprises one or more amino acid substitutions in the Fc region at an amino acid residue selected from the group consisting of C127S, E318A, E345R, E430G, F234A, G237A, K322A, L235A, L235E, L236E, L243A, L328F, M252Y, P331S, S228P, S229P, S254T, S267E, S440Y, T256E, and any combination thereof, wherein the numbering of the residues is according to EU numbering.

The antibody of any one of claims 1-54, wherein the antibody comprises one or more amino acid substitutions in the Fc region at a residue position selected from the group consisting of A330L, A330S, C127S, E345R, E430G, K322A, L234A, L234F, L235A, L235E, L243A, L328F, P331S, S267E, S440Y, and any combination thereof, wherein the numbering of the amino acid residues is according to EU or Kabat numbering. In some cases, the IgG Fc amino acid sequences is selected from the group consisting of SEQ ID NOs: 213-233.

The present disclosure also relates to pharmaceutical compositions comprising the anti-GPNMB antibody as described herein and a pharmaceutically acceptable carrier, as well as to an isolated nucleic acid comprising a nucleic acid sequence encoding an anti-GPNMB antibody as described herein, isolated vectors comprising the nucleic acids, and isolated host cells comprising the nucleic acids or vectors. The disclosure further relates to methods of producing an anti-GPNMB antibody, comprising culturing the host cell so that the antibody is produced, and optionally further recovering the antibody produced by the cell.

The present disclosure also relates to methods of treating a cancer in a subject in need thereof, the method comprising administering to an individual in need thereof a therapeutically effective amount of an anti-GPNMB antibody herein, thereby treating the cancer. In some cases, the methods further comprise administering one or more therapeutic agents, such as a checkpoint inhibitor, such as a PD1, PD-L1, and PD-L2 inhibitor, such as an anti-PD-L1 antibody, an anti-PD-L2 antibody, and an anti-PD-1 antibody. In some cases, the cancer is cancer is sarcoma, bladder cancer, breast cancer, colon cancer, endometrial cancer, kidney cancer, renal cancer, leukemia, lung cancer, non-small cell lung cancer, melanoma, lymphoma, pancreatic cancer, prostate cancer, ovarian cancer, stomach cancer, thyroid cancer, cancer of the uterus, liver cancer, cervical cancer, testicular cancer, squamous cell carcinoma, glioma, glioblastoma, adenoma, or neuroblastoma. In some cases, the cancer is glioblastoma multiforme, bladder carcinoma, esophageal carcinoma, or triple-negative breast carcinoma.

The disclosure further relates to methods of treating a neurodegenerative disease or disorder in a subject in need thereof, the method comprising administering to an individual in need thereof a therapeutically effective amount of an anti-GPNMB antibody herein, thereby treating the disease or disorder. In some cases, the disease or disorder is selected from Parkinson's disease, Alzheimer's disease, and ALS.

The disclosure also relates to methods of treating a lysosomal storage disease or disorder in a subject in need thereof, the method comprising administering to an individual in need thereof a therapeutically effective amount of an anti-GPNMB antibody herein, thereby treating the disease or disorder. In some cases, the lysosomal storage disease is Gaucher's disease.

The disclosure also relates to methods of detecting the presence of GPNMB in a sample in vitro or an individual, the method comprising an anti-GPNMB antibody herein. In some cases, the methods further comprise quantification of antigen-bound anti-GPNMB antibody.

It is to be understood that one, some, or all of the properties of the various embodiments described herein may be combined to form other embodiments of the present disclosure. These and other aspects of the disclosure will become apparent to one of skill in the art. These and other aspects of the disclosure are further described by the detailed description that follows. In some aspects, provided herein is a method for detecting GPNMB in a sample comprising contacting said sample with any of the anti-GPNMB antibodies described herein.

### BRIEF DESCRIPTION OF THE DRAWINGS

The present US provisional application includes at least one drawing executed in color. In the event that a nonprovisional or PCT application claiming priority to this US provisional application and incorporating the contents of this provisional application publishes in the future, copies of this provisional patent application including the color drawings will be provided by the Office upon request and payment of the necessary fee.

FIG. 1 sets forth an amino acid alignment of human (SEQ ID NO: 1), mouse (SEQ ID NO: 435), and cynomolgus (SEQ ID NO:434) GPNMB.

FIGS. 2A and 2B set forth data showing anti-GPNMB antibodies of the present disclosure binding to Freestyle293 cells expressing human GPNMB and binding to B16F10 cells expressing mouse GPNMB, respectively. FIG. 2C sets forth data showing anti-GPNMB antibodies of the present disclosure binding to U937 cells expressing GPNMB.

FIG. 3A and FIG. 3B set forth data showing titration curves of anti-GPNMB antibodies of the present disclosure binding to Freestyle293 cells expressing human GPNMB and binding to B16F10 cells expressing mouse GPNMB, respectively.

FIG. 4A FIG. 4J set forth data showing the binding of soluble mouse GPNMB-Fc or soluble human GPNMB-Fc polypeptide constructs binding to various cell types.

FIG. 5A-FIG. 5C set forth data showing the effect of anti-GPNMB antibodies of the present disclosure on blocking or enhancing the binding of soluble mouse GPNMB-Fc or soluble human GPNMB-Fc polypeptide constructs to SVEC cells.

FIG. 6A-FIG. 6G set forth data showing anti-GPNMB antibodies of the present disclosure increasing cell surface expression of PDL1, CD40, and CD80 in macrophages. FIG. 6H sets forth data showing the effect of JAK kinase inhibitor on the increase in cell surface PDL1 expression induced by anti-GPNMB antibodies of the present disclosure.

FIG. 7A and FIG. 7B set forth data showing the effect of anti-GPNMB antibodies of the present disclosure on increasing glucocerebrosidase activity in macrophages.

FIG. 8A and FIG. 8B set forth data showing anti-GPNMB antibodies of the present disclosure induce cell surface GPNMB internalization.

FIG. 9 sets forth data showing binning results of anti-GPNMB antibodies of the present disclosure binding to GPNMB.

FIG. 10 sets forth data showing various GPNMB chimeric polypeptide constructs comprising human and mouse GPNMB domains.

FIG. 11A-FIG. 11C set forth data showing that genetically depleted GPNMB in M1 and M2 polarized macrophages resulted in an increase in the interferon response pathway of chemokines and cytokines.

FIG. 12A and FIG. 12B set forth data showing the effect of anti-GPNMB antibody GPN-61 on differential gene expression of various genes associated with the interferon signaling pathway in human macrophages.

FIG. 13A and FIG. 13B set forth data showing reduced tumor growth in GPNMB knock-out mice and GPNMB heterozygous knock-out mice in an in vivo mouse tumor model.

FIG. 14A and FIG. 14B set forth data showing GPNMB expression in macrophages and monocytes as measured by scRNAseq analysis. FIG. 14C sets forth data showing GPNMB heterozygosity resulted in reduction in the number of suppressive-like monocytes (myeloid-derived suppressive cells, CD14+) from Cluster 2. FIG. 14D-FIG. 14F set forth data showing GPNMB heterozygosity resulted in differential gene expression patterns in Cluster 3 (CD8+ T-cells), Cluster 0 (ClQC+ macrophages), and Cluster 9 (proliferating T-cells), respectively.

FIG. 15A and FIG. 15B set forth data showing anti-GPNMB antibody GPN-61 reduced tumor volumes in vivo in a mouse tumor model.

FIG. 16A and FIG. 16B set forth data showing reduced glucocerebrosidase activity in granulocytes and monocytes/macrophages, respectively, in wild type and progranulin knock-out mice.

FIG. 16C sets forth data showing treatment of progranulin knock-out mice with anti-GPNMB antibody GPN-61 resulted in higher glucocerebrosidase activity in monocytes compared to that observed in GRN−/− mice treated with isotype control antibody.

FIG. 17A and FIG. 17B set forth data showing treatment of either wildtype mice or progranulin knock-out mice with anti-GPNMB antibody GPN-61 resulted in increased levels of IL-12p40 and CCL5, respectively, in serum compared to that observed in isotype control antibody treated animals.

FIG. 18 sets forth data showing a heatmap comparing differentially expressed genes from GPNMB knockout mice (WT-KO), GPNMB knockout mice treated with CBE (Gpnmb KO CBE), and wildtype mice treated with CBE (Gpnmb WT CBE).

FIG. 19A-FIG. 19D set forth data showing a comparison of select antiviral, interferon-related genes showing differential expression (Stat2, Irf9, Cxcl16, and Ifit3b) in wildtype (WT) mice and in GPNMB knockout mice (Gpnmb KO) treated with CBE.

FIG. 20A sets forth data showing increased expression of complement component 1q (C1q), glial fibrillary acidic protein (GFAP), ionized calcium-binding adapter molecule 1 (IBA1), and cathepsin D (CatD) in the cortex of wildtype or GPNMB knockout mice treated with or without conduritol B-epoxide (CBE); FIG. 20B-FIG. 20E set forth data showing relative intensities of the expressed proteins from FIG. 20A; FIG. 20F sets forth data showing increased serum CXC motif chemokine ligand 1 (CXCL1) in wildtype or GPNMB knockout mice treated with or without CBE.

FIG. 21A sets forth data showing increased IL-1β expression in human macrophages stimulated with LPS+nigericin (to activate inflammasomes); FIG. 21B set for data showing anti-GPNMB antibodies of the present disclosure inhibited inflammasome activation in human macrophages as measured by changes in IL-1β expression.

FIG. 22A and FIG. 22B set forth data showing anti-GPNMB antibodies of the present disclosure reduced expression of IL-1β in human macrophages following inflammasome activation with LPS+nigericin.

FIG. 23A and FIG. 23B set forth data showing the effects of anti-GPNMB antibodies of the present disclosure having different Fc variants at reducing inflammasome activation in human macrophages.

FIG. 24A and FIG. 24B set forth data showing increased lysosome stress response (as measured by Lysotracker fluorescence staining) in human macrophages stimulated with rapamycin; FIG. 24C and FIG. 24D set forth data showing differences in lysosome stress response in mouse bone marrow derived macrophages obtained from wildtype or GPNMB knockout mice in the presence or absence of rapamycin; FIG. 24E and FIG. 24F set forth data showing the effect of anti-GPNMB antibodies of the present disclosure on lysosome stress response in human macrophages.

FIG. 25A-FIG. 25D set forth data showing affinity matured anti-GPNMB antibodies of the present disclosure were effective at reducing rapamycin-induced lysosome stress in human macrophages.

FIG. 26A-FIG. 26C set forth data showing the effects of anti-GPNMB antibodies of the present disclosure having different Fc variants on reducing rapamycin-induced lysosome stress in human macrophages.

FIG. 27A-FIG. 27D set forth data showing affinity matured anti-GPNMB antibodies of the present disclosure increased expression of PDL1 and CD40 in human macrophages.

FIG. 28A-FIG. 28D set forth data showing the effects of anti-GPNMB antibodies of the present disclosure having different Fc variants on increasing expression of PDL1 and CD40 in human macrophages.

FIG. 29A-FIG. 29C set forth data showing anti-GPNMB antibodies of the present disclosure having different Fc variants increased GCase activity in human macrophages.

FIG. 30A-FIG. 30C set forth data showing anti-GPNMB antibodies of the present disclosure reduced expression of mature forms of GPNMB protein in human macrophages.

FIG. 31A-FIG. 31C set forth data showing anti-GPNMB antibodies of the present disclosure reduced expression of mature forms of GPNMB protein in human macrophages.

FIG. 32A-FIG. 32C set forth data showing anti-GPNMB antibodies GPN-41 and GPN-65 reduced expression of both mature and immature forms of GPNMB protein in human macrophages.

FIG. 33A and FIG. 33B set forth data showing anti-GPNMB antibodies of the present disclosure reduced LAMP-2 levels in splenocytes, macrophages, monocytes, and neutrophils.

FIG. 34A-FIG. 34D set forth data showing anti-GPNMB antibodies of the present disclosure increased GCase activity in primary microglia, splenic macrophages, neutrophils, monocytes, and macrophages.

### DETAILED DESCRIPTION OF THE PRESENT DISCLOSURE

The present disclosure relates to anti-GPNMB antibodies (e.g., monoclonal antibodies); methods of making and using such antibodies; pharmaceutical compositions comprising such antibodies; nucleic acids encoding such antibodies; and host cells comprising nucleic acids encoding such antibodies.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### Definitions

The terms “GPNMB” or “GPNMB polypeptide” or “GPNMB protein” are used interchangeably herein refer herein to any native GPNMB from any vertebrate source, including mammals such as primates (e.g., humans and cynomolgus monkeys (cynos)) and rodents (e.g., mice and rats), unless otherwise indicated. GPNMB is also referred to as osteoactivin (rat ortholog), dendritic cell-heparin integrin ligand (DC-HIL, mouse ortholog), or hematopoietic growth factor inducible neurokinin-1 type (HGFIN). In some embodiments, the term encompasses both wild-type sequences and naturally occurring variant sequences, e.g., splice variants or allelic variants. In some embodiments, the term encompasses “full-length,” unprocessed GPNMB as well as any form of GPNMB that results from processing in the cell. In some embodiments, the GPNMB is human GPNMB. As used herein, the term “human GPNMB” refers to a polypeptide with the amino acid sequence of SEQ ID NO: 1.

The terms “anti-GPNMB antibody,” an “antibody that binds to GPNMB,” and “antibody that specifically binds GPNMB” refer to an antibody that is capable of binding GPNMB with sufficient affinity such that the antibody is useful as a diagnostic and/or therapeutic agent in targeting GPNMB. In one embodiment, the extent of binding of an anti-GPNMB antibody to an unrelated, non-GPNMB polypeptide is less than about 10% of the binding of the antibody to GPNMB as measured, e.g., by a radioimmunoassay (RIA). In certain embodiments, an antibody that binds to GPNMB has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹ M, e.g., from 10⁻⁹ M to 10⁻¹³ M). In certain embodiments, an anti-GPNMB antibody binds to an epitope of GPNMB that is conserved among GPNMB from different species.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

The term “immunoglobulin” (Ig) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antigen-binding antibody fragments so long as they exhibit the desired biological activity.

“Native antibodies” are usually heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The light chain from any vertebrate species can be assigned to one of two clearly distinct types, called kappa (“κ”) and lambda (“λ”), based on the amino acid sequences of their constant domains.

Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody, such as an anti-GPNMB antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen-binding sites.

The term “variable” refers to the fact that certain segments of the variable domains differ extensively in sequence among antibodies, such as anti-GPNMB antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-GPNMB antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypeptide(s), and/or cell(s), the hybridoma methods, B-cell cloning methods, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-GPNMB antibody, in its substantially intact form, as opposed to an antibody fragment. Specifically, full-length antibodies include those with 2 light chains and 2 heavy chains including an Fc region. The constant domains may be native sequence constant domains (e.g., human native sequence constant domains) or amino acid sequence variants thereof. In some cases, the intact antibody may have one or more effector functions.

The terms “monovalent antibody” or “monoarm antibody” refers to an antibody having a single antigen-binding recognition domain that is specific to a target antigen (i.e., the antibody comprises no more than one antigen-binding domain). In some embodiments, a single antigen-binding domain comprises a single variable region heavy chain polypeptide and a single variable region light chain polypeptide. An antibody that is “monovalent” for a target comprises no more than one antigen-binding domain for that target.

An “antibody fragment” refers to a molecule other than an intact antibody that comprises a portion of an intact antibody that binds the antigen to which the intact antibody binds. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., the hypervariable regions (HVR)).

Papain digestion of antibodies, such as anti-GPNMB antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Functional fragments” of antibodies, such as anti-GPNMB antibodies of the present disclosure, comprise a portion of an intact antibody, generally including the antigen-binding or variable region of the intact antibody or the Fc region of an antibody which retains or has modified FcR binding capability.

Examples of antibody fragments include linear antibody, single-chain antibody molecules and multispecific antibodies formed from antibody fragments.

The term “diabodies” refers to small antibody fragments prepared by constructing scFv fragments with short linkers (about 5-10 residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-GPNMB antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-GPNMB antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-GPNMB antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-display libraries. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-GPNMB antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino-acid modification” at a specified position, e.g., of an anti-GPNMB antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Fv” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen-binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least 80% homology with a native sequence Fc region and/or with an Fc region of a parent polypeptide, and most preferably at least 90% homology therewith, more preferably at least 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “compete” when used in the context of antibodies that compete for the same epitope or overlapping epitopes means competition between antibody as determined by an assay in which the antibody being tested prevents or inhibits (e.g., reduces) specific binding of a reference molecule (e.g., a ligand, or a reference antibody) to a common antigen (e.g., GPNMB or a fragment thereof). Numerous types of competitive binding assays can be used to determine if antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit (e.g., reduce) specific binding of a reference antibody to a common antigen by at least 20%, 30%, 40%, 50%, 60%, 70%, 80%, 85%, 90%, 95%, 97.5%, and/or near 100%.

As used herein, an “interaction” between a GPNMB polypeptide and a second polypeptide encompasses, without limitation, protein-protein interaction, a physical interaction, a chemical interaction, binding, covalent binding, and ionic binding. As used herein, an antibody “inhibits interaction” between two polypeptides when the antibody disrupts, reduces, or completely eliminates an interaction between the two polypeptides. An antibody of the present disclosure, thereof, “inhibits interaction” between two polypeptides when the antibody thereof binds to one of the two polypeptides. In some embodiments, the interaction can be inhibited by at least any of 20%, 30%, 40%, 50%, 60%, 70%, 80%, 85%, 90%, 95%, 97.5%, and/or near 100%.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of polypeptides and/or macromolecules.

An “isolated” antibody, such as an isolated anti-GPNMB antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

An “isolated” nucleic acid molecule encoding an antibody, such as an anti-GPNMB antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic result. An effective amount can be provided in one or more administrations. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” or “in combination” with another compound or composition includes simultaneous administration and/or administration at different times.

Administration in conjunction or in combination also encompasses administration as a co-formulation or administration as separate compositions, including at different dosing frequencies or intervals, and using the same route of administration or different routes of administration. In some embodiments, administration in conjunction is administration as a part of the same treatment regimen.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### Anti-GPNMB Antibodies

Provided herein are monovalent anti-GPNMB antibodies. Antibodies provided herein are useful, e.g., for the treatment of the GPNMB associated disorders.

In one aspect, the present disclosure provides isolated (e.g., monoclonal) antibodies that bind to an epitope within a GPNMB protein or polypeptide of the present disclosure. GPNMB proteins or polypeptides of the present disclosure include, without limitation, a mammalian GPNMB protein or polypeptide, human GPNMB protein or polypeptide, mouse (murine) GPNMB protein or polypeptide, and cynomolgus GPNMB protein or polypeptide. GPNMB proteins and polypeptides of the present disclosure include naturally occurring variants of GPNMB. In some embodiments, GPNMB proteins and polypeptides of the present disclosure are membrane bound. In some embodiments, GPNMB proteins and polypeptides of the present disclosure are a soluble extracellular domain of GPNMB.

In some embodiments, GPNMB is expressed in a cell. In some embodiments, GPNMB is expressed in myeloid cells, including without limitation, phagocytic cells, microglia, macrophages, dendritic cells, osteoclasts, oligodentrocytes, and melanocytes. Additionally, GPNMB displays ectopic or overexpression in numerous cancers (Linger et al, 2008, Adv Cancer Res, 100:35-83).

In some aspects, anti-GPNMB antibodies of the present disclosure bind human GPNMB, bind mouse GPNMB, bind cynomolgus GMNMB, bind both human and mouse GPNMB, or bind human, mouse, and cynomolgus GPNMB.

In some aspects, anti-GPNMB antibodies of the present disclosure bind human GPNMB with an affinity of about 0.4 nM to about 120 nM. In some aspects, anti-GPNMB antibodies of the present disclosure bind mouse GPNMB with an affinity of about 0.3 nM to about 5 nM. In some aspects, anti-GPNMB antibodies of the present disclosure bind cynomolgus GPNMB with an affinity of about 0.4 nM to about 1.04 nM. In other aspects, anti-GPNMB antibodies of the present disclosure bind human GPNMB with an affinity of about 0.14 to about 0.65 nM and bind mouse GPNMB with an affinity of about 0.18 nM to about 0.44 nM.

In some aspects, anti-GPNMB antibodies of the present disclosure are antagonistic antibodies. In some aspects, anti-GPNMB antibodies of the present disclosure increase expression levels of PDL1 (e.g., in macrophages); increase expression levels of CD40 (e.g., in macrophages); increase expression levels of CD80 (e.g., in macrophages); increase serum cytokine expression levels of IL-12p40 and CCL5. In some aspects, anti-GPNMB antibodies of the present disclosure promote macrophage activation.

In some aspects, anti-GPNMB antibodies of the present disclosure increase expression levels of GCase (e.g., in macrophages). In some aspects, anti-GPNMB antibodies of the present disclosure are effective at overcoming a decrease in GCase activity associated with reduced progranulin levels.

In some aspects, anti-GPNMB antibodies of the present disclosure decrease GPNMB expression levels in cells (e.g., in macrophages). In some aspects, anti-GPNMB antibodies of the present disclosure decrease LAMP2 expression levels in cells (e.g., in macrophages, monocytes, neutrophils).

In some aspects, anti-GPNMB antibodies of the present disclosure inhibit or reduce inflammasome activation. In some aspects, anti-GPNMB antibodies of the present disclosure inhibit IL-1β expression or release.

In some aspects, anti-GPNMB antibodies of the present disclosure reduce neural inflammation. In some aspects, anti-GPNMB antibodies of the present disclosure reduce expression of C1q, GFAP, IBA1, and CTSD associated with neural inflammation.

In some aspects, anti-GPNMB antibodies of the present disclosure reduce lysosomal stress.

### GPNMB Protein

Human GPNMB is a type 1 transmembrane glycoprotein that, as a result of alternative splicing, occurs as two polypeptide isoforms, one of 572 amino acids and a shorter of 560 amino acids. GPNMB has 12 glycosylation sites, a polycystic kidney disease (PKD) domain, an integrin-recognition (RGD) motif, an immunoreceptor tyrosine-based activation-like motif (ITAM-like), and a lysosomal targeting (dileucine) motif (Abdelmagid et al, 2008, Exp Cell Res, 314:2334-2351). GPNMB can be cleaved by the metalloproteinase ADAM10, releasing a soluble fragment that can bind to various receptors and trigger a cellular response (Rose et al, 2010, PLoS One, 5(8):e12093).

### GPNMB, Lysosomal Dysfunction, and Parkinson's Disease

Glucocerebrosidase is a lysosomal β-glucosidase-degrading glucosylceramide. Inherited deficiency of glucocerebrosidase is the cause of autosomal recessive Gaucher's disease, the most common lysosomal storage disease (Brady et al, 1966, J Clin Invest, 45:1112-1115). This disease is caused by mutations in the lysosomal hydrolase β-glucosidase glycocerebrosidase, causing an accumulation of its substrate glycosylceramide (van der Lienden et al, 2018, Int Journal of Molecular Sciences, 20:66).Genetic deficiency of glucocerebrosidase is also a risk factor for Parkinson's disease and Lewy body dementia (Sidransky et al, 2009, N Eng J Med, 361:1651-1661; Tsuang et al, 2012, Neurology, 79:1944-1950). Conduritol β-epoxide (CBE) is an irreversible inhibitor of glucocerebrosidase and is used to generate in vitro and in vivo models for investigating Gaucher diseases and Parkinson's disease. Systemic inhibition of lysosomal glucocerebrosidase using conduritol-β-epoxide (CBE) is associated with specific Parkinson's disease-relevant pathologies, including accumulation of α-synuclein aggregates, elevations of glycosphingolipids, and widespread neuroinflammation (Rocha et al, 2015, Antioxid Redox Signal, 23:550-564).

Parkinson's disease is a progressive disorder that affects movement, and it is recognized as the second most common neurodegenerative disease after Alzheimer's disease. Common symptoms of Parkinson's disease include resting tremor, rigidity, and bradykinesia, and non-motor symptoms, such as depression, constipation, pain, sleep disorders, genitourinary problems, cognitive decline, and olfactory dysfunction, are also increasingly being associated with this disorder. A key feature of

Recent studies have shown a link between mutations in the glucocerebrosidase gene and increased risk of Parkinson's disease, with more severe mutations imparting higher levels of risk. Reduced glucocerebrosidase activity has been reported in the substantia nigra, cerebellum, and caudate of Parkinson's disease patients (Alcalay et al., 2015, Brain 138:2648).

Glucocerebrosidase mutations result in a gain of toxic function and/or altered cellular function due to a diversion of cellular resources (Gregg et al., 2012, Ann. Neurol. 72:455-46; Schondorf et al, 2014, Nat. Commun. 5:4028; Kilpatrick et al., 2016, Cell Calcium. 59:12-20; Cullen et al., 2011, Ann. Neurol. 69:940-953). Studies in rodent models of Parkinson's disease have also suggested a link between glucocerebrosidase activity and α-synuclein accumulation (Rocha et al., 2015, Antioxidants & Redox Signaling 23: 550; Rocha et al., 2015, Neurobiology of Disease 82:495).

Increased expression of GPNMB in the brain has been detected in a diverse number of neurodegenerative disorders, including Parkinson's disease, Alzheimer's disease, and ALS (Huttenrauch et al, 2018, Acta Neuropathol Commun, 6:108; Tanaka et al, 2012, Sci Rep, 2:573; Ono et al, 2016, Sci Rep, 6:23241; Moloney et al, 2018, Neurobiol Dis, 120:1-11). GPNMB levels are elevated in the substantia nigra of sporadic Parkinson's disease patients (Moloney et al, 2018, Neurobiol Dis, 120:1-11). Furthermore, the single nucleotide polymorphism (SNP) rs199347, a known top risk SNP for Parkinson's disease, is located in the GPNMB gene and results in increased GPNMB expression (Murthy et al, 2017, Neurogenetics, 18:121-133).

The present disclosure shows that reduction in GPNMB activity or expression with anti-GNPMB antibodies increases glucocerebrosidase activity.

### GPNMB and Cancer

GPNMB expression and function have been associated with cancer. GPNMB was first identified as a gene that was differentially expressed among melanoma cells lines with high and low metastatic potential (Weterman et al, 1995, Int J Cancer, 60:73-81; Tse et al, 2006, Clin Cancer Res, 12:1373-1382; Kuan et al, 2006, Clinical Cancer Research, 12:1970-1982; Rose et al, 2007, Molecular Cancer Research, 5:1001-1014). GPNMB expression has also been described in liver cancer, squamous cell lung carcinoma, and soft tissue tumors (Onaga et al, 2003, J Hepatol, 39:779-785; Borczuk et al, 2003, Am J Pathol, 163:1949-1960; Nielsen et al, 2002, Lancet, 359:1301-1307). Ectopic expression of GPNMB in cancer cells increased their in vitro invasiveness and promoted their metastasis in vivo (Onaga et al, 2003, J Hepatol, 39:779-785; Rich and Shi, 2003, J Biol Chem, 278:15951-15957).

GPNMB is highly expressed in multiple tumor types, including triple negative breast cancers, uveal and cutaneous melanoma, glioblastomas, hepatocellular carcinoma, prostate cancer, osteosarcoma, lung cancer, bladder cancer, and lymphangioleiomyomatosis (Taya and Hammes, 2018, Steroids, 133:102-107). It has been shown that GPNMB promotes the migration, invasion, and metastasis of tumor cells. GPNMB's role in driving tumor progression via its ability to dampen the inflammatory response around cancerous growth (Maric et al, 2013, One Targets Therapy, 6:839-852).

An antibody to GPNMB (called glembatumumab vedotin) was evaluated in phase I/II clinical trials as a treatment for advanced breast cancer and melanoma; however, these clinical trials were discontinued in 2018 after the drug candidate failed to improve the survival rate and stop tumor progression (Saade et al, 2021, Frontiers in Immunol, 12:674739). The present disclosure shows that reduction in GPNMB activity or expression with anti-GNPMB antibodies reduces tumor growth in a mouse tumor model.

### Exemplary Anti-GPNMB Antibodies and Certain Other Antibody Embodiments

In some aspects, provided herein are anti-GPNMB antibodies comprising at least one, two, three, four, five, or six HVRs selected from: (a) HVR-H1 comprising an amino acid sequence selected the group consisting of SEQ ID NOs:5, 8, 11, 14, 17, 20, 23, 26, 29, 32, 35, 38, 41, 44, 47, 50, 53, 56, 59, 62, 65, 68, 71, 74, and 77; (b) HVR-H2 comprising an amino acid sequence selected from the group consisting of SEQ ID NOs:6, 9, 12, 15, 18, 21, 24, 27, 30, 33, 36, 39, 42, 45, 48, 51, 54, 57, 60, 63, 66, 69, 72, 75, and 78; (c) HVR-H3 comprising an amino acid sequence selected from the group consisting of SEQ ID NOs:7, 10, 13, 16, 19, 22, 25, 28, 31, 34, 37, 40, 43, 46, 49, 52, 55, 58, 61, 64, 67, 70, 73, 76, and 79; (d) HVR-L1 comprising an amino acid sequence selected from the group consisting of SEQ ID NOs:83, 86, 89, 92, 95, 98, 101, 104, 107, 110, 113, 116, 119, 122, 125, 128, 131, 134, 137, 140, 143, 146, 149, 152, and 155; (e) HVR-L2 comprising an amino acid sequence selected from the group consisting of SEQ ID NOs:84, 87, 90, 93, 96, 99, 102, 105, 108, 111, 114, 117, 120, 123, 126, 129, 132, 135, 138, 141, 144, 147, 150, 153, and 156; and (f) HVR-L3 comprising an amino acid sequence selected from the group consisting of SEQ ID NOs:85, 88, 91, 94, 97, 100, 103, 106, 109, 112, 115, 118, 121, 124, 127, 130, 133, 136, 139, 142, 145, 148, 151, 154, and 157.

In some aspects, provided herein are anti-GPNMB antibodies comprising at least one, at least two, or all three V_{H} HVR sequences selected from (a) HVR-H1 comprising an amino acid sequence selected the group consisting of SEQ ID NOs:5, 8, 11, 14, 17, 20, 23, 26, 29, 32, 35, 38, 41, 44, 47, 50, 53, 56, 59, 62, 65, 68, 71, 74, and 77; (b) HVR-H2 comprising an amino acid sequence selected from the group consisting of SEQ ID NOs:6, 9, 12, 15, 18, 21, 24, 27, 30, 33, 36, 39, 42, 45, 48, 51, 54, 57, 60, 63, 66, 69, 72, 75, and 78; (c) HVR-H3 comprising an amino acid sequence selected from the group consisting of SEQ ID NOs:7, 10, 13, 16, 19, 22, 25, 28, 31, 34, 37, 40, 43, 46, 49, 52, 55, 58, 61, 64, 67, 70, 73, 76, and 79.

In some aspects, provided herein are anti-GPNMB antibodies comprising at least one, at least two, or all three V_{L} HVR sequences selected from (a) HVR-L1 comprising an amino acid sequence selected from the group consisting of SEQ ID NOs:83, 86, 89, 92, 95, 98, 101, 104, 107, 110, 113, 116, 119, 122, 125, 128, 131, 134, 137, 140, 143, 146, 149, 152, and 155; (b) HVR-L2 comprising an amino acid sequence selected from the group consisting of SEQ ID NOs:84, 87, 90, 93, 96, 99, 102, 105, 108, 111, 114, 117, 120, 123, 126, 129, 132, 135, 138, 141, 144, 147, 150, 153, and 156; and (c) HVR-L3 comprising an amino acid sequence selected from the group consisting of SEQ ID NOs:85, 88, 91, 94, 97, 100, 103, 106, 109, 112, 115, 118, 121, 124, 127, 130, 133, 136, 139, 142, 145, 148, 151, 154, and 157.

In some aspects, provided herein are anti-GPNMB antibodies comprising (a) a V_{H} domain comprising at least one, at least two, or all three V_{H} HVR sequences selected from (i) HVR-H1 comprising an amino acid sequence selected from the group consisting of SEQ ID NOs: 5, 8, 11, 14, 17, 20, 23, 26, 29, 32, 35, 38, 41, 44, 47, 50, 53, 56, 59, 62, 65, 68, 71, 74, and 77; (ii) HVR-H2 comprising an amino acid sequence selected from the group consisting of SEQ ID NOs: 6, 9, 12, 15, 18, 21, 24, 27, 30, 33, 36, 39, 42, 45, 48, 51, 54, 57, 60, 63, 66, 69, 72, 75, and 78; and (iii) HVR-H3 comprising an amino acid sequence selected from the group consisting of SEQ ID NOs: 7, 10, 13, 16, 19, 22, 25, 28, 31, 34, 37, 40, 43, 46, 49, 52, 55, 58, 61, 64, 67, 70, 73, 76, and 79; and (b) a V_{L} domain comprising at least one, at least two, or all three V_{L} HVR sequences selected from (i) HVR-L1 comprising an amino acid sequence selected from the group consisting of SEQ ID NOs: 83, 86, 89, 92, 95, 98, 101, 104, 107, 110, 113, 116, 119, 122, 125, 128, 131, 134, 137, 140, 143, 146, 149, 152, and 155; (ii) HVR-L2 comprising an amino acid sequence selected from the group consisting of SEQ ID NOs: 84, 87, 90, 93, 96, 99, 102, 105, 108, 111, 114, 117, 120, 123, 126, 129, 132, 135, 138, 141, 144, 147, 150, 153, and 156; and (iii) HVR-L3 comprising an amino acid sequence selected from the group consisting of SEQ ID NOs: 85, 88, 91, 94, 97, 100, 103, 106, 109, 112, 115, 118, 121, 124, 127, 130, 133, 136, 139, 142, 145, 148, 151, 154, and 157.

In some aspects, provided herein are anti-GPNMB antibodies comprising: (a) HVR-H1 comprising the amino acid sequence of SEQ ID NO:5; (b) HVR-H2 comprising the amino acid sequence of SEQ ID NO:6; (c) HVR-H3 comprising the amino acid sequence of SEQ ID NO:7; (d) HVR-L1 comprising the amino acid sequence of SEQ ID NO:83; (e) HVR-L2 comprising the amino acid sequence of SEQ ID NO:84; and (f) HVR-L3 comprising the amino acid sequence of SEQ ID NO:85; (a) HVR-H1 comprising the amino acid sequence of SEQ ID NO:8; (b) HVR-H2 comprising the amino acid sequence of SEQ ID NO:9; (c) HVR-H3 comprising the amino acid sequence of SEQ ID NO:10; (d) HVR-L1 comprising the amino acid sequence of SEQ ID NO:86; (e) HVR-L2 comprising the amino acid sequence of SEQ ID NO:87; and (f) HVR-L3 comprising the amino acid sequence of SEQ ID NO:88; (a) HVR-H1 comprising the amino acid sequence of SEQ ID NO:11; (b) HVR-H2 comprising the amino acid sequence of SEQ ID NO:12; (c) HVR-H3 comprising the amino acid sequence of SEQ ID NO:13; (d) HVR-L1 comprising the amino acid sequence of SEQ ID NO:89; (e) HVR-L2 comprising the amino acid sequence of SEQ ID NO:90; and (f) HVR-L3 comprising the amino acid sequence of SEQ ID NO:91; (a) HVR-H1 comprising the amino acid sequence of SEQ ID NO:14; (b) HVR-H2 comprising the amino acid sequence of SEQ ID NO:15; (c) HVR-H3 comprising the amino acid sequence of SEQ ID NO:16; (d) HVR-L1 comprising the amino acid sequence of SEQ ID NO:92; (e) HVR-L2 comprising the amino acid sequence of SEQ ID NO:93; and (f) HVR-L3 comprising the amino acid sequence of SEQ ID NO:94; (a) HVR-H1 comprising the amino acid sequence of SEQ ID NO:17; (b) HVR-H2 comprising the amino acid sequence of SEQ ID NO:18; (c) HVR-H3 comprising the amino acid sequence of SEQ ID NO:19; (d) HVR-L1 comprising the amino acid sequence of SEQ ID NO:95; (e) HVR-L2 comprising the amino acid sequence of SEQ ID NO:96; and (f) HVR-L3 comprising the amino acid sequence of SEQ ID NO:97; (a) HVR-H1 comprising the amino acid sequence of SEQ ID NO:20; (b) HVR-H2 comprising the amino acid sequence of SEQ ID NO:21; (c) HVR-H3 comprising the amino acid sequence of SEQ ID NO:22; (d) HVR-L1 comprising the amino acid sequence of SEQ ID NO:98; (e) HVR-L2 comprising the amino acid sequence of SEQ ID NO:99; and (f) HVR-L3 comprising the amino acid sequence of SEQ ID NO:100; (a) HVR-H1 comprising the amino acid sequence of SEQ ID NO:23; (b) HVR-H2 comprising the amino acid sequence of SEQ ID NO:24; (c) HVR-H3 comprising the amino acid sequence of SEQ ID NO:25; (d) HVR-L1 comprising the amino acid sequence of SEQ ID NO:101; (e) HVR-L2 comprising the amino acid sequence of SEQ ID NO:102; and (f) HVR-L3 comprising the amino acid sequence of SEQ ID NO:103; (a) HVR-H1 comprising the amino acid sequence of SEQ ID NO:26; (b) HVR-H2 comprising the amino acid sequence of SEQ ID NO:27; (c) HVR-H3 comprising the amino acid sequence of SEQ ID NO:28; (d) HVR-L1 comprising the amino acid sequence of SEQ ID NO:104; (e) HVR-L2 comprising the amino acid sequence of SEQ ID NO:105; and (f) HVR-L3 comprising the amino acid sequence of SEQ ID NO:106; (a) HVR-H1 comprising the amino acid sequence of SEQ ID NO:29; (b) HVR-H2 comprising the amino acid sequence of SEQ ID NO:30; (c) HVR-H3 comprising the amino acid sequence of SEQ ID NO:31; (d) HVR-L1 comprising the amino acid sequence of SEQ ID NO:107; (e) HVR-L2 comprising the amino acid sequence of SEQ ID NO:108; and (f) HVR-L3 comprising the amino acid sequence of SEQ ID NO:109; (a) HVR-H1 comprising the amino acid sequence of SEQ ID NO:32; (b) HVR-H2 comprising the amino acid sequence of SEQ ID NO:33; (c) HVR-H3 comprising the amino acid sequence of SEQ ID NO:34; (d) HVR-L1 comprising the amino acid sequence of SEQ ID NO:110; (e) HVR-L2 comprising the amino acid sequence of SEQ ID NO:111; and (f) HVR-L3 comprising the amino acid sequence of SEQ ID NO:112; (a) HVR-H1 comprising the amino acid sequence of SEQ ID NO:35; (b) HVR-H2 comprising the amino acid sequence of SEQ ID NO:36; (c) HVR-H3 comprising the amino acid sequence of SEQ ID NO:37; (d) HVR-L1 comprising the amino acid sequence of SEQ ID NO:113; (e) HVR-L2 comprising the amino acid sequence of SEQ ID NO:114; and (f) HVR-L3 comprising the amino acid sequence of SEQ ID NO:115; (a) HVR-H1 comprising the amino acid sequence of SEQ ID NO:38; (b) HVR-H2 comprising the amino acid sequence of SEQ ID NO:39; (c) HVR-H3 comprising the amino acid sequence of SEQ ID NO:40; (d) HVR-L1 comprising the amino acid sequence of SEQ ID NO:116; (e) HVR-L2 comprising the amino acid sequence of SEQ ID NO:117; and (f) HVR-L3 comprising the amino acid sequence of SEQ ID NO:118; (a) HVR-H1 comprising the amino acid sequence of SEQ ID NO:41; (b) HVR-H2 comprising the amino acid sequence of SEQ ID NO:42; (c) HVR-H3 comprising the amino acid sequence of SEQ ID NO:43; (d) HVR-L1 comprising the amino acid sequence of SEQ ID NO:119; (e) HVR-L2 comprising the amino acid sequence of SEQ ID NO:120; and (f) HVR-L3 comprising the amino acid sequence of SEQ ID NO:121; (a) HVR-H1 comprising the amino acid sequence of SEQ ID NO:44; (b) HVR-H2 comprising the amino acid sequence of SEQ ID NO:45; (c) HVR-H3 comprising the amino acid sequence of SEQ ID NO:46; (d) HVR-L1 comprising the amino acid sequence of SEQ ID NO:122; (e) HVR-L2 comprising the amino acid sequence of SEQ ID NO:123; and (f) HVR-L3 comprising the amino acid sequence of SEQ ID NO:124; (a) HVR-H1 comprising the amino acid sequence of SEQ ID NO:47; (b) HVR-H2 comprising the amino acid sequence of SEQ ID NO:48; (c) HVR-H3 comprising the amino acid sequence of SEQ ID NO:49; (d) HVR-L1 comprising the amino acid sequence of SEQ ID NO:125; (e) HVR-L2 comprising the amino acid sequence of SEQ ID NO:126; and (f) HVR-L3 comprising the amino acid sequence of SEQ ID NO:127; (a) HVR-H1 comprising the amino acid sequence of SEQ ID NO:50; (b) HVR-H2 comprising the amino acid sequence of SEQ ID NO:51; (c) HVR-H3 comprising the amino acid sequence of SEQ ID NO:52; (d) HVR-L1 comprising the amino acid sequence of SEQ ID NO:128; (e) HVR-L2 comprising the amino acid sequence of SEQ ID NO:129; and (f) HVR-L3 comprising the amino acid sequence of SEQ ID NO:130; (a) HVR-H1 comprising the amino acid sequence of SEQ ID NO:53; (b) HVR-H2 comprising the amino acid sequence of SEQ ID NO:54; (c) HVR-H3 comprising the amino acid sequence of SEQ ID NO:55; (d) HVR-L1 comprising the amino acid sequence of SEQ ID NO:131; (e) HVR-L2 comprising the amino acid sequence of SEQ ID NO:132; and (f) HVR-L3 comprising the amino acid sequence of SEQ ID NO:133; (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:58; (d) HVR-L1 comprising the amino acid sequence of SEQ ID NO:134; (e) HVR-L2 comprising the amino acid sequence of SEQ ID NO:135; and (f) HVR-L3 comprising the amino acid sequence of SEQ ID NO:136; (a) HVR-H1 comprising the amino acid sequence of SEQ ID NO:59; (b) HVR-H2 comprising the amino acid sequence of SEQ ID NO:60; (c) HVR-H3 comprising the amino acid sequence of SEQ ID NO:61; (d) HVR-L1 comprising the amino acid sequence of SEQ ID NO:137; (e) HVR-L2 comprising the amino acid sequence of SEQ ID NO:138; and (f) HVR-L3 comprising the amino acid sequence of SEQ ID NO:139; (a) HVR-H1 comprising the amino acid sequence of SEQ ID NO:62; (b) HVR-H2 comprising the amino acid sequence of SEQ ID NO:63; (c) HVR-H3 comprising the amino acid sequence of SEQ ID NO:64; (d) HVR-L1 comprising the amino acid sequence of SEQ ID NO:140; (e) HVR-L2 comprising the amino acid sequence of SEQ ID NO:141; and (f) HVR-L3 comprising the amino acid sequence of SEQ ID NO:142; (a) HVR-H1 comprising the amino acid sequence of SEQ ID NO:65; (b) HVR-H2 comprising the amino acid sequence of SEQ ID NO:66; (c) HVR-H3 comprising the amino acid sequence of SEQ ID NO:67; (d) HVR-L1 comprising the amino acid sequence of SEQ ID NO:143; (e) HVR-L2 comprising the amino acid sequence of SEQ ID NO:144; and (f) HVR-L3 comprising the amino acid sequence of SEQ ID NO:145; (a) HVR-H1 comprising the amino acid sequence of SEQ ID NO:68; (b) HVR-H2 comprising the amino acid sequence of SEQ ID NO:69; (c) HVR-H3 comprising the amino acid sequence of SEQ ID NO:70; (d) HVR-L1 comprising the amino acid sequence of SEQ ID NO:146; (e) HVR-L2 comprising the amino acid sequence of SEQ ID NO:147; and (f) HVR-L3 comprising the amino acid sequence of SEQ ID NO:148; (a) HVR-H1 comprising the amino acid sequence of SEQ ID NO:71; (b) HVR-H2 comprising the amino acid sequence of SEQ ID NO:72; (c) HVR-H3 comprising the amino acid sequence of SEQ ID NO:73; (d) HVR-L1 comprising the amino acid sequence of SEQ ID NO:149; (e) HVR-L2 comprising the amino acid sequence of SEQ ID NO:150; and (f) HVR-L3 comprising the amino acid sequence of SEQ ID NO:151; (a) HVR-H1 comprising the amino acid sequence of SEQ ID NO:74; (b) HVR-H2 comprising the amino acid sequence of SEQ ID NO:75; (c) HVR-H3 comprising the amino acid sequence of SEQ ID NO:76; (d) HVR-L1 comprising the amino acid sequence of SEQ ID NO:152; (e) HVR-L2 comprising the amino acid sequence of SEQ ID NO:153; and (f) HVR-L3 comprising the amino acid sequence of SEQ ID NO:154; and (a) HVR-H1 comprising the amino acid sequence of SEQ ID NO:77; (b) HVR-H2 comprising the amino acid sequence of SEQ ID NO:78; (c) HVR-H3 comprising the amino acid sequence of SEQ ID NO:79; (d) HVR-L1 comprising the amino acid sequence of SEQ ID NO:155; (e) HVR-L2 comprising the amino acid sequence of SEQ ID NO:156; and (f) HVR-L3 comprising the amino acid sequence of SEQ ID NO:157.

In another aspect, an anti-GPNMB antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:161, 163, 165, 167, 169, 171, 173, 175, 177, 179, 181, 183, 185, 187, 189, 191, 193, 195, 197, 199, 201, 203, 205, 207, and 209. In certain aspects, a V_{H} sequence having at least 90%, 91%, 92%, 93%, 94%, 95%, 96%, 97%, 98%, or 99% identity to an amino acid sequence selected from the group consisting of SEQ ID NOs: 161, 163, 165, 167, 169, 171, 173, 175, 177, 179, 181, 183, 185, 187, 189, 191, 193, 195, 197, 199, 201, 203, 205, 207, and 209 contains substitutions (e.g., conservative substitutions), insertions, or deletions relative to the reference sequence, but an anti-GPNMB antibody comprising that sequence retains the ability to bind to GPNMB. In certain aspects, a total of 1 to 10 amino acids have been substituted, inserted, and/or deleted in SEQ ID NO: 161, 163, 165, 167, 169, 171, 173, 175, 177, 179, 181, 183, 185, 187, 189, 191, 193, 195, 197, 199, 201, 203, 205, 207, and 209. In certain aspects, substitutions, insertions, or deletions occur in regions outside the HVRs (i.e., in the FRs). Optionally, the anti-GPNMB antibody comprises the V_{H} sequence of SEQ ID NO: 161, 163, 165, 167, 169, 171, 173, 175, 177, 179, 181, 183, 185, 187, 189, 191, 193, 195, 197, 199, 201, 203, 205, 207, and 209, including post-translational modifications of that sequence. In a particular aspect, the V_{H} comprises one, two or three HVRs selected from: (a) HVR-H1 comprising an amino acid sequence selected from the group consisting of SEQ ID NOs: 5, 8, 11, 14, 17, 20, 23, 26, 29, 32, 35, 38, 41, 44, 47, 50, 53, 56, 59, 62, 65, 68, 71, 74, and 77; (b) HVR-H2 comprising an amino acid sequence selected from the group consisting of SEQ ID NOs: 6, 9, 12, 15, 18, 21, 24, 27, 30, 33, 36, 39, 42, 45, 48, 51, 54, 57, 60, 63, 66, 69, 72, 75, and 78; and (c) HVR-H3 comprising an amino acid sequence selected from the group consisting of SEQ ID NOs: 7, 10, 13, 16, 19, 22, 25, 28, 31, 34, 37, 40, 43, 46, 49, 52, 55, 58, 61, 64, 67, 70, 73, 76, and 79.

In another aspect, an anti-GPNMB antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs:162, 164, 166, 168, 170, 172, 174, 176, 178, 180, 182, 184, 186, 188, 190, 192, 194, 196, 198, 200, 202, 204, 206, 208, and 210. In certain aspects, a V_{L} sequence having at least 90%, 91%, 92%, 93%, 94%, 95%, 96%, 97%, 98%, or 99% identity to an amino acid sequence selected from the group consisting of SEQ ID NOs: 162, 164, 166, 168, 170, 172, 174, 176, 178, 180, 182, 184, 186, 188, 190, 192, 194, 196, 198, 200, 202, 204, 206, 208, and 210, and contains substitutions (e.g., conservative substitutions), insertions, or deletions relative to the reference sequence, but an anti-GPNMB antibody comprising that sequence retains the ability to bind to GPNMB. In some aspects, a total of 1 to 10 amino acids have been substituted, inserted and/or deleted in SEQ ID NO: 162, 164, 166, 168, 170, 172, 174, 176, 178, 180, 182, 184, 186, 188, 190, 192, 194, 196, 198, 200, 202, 204, 206, 208, or 210. In certain aspects, a total of 1 to 5 amino acids have been substituted, inserted and/or deleted in SEQ ID NO: 162, 164, 166, 168, 170, 172, 174, 176, 178, 180, 182, 184, 186, 188, 190, 192, 194, 196, 198, 200, 202, 204, 206, 208, or 210. In certain aspects, the substitutions, insertions, or deletions occur in regions outside the HVRs (i.e., in the FRs). Optionally, the anti-GPNMB antibody comprises the V_{L} sequence of SEQ ID NO: 162, 164, 166, 168, 170, 172, 174, 176, 178, 180, 182, 184, 186, 188, 190, 192, 194, 196, 198, 200, 202, 204, 206, 208, or 210, including post-translational modifications of that sequence. In a particular aspect, the V_{L} comprises one, two or three HVRs selected from (a) HVR-L1 comprising an amino acid sequence selected from the group consisting of SEQ ID NOs: 83, 86, 89, 92, 95, 98, 101, 104, 107, 110, 113, 116, 119, 122, 125, 128, 131, 134, 137, 140, 143, 146, 149, 152, and 155; (b) HVR-L2 comprising an amino acid sequence selected from the group consisting of SEQ ID NOs: 84, 87, 90, 93, 96, 99, 102, 105, 108, 111, 114, 117, 120, 123, 126, 129, 132, 135, 138, 141, 144, 147, 150, 153, and 156; and (c) HVR-L3 comprising an amino acid sequence selected from the group consisting of SEQ ID NOs: 85, 88, 91, 94, 97, 100, 103, 106, 109, 112, 115, 118, 121, 124, 127, 130, 133, 136, 139, 142, 145, 148, 151, 154, and 157.

In some aspects, an anti-GPNMB antibody is provided, wherein the antibody comprises a V_{H} as in any of the aspects provided above, and a V_{L} as in any of the aspects provided above. In some aspects, provided herein are anti-GPNMB antibodies, wherein the antibody comprises a V_{H} as in any of the aspects provided above, and a V_{L} as in any of the aspects provided above. In one aspect, the antibody comprises the V_{H} and V_{L} sequences in SEQ ID NOs: 161, 163, 165, 167, 169, 171, 173, 175, 177, 179, 181, 183, 185, 187, 189, 191, 193, 195, 197, 199, 201, 203, 205, 207, and 209, and SEQ ID NOs: 162, 164, 166, 168, 170, 172, 174, 176, 178, 180, 182, 184, 186, 188, 190, 192, 194, 196, 198, 200, 202, 204, 206, 208, and 210, respectively, including post-translational modifications of those sequences.

In some aspects, provided herein are anti-GPNMB antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO: 161 and V_{L} comprising the amino acid sequence of SEQ ID NO: 162; V_{H} comprising the amino acid sequence of SEQ ID NO: 163 and V_{L} comprising the amino acid sequence of SEQ ID NO: 164; V_{H} comprising the amino acid sequence of SEQ ID NO: 165 and V_{L} comprising the amino acid sequence of SEQ ID NO: 166; V_{H} comprising the amino acid sequence of SEQ ID NO: 167 and V_{L} comprising the amino acid sequence of SEQ ID NO: 168; V_{H} comprising the amino acid sequence of SEQ ID NO: 169 and V_{L} comprising the amino acid sequence of SEQ ID NO: 170; V_{H} comprising the amino acid sequence of SEQ ID NO: 171 and V_{L} comprising the amino acid sequence of SEQ ID NO: 172; V_{H} comprising the amino acid sequence of SEQ ID NO: 173 and V_{L} comprising the amino acid sequence of SEQ ID NO: 174; V_{H} comprising the amino acid sequence of SEQ ID NO: 175 and V_{L} comprising the amino acid sequence of SEQ ID NO: 176; V_{H} comprising the amino acid sequence of SEQ ID NO: 177 and V_{L} comprising the amino acid sequence of SEQ ID NO: 178; V_{H} comprising the amino acid sequence of SEQ ID NO: 179 and V_{L} comprising the amino acid sequence of SEQ ID NO:180; V_{H} comprising the amino acid sequence of SEQ ID NO: 181 and V_{L} comprising the amino acid sequence of SEQ ID NO: 182; V_{H} comprising the amino acid sequence of SEQ ID NO: 183 and V_{L} comprising the amino acid sequence of SEQ ID NO: 184; V_{H} comprising the amino acid sequence of SEQ ID NO: 185 and V_{L} comprising the amino acid sequence of SEQ ID NO: 186; V_{H} comprising the amino acid sequence of SEQ ID NO: 187 and V_{L} comprising the amino acid sequence of SEQ ID NO: 188; V_{H} comprising the amino acid sequence of SEQ ID NO: 189 and V_{L} comprising the amino acid sequence of SEQ ID NO: 190; V_{H} comprising the amino acid sequence of SEQ ID NO: 191 and V_{L} comprising the amino acid sequence of SEQ ID NO: 192; V_{H} comprising the amino acid sequence of SEQ ID NO: 193 and V_{L} comprising the amino acid sequence of SEQ ID NO: 194; V_{H} comprising the amino acid sequence of SEQ ID NO: 195 and V_{L} comprising the amino acid sequence of SEQ ID NO: 196; V_{H} comprising the amino acid sequence of SEQ ID NO: 197 and V_{L} comprising the amino acid sequence of SEQ ID NO: 198; V_{H} comprising the amino acid sequence of SEQ ID NO: 199 and V_{L} comprising the amino acid sequence of SEQ ID NO:200; V_{H} comprising the amino acid sequence of SEQ ID NO:201 and V_{L} comprising the amino acid sequence of SEQ ID NO:202; V_{H} comprising the amino acid sequence of SEQ ID NO:203 and V_{L} comprising the amino acid sequence of SEQ ID NO:204; V_{H} comprising the amino acid sequence of SEQ ID NO:205 and V_{L} comprising the amino acid sequence of SEQ ID NO:206; V_{H} comprising the amino acid sequence of SEQ ID NO:207 and V_{L} comprising the amino acid sequence of SEQ ID NO:208; and V_{H} comprising the amino acid sequence of SEQ ID NO:209 and V_{L} comprising the amino acid sequence of SEQ ID NO:210.

In some aspects, an anti-GPNMB antibody of the present disclosure competitively inhibits binding of at least one reference antibody selected from anti-GPNMB antibody GPN-01, GPN-03, GPN-06, GPN-07, GPN-08, GPN-09, GPN-11, GPN-22, GPN-24, GPN-25, GPN-26, GPN-30, GPN-31, GPN-33, GPN-34, GPN-35, GPN-37, GPN-38, GPN-41, GPN-42, GPN-43, GPN-48, GPN-52, GPN-61, and GPN-65 and any combination thereof, for binding to GPNMB.

In some aspects, an anti-GPNMB antibody of the present disclosure binds to an epitope of human GPNMB that is the same as or overlaps with the GPNMB epitope bound by at least one reference antibody selected from anti-GPNMB antibody GPN-01, GPN-03, GPN-06, GPN-07, GPN-08, GPN-09, GPN-11, GPN-22, GPN-24, GPN-25, GPN-26, GPN-30, GPN-31, GPN-33, GPN-34, GPN-35, GPN-37, GPN-38, GPN-41, GPN-42, GPN-43, GPN-48, GPN-52, GPN-61, and GPN-65. Detailed exemplary methods for mapping an epitope to which an antibody binds are provided in Morris (1996) “Epitope Mapping Protocols,” in Methods in Molecular Biology vol. 66 (Humana Press, Totowa, NJ).

In some aspects, an anti-GPNMB antibody of the present disclosure competitively inhibits binding of at least one reference antibody, or binds to an epitope of human GPNMB that is the same as or overlaps with the GPNMB epitope bound by at least one reference antibody, wherein the reference antibody is an anti-GPNMB antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:161 and V_{L} comprising the amino acid sequence of SEQ ID NO: 162; V_{H} comprising the amino acid sequence of SEQ ID NO: 163 and V_{L} comprising the amino acid sequence of SEQ ID NO: 164; V_{H} comprising the amino acid sequence of SEQ ID NO: 165 and V_{L} comprising the amino acid sequence of SEQ ID NO: 166; V_{H} comprising the amino acid sequence of SEQ ID NO: 167 and V_{L} comprising the amino acid sequence of SEQ ID NO: 168; V_{H} comprising the amino acid sequence of SEQ ID NO: 169 and V_{L} comprising the amino acid sequence of SEQ ID NO: 170; V_{H} comprising the amino acid sequence of SEQ ID NO: 171 and V_{L} comprising the amino acid sequence of SEQ ID NO: 172; V_{H} comprising the amino acid sequence of SEQ ID NO: 173 and V_{L} comprising the amino acid sequence of SEQ ID NO: 174; V_{H} comprising the amino acid sequence of SEQ ID NO: 175 and V_{L} comprising the amino acid sequence of SEQ ID NO: 176; V_{H} comprising the amino acid sequence of SEQ ID NO: 177 and V_{L} comprising the amino acid sequence of SEQ ID NO: 178; V_{H} comprising the amino acid sequence of SEQ ID NO: 179 and V_{L} comprising the amino acid sequence of SEQ ID NO: 180; V_{H} comprising the amino acid sequence of SEQ ID NO: 181 and V_{L} comprising the amino acid sequence of SEQ ID NO: 182; V_{H} comprising the amino acid sequence of SEQ ID NO: 183 and V_{L} comprising the amino acid sequence of SEQ ID NO: 184; V_{H} comprising the amino acid sequence of SEQ ID NO: 185 and V_{L} comprising the amino acid sequence of SEQ ID NO: 186; V_{H} comprising the amino acid sequence of SEQ ID NO: 187 and V_{L} comprising the amino acid sequence of SEQ ID NO: 188; V_{H} comprising the amino acid sequence of SEQ ID NO: 189 and V_{L} comprising the amino acid sequence of SEQ ID NO: 190; V_{H} comprising the amino acid sequence of SEQ ID NO: 191 and V_{L} comprising the amino acid sequence of SEQ ID NO: 192; V_{H} comprising the amino acid sequence of SEQ ID NO: 193 and V_{L} comprising the amino acid sequence of SEQ ID NO: 194; V_{H} comprising the amino acid sequence of SEQ ID NO: 195 and V_{L} comprising the amino acid sequence of SEQ ID NO: 196; V_{H} comprising the amino acid sequence of SEQ ID NO: 197 and V_{L} comprising the amino acid sequence of SEQ ID NO: 198; V_{H} comprising the amino acid sequence of SEQ ID NO: 199 and V_{L} comprising the amino acid sequence of SEQ ID NO:200; V_{H} comprising the amino acid sequence of SEQ ID NO:201 and V_{L} comprising the amino acid sequence of SEQ ID NO:202; V_{H} comprising the amino acid sequence of SEQ ID NO:203 and V_{L} comprising the amino acid sequence of SEQ ID NO:204; V_{H} comprising the amino acid sequence of SEQ ID NO:205 and V_{L} comprising the amino acid sequence of SEQ ID NO:206; V_{H} comprising the amino acid sequence of SEQ ID NO:207 and V_{L} comprising the amino acid sequence of SEQ ID NO:208; and V_{H} comprising the amino acid sequence of SEQ ID NO:209 and V_{L} comprising the amino acid sequence of SEQ ID NO:210.

In some aspects, provided herein are anti-GPNMB antibodies comprising at least one, two, three, four, five, or six HVRs selected from: (a) HVR-H1 comprising an amino acid sequence selected the group consisting of SEQ ID NOs:71 and 23; (b) HVR-H2 comprising an amino acid sequence selected from the group consisting of SEQ ID NOs:255, 256, 257, 258, 259, and 260; (c) HVR-H3 comprising an amino acid sequence selected from the group consisting of SEQ ID NOs:73, 261, 19, and 262; (d) HVR-L1 comprising an amino acid sequence selected from the group consisting of SEQ ID NOs:263, 264, 265, 266, 267, and 268; (e) HVR-L2 comprising an amino acid sequence selected from the group consisting of SEQ ID NOs:269, 270, 271, 272, 273, 96, 274, 275, 276, and 277; and (f) HVR-L3 comprising an amino acid sequence selected from the group consisting of SEQ ID NOs:151, 278, 279, 280, 97, and 281.

In some aspects, provided herein are anti-GPNMB antibodies comprising at least one, at least two, or all three V_{H} HVR sequences selected from (a) HVR-H1 comprising an amino acid sequence selected the group consisting of SEQ ID NOs: 71 and 23; (b) HVR-H2 comprising an amino acid sequence selected from the group consisting of SEQ ID NOs: 255, 256, 257, 258, 259, and 260; (c) HVR-H3 comprising an amino acid sequence selected from the group consisting of SEQ ID NOs: 73, 261, 19, and 262.

In some aspects, provided herein are anti-GPNMB antibodies comprising at least one, at least two, or all three V_{L} HVR sequences selected from (a) HVR-L1 comprising an amino acid sequence selected from the group consisting of SEQ ID NOs:263, 264, 265, 266, 267, and 268; (b) HVR-L2 comprising an amino acid sequence selected from the group consisting of SEQ ID NOs: 269, 270, 271, 272, 273, 96, 274, 275, 276, and 277; and (c) HVR-L3 comprising an amino acid sequence selected from the group consisting of SEQ ID NOs: 269, 270, 271, 272, 273, 96, 274, 275, 276, and 277.

In some aspects, provided herein are anti-GPNMB antibodies comprising (a) a V_{H} domain comprising at least one, at least two, or all three V_{H} HVR sequences selected from (i) HVR-H1 comprising an amino acid sequence selected from the group consisting of SEQ ID NOs: 71 and 23; (ii) HVR-H2 comprising an amino acid sequence selected from the group consisting of SEQ ID NOs: 255, 256, 257, 258, 259, and 260; and (iii) HVR-H3 comprising an amino acid sequence selected from the group consisting of SEQ ID NOs: 73, 261, 19, and 262; and (b) a V_{L} domain comprising at least one, at least two, or all three V_{L} HVR sequences selected from (i) HVR-L1 comprising an amino acid sequence selected from the group consisting of SEQ ID NOs: 263, 264, 265, 266, 267, and 268; (ii) HVR-L2 comprising an amino acid sequence selected from the group consisting of SEQ ID NOs: 263, 264, 265, 266, 267, and 268; and (iii) HVR-L3 comprising an amino acid sequence selected from the group consisting of SEQ ID NOs: 151, 278, 279, 280, 97, and 281.

In some aspects, provided herein are anti-GPNMB antibodies comprising: (a) HVR-H1 comprising the amino acid sequence of SEQ ID NO:71; (b) HVR-H2 comprising the amino acid sequence of SEQ ID NO:255; (c) HVR-H3 comprising the amino acid sequence of SEQ ID NO:73; (d) HVR-L1 comprising the amino acid sequence of SEQ ID NO:263; (e) HVR-L2 comprising the amino acid sequence of SEQ ID NO:269; and (f) HVR-L3 comprising the amino acid sequence of SEQ ID NO:151; (a) HVR-H1 comprising the amino acid sequence of SEQ ID NO:71; (b) HVR-H2 comprising the amino acid sequence of SEQ ID NO:256; (c) HVR-H3 comprising the amino acid sequence of SEQ ID NO:73; (d) HVR-L1 comprising the amino acid sequence of SEQ ID NO:264; (e) HVR-L2 comprising the amino acid sequence of SEQ ID NO:270; and (f) HVR-L3 comprising the amino acid sequence of SEQ ID NO:278; (a) HVR-H1 comprising the amino acid sequence of SEQ ID NO:71; (b) HVR-H2 comprising the amino acid sequence of SEQ ID NO:257; (c) HVR-H3 comprising the amino acid sequence of SEQ ID NO:73; (d) HVR-L1 comprising the amino acid sequence of SEQ ID NO:265; (e) HVR-L2 comprising the amino acid sequence of SEQ ID NO:271; and (f) HVR-L3 comprising the amino acid sequence of SEQ ID NO:278; (a) HVR-H1 comprising the amino acid sequence of SEQ ID NO:71; (b) HVR-H2 comprising the amino acid sequence of SEQ ID NO:257; (c) HVR-H3 comprising the amino acid sequence of SEQ ID NO:261; (d) HVR-L1 comprising the amino acid sequence of SEQ ID NO:265; (e) HVR-L2 comprising the amino acid sequence of SEQ ID NO:271; and (f) HVR-L3 comprising the amino acid sequence of SEQ ID NO:278; (a) HVR-H1 comprising the amino acid sequence of SEQ ID NO:71; (b) HVR-H2 comprising the amino acid sequence of SEQ ID NO:258; (c) HVR-H3 comprising the amino acid sequence of SEQ ID NO:73; (d) HVR-L1 comprising the amino acid sequence of SEQ ID NO:266; (e) HVR-L2 comprising the amino acid sequence of SEQ ID NO:272; and (f) HVR-L3 comprising the amino acid sequence of SEQ ID NO:278; (a) HVR-H1 comprising the amino acid sequence of SEQ ID NO:71; (b) HVR-H2 comprising the amino acid sequence of SEQ ID NO:257; (c) HVR-H3 comprising the amino acid sequence of SEQ ID NO:73; (d) HVR-L1 comprising the amino acid sequence of SEQ ID NO:264; (e) HVR-L2 comprising the amino acid sequence of SEQ ID NO:273; and (f) HVR-L3 comprising the amino acid sequence of SEQ ID NO:278; (a) HVR-H1 comprising the amino acid sequence of SEQ ID NO:71; (b) HVR-H2 comprising the amino acid sequence of SEQ ID NO:257; (c) HVR-H3 comprising the amino acid sequence of SEQ ID NO:261; (d) HVR-L1 comprising the amino acid sequence of SEQ ID NO:264; (e) HVR-L2 comprising the amino acid sequence of SEQ ID NO:273; and (f) HVR-L3 comprising the amino acid sequence of SEQ ID NO:278; (a) HVR-H1 comprising the amino acid sequence of SEQ ID NO:71; (b) HVR-H2 comprising the amino acid sequence of SEQ ID NO:257; (c) HVR-H3 comprising the amino acid sequence of SEQ ID NO:73; (d) HVR-L1 comprising the amino acid sequence of SEQ ID NO:264; (e) HVR-L2 comprising the amino acid sequence of SEQ ID NO:272; and (f) HVR-L3 comprising the amino acid sequence of SEQ ID NO:279; (a) HVR-H1 comprising the amino acid sequence of SEQ ID NO:71; (b) HVR-H2 comprising the amino acid sequence of SEQ ID NO:257; (c) HVR-H3 comprising the amino acid sequence of SEQ ID NO:261; (d) HVR-L1 comprising the amino acid sequence of SEQ ID NO:264; (e) HVR-L2 comprising the amino acid sequence of SEQ ID NO:272; and (f) HVR-L3 comprising the amino acid sequence of SEQ ID NO:278; (a) HVR-H1 comprising the amino acid sequence of SEQ ID NO:71; (b) HVR-H2 comprising the amino acid sequence of SEQ ID NO:258; (c) HVR-H3 comprising the amino acid sequence of SEQ ID NO: 73; (d) HVR-L1 comprising the amino acid sequence of SEQ ID NO:264; (e) HVR-L2 comprising the amino acid sequence of SEQ ID NO:272; and (f) HVR-L3 comprising the amino acid sequence of SEQ ID NO:278; (a) HVR-H1 comprising the amino acid sequence of SEQ ID NO:71; (b) HVR-H2 comprising the amino acid sequence of SEQ ID NO:257; (c) HVR-H3 comprising the amino acid sequence of SEQ ID NO:73; (d) HVR-L1 comprising the amino acid sequence of SEQ ID NO:264; (e) HVR-L2 comprising the amino acid sequence of SEQ ID NO:272; and (f) HVR-L3 comprising the amino acid sequence of SEQ ID NO:280; (a) HVR-H1 comprising the amino acid sequence of SEQ ID NO:23; (b) HVR-H2 comprising the amino acid sequence of SEQ ID NO:259; (c) HVR-H3 comprising the amino acid sequence of SEQ ID NO: 19; (d) HVR-L1 comprising the amino acid sequence of SEQ ID NO:267; (e) HVR-L2 comprising the amino acid sequence of SEQ ID NO:96; and (f) HVR-L3 comprising the amino acid sequence of SEQ ID NO:97; (a) HVR-H1 comprising the amino acid sequence of SEQ ID NO:23; (b) HVR-H2 comprising the amino acid sequence of SEQ ID NO:260; (c) HVR-H3 comprising the amino acid sequence of SEQ ID NO:262; (d) HVR-L1 comprising the amino acid sequence of SEQ ID NO:268; (e) HVR-L2 comprising the amino acid sequence of SEQ ID NO:274; and (f) HVR-L3 comprising the amino acid sequence of SEQ ID NO:281; (a) HVR-H1 comprising the amino acid sequence of SEQ ID NO:23; (b) HVR-H2 comprising the amino acid sequence of SEQ ID NO:260; (c) HVR-H3 comprising the amino acid sequence of SEQ ID NO:262; (d) HVR-L1 comprising the amino acid sequence of SEQ ID NO:268; (e) HVR-L2 comprising the amino acid sequence of SEQ ID NO:274; and (f) HVR-L3 comprising the amino acid sequence of SEQ ID NO:97; (a) HVR-H1 comprising the amino acid sequence of SEQ ID NO:23; (b) HVR-H2 comprising the amino acid sequence of SEQ ID NO:260; (c) HVR-H3 comprising the amino acid sequence of SEQ ID NO:262; (d) HVR-L1 comprising the amino acid sequence of SEQ ID NO:268; (e) HVR-L2 comprising the amino acid sequence of SEQ ID NO:275; and (f) HVR-L3 comprising the amino acid sequence of SEQ ID NO:281; (a) HVR-H1 comprising the amino acid sequence of SEQ ID NO:23; (b) HVR-H2 comprising the amino acid sequence of SEQ ID NO:260; (c) HVR-H3 comprising the amino acid sequence of SEQ ID NO:262; (d) HVR-L1 comprising the amino acid sequence of SEQ ID NO:267; (e) HVR-L2 comprising the amino acid sequence of SEQ ID NO:276; and (f) HVR-L3 comprising the amino acid sequence of SEQ ID NO:97; (a) HVR-H1 comprising the amino acid sequence of SEQ ID NO:23; (b) HVR-H2 comprising the amino acid sequence of SEQ ID NO:260; (c) HVR-H3 comprising the amino acid sequence of SEQ ID NO:262; (d) HVR-L1 comprising the amino acid sequence of SEQ ID NO:268; (e) HVR-L2 comprising the amino acid sequence of SEQ ID NO:277; and (f) HVR-L3 comprising the amino acid sequence of SEQ ID NO:97; and (a) HVR-H1 comprising the amino acid sequence of SEQ ID NO:23; (b) HVR-H2 comprising the amino acid sequence of SEQ ID NO:260; (c) HVR-H3 comprising the amino acid sequence of SEQ ID NO:262; (d) HVR-L1 comprising the amino acid sequence of SEQ ID NO:267; (e) HVR-L2 comprising the amino acid sequence of SEQ ID NO:96; and (f) HVR-L3 comprising the amino acid sequence of SEQ ID NO:97.

In another aspect, an anti-GPNMB antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:234, 235, 236, 237, 238, 239, and 240. In certain aspects, a V_{H} sequence having at least 90%, 91%, 92%, 93%, 94%, 95%, 96%, 97%, 98%, or 99% identity to an amino acid sequence selected from the group consisting of SEQ ID NOs: 234, 235, 236, 237, 238, 239, and 240 contains substitutions (e.g., conservative substitutions), insertions, or deletions relative to the reference sequence, but an anti-GPNMB antibody comprising that sequence retains the ability to bind to GPNMB. In certain aspects, a total of 1 to 10 amino acids have been substituted, inserted, and/or deleted in SEQ ID NO: 234, 235, 236, 237, 238, 239, or 240. In certain aspects, substitutions, insertions, or deletions occur in regions outside the HVRs (i.e., in the FRs). Optionally, the anti-GPNMB antibody comprises the V_{H} sequence of SEQ ID NO: 234, 235, 236, 237, 238, 239, or 240, including post-translational modifications of that sequence. In a particular aspect, the V_{H} comprises one, two or three HVRs selected from: (a) HVR-H1 comprising an amino acid sequence selected from the group consisting of SEQ ID NOs: 71 and 23; (b) HVR-H2 comprising an amino acid sequence selected from the group consisting of SEQ ID NOs: 255, 256, 257, 258, 259, and 260; and (c) HVR-H3 comprising an amino acid sequence selected from the group consisting of SEQ ID NOs: 73, 261, 19, and 262.

In another aspect, an anti-GPNMB antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs: 241, 242, 243, 244, 245, 246, 247, 248, 249, 250, 251, 252, 253, and 254. In certain aspects, a V_{L} sequence having at least 90%, 91%, 92%, 93%, 94%, 95%, 96%, 97%, 98%, or 99% identity to an amino acid sequence selected from the group consisting of SEQ ID NOs: 241, 242, 243, 244, 245, 246, 247, 248, 249, 250, 251, 252, 253, and 254, and contains substitutions (e.g., conservative substitutions), insertions, or deletions relative to the reference sequence, but an anti-GPNMB antibody comprising that sequence retains the ability to bind to GPNMB. In some aspects, a total of 1 to 10 amino acids have been substituted, inserted and/or deleted in SEQ ID NO: 241, 242, 243, 244, 245, 246, 247, 248, 249, 250, 251, 252, 253, or 254. In certain aspects, a total of 1 to 5 amino acids have been substituted, inserted and/or deleted in SEQ ID NO: 241, 242, 243, 244, 245, 246, 247, 248, 249, 250, 251, 252, 253, or 254. In certain aspects, the substitutions, insertions, or deletions occur in regions outside the HVRs (i.e., in the FRs). Optionally, the anti-GPNMB antibody comprises the V_{L} sequence of SEQ ID NO: 241, 242, 243, 244, 245, 246, 247, 248, 249, 250, 251, 252, 253, or 254, including post-translational modifications of that sequence. In a particular aspect, the V_{L} comprises one, two or three HVRs selected from (a) HVR-L1 comprising an amino acid sequence selected from the group consisting of SEQ ID NOs: 263, 264, 265, 266, 267, and 268; (b) HVR-L2 comprising an amino acid sequence selected from the group consisting of SEQ ID NOs: 263, 264, 265, 266, 267, and 268; and (c) HVR-L3 comprising an amino acid sequence selected from the group consisting of SEQ ID NOs: 151, 278, 279, 280, 97, and 281.

In some aspects, an anti-GPNMB antibody is provided, wherein the antibody comprises a V_{H} as in any of the aspects provided above, and a V_{L} as in any of the aspects provided above. In some aspects, provided herein are anti-GPNMB antibodies, wherein the antibody comprises a V_{H} as in any of the aspects provided above, and a V_{L} as in any of the aspects provided above. In one aspect, the antibody comprises the V_{H} and V_{L} sequences in SEQ ID NOs: 234, 235, 236, 237, 238, 239, and 240, and SEQ ID NOs: 241, 242, 243, 244, 245, 246, 247, 248, 249, 250, 251, 252, 253, or 254, respectively, including post-translational modifications of those sequences.

In some aspects, provided herein are anti-GPNMB antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:234 and V_{L} comprising the amino acid sequence of SEQ ID NO:241; V_{H} comprising the amino acid sequence of SEQ ID NO:235 and V_{L} comprising the amino acid sequence of SEQ ID NO:242; V_{H} comprising the amino acid sequence of SEQ ID NO:236 and V_{L} comprising the amino acid sequence of SEQ ID NO:243; V_{H} comprising the amino acid sequence of SEQ ID NO:237 and V_{L} comprising the amino acid sequence of SEQ ID NO:243; V_{H} comprising the amino acid sequence of SEQ ID NO:238 and V_{L} comprising the amino acid sequence of SEQ ID NO:244; V_{H} comprising the amino acid sequence of SEQ ID NO:236 and V_{L} comprising the amino acid sequence of SEQ ID NO:245; V_{H} comprising the amino acid sequence of SEQ ID NO:237 and V_{L} comprising the amino acid sequence of SEQ ID NO:245; V_{H} comprising the amino acid sequence of SEQ ID NO:236 and V_{L} comprising the amino acid sequence of SEQ ID NO:246; V_{H} comprising the amino acid sequence of SEQ ID NO:237 and V_{L} comprising the amino acid sequence of SEQ ID NO:247; V_{H} comprising the amino acid sequence of SEQ ID NO:238 and V_{L} comprising the amino acid sequence of SEQ ID NO:247; V_{H} comprising the amino acid sequence of SEQ ID NO:236 and V_{L} comprising the amino acid sequence of SEQ ID NO:248; V_{H} comprising the amino acid sequence of SEQ ID NO:239 and V_{L} comprising the amino acid sequence of SEQ ID NO:249; V_{H} comprising the amino acid sequence of SEQ ID NO:240 and V_{L} comprising the amino acid sequence of SEQ ID NO:250; V_{H} comprising the amino acid sequence of SEQ ID NO:240 and V_{L} comprising the amino acid sequence of SEQ ID NO:251; V_{H} comprising the amino acid sequence of SEQ ID NO:240 and V_{L} comprising the amino acid sequence of SEQ ID NO:252; V_{H} comprising the amino acid sequence of SEQ ID NO:240 and V_{L} comprising the amino acid sequence of SEQ ID NO:253; V_{H} comprising the amino acid sequence of SEQ ID NO:240 and V_{L} comprising the amino acid sequence of SEQ ID NO:254; and V_{H} comprising the amino acid sequence of SEQ ID NO:240 and V_{L} comprising the amino acid sequence of SEQ ID NO:249.

In some aspects, an anti-GPNMB antibody of the present disclosure competitively inhibits binding of at least one reference antibody selected from anti-GPNMB antibody GPN-81, GPN-82, GPN-83, GPN-84, GPN-85, GPN-86, GPN-87, GPN-88, GPN-89, GPN-90, GPN-91, GPN-92, GPN-93, GPN-94, GPN-95, GPN-96, GPN-97, and GPN-98 and any combination thereof, for binding to GPNMB.

In some aspects, an anti-GPNMB antibody of the present disclosure binds to an epitope of human GPNMB that is the same as or overlaps with the GPNMB epitope bound by at least one reference antibody selected from anti-GPNMB antibody GPN-81, GPN-82, GPN-83, GPN-84, GPN-85, GPN-86, GPN-87, GPN-88, GPN-89, GPN-90, GPN-91, GPN-92, GPN-93, GPN-94, GPN-95, GPN-96, GPN-97, and GPN-98.

In some aspects, an anti-GPNMB antibody of the present disclosure competitively inhibits binding of at least one reference antibody, or binds to an epitope of human GPNMB that is the same as or overlaps with the GPNMB epitope bound by at least one reference antibody, wherein the reference antibody is an anti-GPNMB antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:234 and V_{L} comprising the amino acid sequence of SEQ ID NO:241; V_{H} comprising the amino acid sequence of SEQ ID NO:235 and V_{L} comprising the amino acid sequence of SEQ ID NO:242; V_{H} comprising the amino acid sequence of SEQ ID NO:236 and V_{L} comprising the amino acid sequence of SEQ ID NO:243; V_{H} comprising the amino acid sequence of SEQ ID NO:237 and V_{L} comprising the amino acid sequence of SEQ ID NO:243; V_{H} comprising the amino acid sequence of SEQ ID NO:238 and V_{L} comprising the amino acid sequence of SEQ ID NO:244; V_{H} comprising the amino acid sequence of SEQ ID NO:236 and V_{L} comprising the amino acid sequence of SEQ ID NO:245; V_{H} comprising the amino acid sequence of SEQ ID NO:237 and V_{L} comprising the amino acid sequence of SEQ ID NO:245; V_{H} comprising the amino acid sequence of SEQ ID NO:236 and V_{L} comprising the amino acid sequence of SEQ ID NO:246; V_{H} comprising the amino acid sequence of SEQ ID NO:237 and V_{L} comprising the amino acid sequence of SEQ ID NO:247; V_{H} comprising the amino acid sequence of SEQ ID NO:238 and V_{L} comprising the amino acid sequence of SEQ ID NO:247; V_{H} comprising the amino acid sequence of SEQ ID NO:236 and V_{L} comprising the amino acid sequence of SEQ ID NO:248; V_{H} comprising the amino acid sequence of SEQ ID NO:239 and V_{L} comprising the amino acid sequence of SEQ ID NO:249; V_{H} comprising the amino acid sequence of SEQ ID NO:240 and V_{L} comprising the amino acid sequence of SEQ ID NO:250; V_{H} comprising the amino acid sequence of SEQ ID NO:240 and V_{L} comprising the amino acid sequence of SEQ ID NO:251; V_{H} comprising the amino acid sequence of SEQ ID NO:240 and V_{L} comprising the amino acid sequence of SEQ ID NO:252; V_{H} comprising the amino acid sequence of SEQ ID NO:240 and V_{L} comprising the amino acid sequence of SEQ ID NO:253; V_{H} comprising the amino acid sequence of SEQ ID NO:240 and V_{L} comprising the amino acid sequence of SEQ ID NO:254; and V_{H} comprising the amino acid sequence of SEQ ID NO:240 and V_{L} comprising the amino acid sequence of SEQ ID NO:249.

In some aspects, provided herein are anti-GPNMB antibodies comprising a heavy chain and a light chain, wherein: the heavy chain comprises the amino acid sequence of SEQ ID NO:290 and the light chain comprises the amino acid sequence of SEQ ID NO:416; the heavy chain comprises the amino acid sequence of SEQ ID NO:291 and the light chain comprises the amino acid sequence of SEQ ID NO:416; the heavy chain comprises the amino acid sequence of SEQ ID NO:292 and the light chain comprises the amino acid sequence of SEQ ID NO:416; the heavy chain comprises the amino acid sequence of SEQ ID NO:293 and the light chain comprises the amino acid sequence of SEQ ID NO:416; the heavy chain comprises the amino acid sequence of SEQ ID NO:294 and the light chain comprises the amino acid sequence of SEQ ID NO:416; the heavy chain comprises the amino acid sequence of SEQ ID NO:295 and the light chain comprises the amino acid sequence of SEQ ID NO:416; the heavy chain comprises the amino acid sequence of SEQ ID NO:296 and the light chain comprises the amino acid sequence of SEQ ID NO:416; the heavy chain comprises the amino acid sequence of SEQ ID NO:297 and the light chain comprises the amino acid sequence of SEQ ID NO:416; the heavy chain comprises the amino acid sequence of SEQ ID NO:298 and the light chain comprises the amino acid sequence of SEQ ID NO:416; the heavy chain comprises the amino acid sequence of SEQ ID NO:299 and the light chain comprises the amino acid sequence of SEQ ID NO:416; the heavy chain comprises the amino acid sequence of SEQ ID NO:300 and the light chain comprises the amino acid sequence of SEQ ID NO:416; the heavy chain comprises the amino acid sequence of SEQ ID NO:301 and the light chain comprises the amino acid sequence of SEQ ID NO:416; the heavy chain comprises the amino acid sequence of SEQ ID NO:302 and the light chain comprises the amino acid sequence of SEQ ID NO:416; the heavy chain comprises the amino acid sequence of SEQ ID NO:303 and the light chain comprises the amino acid sequence of SEQ ID NO:416; the heavy chain comprises the amino acid sequence of SEQ ID NO:304 and the light chain comprises the amino acid sequence of SEQ ID NO:416; the heavy chain comprises the amino acid sequence of SEQ ID NO:305 and the light chain comprises the amino acid sequence of SEQ ID NO:416; the heavy chain comprises the amino acid sequence of SEQ ID NO:306 and the light chain comprises the amino acid sequence of SEQ ID NO:416; or the heavy chain comprises the amino acid sequence of SEQ ID NO:307 and the light chain comprises the amino acid sequence of SEQ ID NO:416.

In some aspects, provided herein are anti-GPNMB antibodies comprising a heavy chain and a light chain, wherein: the heavy chain comprises the amino acid sequence of SEQ ID NO:308 and the light chain comprises the amino acid sequence of SEQ ID NO:417; the heavy chain comprises the amino acid sequence of SEQ ID NO:309 and the light chain comprises the amino acid sequence of SEQ ID NO:417; the heavy chain comprises the amino acid sequence of SEQ ID NO:310 and the light chain comprises the amino acid sequence of SEQ ID NO:417; the heavy chain comprises the amino acid sequence of SEQ ID NO:311 and the light chain comprises the amino acid sequence of SEQ ID NO:417; the heavy chain comprises the amino acid sequence of SEQ ID NO:312 and the light chain comprises the amino acid sequence of SEQ ID NO:417; the heavy chain comprises the amino acid sequence of SEQ ID NO:313 and the light chain comprises the amino acid sequence of SEQ ID NO:417; the heavy chain comprises the amino acid sequence of SEQ ID NO:314 and the light chain comprises the amino acid sequence of SEQ ID NO:417; the heavy chain comprises the amino acid sequence of SEQ ID NO:315 and the light chain comprises the amino acid sequence of SEQ ID NO:417; the heavy chain comprises the amino acid sequence of SEQ ID NO:316 and the light chain comprises the amino acid sequence of SEQ ID NO:417; the heavy chain comprises the amino acid sequence of SEQ ID NO:317 and the light chain comprises the amino acid sequence of SEQ ID NO:417; the heavy chain comprises the amino acid sequence of SEQ ID NO:318 and the light chain comprises the amino acid sequence of SEQ ID NO:417; the heavy chain comprises the amino acid sequence of SEQ ID NO:319 and the light chain comprises the amino acid sequence of SEQ ID NO:417; the heavy chain comprises the amino acid sequence of SEQ ID NO:320 and the light chain comprises the amino acid sequence of SEQ ID NO:417; the heavy chain comprises the amino acid sequence of SEQ ID NO:321 and the light chain comprises the amino acid sequence of SEQ ID NO:417; the heavy chain comprises the amino acid sequence of SEQ ID NO:322 and the light chain comprises the amino acid sequence of SEQ ID NO:417; the heavy chain comprises the amino acid sequence of SEQ ID NO:323 and the light chain comprises the amino acid sequence of SEQ ID NO:417; the heavy chain comprises the amino acid sequence of SEQ ID NO:324 and the light chain comprises the amino acid sequence of SEQ ID NO:417; or the heavy chain comprises the amino acid sequence of SEQ ID NO:325 and the light chain comprises the amino acid sequence of SEQ ID NO:417.

In some aspects, provided herein are anti-GPNMB antibodies comprising a heavy chain and a light chain, wherein: the heavy chain comprises the amino acid sequence of SEQ ID NO:326 and the light chain comprises the amino acid sequence of SEQ ID NO:418; the heavy chain comprises the amino acid sequence of SEQ ID NO:327 and the light chain comprises the amino acid sequence of SEQ ID NO:418; the heavy chain comprises the amino acid sequence of SEQ ID NO:328 and the light chain comprises the amino acid sequence of SEQ ID NO:418; the heavy chain comprises the amino acid sequence of SEQ ID NO:329 and the light chain comprises the amino acid sequence of SEQ ID NO:418; the heavy chain comprises the amino acid sequence of SEQ ID NO:330 and the light chain comprises the amino acid sequence of SEQ ID NO:418; the heavy chain comprises the amino acid sequence of SEQ ID NO:331 and the light chain comprises the amino acid sequence of SEQ ID NO:418; the heavy chain comprises the amino acid sequence of SEQ ID NO:332 and the light chain comprises the amino acid sequence of SEQ ID NO:418; the heavy chain comprises the amino acid sequence of SEQ ID NO:333 and the light chain comprises the amino acid sequence of SEQ ID NO:418; the heavy chain comprises the amino acid sequence of SEQ ID NO:334 and the light chain comprises the amino acid sequence of SEQ ID NO:418; the heavy chain comprises the amino acid sequence of SEQ ID NO:335 and the light chain comprises the amino acid sequence of SEQ ID NO:418; the heavy chain comprises the amino acid sequence of SEQ ID NO:336 and the light chain comprises the amino acid sequence of SEQ ID NO:418; the heavy chain comprises the amino acid sequence of SEQ ID NO:337 and the light chain comprises the amino acid sequence of SEQ ID NO:418; the heavy chain comprises the amino acid sequence of SEQ ID NO:338 and the light chain comprises the amino acid sequence of SEQ ID NO:418; the heavy chain comprises the amino acid sequence of SEQ ID NO:339 and the light chain comprises the amino acid sequence of SEQ ID NO:418; the heavy chain comprises the amino acid sequence of SEQ ID NO:340 and the light chain comprises the amino acid sequence of SEQ ID NO:418; the heavy chain comprises the amino acid sequence of SEQ ID NO:341 and the light chain comprises the amino acid sequence of SEQ ID NO:418; the heavy chain comprises the amino acid sequence of SEQ ID NO:342 and the light chain comprises the amino acid sequence of SEQ ID NO:418; or the heavy chain comprises the amino acid sequence of SEQ ID NO:343 and the light chain comprises the amino acid sequence of SEQ ID NO:418.

In some aspects, provided herein are anti-GPNMB antibodies comprising a heavy chain and a light chain, wherein: the heavy chain comprises the amino acid sequence of SEQ ID NO:344 and the light chain comprises the amino acid sequence of SEQ ID NO:419; the heavy chain comprises the amino acid sequence of SEQ ID NO:345 and the light chain comprises the amino acid sequence of SEQ ID NO:419; the heavy chain comprises the amino acid sequence of SEQ ID NO:346 and the light chain comprises the amino acid sequence of SEQ ID NO:419; the heavy chain comprises the amino acid sequence of SEQ ID NO:347 and the light chain comprises the amino acid sequence of SEQ ID NO:419; the heavy chain comprises the amino acid sequence of SEQ ID NO:348 and the light chain comprises the amino acid sequence of SEQ ID NO:419; the heavy chain comprises the amino acid sequence of SEQ ID NO:349 and the light chain comprises the amino acid sequence of SEQ ID NO:419; the heavy chain comprises the amino acid sequence of SEQ ID NO:350 and the light chain comprises the amino acid sequence of SEQ ID NO:419; the heavy chain comprises the amino acid sequence of SEQ ID NO:351 and the light chain comprises the amino acid sequence of SEQ ID NO:419; the heavy chain comprises the amino acid sequence of SEQ ID NO:352 and the light chain comprises the amino acid sequence of SEQ ID NO:419; the heavy chain comprises the amino acid sequence of SEQ ID NO:353 and the light chain comprises the amino acid sequence of SEQ ID NO:419; the heavy chain comprises the amino acid sequence of SEQ ID NO:354 and the light chain comprises the amino acid sequence of SEQ ID NO:419; the heavy chain comprises the amino acid sequence of SEQ ID NO:355 and the light chain comprises the amino acid sequence of SEQ ID NO:419; the heavy chain comprises the amino acid sequence of SEQ ID NO:356 and the light chain comprises the amino acid sequence of SEQ ID NO:419; the heavy chain comprises the amino acid sequence of SEQ ID NO:357 and the light chain comprises the amino acid sequence of SEQ ID NO:419; the heavy chain comprises the amino acid sequence of SEQ ID NO:358 and the light chain comprises the amino acid sequence of SEQ ID NO:419; the heavy chain comprises the amino acid sequence of SEQ ID NO:359 and the light chain comprises the amino acid sequence of SEQ ID NO:419; the heavy chain comprises the amino acid sequence of SEQ ID NO:360 and the light chain comprises the amino acid sequence of SEQ ID NO:419; or the heavy chain comprises the amino acid sequence of SEQ ID NO:361 and the light chain comprises the amino acid sequence of SEQ ID NO:419.

In some aspects, provided herein are anti-GPNMB antibodies comprising a heavy chain and a light chain, wherein: the heavy chain comprises the amino acid sequence of SEQ ID NO:362 and the light chain comprises the amino acid sequence of SEQ ID NO:420; the heavy chain comprises the amino acid sequence of SEQ ID NO:363 and the light chain comprises the amino acid sequence of SEQ ID NO:420; the heavy chain comprises the amino acid sequence of SEQ ID NO:364 and the light chain comprises the amino acid sequence of SEQ ID NO:420; the heavy chain comprises the amino acid sequence of SEQ ID NO:365 and the light chain comprises the amino acid sequence of SEQ ID NO:420; the heavy chain comprises the amino acid sequence of SEQ ID NO:366 and the light chain comprises the amino acid sequence of SEQ ID NO:420; the heavy chain comprises the amino acid sequence of SEQ ID NO:367 and the light chain comprises the amino acid sequence of SEQ ID NO:420; the heavy chain comprises the amino acid sequence of SEQ ID NO:368 and the light chain comprises the amino acid sequence of SEQ ID NO:420; the heavy chain comprises the amino acid sequence of SEQ ID NO:369 and the light chain comprises the amino acid sequence of SEQ ID NO:420; the heavy chain comprises the amino acid sequence of SEQ ID NO:370 and the light chain comprises the amino acid sequence of SEQ ID NO:420; the heavy chain comprises the amino acid sequence of SEQ ID NO:371 and the light chain comprises the amino acid sequence of SEQ ID NO:420; the heavy chain comprises the amino acid sequence of SEQ ID NO:372 and the light chain comprises the amino acid sequence of SEQ ID NO:420; the heavy chain comprises the amino acid sequence of SEQ ID NO:373 and the light chain comprises the amino acid sequence of SEQ ID NO:420; the heavy chain comprises the amino acid sequence of SEQ ID NO:374 and the light chain comprises the amino acid sequence of SEQ ID NO:420; the heavy chain comprises the amino acid sequence of SEQ ID NO:375 and the light chain comprises the amino acid sequence of SEQ ID NO:420; the heavy chain comprises the amino acid sequence of SEQ ID NO:376 and the light chain comprises the amino acid sequence of SEQ ID NO:420; the heavy chain comprises the amino acid sequence of SEQ ID NO:377 and the light chain comprises the amino acid sequence of SEQ ID NO:420; the heavy chain comprises the amino acid sequence of SEQ ID NO:378 and the light chain comprises the amino acid sequence of SEQ ID NO:420; or the heavy chain comprises the amino acid sequence of SEQ ID NO:379 and the light chain comprises the amino acid sequence of SEQ ID NO:420.

In some aspects, provided herein are anti-GPNMB antibodies comprising a heavy chain and a light chain, wherein: the heavy chain comprises the amino acid sequence of SEQ ID NO:326 and the light chain comprises the amino acid sequence of SEQ ID NO:421; the heavy chain comprises the amino acid sequence of SEQ ID NO:327 and the light chain comprises the amino acid sequence of SEQ ID NO:421; the heavy chain comprises the amino acid sequence of SEQ ID NO:328 and the light chain comprises the amino acid sequence of SEQ ID NO:421; the heavy chain comprises the amino acid sequence of SEQ ID NO:329 and the light chain comprises the amino acid sequence of SEQ ID NO:421; the heavy chain comprises the amino acid sequence of SEQ ID NO:330 and the light chain comprises the amino acid sequence of SEQ ID NO:421; the heavy chain comprises the amino acid sequence of SEQ ID NO:331 and the light chain comprises the amino acid sequence of SEQ ID NO:421; the heavy chain comprises the amino acid sequence of SEQ ID NO:332 and the light chain comprises the amino acid sequence of SEQ ID NO:421; the heavy chain comprises the amino acid sequence of SEQ ID NO:333 and the light chain comprises the amino acid sequence of SEQ ID NO:421; the heavy chain comprises the amino acid sequence of SEQ ID NO:334 and the light chain comprises the amino acid sequence of SEQ ID NO:421; the heavy chain comprises the amino acid sequence of SEQ ID NO:335 and the light chain comprises the amino acid sequence of SEQ ID NO:421; the heavy chain comprises the amino acid sequence of SEQ ID NO:336 and the light chain comprises the amino acid sequence of SEQ ID NO:421; the heavy chain comprises the amino acid sequence of SEQ ID NO:337 and the light chain comprises the amino acid sequence of SEQ ID NO:421; the heavy chain comprises the amino acid sequence of SEQ ID NO:338 and the light chain comprises the amino acid sequence of SEQ ID NO:421; the heavy chain comprises the amino acid sequence of SEQ ID NO:339 and the light chain comprises the amino acid sequence of SEQ ID NO:421; the heavy chain comprises the amino acid sequence of SEQ ID NO:340 and the light chain comprises the amino acid sequence of SEQ ID NO:421; the heavy chain comprises the amino acid sequence of SEQ ID NO:341 and the light chain comprises the amino acid sequence of SEQ ID NO:421; the heavy chain comprises the amino acid sequence of SEQ ID NO:342 and the light chain comprises the amino acid sequence of SEQ ID NO:421; or the heavy chain comprises the amino acid sequence of SEQ ID NO:343 and the light chain comprises the amino acid sequence of SEQ ID NO:421.

In some aspects, provided herein are anti-GPNMB antibodies comprising a heavy chain and a light chain, wherein: the heavy chain comprises the amino acid sequence of SEQ ID NO:344 and the light chain comprises the amino acid sequence of SEQ ID NO:422; the heavy chain comprises the amino acid sequence of SEQ ID NO:345 and the light chain comprises the amino acid sequence of SEQ ID NO:422; the heavy chain comprises the amino acid sequence of SEQ ID NO:346 and the light chain comprises the amino acid sequence of SEQ ID NO:422; the heavy chain comprises the amino acid sequence of SEQ ID NO:347 and the light chain comprises the amino acid sequence of SEQ ID NO:422; the heavy chain comprises the amino acid sequence of SEQ ID NO:348 and the light chain comprises the amino acid sequence of SEQ ID NO:422; the heavy chain comprises the amino acid sequence of SEQ ID NO:349 and the light chain comprises the amino acid sequence of SEQ ID NO:422; the heavy chain comprises the amino acid sequence of SEQ ID NO:350 and the light chain comprises the amino acid sequence of SEQ ID NO:422; the heavy chain comprises the amino acid sequence of SEQ ID NO:351 and the light chain comprises the amino acid sequence of SEQ ID NO:422; the heavy chain comprises the amino acid sequence of SEQ ID NO:352 and the light chain comprises the amino acid sequence of SEQ ID NO:422; the heavy chain comprises the amino acid sequence of SEQ ID NO:353 and the light chain comprises the amino acid sequence of SEQ ID NO:422; the heavy chain comprises the amino acid sequence of SEQ ID NO:354 and the light chain comprises the amino acid sequence of SEQ ID NO:422; the heavy chain comprises the amino acid sequence of SEQ ID NO:355 and the light chain comprises the amino acid sequence of SEQ ID NO:422; the heavy chain comprises the amino acid sequence of SEQ ID NO:356 and the light chain comprises the amino acid sequence of SEQ ID NO:422; the heavy chain comprises the amino acid sequence of SEQ ID NO:357 and the light chain comprises the amino acid sequence of SEQ ID NO:422; the heavy chain comprises the amino acid sequence of SEQ ID NO:358 and the light chain comprises the amino acid sequence of SEQ ID NO:422; the heavy chain comprises the amino acid sequence of SEQ ID NO:359 and the light chain comprises the amino acid sequence of SEQ ID NO:422; the heavy chain comprises the amino acid sequence of SEQ ID NO:360 and the light chain comprises the amino acid sequence of SEQ ID NO:422; or the heavy chain comprises the amino acid sequence of SEQ ID NO:361 and the light chain comprises the amino acid sequence of SEQ ID NO:422.

In some aspects, provided herein are anti-GPNMB antibodies comprising a heavy chain and a light chain, wherein: the heavy chain comprises the amino acid sequence of SEQ ID NO:326 and the light chain comprises the amino acid sequence of SEQ ID NO:423; the heavy chain comprises the amino acid sequence of SEQ ID NO:327 and the light chain comprises the amino acid sequence of SEQ ID NO:423; the heavy chain comprises the amino acid sequence of SEQ ID NO:328 and the light chain comprises the amino acid sequence of SEQ ID NO:423; the heavy chain comprises the amino acid sequence of SEQ ID NO:329 and the light chain comprises the amino acid sequence of SEQ ID NO:423; the heavy chain comprises the amino acid sequence of SEQ ID NO:330 and the light chain comprises the amino acid sequence of SEQ ID NO:423; the heavy chain comprises the amino acid sequence of SEQ ID NO:331 and the light chain comprises the amino acid sequence of SEQ ID NO:423; the heavy chain comprises the amino acid sequence of SEQ ID NO:332 and the light chain comprises the amino acid sequence of SEQ ID NO:423; the heavy chain comprises the amino acid sequence of SEQ ID NO:333 and the light chain comprises the amino acid sequence of SEQ ID NO:423; the heavy chain comprises the amino acid sequence of SEQ ID NO:334 and the light chain comprises the amino acid sequence of SEQ ID NO:423; the heavy chain comprises the amino acid sequence of SEQ ID NO:335 and the light chain comprises the amino acid sequence of SEQ ID NO:423; the heavy chain comprises the amino acid sequence of SEQ ID NO:336 and the light chain comprises the amino acid sequence of SEQ ID NO:423; the heavy chain comprises the amino acid sequence of SEQ ID NO:337 and the light chain comprises the amino acid sequence of SEQ ID NO:423; the heavy chain comprises the amino acid sequence of SEQ ID NO:338 and the light chain comprises the amino acid sequence of SEQ ID NO:423; the heavy chain comprises the amino acid sequence of SEQ ID NO:339 and the light chain comprises the amino acid sequence of SEQ ID NO:423; the heavy chain comprises the amino acid sequence of SEQ ID NO:340 and the light chain comprises the amino acid sequence of SEQ ID NO:423; the heavy chain comprises the amino acid sequence of SEQ ID NO:341 and the light chain comprises the amino acid sequence of SEQ ID NO:423; the heavy chain comprises the amino acid sequence of SEQ ID NO:342 and the light chain comprises the amino acid sequence of SEQ ID NO:423; or the heavy chain comprises the amino acid sequence of SEQ ID NO:343 and the light chain comprises the amino acid sequence of SEQ ID NO:423.

In some aspects, provided herein are anti-GPNMB antibodies comprising a heavy chain and a light chain, wherein: the heavy chain comprises the amino acid sequence of SEQ ID NO:344 and the light chain comprises the amino acid sequence of SEQ ID NO:424; the heavy chain comprises the amino acid sequence of SEQ ID NO:345 and the light chain comprises the amino acid sequence of SEQ ID NO:424; the heavy chain comprises the amino acid sequence of SEQ ID NO:346 and the light chain comprises the amino acid sequence of SEQ ID NO:424; the heavy chain comprises the amino acid sequence of SEQ ID NO:347 and the light chain comprises the amino acid sequence of SEQ ID NO:424; the heavy chain comprises the amino acid sequence of SEQ ID NO:348 and the light chain comprises the amino acid sequence of SEQ ID NO:424; the heavy chain comprises the amino acid sequence of SEQ ID NO:349 and the light chain comprises the amino acid sequence of SEQ ID NO:424; the heavy chain comprises the amino acid sequence of SEQ ID NO:350 and the light chain comprises the amino acid sequence of SEQ ID NO:424; the heavy chain comprises the amino acid sequence of SEQ ID NO:351 and the light chain comprises the amino acid sequence of SEQ ID NO:424; the heavy chain comprises the amino acid sequence of SEQ ID NO:352 and the light chain comprises the amino acid sequence of SEQ ID NO:424; the heavy chain comprises the amino acid sequence of SEQ ID NO:353 and the light chain comprises the amino acid sequence of SEQ ID NO:424; the heavy chain comprises the amino acid sequence of SEQ ID NO:354 and the light chain comprises the amino acid sequence of SEQ ID NO:424; the heavy chain comprises the amino acid sequence of SEQ ID NO:355 and the light chain comprises the amino acid sequence of SEQ ID NO:424; the heavy chain comprises the amino acid sequence of SEQ ID NO:356 and the light chain comprises the amino acid sequence of SEQ ID NO:424; the heavy chain comprises the amino acid sequence of SEQ ID NO:357 and the light chain comprises the amino acid sequence of SEQ ID NO:424; the heavy chain comprises the amino acid sequence of SEQ ID NO:358 and the light chain comprises the amino acid sequence of SEQ ID NO:424; the heavy chain comprises the amino acid sequence of SEQ ID NO:359 and the light chain comprises the amino acid sequence of SEQ ID NO:424; the heavy chain comprises the amino acid sequence of SEQ ID NO:360 and the light chain comprises the amino acid sequence of SEQ ID NO:424; or the heavy chain comprises the amino acid sequence of SEQ ID NO:361 and the light chain comprises the amino acid sequence of SEQ ID NO:424.

In some aspects, provided herein are anti-GPNMB antibodies comprising a heavy chain and a light chain, wherein: the heavy chain comprises the amino acid sequence of SEQ ID NO:362 and the light chain comprises the amino acid sequence of SEQ ID NO:425; the heavy chain comprises the amino acid sequence of SEQ ID NO:363 and the light chain comprises the amino acid sequence of SEQ ID NO:425; the heavy chain comprises the amino acid sequence of SEQ ID NO:364 and the light chain comprises the amino acid sequence of SEQ ID NO:425; the heavy chain comprises the amino acid sequence of SEQ ID NO:365 and the light chain comprises the amino acid sequence of SEQ ID NO:425; the heavy chain comprises the amino acid sequence of SEQ ID NO:366 and the light chain comprises the amino acid sequence of SEQ ID NO:425; the heavy chain comprises the amino acid sequence of SEQ ID NO:367 and the light chain comprises the amino acid sequence of SEQ ID NO:425; the heavy chain comprises the amino acid sequence of SEQ ID NO:368 and the light chain comprises the amino acid sequence of SEQ ID NO:425; the heavy chain comprises the amino acid sequence of SEQ ID NO:369 and the light chain comprises the amino acid sequence of SEQ ID NO:425; the heavy chain comprises the amino acid sequence of SEQ ID NO:370 and the light chain comprises the amino acid sequence of SEQ ID NO:425; the heavy chain comprises the amino acid sequence of SEQ ID NO:371 and the light chain comprises the amino acid sequence of SEQ ID NO:425; the heavy chain comprises the amino acid sequence of SEQ ID NO:372 and the light chain comprises the amino acid sequence of SEQ ID NO:425; the heavy chain comprises the amino acid sequence of SEQ ID NO:373 and the light chain comprises the amino acid sequence of SEQ ID NO:425; the heavy chain comprises the amino acid sequence of SEQ ID NO:374 and the light chain comprises the amino acid sequence of SEQ ID NO:425; the heavy chain comprises the amino acid sequence of SEQ ID NO:375 and the light chain comprises the amino acid sequence of SEQ ID NO:425; the heavy chain comprises the amino acid sequence of SEQ ID NO:376 and the light chain comprises the amino acid sequence of SEQ ID NO:425; the heavy chain comprises the amino acid sequence of SEQ ID NO:377 and the light chain comprises the amino acid sequence of SEQ ID NO:425; the heavy chain comprises the amino acid sequence of SEQ ID NO:378 and the light chain comprises the amino acid sequence of SEQ ID NO:425; or the heavy chain comprises the amino acid sequence of SEQ ID NO:379 and the light chain comprises the amino acid sequence of SEQ ID NO:425.

In some aspects, provided herein are anti-GPNMB antibodies comprising a heavy chain and a light chain, wherein: the heavy chain comprises the amino acid sequence of SEQ ID NO:326 and the light chain comprises the amino acid sequence of SEQ ID NO:426; the heavy chain comprises the amino acid sequence of SEQ ID NO:327 and the light chain comprises the amino acid sequence of SEQ ID NO:426; the heavy chain comprises the amino acid sequence of SEQ ID NO:328 and the light chain comprises the amino acid sequence of SEQ ID NO:426; the heavy chain comprises the amino acid sequence of SEQ ID NO:329 and the light chain comprises the amino acid sequence of SEQ ID NO:426; the heavy chain comprises the amino acid sequence of SEQ ID NO:330 and the light chain comprises the amino acid sequence of SEQ ID NO:426; the heavy chain comprises the amino acid sequence of SEQ ID NO:331 and the light chain comprises the amino acid sequence of SEQ ID NO:426; the heavy chain comprises the amino acid sequence of SEQ ID NO:332 and the light chain comprises the amino acid sequence of SEQ ID NO:426; the heavy chain comprises the amino acid sequence of SEQ ID NO:333 and the light chain comprises the amino acid sequence of SEQ ID NO:426; the heavy chain comprises the amino acid sequence of SEQ ID NO:334 and the light chain comprises the amino acid sequence of SEQ ID NO:426; the heavy chain comprises the amino acid sequence of SEQ ID NO:335 and the light chain comprises the amino acid sequence of SEQ ID NO:426; the heavy chain comprises the amino acid sequence of SEQ ID NO:336 and the light chain comprises the amino acid sequence of SEQ ID NO:426; the heavy chain comprises the amino acid sequence of SEQ ID NO:337 and the light chain comprises the amino acid sequence of SEQ ID NO:426; the heavy chain comprises the amino acid sequence of SEQ ID NO:338 and the light chain comprises the amino acid sequence of SEQ ID NO:426; the heavy chain comprises the amino acid sequence of SEQ ID NO:339 and the light chain comprises the amino acid sequence of SEQ ID NO:426; the heavy chain comprises the amino acid sequence of SEQ ID NO:340 and the light chain comprises the amino acid sequence of SEQ ID NO:426; the heavy chain comprises the amino acid sequence of SEQ ID NO:341 and the light chain comprises the amino acid sequence of SEQ ID NO:426; the heavy chain comprises the amino acid sequence of SEQ ID NO:342 and the light chain comprises the amino acid sequence of SEQ ID NO:426; or the heavy chain comprises the amino acid sequence of SEQ ID NO:343 and the light chain comprises the amino acid sequence of SEQ ID NO:426.

In some aspects, provided herein are anti-GPNMB antibodies comprising a heavy chain and a light chain, wherein: the heavy chain comprises the amino acid sequence of SEQ ID NO:380 and the light chain comprises the amino acid sequence of SEQ ID NO:427; the heavy chain comprises the amino acid sequence of SEQ ID NO:381 and the light chain comprises the amino acid sequence of SEQ ID NO:427; the heavy chain comprises the amino acid sequence of SEQ ID NO:382 and the light chain comprises the amino acid sequence of SEQ ID NO:427; the heavy chain comprises the amino acid sequence of SEQ ID NO:383 and the light chain comprises the amino acid sequence of SEQ ID NO:427; the heavy chain comprises the amino acid sequence of SEQ ID NO:384 and the light chain comprises the amino acid sequence of SEQ ID NO:427; the heavy chain comprises the amino acid sequence of SEQ ID NO:385 and the light chain comprises the amino acid sequence of SEQ ID NO:427; the heavy chain comprises the amino acid sequence of SEQ ID NO:386 and the light chain comprises the amino acid sequence of SEQ ID NO:427; the heavy chain comprises the amino acid sequence of SEQ ID NO:387 and the light chain comprises the amino acid sequence of SEQ ID NO:427; the heavy chain comprises the amino acid sequence of SEQ ID NO:388 and the light chain comprises the amino acid sequence of SEQ ID NO:427; the heavy chain comprises the amino acid sequence of SEQ ID NO:389 and the light chain comprises the amino acid sequence of SEQ ID NO:427; the heavy chain comprises the amino acid sequence of SEQ ID NO:390 and the light chain comprises the amino acid sequence of SEQ ID NO:427; the heavy chain comprises the amino acid sequence of SEQ ID NO:391 and the light chain comprises the amino acid sequence of SEQ ID NO:427; the heavy chain comprises the amino acid sequence of SEQ ID NO:392 and the light chain comprises the amino acid sequence of SEQ ID NO:427; the heavy chain comprises the amino acid sequence of SEQ ID NO:393 and the light chain comprises the amino acid sequence of SEQ ID NO:427; the heavy chain comprises the amino acid sequence of SEQ ID NO:394 and the light chain comprises the amino acid sequence of SEQ ID NO:427; the heavy chain comprises the amino acid sequence of SEQ ID NO:395 and the light chain comprises the amino acid sequence of SEQ ID NO:427; the heavy chain comprises the amino acid sequence of SEQ ID NO:396 and the light chain comprises the amino acid sequence of SEQ ID NO:427; or the heavy chain comprises the amino acid sequence of SEQ ID NO:397 and the light chain comprises the amino acid sequence of SEQ ID NO:427.

In some aspects, provided herein are anti-GPNMB antibodies comprising a heavy chain and a light chain, wherein: the heavy chain comprises the amino acid sequence of SEQ ID NO:398 and the light chain comprises the amino acid sequence of SEQ ID NO:428; the heavy chain comprises the amino acid sequence of SEQ ID NO:399 and the light chain comprises the amino acid sequence of SEQ ID NO:428; the heavy chain comprises the amino acid sequence of SEQ ID NO:400 and the light chain comprises the amino acid sequence of SEQ ID NO:428; the heavy chain comprises the amino acid sequence of SEQ ID NO:401 and the light chain comprises the amino acid sequence of SEQ ID NO:428; the heavy chain comprises the amino acid sequence of SEQ ID NO:402 and the light chain comprises the amino acid sequence of SEQ ID NO:428; the heavy chain comprises the amino acid sequence of SEQ ID NO:403 and the light chain comprises the amino acid sequence of SEQ ID NO:428; the heavy chain comprises the amino acid sequence of SEQ ID NO:404 and the light chain comprises the amino acid sequence of SEQ ID NO:428; the heavy chain comprises the amino acid sequence of SEQ ID NO:405 and the light chain comprises the amino acid sequence of SEQ ID NO:428; the heavy chain comprises the amino acid sequence of SEQ ID NO:406 and the light chain comprises the amino acid sequence of SEQ ID NO:428; the heavy chain comprises the amino acid sequence of SEQ ID NO:407 and the light chain comprises the amino acid sequence of SEQ ID NO:428; the heavy chain comprises the amino acid sequence of SEQ ID NO:408 and the light chain comprises the amino acid sequence of SEQ ID NO:428; the heavy chain comprises the amino acid sequence of SEQ ID NO:409 and the light chain comprises the amino acid sequence of SEQ ID NO:428; the heavy chain comprises the amino acid sequence of SEQ ID NO:410 and the light chain comprises the amino acid sequence of SEQ ID NO:428; the heavy chain comprises the amino acid sequence of SEQ ID NO:411 and the light chain comprises the amino acid sequence of SEQ ID NO:428; the heavy chain comprises the amino acid sequence of SEQ ID NO:412 and the light chain comprises the amino acid sequence of SEQ ID NO:428; the heavy chain comprises the amino acid sequence of SEQ ID NO:413 and the light chain comprises the amino acid sequence of SEQ ID NO:428; the heavy chain comprises the amino acid sequence of SEQ ID NO:414 and the light chain comprises the amino acid sequence of SEQ ID NO:428; or the heavy chain comprises the amino acid sequence of SEQ ID NO:415 and the light chain comprises the amino acid sequence of SEQ ID NO:428.

In some aspects, provided herein are anti-GPNMB antibodies comprising a heavy chain and a light chain, wherein: the heavy chain comprises the amino acid sequence of SEQ ID NO:398 and the light chain comprises the amino acid sequence of SEQ ID NO:429; the heavy chain comprises the amino acid sequence of SEQ ID NO:399 and the light chain comprises the amino acid sequence of SEQ ID NO:429; the heavy chain comprises the amino acid sequence of SEQ ID NO:400 and the light chain comprises the amino acid sequence of SEQ ID NO:429; the heavy chain comprises the amino acid sequence of SEQ ID NO:401 and the light chain comprises the amino acid sequence of SEQ ID NO:429; the heavy chain comprises the amino acid sequence of SEQ ID NO:402 and the light chain comprises the amino acid sequence of SEQ ID NO:429; the heavy chain comprises the amino acid sequence of SEQ ID NO:403 and the light chain comprises the amino acid sequence of SEQ ID NO:429; the heavy chain comprises the amino acid sequence of SEQ ID NO:404 and the light chain comprises the amino acid sequence of SEQ ID NO:429; the heavy chain comprises the amino acid sequence of SEQ ID NO:405 and the light chain comprises the amino acid sequence of SEQ ID NO:429; the heavy chain comprises the amino acid sequence of SEQ ID NO:406 and the light chain comprises the amino acid sequence of SEQ ID NO:429; the heavy chain comprises the amino acid sequence of SEQ ID NO:407 and the light chain comprises the amino acid sequence of SEQ ID NO:429; the heavy chain comprises the amino acid sequence of SEQ ID NO:408 and the light chain comprises the amino acid sequence of SEQ ID NO:429; the heavy chain comprises the amino acid sequence of SEQ ID NO:409 and the light chain comprises the amino acid sequence of SEQ ID NO:429; the heavy chain comprises the amino acid sequence of SEQ ID NO:410 and the light chain comprises the amino acid sequence of SEQ ID NO:429; the heavy chain comprises the amino acid sequence of SEQ ID NO:411 and the light chain comprises the amino acid sequence of SEQ ID NO:429; the heavy chain comprises the amino acid sequence of SEQ ID NO:412 and the light chain comprises the amino acid sequence of SEQ ID NO:429; the heavy chain comprises the amino acid sequence of SEQ ID NO:413 and the light chain comprises the amino acid sequence of SEQ ID NO:429; the heavy chain comprises the amino acid sequence of SEQ ID NO:414 and the light chain comprises the amino acid sequence of SEQ ID NO:429; or the heavy chain comprises the amino acid sequence of SEQ ID NO:415 and the light chain comprises the amino acid sequence of SEQ ID NO:429.

In some aspects, provided herein are anti-GPNMB antibodies comprising a heavy chain and a light chain, wherein: the heavy chain comprises the amino acid sequence of SEQ ID NO:398 and the light chain comprises the amino acid sequence of SEQ ID NO:430; the heavy chain comprises the amino acid sequence of SEQ ID NO:399 and the light chain comprises the amino acid sequence of SEQ ID NO:430; the heavy chain comprises the amino acid sequence of SEQ ID NO:400 and the light chain comprises the amino acid sequence of SEQ ID NO:430; the heavy chain comprises the amino acid sequence of SEQ ID NO:401 and the light chain comprises the amino acid sequence of SEQ ID NO:430; the heavy chain comprises the amino acid sequence of SEQ ID NO:402 and the light chain comprises the amino acid sequence of SEQ ID NO:430; the heavy chain comprises the amino acid sequence of SEQ ID NO:403 and the light chain comprises the amino acid sequence of SEQ ID NO:430; the heavy chain comprises the amino acid sequence of SEQ ID NO:404 and the light chain comprises the amino acid sequence of SEQ ID NO:430; the heavy chain comprises the amino acid sequence of SEQ ID NO:405 and the light chain comprises the amino acid sequence of SEQ ID NO:430; the heavy chain comprises the amino acid sequence of SEQ ID NO:406 and the light chain comprises the amino acid sequence of SEQ ID NO:430; the heavy chain comprises the amino acid sequence of SEQ ID NO:407 and the light chain comprises the amino acid sequence of SEQ ID NO:430; the heavy chain comprises the amino acid sequence of SEQ ID NO:408 and the light chain comprises the amino acid sequence of SEQ ID NO:430; the heavy chain comprises the amino acid sequence of SEQ ID NO:409 and the light chain comprises the amino acid sequence of SEQ ID NO:430; the heavy chain comprises the amino acid sequence of SEQ ID NO:410 and the light chain comprises the amino acid sequence of SEQ ID NO:430; the heavy chain comprises the amino acid sequence of SEQ ID NO:411 and the light chain comprises the amino acid sequence of SEQ ID NO:430; the heavy chain comprises the amino acid sequence of SEQ ID NO:412 and the light chain comprises the amino acid sequence of SEQ ID NO:430; the heavy chain comprises the amino acid sequence of SEQ ID NO:413 and the light chain comprises the amino acid sequence of SEQ ID NO:430; the heavy chain comprises the amino acid sequence of SEQ ID NO:414 and the light chain comprises the amino acid sequence of SEQ ID NO:430; or the heavy chain comprises the amino acid sequence of SEQ ID NO:415 and the light chain comprises the amino acid sequence of SEQ ID NO:430.

In some aspects, provided herein are anti-GPNMB antibodies comprising a heavy chain and a light chain, wherein: the heavy chain comprises the amino acid sequence of SEQ ID NO:398 and the light chain comprises the amino acid sequence of SEQ ID NO:431; the heavy chain comprises the amino acid sequence of SEQ ID NO:399 and the light chain comprises the amino acid sequence of SEQ ID NO:431; the heavy chain comprises the amino acid sequence of SEQ ID NO:400 and the light chain comprises the amino acid sequence of SEQ ID NO:431; the heavy chain comprises the amino acid sequence of SEQ ID NO:401 and the light chain comprises the amino acid sequence of SEQ ID NO:431; the heavy chain comprises the amino acid sequence of SEQ ID NO:402 and the light chain comprises the amino acid sequence of SEQ ID NO:431; the heavy chain comprises the amino acid sequence of SEQ ID NO:403 and the light chain comprises the amino acid sequence of SEQ ID NO:431; the heavy chain comprises the amino acid sequence of SEQ ID NO:404 and the light chain comprises the amino acid sequence of SEQ ID NO:431; the heavy chain comprises the amino acid sequence of SEQ ID NO:405 and the light chain comprises the amino acid sequence of SEQ ID NO:431; the heavy chain comprises the amino acid sequence of SEQ ID NO:406 and the light chain comprises the amino acid sequence of SEQ ID NO:431; the heavy chain comprises the amino acid sequence of SEQ ID NO:407 and the light chain comprises the amino acid sequence of SEQ ID NO:431; the heavy chain comprises the amino acid sequence of SEQ ID NO:408 and the light chain comprises the amino acid sequence of SEQ ID NO:431; the heavy chain comprises the amino acid sequence of SEQ ID NO:409 and the light chain comprises the amino acid sequence of SEQ ID NO:431; the heavy chain comprises the amino acid sequence of SEQ ID NO:410 and the light chain comprises the amino acid sequence of SEQ ID NO:431; the heavy chain comprises the amino acid sequence of SEQ ID NO:411 and the light chain comprises the amino acid sequence of SEQ ID NO:431; the heavy chain comprises the amino acid sequence of SEQ ID NO:412 and the light chain comprises the amino acid sequence of SEQ ID NO:431; the heavy chain comprises the amino acid sequence of SEQ ID NO:413 and the light chain comprises the amino acid sequence of SEQ ID NO:431; the heavy chain comprises the amino acid sequence of SEQ ID NO:414 and the light chain comprises the amino acid sequence of SEQ ID NO:431; or the heavy chain comprises the amino acid sequence of SEQ ID NO:415 and the light chain comprises the amino acid sequence of SEQ ID NO:431.

In some aspects, provided herein are anti-GPNMB antibodies comprising a heavy chain and a light chain, wherein: the heavy chain comprises the amino acid sequence of SEQ ID NO:398 and the light chain comprises the amino acid sequence of SEQ ID NO:432; the heavy chain comprises the amino acid sequence of SEQ ID NO:399 and the light chain comprises the amino acid sequence of SEQ ID NO:432; the heavy chain comprises the amino acid sequence of SEQ ID NO:400 and the light chain comprises the amino acid sequence of SEQ ID NO:432; the heavy chain comprises the amino acid sequence of SEQ ID NO:401 and the light chain comprises the amino acid sequence of SEQ ID NO:432; the heavy chain comprises the amino acid sequence of SEQ ID NO:402 and the light chain comprises the amino acid sequence of SEQ ID NO:432; the heavy chain comprises the amino acid sequence of SEQ ID NO:403 and the light chain comprises the amino acid sequence of SEQ ID NO:432; the heavy chain comprises the amino acid sequence of SEQ ID NO:404 and the light chain comprises the amino acid sequence of SEQ ID NO:432; the heavy chain comprises the amino acid sequence of SEQ ID NO:405 and the light chain comprises the amino acid sequence of SEQ ID NO:432; the heavy chain comprises the amino acid sequence of SEQ ID NO:406 and the light chain comprises the amino acid sequence of SEQ ID NO:432; the heavy chain comprises the amino acid sequence of SEQ ID NO:407 and the light chain comprises the amino acid sequence of SEQ ID NO:432; the heavy chain comprises the amino acid sequence of SEQ ID NO:408 and the light chain comprises the amino acid sequence of SEQ ID NO:432; the heavy chain comprises the amino acid sequence of SEQ ID NO:409 and the light chain comprises the amino acid sequence of SEQ ID NO:432; the heavy chain comprises the amino acid sequence of SEQ ID NO:410 and the light chain comprises the amino acid sequence of SEQ ID NO:432; the heavy chain comprises the amino acid sequence of SEQ ID NO:411 and the light chain comprises the amino acid sequence of SEQ ID NO:432; the heavy chain comprises the amino acid sequence of SEQ ID NO:412 and the light chain comprises the amino acid sequence of SEQ ID NO:432; the heavy chain comprises the amino acid sequence of SEQ ID NO:413 and the light chain comprises the amino acid sequence of SEQ ID NO:432; the heavy chain comprises the amino acid sequence of SEQ ID NO:414 and the light chain comprises the amino acid sequence of SEQ ID NO:432; or the heavy chain comprises the amino acid sequence of SEQ ID NO:415 and the light chain comprises the amino acid sequence of SEQ ID NO:432.

In some aspects, provided herein are anti-GPNMB antibodies comprising a heavy chain and a light chain, wherein: the heavy chain comprises the amino acid sequence of SEQ ID NO:398 and the light chain comprises the amino acid sequence of SEQ ID NO:433; the heavy chain comprises the amino acid sequence of SEQ ID NO:399 and the light chain comprises the amino acid sequence of SEQ ID NO:433; the heavy chain comprises the amino acid sequence of SEQ ID NO:400 and the light chain comprises the amino acid sequence of SEQ ID NO:433; the heavy chain comprises the amino acid sequence of SEQ ID NO:401 and the light chain comprises the amino acid sequence of SEQ ID NO:433; the heavy chain comprises the amino acid sequence of SEQ ID NO:402 and the light chain comprises the amino acid sequence of SEQ ID NO:433; the heavy chain comprises the amino acid sequence of SEQ ID NO:403 and the light chain comprises the amino acid sequence of SEQ ID NO:433; the heavy chain comprises the amino acid sequence of SEQ ID NO:404 and the light chain comprises the amino acid sequence of SEQ ID NO:433; the heavy chain comprises the amino acid sequence of SEQ ID NO:405 and the light chain comprises the amino acid sequence of SEQ ID NO:433; the heavy chain comprises the amino acid sequence of SEQ ID NO:406 and the light chain comprises the amino acid sequence of SEQ ID NO:433; the heavy chain comprises the amino acid sequence of SEQ ID NO:407 and the light chain comprises the amino acid sequence of SEQ ID NO:433; the heavy chain comprises the amino acid sequence of SEQ ID NO:408 and the light chain comprises the amino acid sequence of SEQ ID NO:433; the heavy chain comprises the amino acid sequence of SEQ ID NO:409 and the light chain comprises the amino acid sequence of SEQ ID NO:433; the heavy chain comprises the amino acid sequence of SEQ ID NO:410 and the light chain comprises the amino acid sequence of SEQ ID NO:433; the heavy chain comprises the amino acid sequence of SEQ ID NO:411 and the light chain comprises the amino acid sequence of SEQ ID NO:433; the heavy chain comprises the amino acid sequence of SEQ ID NO:412 and the light chain comprises the amino acid sequence of SEQ ID NO:433; the heavy chain comprises the amino acid sequence of SEQ ID NO:413 and the light chain comprises the amino acid sequence of SEQ ID NO:433; the heavy chain comprises the amino acid sequence of SEQ ID NO:414 and the light chain comprises the amino acid sequence of SEQ ID NO:433; or the heavy chain comprises the amino acid sequence of SEQ ID NO:415 and the light chain comprises the amino acid sequence of SEQ ID NO:433.

In some aspects, the anti-GPNMB antibody according to any of the above aspects is a monoclonal antibody, including a humanized and/or human antibody. In some aspects, the anti-GPNMB antibody is an antibody fragment, e.g., a Fv, Fab, Fab′, scFv, diabody, or F(ab′)2 fragment. In some aspects, the anti-GPNMB antibody is a substantially full-length antibody, e.g., an IgG1 antibody, IgG2a antibody or other antibody class or isotype as defined herein.

In some aspects, an anti-GPNMB antibody according to any of the above aspects may incorporate any of the features, singly or in combination, as described below.

### Anti-GPNMB Antibody Binding Affinity

In some embodiments of any of the antibodies provided herein, the antibody has a dissociation constant (K_{D}) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹ M). Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In one embodiment, Kd is measured by a radiolabeled antigen binding assay (RIA). In some embodiment, an RIA is performed with the Fab version of an antibody of interest and its antigen, for example as described in Chen et al. J. Mol. Biol. 293:865-881(1999)). In some embodiments, K_{D} is measured using a BIACORE surface plasmon resonance assay, for example, an assay using a BIACORE-2000 or a BIACORE-3000 (BIAcore, Inc., Piscataway, NJ) is performed at 25° C. with immobilized antigen CM5 chips at ˜10 response units (RU). In some embodiments, the K_{D} is determined using a monovalent antibody (e.g., a Fab) or a full-length antibody. In some embodiments, the K_{D} is determined using a full-length antibody in a monovalent form.

In some embodiments, an anti-GPNMB antibody of the present disclosure binds to human GPNMB, wherein the K_{D} of binding to human GPNMB is from about 0.4 nM to about 120 nM. In some embodiments, an anti-GPNMB antibody binds to cynomolgus GPNMB, wherein the K_{D} of binding to cynomolgus GPNMB is from about 0.4 nM to about 104 nM. In some embodiments, an anti-GPNMB antibody of the present disclosure binds to murine GPNMB, wherein the K_{D} of binding to murine GPNMB is from about 0.3 nM to about 4.6 nM.

### Antibody Fragments

In some embodiments of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain embodiments, amino acids of an antibody variable domain that can be modified without diminishing the native affinity of the antigen-binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### Human Antibodies

In some embodiments of any of the antibodies provided herein, the antibody is a human antibody.

Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(−2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### Constant Regions Including Fc Regions

In some embodiments of any of the antibodies provided herein, the antibody comprises an Fc. In some embodiments, the Fc is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody induces the one or more GPNMB activities or independently of binding to an Fc receptor. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971; WO 2007/106585).

In some embodiments, the Fc region increases clustering without activating complement as compared to a corresponding antibody comprising an Fc region that does not comprise the amino acid substitutions. In some embodiments, the antibody induces one or more activities of a target specifically bound by the antibody. In some embodiments, the antibody binds to GPNMB.

It may also be desirable to modify an anti-GPNMB antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other embodiments, it may also be desirable to modify an anti-GPNMB antibody of the present disclosure to modify effector function to increase finding selectivity toward the ITIM-containing FcgRIIb (CD32b) to increase clustering of GPNMB antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### Antibody Variants

In some embodiments of any of the antibodies provided herein, amino acid sequence variants of the antibodies are contemplated. For example, it may be desirable to improve the binding affinity and/or other biological properties of the antibody.

### Substitution, Insertion, and Deletion Variants

In some embodiments of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions”.

In certain embodiments of the variant VH and VL sequences provided above, each HVR is unaltered.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue outside the HVRs and not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the disclosure may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see, e.g., Yamane-Ohnuki et al. Biotech. Bioeng. 87: 614 (2004) and Kanda et al. Biotechnol. Bioeng. 94(4):680-688 (2006)).

### Modified Constant Regions

In some embodiments of any of the antibodies provided herein, the antibody Fc is an antibody, Fc isotypes and/or modifications. In some embodiments, the antibody Fc isotype and/or modification is capable of binding to Fc gamma receptor.

In some embodiments of any of the antibodies provided herein, the modified antibody Fc is an IgG1 modified Fc. In some embodiments, the IgG1 modified Fc comprises one or more modifications. For example, in some embodiments, the IgG1 modified Fc comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments of any of the IgG1 modified Fc, the Fc comprises N297A mutation according to EU numbering. In some embodiments of any of the IgG1 modified Fc, the Fc comprises D265A and N297A mutations according to EU numbering. In some embodiments of any of the IgG1 modified Fc, the Fc comprises D270A mutations according to EU numbering. In some embodiments, the IgG1 modified Fc comprises L234A and L235A mutations according to EU numbering. In some embodiments of any of the IgG1 modified Fc, the Fc comprises L234A and G237A mutations according to EU numbering. In some embodiments of any of the IgG1 modified Fc, the Fc comprises L234A, L235A and G237A mutations according to EU numbering. In some embodiments of any of the IgG1 modified Fc, the Fc comprises one or more (including all) of P238D, L328E, E233, G237D, H268D, P271G and A330R mutations according to EU numbering. In some embodiments of any of the IgG1 modified Fc, the Fc comprises one or more of S267E/L328F mutations according to EU numbering. In some embodiments of any of the IgG1 modified Fc, the Fc comprises P238D, L328E, E233D, G237D, H268D, P271G and A330R mutations according to EU numbering. In some embodiments of any of the IgG1 modified Fe, the Fe comprises P238D, L328E, G237D, H268D, P271G and A330R mutations according to EU numbering. In some embodiments of any of the IgG1 modified Fc, the Fc comprises P238D, S267E, L328E, E233D, G237D, H268D, P271G and A330R mutations according to EU numbering. In some embodiments of any of the IgG1 modified Fc, the Fc comprises P238D, S267E, L328E, G237D, H268D, P271G and A330R mutations according to EU numbering. In some embodiments of any of the IgG1 modified Fc, the Fc comprises C226S, C229S, E233P, L234V, and L235A mutations according to EU numbering. In some embodiments of any of the IgG1 modified Fc, the Fc comprises L234F, L235E, and P331S mutations according to EU numbering. In some embodiments of any of the IgG1 modified Fc, the Fc comprises S267E and L328F mutations according to EU numbering. In some embodiments of any of the IgG1 modified Fc, the Fc comprises N325S and L328F mutations according to EU numbering. In some embodiments of any of the IgG1 modified Fc, the Fc comprises S267E mutations according to EU numbering. In some embodiments of any of the IgG1 modified Fc, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some embodiments of any of the IgG1 modified Fc, the Fc includes two or more amino acid substitutions that increase antibody clustering without activating complement as compared to a corresponding antibody having an Fc region that does not include the two or more amino acid substitutions. Accordingly, in some embodiments of any of the IgG1 modified Fc, the IgG1 modified Fc is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some embodiments, the IgG1 modified Fc comprises an amino acid substitution at positions E430G, L243A, L235A, and P331S according to EU numbering. In some embodiments, the IgG1 modified Fc comprises an amino acid substitution at positions E430G and P331S according to EU numbering. In some embodiments, the IgG1 modified Fc comprises an amino acid substitution at positions E430G and K322A according to EU numbering. In some embodiments, the IgG1 modified Fc comprises an amino acid substitution at positions E430G, A330S, and P331S according to EU numbering. In some embodiments, the IgG1 modified Fc comprises an amino acid substitution at positions E430G, K322A, A330S, and P331S according to EU numbering. In some embodiments, the IgG1 modified Fc comprises an amino acid substitution at positions E430G, K322A, and A330S according to EU numbering. In some embodiments, the IgG1 modified Fc comprises an amino acid substitution at positions E430G, K322A, and P331S according to EU numbering.

In some embodiments of any of the IgG1 modified Fc, the IgG1 modified Fc may further comprise herein may be combined with an A330L mutation (Lazar et al. Proc Nat Acad Sci USA, 103:4005-4010 (2006)), or one or more of L234F, L235E, and/or P331S mutations (Sazinsky et al. Proc Natl Acad Sci USA, 105:20167-20172 (2008)), according to the EU numbering convention, to eliminate complement activation. In some embodiments of any of the IgG1 modified Fc, the IgG1 modified Fc may further comprise one or more of A330L, A330S, L234F, L235E, and/or P331S according to EU numbering. In some embodiments of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some embodiments of any of the IgG1 modified Fc, the IgG1 modified Fc may further comprise one or more of E430G, E430S, E430F, E430T, E345K, E345Q, E345R, E345Y, S440Y, and/or S440W according to EU numbering.

In some embodiments of any of the IgG1 modified Fc, the IgG1 modified Fc comprises an amino acid substitution at positions L234A, L235A, and/or P331S according to EU numbering. In some embodiments of any of the IgG1 modified Fc, the IgG1 modified Fc comprises L234A, L235A, and/or P329S according to EU numbering. In some embodiments of any of the IgG1 modified Fc, the IgG1 modified Fc comprises L234A, L235A, and/or P331G according to EU numbering. In some embodiments of any of the IgG1 modified Fc, the IgG1 modified Fc comprises L234A, L235A, and/or P329G according to EU numbering. In some embodiments of any of the IgG1 modified Fc, the IgG1 modified Fc comprises P331S and/or E430G according to EU numbering. In some embodiments of any of the IgG1 modified Fc, the IgG1 modified Fc comprises N325S and/or L328F according to EU numbering. Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-GPNMB antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

In some embodiments of any of the antibodies provided herein, the modified antibody Fc is an IgG2 modified Fc. In some embodiments, the IgG2 modified Fc comprises one or more modifications. For example, in some embodiments, the IgG2 modified Fc comprises one or more amino acid substitutions (e.g., relative to a wild-type Fc region of the same isotype). In some embodiments of any of the IgG2 modified Fc, the one or more amino acid substitutions are selected from V234A (Alegre et al. Transplantation 57:1537-1543 (1994); Xu et al. Cell Immunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1(Suppl. 1):27 (2000); Armour et al. The Haematology Journal 1(Suppl. 1):27 (2000)), C219S, and/or C220S (White et al. Cancer Cell 27, 138-148 (2015)); S267E, L328F (Chu et al. Mol Immunol, 45:3926-3933 (2008)); and M252Y, S254T, and/or T256E according to the EU numbering convention. In some embodiments of any of the IgG2 modified Fc, the Fc comprises an amino acid substitution at positions V234A and G237A according to EU numbering. In some embodiments of any of the IgG2 modified Fc, the Fc comprises an amino acid substitution at positions C219S or C220S according to EU numbering. In some embodiments of any of the IgG2 modified Fc, the Fc comprises an amino acid substitution at positions A330S and P331S according to EU numbering. In some embodiments of any of the IgG2 modified Fc, the Fc comprises an amino acid substitution at positions S267E and L328F according to EU numbering.

In some embodiments of any of the IgG2 modified Fc, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fc, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fc, the Fc comprises a C220S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc comprises a C219S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc includes an IgG2 isotype heavy chain constant domain 1 (CH1) and hinge region (White et al. Cancer Cell 27:138-148 (2015)). In certain embodiments of any of the IgG2 modified Fc, the IgG2 isotype CH1 and hinge region comprise the amino acid sequence of 118-230 according to EU numbering. In some embodiments of any of the IgG2 modified Fc, the antibody Fc region comprises a S267E amino acid substitution, a L328F amino acid substitution, or both, and/or a N297A or N297Q amino acid substitution according to the EU numbering convention.

In some embodiments of any of the IgG2 modified Fc, the Fc further comprises one or more amino acid substitution at positions E430G, E430S, E430F, E430T, E345K, E345Q, E345R, E345Y, S440Y, and S440W according to EU numbering. In some embodiments of any of the IgG2 modified Fc, the Fc may further comprise one or more mutations to enhance the antibody half-life in human serum (e.g., one or more (including all) of M252Y, S254T, and T256E mutations according to the EU numbering convention). In some embodiments of any of the IgG2 modified Fc, the Fc may further comprise A330S and P331S.

In some embodiments of any of the IgG2 modified Fc, the Fc is an IgG2/4 hybrid Fc. In some embodiments, the IgG2/4 hybrid Fc comprises IgG2 aa 118 to 260 and IgG4 aa 261 to 447. In some embodiments of any IgG2 modified Fc, the Fc comprises one or more amino acid substitutions at positions H268Q, V309L, A330S, and P331S according to EU numbering.

In some embodiments of any of the IgG1 and/or IgG2 modified Fc, the Fc comprises one or more additional amino acid substitutions selected from A330L, L234F; L235E, or P331S according to EU numbering; and any combination thereof.

In certain embodiments of any of the IgG1 and/or IgG2 modified Fc, the Fc comprises one or more amino acid substitutions at a residue position selected from C127S, L234A, L234F, L235A, L235E, S267E, K322A, L328F, A330S, P3311S, E345R, E430G, S440Y, and any combination thereof according to EU numbering. In some embodiments of any of the IgG1 and/or IgG2 modified Fc, the Fc comprises an amino acid substitution at positions E430G, L243A, L235A, and P331S according to EU numbering. In some embodiments of any of the IgG1 and/or IgG2 modified Fc, the Fc comprises an amino acid substitution at positions E430G and P331S according to EU numbering. In some embodiments of any of the IgG1 and/or IgG2 modified Fc, the Fc comprises an amino acid substitution at positions E430G and K322A according to EU numbering. In some embodiments of any of the IgG1 and/or IgG2 modified Fc, the Fc comprises an amino acid substitution at positions E430G, A330S, and P331S according to EU numbering. In some embodiments of any of the IgG1 and/or IgG2 modified Fc, the Fc comprises an amino acid substitution at positions E430G, K322A, A330S, and P331S according to EU numbering. In some embodiments of any of the IgG1 and/or IgG2 modified Fc, the Fc comprises an amino acid substitution at positions E430G, K322A, and A330S according to EU numbering. In some embodiments of any of the IgG1 and/or IgG2 modified Fc, the Fc comprises an amino acid substitution at positions E430G, K322A, and P331S according to EU numbering. In some embodiments of any of the IgG1 and/or IgG2 modified Fc, the Fc comprises an amino acid substitution at positions S267E and L328F according to EU numbering. In some embodiments of any of the IgG1 and/or IgG2 modified Fc, the Fc comprises an amino acid substitution at position C127S according to EU numbering. In some embodiments of any of the IgG1 and/or IgG2 modified Fc, the Fc comprises an amino acid substitution at positions E345R, E430G and S440Y according to EU numbering.

In some embodiments of any of the antibodies provided herein, the modified antibody Fc is an IgG4 modified Fc. In some embodiments, the IgG4 modified Fc comprises one or more modifications. For example, in some embodiments, the IgG4 modified Fc comprises one or more amino acid substitutions (e.g., relative to a wild-type Fc region of the same isotype). In some embodiments of any of the IgG4 modified Fc, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some embodiments of any of the IgG4 modified Fc, the Fc may further comprise L235A, G237A, and E318A according to the EU numbering convention. In some embodiments of any of the IgG4 modified Fc, the Fc may further comprise S228P and L235E according to the EU numbering convention. In some embodiments of any of the IgG4 modified Fc, the IgG4 modified Fc may further comprise S267E and L328F according to the EU numbering convention.

In some embodiments of any of the IgG4 modified Fc, the IgG4 modified Fc comprises or may be combined with an S228P mutation according to the EU numbering convention (Angal et al. Mol Immunol. 30:105-108 (1993)) and/or with one or more mutations described in (Peters et al. J Biol Chem. 287(29):24525-33 (2012)) to enhance antibody stabilization.

In some embodiments of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some embodiments of any of the IgG4 modified Fc, the Fc comprises L235E according to EU numbering. In certain embodiments of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fc, the Fc comprises an amino acid substitution at positions E430G, L243A, L235A, and P331S according to EU numbering. In some embodiments of any of the IgG4 modified Fc, the Fc comprises an amino acid substitution at positions E430G and P331S according to EU numbering. In some embodiments of any of the IgG4 modified Fc, the Fc comprises an amino acid substitution at positions E430G and K322A according to EU numbering. In some embodiments of any of the IgG4 modified Fc, the Fc comprises an amino acid substitution at position E430 according to EU numbering. In some embodiments of any of the IgG4 modified Fc, the Fc region comprises an amino acid substitution at positions E430G and K322A according to EU numbering. In some embodiments of any of the IgG4 modified Fc, the Fc comprises an amino acid substitution at positions S267E and L328F according to EU numbering. In some embodiments of any of the IgG4 modified Fc, the Fc comprises an amino acid substitution at position C127S according to EU numbering. In some embodiments of any of the IgG4 modified Fc, the Fc comprises an amino acid substitution at positions E345R, E430G and S440Y according to EU numbering.

### Other Antibody Modifications

In some embodiments of any of the antibodies, the antibody is a derivative. The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen-binding protein can have a greater circulating half-life than an antigen-binding protein that is not chemically modified. In certain embodiments, a chemically modified antigen-binding protein can have improved targeting capacity for desired cells, tissues, and/or organs. In some embodiments, a derivative antigen-binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative antigen-binding protein comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1, 3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments, PEG is used to improve the therapeutic capacity for an antigen-binding protein. In certain embodiments, PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere, J Adv. Drug Res., 15:29 (1986); and Evans et al. J. Med. Chem., 30:1229 (1987), which are incorporated herein by reference for any purpose. Such compounds are often developed with the aid of computerized molecular modeling. Peptide mimetics that are structurally similar to therapeutically useful peptides can be used to produce a similar therapeutic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable peptides. In addition, constrained peptides comprising a consensus sequence or a substantially identical consensus sequence variation can be generated by methods known in the art (Rizo and Gierasch Ann. Rev. Biochem., 61:387 (1992), incorporated herein by reference for any purpose); for example, by adding internal cysteine residues capable of forming intramolecular disulfide bridges which cyclize the peptide.

Drug conjugation involves coupling of a biological active cytotoxic (anticancer) payload or drug to an antibody that specifically targets a certain tumor marker (e.g. a polypeptide that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue OncLive Jul. 5, 2012; ADC Review on antibody-drug conjugates; and Ducry et al. Bioconjugate Chemistry 21 (1):5-13 (2010).

### Nucleic Acids, Vectors, and Host Cells

Anti-GPNMB antibodies of the present disclosure may be produced using recombinant methods and compositions, e.g., as described in U.S. Pat. No. 4,816,567. In some embodiments, isolated nucleic acids having a nucleotide sequence encoding any of the anti-GPNMB antibodies of the present disclosure are provided. Such nucleic acids may encode an amino acid sequence comprising the V_{L} and/or an amino acid sequence comprising the V_{H} of the anti-GPNMB antibody (e.g., the light and/or heavy chains of the antibody). In some embodiments, one or more vectors (e.g., expression vectors) comprising such nucleic acids are provided. In some embodiments, a host cell comprising such nucleic acid is also provided. In some embodiments, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody.

In some embodiments, the host cell comprises (e.g., has been transduced with): (1) a nucleic acid that encodes an amino acid sequence comprising a light chain of an antibody, wherein the light chain comprises a V_{L} and (2) a nucleic acid that encodes an amino acid sequence comprising a heavy chain of an antibody, wherein the heavy chain comprises a V_{H}, wherein the VL and the V_{H} form an antigen-binding domain that binds to GPNMB In some embodiments, the host cell comprises (e.g., has been transduced with): (1) a nucleic acid that encodes an amino acid sequence comprising a light chain of an antibody, wherein the light chain comprises a V_{L}, (2) a nucleic acid that encodes an amino acid sequence comprising a heavy chain of an antibody, wherein the heavy chain comprises a V_{H}, and (3) a nucleic acid that encodes a fragment of a heavy chain, wherein the heavy chain not comprise a V_{H} (e.g., a fragment of a heavy chain comprising a CH2 and a CH3 domain), wherein the V_{L} and the V_{H} form an antigen-binding domain that binds to GPNMB. The nucleic acids can be within the same vector or can be in different vectors.

In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-GPNMB antibody of the present disclosure are provided. In some embodiments, the method includes culturing a host cell of the present disclosure comprising a nucleic acid encoding the anti-GPNMB antibody, under conditions suitable for expression of the antibody. In some embodiments, the antibody is subsequently recovered from the host cell (or host cell culture medium).

For recombinant production of an anti-GPNMB antibody of the present disclosure, a nucleic acid encoding the anti-GPNMB antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-GPNMB antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-GPNMB antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross Nat. Biotech. 22:1409-1414 (2004); and Li et al. Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### Pharmaceutical Compositions/Formulations

Provided herein are pharmaceutical compositions and/or pharmaceutical formulations comprising the anti-GPNMB antibodies of the present disclosure and a pharmaceutically acceptable carrier.

In some embodiments, the antibody or antigen-binding fragment thereof having the desired degree of purity is present in a formulation comprising, e.g., a physiologically acceptable carrier, excipient or stabilizer (Remington's Pharmaceutical Sciences (1990) Mack Publishing Co., Easton, PA). In some embodiments, pharmaceutically acceptable carriers preferably are nontoxic to recipients at the dosages and concentrations employed.

Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some aspects, a pharmaceutical composition comprises an anti-GPNMB antibody or antigen-binding fragment thereof as described herein, and a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)).

In some embodiments, a pharmaceutically acceptable carrier preferably is nontoxic to recipients at the dosages and concentrations employed. The pharmaceutical compositions and/or pharmaceutical formulations to be used for in vivo administration can be sterile. This is readily accomplished by filtration through, e.g., sterile filtration membranes.

Pharmaceutical compositions and/or pharmaceutical formulations provided herein are useful as a medicament, e.g., for treating cancer or for treating a neurodegenerative disorder.

### Therapeutic Uses

As disclosed herein, anti-GPNMB antibodies of the present disclosure may be used for treating diseases, disorders, and conditions. In some embodiments, the present disclosure provides methods for treating an individual having a neurodegenerative disorder, such as, for example, Parkinson's disease, comprising administering to the individual a therapeutically effective amount of an anti-GPNMB antibody of the present disclosure. In other embodiments, the present disclosure provides methods for treating an individual with a lysosomal storage disease comprising administering to the individual a therapeutically effective amount of an anti-GPNMB antibody of the present disclosure. In some embodiments, the lysosomal storage disease is Gaucher's disease. In other embodiments, the present disclosure provides methods for treating an individual having cancer comprising administering to the individual a therapeutically effective amount of an anti-GPNMB antibody of the present disclosure.

Ectopic or expression of GPNMB has been observed in various tumors; overexpression and activation of GPNMB has been implicated in lymphoid leukemia, lymphoma, adenoma, melanoma, gastric, prostate, and breast cancers; and GPNMB overexpression has been associated with metastasis. Accordingly, modulating the activity of GPNMB with an anti-GPNMB antibody of the present disclosure is an effective means of treating cancer.

In certain aspects, provided herein are methods for treating cancer in a subject in need thereof, the method comprising administering to the subject an anti-GPNMB antibody of the present disclosure, or a pharmaceutical composition comprising an anti-GPNMB antibody of the present disclosure. In some embodiments, a method is provided for treating cancer in a subject in need thereof, the method comprising administering to the subject an anti-GPNMB antibody of the present disclosure.

In some embodiments, the cancer is selected from sarcoma, bladder cancer, breast cancer, colon cancer, endometrial cancer, kidney cancer, renal cancer, leukemia, lung cancer, non-small cell lung cancer, melanoma, lymphoma, pancreatic cancer, prostate cancer, ovarian cancer, stomach cancer, thyroid cancer, cancer of the uterus, liver cancer, cervical cancer, testicular cancer, squamous cell carcinoma, glioma, glioblastoma, adenoma, and neuroblastoma. In some embodiments, the cancer is selected from glioblastoma multiforme, bladder carcinoma, and esophageal carcinoma. In some embodiments, the cancer is triple-negative breast carcinoma. In some embodiments, the cancer may be a primary tumor. In some embodiments, the cancer may be a metastatic tumor at a second site derived from any of the above types of cancer. In some embodiments, an anti-GPNMB antibody of the present disclosure is useful for treating cancer in s subject in need thereof, wherein the cancer expresses GPNMB.

In some embodiments, an anti-GPNMB antibody of the present disclosure may be administered in conjunction with one or more therapeutic agents that act as a checkpoint inhibitor. In some embodiments, the method further includes administering to the individual at least one antibody that specifically binds to an inhibitory immune checkpoint molecule, and/or another standard or investigational anti-cancer therapy. In some embodiments, the inhibitory checkpoint molecule is selected from PD1, PD-L1, and PD-L2. In some embodiments, the at least one antibody that specifically binds to an inhibitory checkpoint molecule is administered in combination with an anti-GPNMB antibody of the present disclosure.

In some embodiments, the at least one antibody that specifically binds to an inhibitory checkpoint molecule is selected from an anti-PD-L1 antibody, an anti-PD-L2 antibody, and an anti-PD-1 antibody.

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

### Articles of Manufacture

Provided herein are articles of manufacture (e.g., kit) comprising a monovalent anti-GPNMB antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including. In some embodiments, the second agent is a pharmaceutically active agent.

In some embodiments of any of the articles of manufacture, the article of manufactures further include instructions for use in accordance with the methods of this disclosure. The instructions generally include information as to dosage, dosing schedule, and route of administration for the intended treatment. In some embodiments, these instructions comprise a description of administration of the isolated antibody of the present disclosure (e.g., an anti-GPNMB antibody described herein) to treat an individual having a disease, disorder, or injury, such as for example cancer or a neurodegenerative disorder (e.g., Parkinson's disease), according to any methods of this disclosure. In some embodiments, the instructions include instructions for use of the anti-GPNMB antibody and the second agent (e.g., second pharmaceutically active agent).

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Production of Recombinant GPNMB Polypeptides and GPNMB-Expressing Cell Lines

The amino acid sequence for human GPNMB preprotein is set forth below in SEQ ID NO: 1. Human GPNMB contains a signal peptide at amino residues 1-22; an extracellular domain at amino acid residues 23-498; a putative integrin binding site at amino residues 64-66; a PKD (polycystic kidney disease) domain at amino residues 240-327; a transmembrane domain at amino residues 499-519; and an intracellular domain at amino residues 520-572 of SEQ ID NO: 1.

Full-length Human GPNMB Amino Acid Sequence (SEQ
ID NO: 1):
MECLYYFLGFLLLAARLPLDAAKRFHDVLGNERPSAYMREHNQLNGWSS
DENDWNEKLYPVWKRGDMRWKNSWKGGRVQAVLTSDSPALVGSNITFAV
NLIFPRCQKEDANGNIVYEKNCRNEAGLSADPYVYNWTAWSEDSDGENG
TGQSHHNVFPDGKPFPHHPGWRRWNFIYVFHTLGQYFQKLGRCSVRVSV
NTANVTLGPQLMEVTVYRRHGRAYVPIAQVKDVYVVTDQIPVFVTMFQK
NDRNSSDETFLKDLPIMFDVLIHDPSHFLNYSTINYKWSFGDNTGLFVS
TNHTVNHTYVLNGTFSLNLTVKAAAPGPCPPPPPPPRPSKPTPSLATTL
KSYDSNTPGPAGDNPLELSRIPDENCQINRYGHFQATITIVEGILEVNI
IQMTDVLMPVPWPESSLIDFVVTCQGSIPTEVCTIISDPTCEITQNTVC
SPVDVDEMCLLTVRRTFNGSGTYCVNLTLGDDTSLALTSTLISVPDRDP
ASPLRMANSALISVGCLAIFVTVISLLVYKKHKEYNPIENSPGNVVRSK
GLSVFLNRAKAVFFPGNQEKDPLLKNQEFKGVS

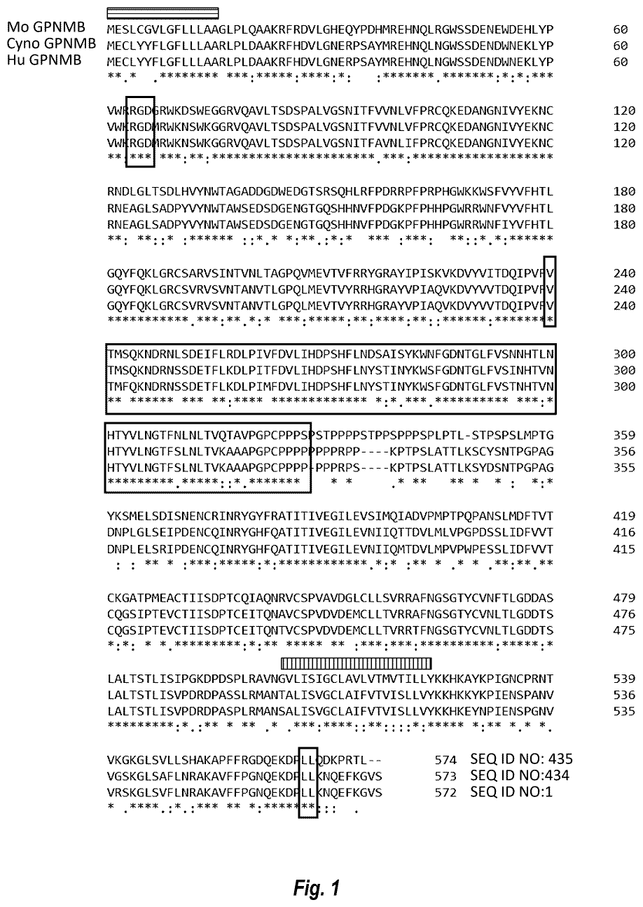

To assess amino acid identity between human, mouse, and cynomolgus GPNMB, an alignment of the amino acid sequences of the primary isoforms of human (Hu) and mouse (Mo) GPNMB and of human and cynomolgus (Cy) GPNMB were generated by 2-way blast analysis. Alignment of the amino acid sequences of the primary isoforms of human and mouse GPNMB showed that the extracellular domain of both proteins (excluding leader sequence) share ˜70% identity (FIG. 1). Alignment of the amino acid sequences of the primary isoforms of human and cynomolgus GPNMB showed that the extracellular domain of both human and cynomolgus proteins (excluding the leader sequence) share 96% identity (FIG. 1). High amino acid sequence homology among these results in conservation of several protein motifs that may determine protein function, such as an integrin-binding site (small, boxed area in FIG. 1 corresponding to amino acids RGD), a PKD domain (large boxed area in FIG. 1), and a cytoplasmic dileucine motif (located at the C-terminal region of the protein).

A stable cell line expressing full-length untagged human GPNMB was established by transfecting cells with transposon-based expression vectors. Briefly, Freestyle293 cells were transfected with transposase mRNA and an expression vector encoding human GPNMB flanked by transposition elements. Upon co-transfection of vector DNA and transposase mRNA, the transiently expressed enzyme catalyzes high-efficiency and precise integration of the transposon cassette. The resulting pool of transfected cells was stained with anti-human GPNMB-APC (allophycocyanin) and sorted for high GPNMB fluorescence signal. Stable expression of GPNMB protein in this cell line was maintained through puromycin selection. These cells, called Freestyle293-huGPNMB+ (also referred to as FS293-huGPNMB+), were subsequently used to validate binding of anti-human GPNMB antibody clones of the present disclosure to cell surface expressed antigen.

Mammalian expression of soluble GPNMB polypeptides was performed by cloning synthetic genes based on GPNMB cDNA sequences into mammalian expression vectors, followed by transient transfection and expression in Expi293 cells and HEK293 cells. Nucleic acid constructs used herein included the native signal peptide and a C-terminal polyhistidine motif for His-tagged constructs, a polyhistine-avi tag, or included an Fc domain for Fc-tagged constructs. Briefly, expression vectors containing the GPNMB polypeptide of interest were transfected by complexing with a transfection reagent followed by exposure to HEK293 cells for one hour followed by dilution of culture media to a final density of 4 million cells per ml. The cells were then cultured for 7 days with fresh feed media added every 48 hours. After 7 days, the supernatants were collected following centrifugation. Purification of the expressed recombinant polypeptides was performed using protein Ni-sepharose and, if necessary, a SEC column purification to reach >95% non-aggregated monomer content.

For transient expression in Expi293 cells, the following was performed. Synthetic genes based on GPNMB cDNA were cloned into mammalian expression vectors, followed by transient transfection and expression in Expi293 cells. Constructs included a heterologous signal peptide and C-terminal Avi-His tag to allow for purification and biotinylation. Briefly, expression plasmids encoding GPNMB constructs were transfected using the Expifectamine 293 Transfection kit (ThermoFisher A14524) according to the manufacturer's specifications. Five days after transfection the culture supernatants were harvested, clarified by centrifugation, and purified using HisPur Ni-NTA resin (Thermo Scientific 88223) in a drip-column format. Briefly, 200 ml of culture supernatant was filtered using 0.2 μm filtration unit and 3 ml of resin slurry in PBS was added to the filtered supernatant. The sample was incubated overnight with shaking at 4° C. and the following day the beads were loaded onto 20 ml drip columns, washed with 10 ml of His-Select wash buffer (Millipore Sigma H5288), and eluted with 5 ml of His-Pur elution buffer (Millipore Sigma H5413). Eluate was buffer exchanged into PBS using Amicon Ultra-15 centrifugal filter units (Millipore UFC9010). Quantification of the antibody concentration was determined by measuring the absorbance at 280 nm using the Nanodrop 8000 (ThermoFisher). Purity of the antigens was determined by SDS-PAGE. The antigens were analyzed with size exclusion chromatography (SEC) for aggregation. In some instances, the antigens were biotinylated using an BirA biotin-protein ligase kit (AVIDITY), according to the manufacturer's instructions.”

The amino acid sequences of soluble (extracellular domains) human, cynomolgus, and mouse GPNMB polypeptides are set forth below.

Soluble Human GPNMB Amino Acid Sequence (SEQ
ID NO: 2):
MECLYYFLGFLLLAARLPLDAAKRFHDVLGNERPSAYMREHNQLNGWSS
DENDWNEKLYPVWKRGDMRWKNSWKGGRVQAVLTSDSPALVGSNITFAV
NLIFPRCQKEDANGNIVYEKNCRNEAGLSADPYVYNWTAWSEDSDGENG
TGQSHHNVFPDGKPFPHHPGWRRWNFIYVFHTLGQYFQKLGRCSVRVSV
NTANVTLGPQLMEVTVYRRHGRAYVPIAQVKDVYVVTDQIPVFVTMFQK
NDRNSSDETFLKDLPIMFDVLIHDPSHFLNYSTINYKWSFGDNTGLFVS
TNHTVNHTYVLNGTFSLNLTVKAAAPGPCPPPPPPPRPSKPTPSLATTL
KSYDSNTPGPAGDNPLELSRIPDENCQINRYGHFQATITIVEGILEVNI
IQMTDVLMPVPWPESSLIDFVVTCQGSIPTEVCTIISDPTCEITQNTVC
SPVDVDEMCLLTVRRTFNGSGTYCVNLTLGDDTSLALTSTLISVPDRDP
ASPLRMAN
Soluble Cynomolgus GPNMB Amino Acid Sequence (SEQ
ID NO: 3):
MECLYYFLGFLLLAARLPLDAAKRFHDVLGNERPSAYMREHNQLNGWSS
DENDWNEKLYPVWKRGDMRWKNSWKGGRVQAVLTSDSPALVGSNITFVV
NLVFPRCQKEDANGNIVYEKNCRNEAGLSADPYVYNWTAWSEDSDGENG
TGQSHHNVFPDGKPFPHHPGWRRWNFVYVFHTLGQYFQKLGRCSVRVSV
NTANVTLGPQLMEVTVYRRHGRAYVPIAQVKDVYVVTDQIPVFVTMSQK
NDRNSSDETFLKDLPITFDVLIHDPSHFLNYSTINYKWSFGDNTGLFVS
INHTVNHTYVLNGTFSLNLTVKAAAPGPCPPPPPPPPRPPKPTPSLGPA
GDNPLGLSEIPDENCQINRYGHFQATITIVEGILEVNIIQTTDVLMLVP
GPDSSLIDFVVTCQGSIPTEVCTIISDPTCEITQNAVCSPVDVDEMCLL
TVRRAFNGSGTYCVNLTLGDDTSLALTSTLISVPDRDPASSLRMANSGG
GGLNDIFEAQKIEWHEHHHHHHHHH
Soluble Mouse GPNMB Amino Acid Sequence (SEQ ID
NO: 4):
MESLCGVLGFLLLAAGLPLQAAKRFRDVLGHEQYPDHMREHNQLRGWSS
DENEWDEHLYPVWRRGDGRWKDSWEGGRVQAVLTSDSPALVGSNITFVV
NLVFPRCQKEDANGNIVYEKNCRNDLGLTSDLHVYNWTAGADDGDWEDG
TSRSQHLRFPDRRPFPRPHGWKKWSFVYVFHTLGQYFQKLGRCSARVSI
NTVNLTAGPQVMEVTVFRRYGRAYIPISKVKDVYVITDQIPVFVTMSQK
NDRNLSDEIFLRDLPIVFDVLIHDPSHFLNDSAISYKWNFGDNTGLFVS
NNHTLNHTYVLNGTFNLNLTVQTAVPGPCPPPSPSTPPPPSTPPSPPPS
PLPTLSTPSPSLMPTGYKSMELSDISNENCRINRYGYFRATITIVEGIL
EVSIMQIADVPMPTPQPANSLMDFTVTCKGATPMEACTIISDPTCQIAQ
NRVCSPVAVDGLCLLSVRRAFNGSGTYCVNFTLGDDASLALTSTLISIP
GKDPDSPLRAVN

### Example 2: Generation of Anti-GPNMB Hybridoma Antibodies

In order to obtain antibodies against GPNMB, the following procedures were used to generate hybridomas. BALB/c mice, CD-1 mice, GPNMB knockout (KO) mice, or Sprague Dawley rats (Charles River Laboratories, Wilmington, MA or Taconic Biosciences, Rennselaer, NY) were immunized twice a week by subcutaneous or intraperitoneal injections of purified extracellular domain polypeptides of human and/or mouse GPNMB (obtained as described above in Example 1) with or without adjuvant. After a total of 7-10 injections and three days following the final boost, the lymph nodes were harvested from the mice for hybridoma cell line generation. Sera from the animals were analyzed for reactivity to GPNMB by FACS analysis on Freestyle293 cells overexpressing human or mouse GPNMB or on B16F10 melanoma cell lines expressing mouse GPNMB. Sera titers were also determined by ELISA on human, cynomolgus, and/or mouse GPNMB His-tagged or GPNMB Avi-His-tagged polypeptides.

Lymphocytes from animals whose sera demonstrated strong reactive binding to Freestyle293 cells overexpressing human or mouse GPNMB were isolated and fused with SP2/mIL-6 (CRL-2016, American Type Culture Collection, Rockville, MD) or Sp2ab (ENZ-70008, Enzo Life Sciences, Farmingdale, NY) mouse myeloma cells via electrofusion (Hybrimune, BTX, Holliston, MA) and incubated at 37° C., 5% CO₂, overnight in Clonacell-HY Medium C (Stemcell Technologies, Vancouver, BC, Canada, Cat #03803).

The following day, the fused cells were centrifuged and resuspended in Clonacell-HY Medium E (Stemcell Technologies, Cat #03805) with HAT (Sigma Aldrich, Cat #H0262). The cells were seeded into T225 flasks and grown for 5-6 days at 37° C., 5% CO₂, and then IgG+ hybridomas were single cell sorted into 96-well plates using a FACSAria II cell sorter (BD Biosciences, San Jose, CA). Alternatively, the recovered fusions were resuspended into 10 ml of ClonaCell-HY Medium C with anti-mouse IgG Fc-FITC (Jackson ImmunoResearch, West Grove, PA) and then gently mixed with 90 ml of methylcellulose-based ClonaCell-HY Medium D (Stemcell Technologies, Cat #03804) containing HAT components. The cells were plated into Nunc OmniTrays (Thermo Fisher Scientific, Rochester, NY) and allowed to grow at 37° C., 5% CO₂ for 7 days. The Clonepix 2 (Molecular Devices, Sunnyvale, CA) system was used to select and transfer IgG positive colonies into 96-well plates with high glucose DMEM culture media containing 10% Fetal Cone II serum (Hyclone SH30066.03, Cytiva, Malborough, MA), 1× GlutaMAX (Gibco 35050061, Thermo Fisher Scientific, Waltham, MA) and 20% Clonacell-HY Medium E (Stemcell Technologies, Cat #03805). In total, 6,336 IgG positive hybridoma clones were isolated. After 7-10 days in culture, tissue culture supernatants from the hybridomas were screened by FACS on Freestyle293 cells overexpressing human GPNMB or B16F10 cells expressing mouse GPNMB (as described below).

### Example 3: Screening of Anti-GPNMB Antibody Hybridoma Supernatants by FACS

The IgG positive hybridoma supernatants identified as described above were initially screened by FACS for their ability to differentially bind Freestyle293 cells overexpressing human GPNMB and B16F10 cells expressing mouse GPNMB compared to binding observed using an isotype control antibody. The cells were harvested, washed, and labeled with 1-5 μg/ml of PacBlue and/or FITC dyes (ThermoFisher) to create uniquely barcoded cell populations. Barcoded cells were aliquoted into 96-well U-bottom plates and incubated with 50 μl of hybridoma cell culture supernatant on ice for 30 minutes. After this primary incubation, the supernatants were removed via centrifugation, the cells were washed twice with 175 μl of ice-cold FACS buffer (PBS+1% FBS+2 mM EDTA), and the cells were then further incubated on ice for 10-15 minutes with Zombie NIR dye (Biolegend, San Diego, CA, Cat #423105), diluted 1:1000 in PBS, to exclude dead cells. The cells were then incubated on ice for 20 minutes with anti-mouse IgG Fc-allophycocyanin (APC) or anti-rat IgG Fc-APC (Jackson Labs, Cat #115-136-071 and Cat #112-136-071, respectively), diluted 1:1000 in FACS buffer. Following this secondary antibody incubation, the cells were again washed twice with ice-cold FACS buffer and fixed in 1% paraformaldehyde. Binding intensity on cells was analyzed using the FACS Canto system (BD Biosciences), with sorting gates drawn to exclude dead cells. For each barcoded cell population, the ratio of APC geometric mean fluorescence intensity (gMFI) divided by secondary antibody gMFI was calculated for each anti-GPNMB hybridoma supernatant. In total, 1,062 clones were determined to bind to Freestyle293 cells stably overexpressing human GPNMB and/or B16F10 cells expressing mouse GPNMB by FACS.

### Example 4: Molecular Cloning of Anti-GPNMB Antibodies

Anti-GPNMB antibodies obtained from the hybridomas described above were selected for sequencing as follows. 1-2×10⁵ hybridoma cells were harvested, washed with PBS, and resuspended in 200 μl of RNAlater (Invitrogen, Cat #AM7021). Samples were stored at −80° C. and sent to Abterra Biosciences (San Diego, CA) for sequencing. Briefly, RNA was extracted, and cDNA synthesis was performed. The variable regions of IgG/IgM, IgK, and IgL were amplified using proprietary primers in a 5′ RACE strategy. Hybridoma variable region amplicons were sequenced on the Illumina MiSeq platform (Illumina, San Diego, CA) and analysis of the reads were done on Abterra's Reptor analysis pipeline.

Amino acid sequences of the variable heavy chains and variable light chains of anti-GPNMB antibodies of the present disclosure identified herein are provided below in Table 1.

TABLE 1

| | | SEQ | | SEQ |
|---|---|---|---|---|
| | | ID | | ID |
| Antibody | Heavy Chain Variable | NO: | Light Chain Variable | NO: |
| GPN-01 | QVQLQQSGAELAKPGASVKLSC | 161 | DIVLTQSPASLAVSLGQRATI | 162 |
| | KASGYNFTNYCIHWVKQRPGQG | | SCRASKSVSTSTYSYMHWYQQ | |
| | LEWIGYINPSTGYTEYNQKFKD | | KPGQPPKLLIYLASNLESGVP | |
| | KATLTADKSSTTAYMQLSSLTS | | ARFSGSGSGTDFTLNIHPVEE | |
| | EDSAVYYCARGRYDEFTYWGQG | | EDAATYYCQHSRELPPTFGGG | |
| | TLVTVSA | | TKLEIK | |
| GPN-03 | QVQLQQSGPELVKPGASVKLSC | 163 | DVVMTQTPLSLPVSLGDQASI | 164 |
| | KASGYTFTNYYMYWVKQRPGQG | | SCRSSQSLVHSDGNTYLHWYL | |
| | LEWIGEINPRNGGTNFNEKFKS | | QKPGQSPKLLIYKVSTRFSGV | |
| | KATLTVDKSSSTAYMQLSSLTE | | PDRFSGSGSGTDFTLKISRVE | |
| | EDSAVYYCTTGLAYWGQGTLVT | | AEDLGVYFCSQSTHVPWTFGG | |
| | VSA | | GTKLEIK | |
| GPN-06 | QVQLQQSGAELVKPGASVKLSC | 165 | DIQMTQSPASLSASVGETVTI | 166 |
| | KASGYTFTNYYMYWVKQRPGQG | | TCRASENIYSYLVWYQQNQGK | |
| | LEWIGEINTRNGGTNFNEKFKS | | SPQLLVYNAKTLAEGVPSRFS | |
| | KATLTVDKSSSTAYMQLSSLTS | | GSGSGTQFSLKINSLQPEDFG | |
| | EDSAVYYCTTGFAYWGQGTLVT | | TYYCQHHYGTPFTFGSGTKLE | |
| | VSA | | IK | |
| GPN-07 | QVQLQQSGAEQVKPGASVKLSC | 167 | DIQMTQSPSSLSASLGERVSL | 168 |
| | KASGYTFTSYYMYWVKQRPGQG | | TCRASQEMSGHLSWLQQKPDG | |
| | LEWIGEINPSNGGTYFNEKFKS | | TIKRLIYAASTLDSGVPKRFS | |
| | KATLTVDKSSSTAYMHLSSLTS | | GSRSGSDYSLTISSLESEDFA | |
| | EDSAVYYCTRGGTYYAYWGQGT | | DYYCLQYASYPYTFGGGTKLE | |
| | LVTVSA | | IK | |
| GPN-08 | QVQLQQPGAELVKPGASVKLSC | 169 | DIVMTQSHKFMSTSVGDRVSI | 170 |
| | RASGYTFTSYWLHWVKQRPGQG | | TCKASQDVSPSVAWYQQRPGQ | |
| | LEWIGEINPSDGRTNYIEKVKT | | SPKLLIYSASYRYTGVPDRFT | |
| | KATLTVDKSSSTAYMQLSSLTS | | GSGSGTDFTFTISSVQAEDLA | |
| | EDSAVYYCARSGGNYAWFAYWG | | VYYCQQHYSIPLTFGAGTKLE | |
| | QGTLVTVSA | | LK | |
| GPN-09 | QVQLQQSGAELVKPGASVKLSC | 171 | DIQMTQSPSSLSASLGERVSL | 172 |
| | KASGYTFTSYYIYWVKQRPGQG | | TCRASQEISGHLGWLQQKPDG | |
| | LEWIGEINPSNGGTYFNEKFKN | | TIKRLIYAALTLDSGVPKRFS | |
| | KATLTVDKSSSTAYMQLSSLTS | | GSRSGSDYSLTISSLESEDFA | |
| | EDSAVYYCTRGGNYYAYWGQGT | | DYYCLQYASYPYTFGGGTKLE | |
| | LVTVSA | | IK | |
| GPN-11 | QVQLQQPGAELVKPGASVKLSC | 173 | DIQMTQSPASLSVSVGETVTI | 174 |
| | KASGYTFTSYWMHWVKQRPGQG | | TCRASENIYSNLAWYQQKQGK | |
| | LEWIGEINSSNGRTNYNEKFKT | | SPQLLVYAATNLADGVPSRFS | |
| | KATLTVDRSSSTAYMQLSSLTS | | GSGSGTQYSLKINSLQSEDEG | |
| | EDSAVYYCARLGYYSGSPYPYY | | SYYCQHFWNTPPTFGGGTKLE | |
| | AMDCWGQGTSVTVSS | | IK | |
| GPN-22 | QVQLQQPGAERVKPGASVKMSC | 175 | DIQMTQSPSSLSASLGERVSL | 176 |
| | KASGYTFTSYWITWVKQRPGQG | | TCRTSQEISGYLSWLQQKPDG | |
| | LEWIGDIFPGSGSTNYHEKFKS | | TIKRLIYSTSTLDSGVPKRFS | |
| | KATLTVDTSSSTAYMQLSSLTS | | GSRSGSDYSLTISSLESEDFA | |
| | EDSAVYYCSRGYFGSSESDYWG | | DYYCLQYASSPPTFGSGTKLE | |
| | QGTSVTVSS | | IK | |
| GPN-24 | QVQLQQPGAELVKPGASVKMSC | 177 | DIQMTQSPSSLSASLGERVSL | 178 |
| | KASGYTFTSYWITWVKQRPGQG | | TCRASQEISGYLSWLQQKPDG | |
| | LEWIGDIHPGSGRSNYNEKFKN | | TIKRLIYSTSTLDSGVPKRFS | |
| | KATLNVDTSSSTVYMQLSSLTS | | GSRSGSDYSLTISSLESEDFA | |
| | EDSAVYYCARDYLFAYWGQGTL | | DYYCLQYTSSPPTFGGGTKLE | |
| | VTVSA | | IK | |
| GPN-25 | EVQLQQSGPELVKPGASVKISC | 179 | DIQMTQSPSSLSASLGERVSF | 180 |
| | KASGYSFTGYYMNWVKQSPEKS | | ICRASQEISGHLSWLQQKPDG | |
| | LEWIGEINPGSGITTYNQKFKA | | TIKRLIYAASTLDSGVPKRFS | |
| | KATLTVDKSSSTAYLQLKSLTS | | GSRSGSDYSLTISSLESEDFA | |
| | EDSAVYYCARPASWFAYWGQGT | | DYYCLQYASYPYTFGGGTKLE | |
| | LVTVSA | | IK | |
| GPN-26 | EVQLQQSGPELVKPGASVKISC | 181 | DIQMTQSPSSLSASLGERVSL | 182 |
| | KASGYSFTGYYMNWVRQSPEKS | | TCRASQEISGHLSWLQQKPDG | |
| | LEWIGEINPSTGITTYNQKFKA | | TIKRLIYAASTLDSGVPKRFS | |
| | KATLTVDKSSNTAFMQLKSLTS | | GSRSGSDYSLTISSLESEDFA | |
| | EDSAVYYCARPASWFDYWGQGT | | DYYCLQYASYPYTFGGGTKLE | |
| | LVTVSA | | IK | |
| GPN-30 | EVQLQQSGPELVNPGASVRISC | 183 | DIQMTQSPSSLSASLGERGSL | 184 |
| | KASGYSFTGYYMNWVKQSPEKS | | TCRASQEISGHLSWLQQKPDG | |
| | LEWIGEINPNTGFTTYNQNFKA | | TIKRLIYAASTLDSGVPKRFS | |
| | KATLTVDKSSSTAYIQLKSLTS | | GSRSGSDYSLTISSLESEDFA | |
| | EDSAVYYCTRPSPSFDYWGQGT | | DYYCLQYASYPYTFGGGTKLE | |
| | TLTVSS | | IK | |
| GPN-31 | EVQLQQSGPELVKPGASVKISC | 185 | DIQMTQSPSSLSASLGERVSL | 186 |
| | KASGYSFTGYYMNWVKQSPEKN | | TCRASQEISGHLSWLQQKPDG | |
| | LEWIGEINPNTGGTTYNQKFKA | | TIKRLIYAASTLDSGVPKRFS | |
| | KATLTVDKSSTTAYMQLKSLTS | | GRRSGSDYSLTISSLESEDFA | |
| | EDSAVYYCTRPASWFDYWGQGT | | DYYCLQYASYPYTFGGGTKLA | |
| | LVTVSA | | IK | |
| GPN-33 | EVQLVESGGSLVQPGGSLKLSC | 187 | DIVMTQTPLSLSVAIGQSASI | 188 |
| | VASGYTFSNYWMDWVRQTPGKS | | SCKSSQSLKFSDGKTYLNWVF | |
| | LEWIGEINTDGSKTNYAPSIKD | | QSPGQSPKRLIYQVSKLDSGV | |
| | RFTISRDNAKSTLYLQMSNVKA | | PDRFSGTGSETDFILKISRVE | |
| | DDTAIYYCTRGPGAWGQGTSVT | | AEDLGVYYCCQGSYSPWTIGG | |
| | VSS | | GTKLELK | |
| GPN-34 | EVKLVESGGGLVQPGRSLKLSC | 189 | DIQMTQSPSSMSVSLGDTVTI | 190 |
| | AASGFNFYDYWMGWVRQAPGKG | | PCRASQDVGSYVNWFQQKPGK | |
| | LEWIGEINKDSRTINYTPELKD | | SPRHMIYRATNLADGVPSRFS | |
| | RFTISRDNAQNTLYLQMSKLGS | | GSRSGADYSLTISSLESEDVA | |
| | EDTAIYYCAREGRVPFDYWGQG | | DYHCQQYAEYPLTFGSGTKLE | |
| | VMVTVSS | | IK | |
| GPN-35 | EVQLVESGGGSVRPGRSLKLSC | 191 | DIQMTQSPASLSASLGETVSI | 192 |
| | AAAGFTFSDYYMTWVRQAPTKG | | ECLASEDIYNYLTWYQQKPGK | |
| | LEWVASINHDGGGTYYRDSVKG | | SPQVLIYSASGLQDGVPSRFS | |
| | RFTISRDNARSSLYLQMDSLRS | | GSGSGRQYSLKINGLESEDAA | |
| | EDTGTYYCTTRAGWHWGQGVMV | | TYFCLQDSEYPLTFGSGTKLE | |
| | TVSS | | IK | |
| GPN-37 | EVQLVESGGGLVQPGRSLKLSC | 193 | DIQMTQSPASLSASLGETVTI | 194 |
| | AASGFTFSSKSGMAWVRQAPTK | | ECLASEDIYSSLAWYQQKPGK | |
| | GLEWVASISNGGGDTYYRDSVK | | SPQLLIYDASSLQDGVPSRFS | |
| | GRFTISRDNAKNTQYLQMDSLR | | GSESGTQYSLEINSLQSEDAA | |
| | PEDTATYYCARHRDYGYDAYED | | TYFCQQHHDYPYTFGAGTKLE | |
| | YWGQGVMVTVSS | | LK | |
| GPN-38 | QVQLQQPGAELVRPGTSVRLSC | 195 | DIQMTQSPASLSTSVGEIVTI | 196 |
| | KASGYTFTSYWIHWVKQRPGRG | | TCRASENIYSYLTWYQQKQGK | |
| | LEWIGVIDPSDSYTNYNQQFNV | | SPQLLVYNAKTLAEGVPSRFS | |
| | KATLTVDTSSSAAYMQLSSLTS | | GSGSGTQFSLKINSLQPEDFG | |
| | EDSAVYYCARGLITAVNWYFDV | | TYYCQHHYGTPLTFGSGTKLE | |
| | WGTGTTVTVSS | | IK | |
| GPN-41 | QLQLQQSGTEVVKPGASVKIPC | 197 | SIVMTQTPKFLLVSAGDRVTI | 198 |
| | KASGYAFSSYWMNWVKQRPGKG | | TCKASQSVSNDVAWFQQKPGQ | |
| | LEWIGQIYPGDGDTNDNGKFEG | | SPELLIYSASNRYTGVPDRFT | |
| | KATLTVDKSSNTAYIQLSSLTS | | ASGYGTDFTFTISAVQAEDLA | |
| | EDSAVYFCARSPLHLRGSMDYW | | VYFCQQNYYSPFTFGGGTRLE | |
| | GQGTSVTVSS | | IK | |
| GPN-42 | DVKLVESGEDLVKPGGSLKLSC | 199 | DVMMTQTPLSLPVSLGDQASI | 200 |
| | AASGFTFSSYAMSWVRQTPEKR | | SCRSSQSLVHSNGNTYLHWFL | |
| | LEWVAYISSGGDYIYYADTVKG | | QKPGQSPKLLIFRVSNRFSGV | |
| | RFTISRDNARNTLYLQMSSLKS | | PDRFSGRGSGTDFTLKISRVE | |
| | EDTAMYYCTRDRYYDYDVRDYY | | AEDLGVYFCSQTTHVPRTFGG | |
| | TMDYWGQGTSVTVSS | | GTTLEIK | |
| GPN-43 | EVQLQQSGPELVKPGASVKISC | 201 | DIQMTQSPASLSASVGETVTI | 202 |
| | KASGYTFTDYYMNWVRQSHGKS | | TCRASENIYSFLVWYQQKQGK | |
| | LEWIGDINPYHGGSTYNQKIKV | | SPQLLVYNAKTLAEGVPSRFS | |
| | KATLTVDKSSSTAYMELRSLTS | | GSGSGTQFSLKISSLQPEDFG | |
| | EDSAVYYCARNWPYFDYWGQGT | | SYYCQHHFGTPPYTFGGGTKL | |
| | TLTVSS | | EIK | |
| GPN-48 | QVQLQQSGPELVKPGASVKLSC | 203 | DIVMTQSHKFMSTSVGDRVNI | 204 |
| | RASGYGFSSFWMNWVKQRPGKG | | TCKASQDVNSAIAWYQQKPGQ | |
| | LEWIGRIYPGKGDTLYNGKFKD | | SPKLLIYSASYRFIGVPDRFT | |
| | KATLTADESSSTAYMQLSSLTS | | GSGSGTGFTFTISSVQAEDLA | |
| | EDSAVYFCARSIYYYDYWYFDV | | VYYCQQHYTTPLTFGAGTKLE | |
| | WDTGTTVTVSS | | LK | |
| GPN-52 | DVQLQESGPGLVKPSQTVFLTC | 205 | DIVLTQSPASLAVSLGQRATI | 206 |
| | TVTGISITTGNYRWSWIRQFPG | | SCRASESVDYYGIGFMNWFQQ | |
| | NKLEWIGYIYYSGTITYNPSLT | | KPGQPPKLLIYAASNQGSGVP | |
| | SRITITRDTPKNEFFLEMNSLT | | ARFSGSGSGTDFSLNIHPMEE | |
| | AEDTATYYCARDLTHWYFDVWG | | DDTAMYFCQQSKEVPPTFGGG | |
| | TGTTVTVSS | | TKLEIK | |
| GPN-61 | DVQLQESGPGLVKPSQTVFLTC | 207 | DIVLTQSPASLAVSLGQRATI | 208 |
| | TVTGISITTGNYRWSWIRQFPG | | SCRASENIDYYGVSFMNWFQQ | |
| | NKLEWIGYIYYSGTITYNPSLT | | KPGQPPKLLIYAASNQGSGVP | |
| | SRTTITRDTPKNQFFLEMSSLT | | ARFSGSGSGTDFSLNIHPLEE | |
| | AEDTATYFCARDLRHWYFDVWG | | DDTAMYFCQQSQEVPPTFGGG | |
| | TGTTVTVSS | | TKLEIK | |
| GPN-65 | QLQLQQSGTEVVKPGASVKIPC | 209 | SIVMTQTPKELLVSAGDRVTI | 210 |
| | KASGYAFSSYWMNWVKQRPGKG | | TCKASQSVSNDVAWFQQKPGQ | |
| | LEWIGQIYPGDGDTNDNGKFEG | | SPELLIYSASNRYTGVPDRFT | |
| | KATLTVDKSSNTAYIQLSSLTS | | GSGYGTDFTFTISTVQAEDLA | |
| | EDSAVYFCARSPLHLRGSMDYW | | VYFCQQNYYSPFTFGGGTRLE | |
| | GQGTSVTVSS | | IK | |

The CDR sequences according to Kabat for the anti-GPNMB-antibodies of the present disclosure are provided below in Table 2 (heavy chain) and Table 3 (light chain).

TABLE 2

| | | SEQ | | SEQ | | SEQ |
|---|---|---|---|---|---|---|
| Antibody | HVR-H1 | ID NO: | HVR-H2 | ID NO: | HVR-H3 | ID NO: |
| GPN-01 | NYCIH | 5 | YINPSTGYTEY | 6 | GRYDEFTY | 7 |
| | | | NQKFKD | | | |
| GPN-03 | NYYMY | 8 | EINPRNGGTNF | 9 | GLAY | 10 |
| | | | NEKFKS | | | |
| GPN-06 | NYYMY | 11 | EINTRNGGTNF | 12 | GFAY | 13 |
| | | | NEKFKS | | | |
| GPN-07 | SYYMY | 14 | EINPSNGGTYF | 15 | GGTYYAY | 16 |
| | | | NEKFKS | | | |
| GPN-08 | SYWLH | 17 | EINPSDGRTNY | 18 | SGGNYAWFA | 19 |
| | | | IEKVKT | | Y | |
| GPN-09 | SYYIY | 20 | EINPSNGGTYF | 21 | GGNYYAY | 22 |
| | | | NEKFKN | | | |
| GPN-11 | SYWMH | 23 | EINSSNGRTNY | 24 | LGYYSGSPYP | 25 |
| | | | NEKFKT | | YYAMDC | |
| GPN-22 | SYWIT | 26 | DIFPGSGSTNY | 27 | GYFGSSFSDY | 28 |
| | | | HEKFKS | | | |
| GPN-24 | SYWIT | 29 | DIHPGSGRSNY | 30 | DYLFAY | 31 |
| | | | NEKFKN | | | |
| GPN-25 | GYYMN | 32 | EINPGSGITTY | 33 | PASWFAY | 34 |
| | | | NQKFKA | | | |
| GPN-26 | GYYMN | 35 | EINPSTGITTY | 36 | PASWFDY | 37 |
| | | | NQKFKA | | | |
| GPN-30 | GYYMN | 38 | EINPNTGFTTY | 39 | PSPSFDY | 40 |
| | | | NQNFKA | | | |
| GPN-31 | GYYMN | 41 | EINPNTGGTTY | 42 | PASWFDY | 43 |
| | | | NQKFKA | | | |
| GPN-33 | NYWMD | 44 | EINTDGSKTN | 45 | GPGA | 46 |
| | | | YAPSIKD | | | |
| GPN-34 | DYWMG | 47 | EINKDSRTINY | 48 | EGRVPFDY | 49 |
| | | | TPFLKD | | | |
| GPN-35 | DYYMT | 50 | SINHDGGGTY | 51 | RAGWH | 52 |
| | | | YRDSVKG | | | |
| GPN-37 | SKSGMA | 53 | SISNGGGDTY | 54 | HRDYGYDAY | 55 |
| | | | YRDSVKG | | FDY | |
| GPN-38 | SYWIH | 56 | VIDPSDSYTNY | 57 | GLITAVNWY | 58 |
| | | | NQQFNV | | FDV | |
| GPN-41 | SYWMN | 59 | QIYPGDGDTN | 60 | SPLHLRGSM | 61 |
| | | | DNGKFEG | | DY | |
| GPN-42 | SYAMS | 62 | YISSGGDYIYY | 63 | DRYYDYDVR | 64 |
| | | | ADTVKG | | DYYTMDY | |
| GPN-43 | DYYMN | 65 | DINPYHGGST | 66 | NWPYFDY | 67 |
| | | | YNQKIKV | | | |
| GPN-48 | SFWMN | 68 | RIYPGKGDTL | 69 | SIYYYDYWY | 70 |
| | | | YNGKFKD | | FDV | |
| GPN-52 | TGNYRWS | 71 | YIYYSGTITYN | 72 | DLTHWYFDV | 73 |
| | | | PSLTS | | | |
| GPN-61 | TGNYRWS | 74 | YIYYSGTITYN | 75 | DLRHWYFDV | 76 |
| | | | PSLTS | | | |
| GPN-65 | SYWMN | 77 | QIYPGDGDTN | 78 | SPLHLRGSM | 79 |
| | | | DNGKFEG | | DY | |

TABLE 3

| | | SEQ | | SEQ | | SEQ |
|---|---|---|---|---|---|---|
| Antibody | HVR-L1 | ID NO: | HVR-L2 | ID NO: | HVR-L3 | ID NO: |
| GPN-01 | RASKSVST | 83 | LASNLES | 84 | QHSRELPPT | 85 |
| | STYSYMH | | | | | |
| GPN-03 | RSSQSLVH | 86 | KVSTRFS | 87 | SQSTHVPWT | 88 |
| | SDGNTYL | | | | | |
| | H | | | | | |
| GPN-06 | RASENIYS | 89 | NAKTLAE | 90 | QHHYGTPFT | 91 |
| | YLV | | | | | |
| GPN-07 | RASQEMS | 92 | AASTLDS | 93 | LQYASYPYT | 94 |
| | GHLS | | | | | |
| GPN-08 | KASQDVS | 95 | SASYRYT | 96 | QQHYSIPLT | 97 |
| | PSVA | | | | | |
| GPN-09 | RASQEISG | 98 | AALTLDS | 99 | LQYASYPYT | 100 |
| | HLG | | | | | |
| GPN-11 | RASENIYS | 101 | AATNLAD | 102 | QHFWNTPPT | 103 |
| | NLA | | | | | |
| GPN-22 | RTSQEISG | 104 | STSTLDS | 105 | LQYASSPPT | 106 |
| | YLS | | | | | |
| GPN-24 | RASQEISG | 107 | STSTLDS | 108 | LQYTSSPPT | 109 |
| | YLS | | | | | |
| GPN-25 | RASQEISG | 110 | AASTLDS | 111 | LQYASYPYT | 112 |
| | HLS | | | | | |
| GPN-26 | RASQEISG | 113 | AASTLDS | 114 | LQYASYPYT | 115 |
| | HLS | | | | | |
| GPN-30 | RASQEISG | 116 | AASTLDS | 117 | LQYASYPYT | 118 |
| | HLS | | | | | |
| GPN-31 | RASQEISG | 119 | AASTLDS | 120 | LQYASYPYT | 121 |
| | HLS | | | | | |
| GPN-33 | KSSQSLKF | 122 | QVSKLDS | 123 | CQGSYSPWT | 124 |
| | SDGKTYL | | | | | |
| | N | | | | | |
| GPN-34 | RASQDVG | 125 | RATNLAD | 126 | QQYAEYPLT | 127 |
| | SYVN | | | | | |
| GPN-35 | LASEDIYN | 128 | SASGLQD | 129 | LQDSEYPLT | 130 |
| | YLT | | | | | |
| GPN-37 | LASEDIYS | 131 | DASSLQD | 132 | QQHHDYPYT | 133 |
| | SLA | | | | | |
| GPN-38 | RASENIYS | 134 | NAKTLAE | 135 | QHHYGTPLT | 136 |
| | YLT | | | | | |
| GPN-41 | KASQSVS | 137 | SASNRYT | 138 | QQNYYSPFT | 139 |
| | NDVA | | | | | |
| GPN-42 | RSSQSLVH | 140 | RVSNRFS | 141 | SQTTHVPRT | 142 |
| | SNGNTYL | | | | | |
| | H | | | | | |
| GPN-43 | RASENIYS | 143 | NAKTLAE | 144 | QHHFGTPPYT | 145 |
| | FLV | | | | | |
| GPN-48 | KASQDVN | 146 | SASYRFI | 147 | QQHYTTPLT | 148 |
| | SAIA | | | | | |
| GPN-52 | RASESVD | 149 | AASNQGS | 150 | QQSKEVPPT | 151 |
| | YYGIGFM | | | | | |
| | N | | | | | |
| GPN-61 | RASENIDY | 152 | AASNQGS | 153 | QQSQEVPPT | 154 |
| | YGVSFMN | | | | | |
| GPN-65 | KASQSVS | 155 | SASNRYT | 156 | QQNYYSPFT | 157 |
| | NDVA | | | | | |

### Example 5: Production of Recombinant Anti-GPNMB Antibodies

Anti-GPNMB antibody variable gene regions were synthesized and subcloned into mammalian expression vectors encoding human IgG1 and IgK for the production of chimeric antibodies. The expression plasmids were transiently transfected into Expi293 cells (Invitrogen) in 24-well or 96-well deep well blocks according to the manufacturer's protocol. The cell culture supernatant was harvested at 5 days post transfection. Clarified supernatants were purified using ProPlus Phytip columns (Biotage, Uppsala, Sweden, Cat #PTH 91-20-07) on a Hamilton STAR platform (Hamilton Company, Reno, NV). Briefly, antibodies from the supernatants were captured by protein A coupled on resin-packed tips, washed twice with PBS, eluted with Pierce IgG elution buffer (ThermoFisher, Cat #21004), and neutralized with 1M Tris-HCl pH8 to a final pH of 6.0. Neutralized eluates containing the antibodies were dialyzed into PBS. Quantification of the antibody concentration was determined by measuring the absorbance at 280 nm using the Nanodrop 8000 (ThermoFisher) or Lunatic (Unchained Labs).

### Example 6: Characterization of Recombinant Anti-GPNMB Antibodies Binding to Cells

Recombinant anti-GPNMB antibodies of the present disclosure were examined for their ability to bind human and/or mouse GPNMB expressed on Freestyle293-huGPNMB+ or B16F10 cells, respectively. B16F10 cells are a murine melanoma cell line from a C57BL/6J mouse that endogenously express high levels of mouse GPNMB. The antibodies were also tested for their ability to bind to a human macrophage cell line (U937 cells) engineered to express human GPNMB. Cells were harvested, plated at 100,000 cells/well in a 96-well round bottom plate, and incubated in 100 μl FACS buffer (PBS containing 2% FBS, 2 mM EDTA) containing anti-GPNMB monoclonal antibodies of the present disclosure for 30 minutes on ice. Cells were then washed and incubated in 100 ul FACS buffer containing 1 μg/ml APC-conjugated anti-human IgG secondary antibody for 30 minutes in ice. Cells were washed twice in cold FACS buffer and acquired on a BD FACS Canto. Data analysis and calculation of MFI values was performed with FlowJo (TreeStar) software version 10.0.7.

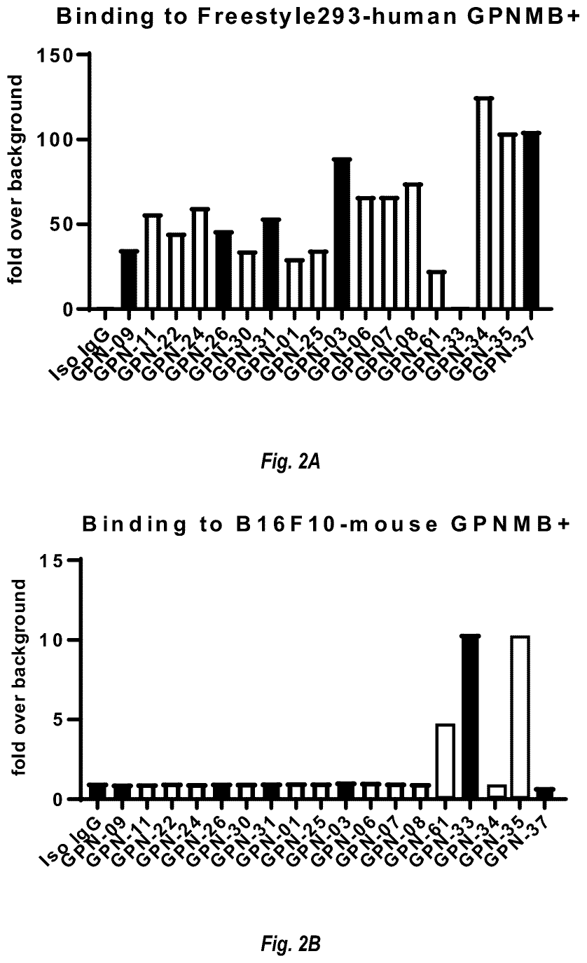

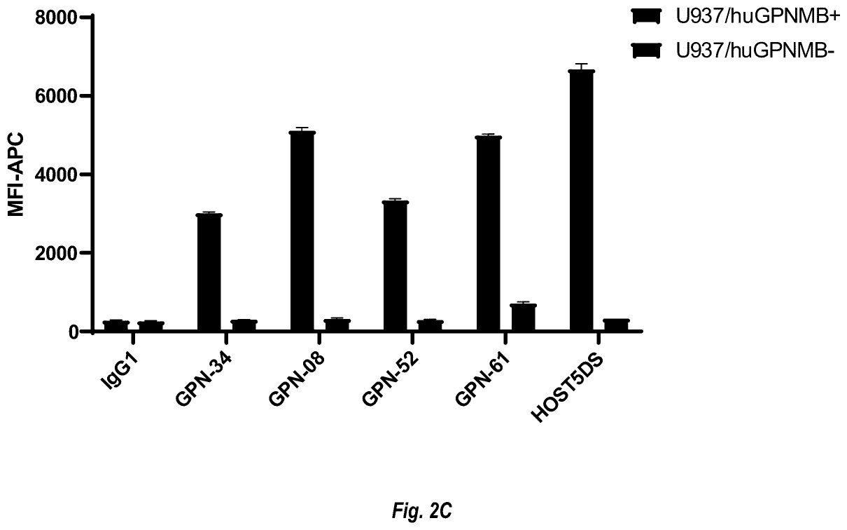

A majority of the anti-GPNMB antibodies of the present disclosure demonstrated binding to Freestyle293-huGPNMB+ or B16F10 cells as indicated by positive MFI (mean fluorescence intensity) values recorded with antibody staining (FIG. 2A and FIG. 2B). The negative isotype control antibody did not bind to either cell type. Likewise, anti-GPNMB antibodies of the present disclosure did not bind to parental Freestyle293 cells, which lack antigen expression (data not shown). Importantly, several anti-GPNMB antibodies of the present disclosure also bound to the human macrophage cell line U937 cells engineered to express human GPNMB (U937/huGPNMB+) (FIG. 2C). In FIG. 2C, the first data point in each pair corresponds to U937/huGPNMB+ cells, and the second data point in each pair corresponds to U937/huGPNMB− cells. No anti-GPNMB antibodies bound to U937 cells deficient in human GPNMB expression (U937/huGPNMB−). These results demonstrated that anti-GPNMB antibodies of the present disclosure bind to GPNMB expressed on human macrophages, the principal target cell population considered important for in vivo efficacy. MFI values (represented as fold over background levels) for cell lines bound by anti-GPNMB antibodies of the present disclosure are provided in Table 4, and typically showed MFI values>10-fold over background levels.

TABLE 4

Cell Binding

| | Freestyle293- | B16F10 |
|---|---|---|
| Antibody | huGPNMB | moGPNMB |
| GPN-01 | 43.2 | 1.0 |
| GPN-25 | 42.7 | 1.0 |
| GPN-24 | 59.9 | 1.0 |
| GPN-11 | 56.2 | 1.0 |
| GPN-31 | 53.8 | 1.0 |
| GPN-22 | 44.8 | 1.0 |
| GPN-30 | 34.4 | 1.0 |
| GPN-09 | 35.1 | 1.0 |
| GPN-07 | 66.5 | 1.0 |
| GPN-34 | 125.3 | 1.1 |
| GPN-08 | 74.4 | 1.0 |
| GPN-03 | 89.2 | 1.1 |
| GPN-06 | 66.4 | 1.0 |
| GPN-52 | 7.6 | 3.4 |
| GPN-61 | 18.6 | 1.7 |
| GPN-26 | 46.3 | 1.0 |
| GPN-33 | 1.1 | 20.9 |
| GPN-35 | 103.9 | 15.4 |
| GPN-37 | 104.9 | 0.8 |
| GPN-38 | 20.7 | 5.1 |
| GPN-41 | 23.6 | 15.0 |
| GPN-42 | 2.0 | 2.6 |
| GPN-43 | 1.5 | 1.6 |
| GPN-48 | 9.7 | 1.7 |
| GPN-65 | 8.1 | 6.9 |

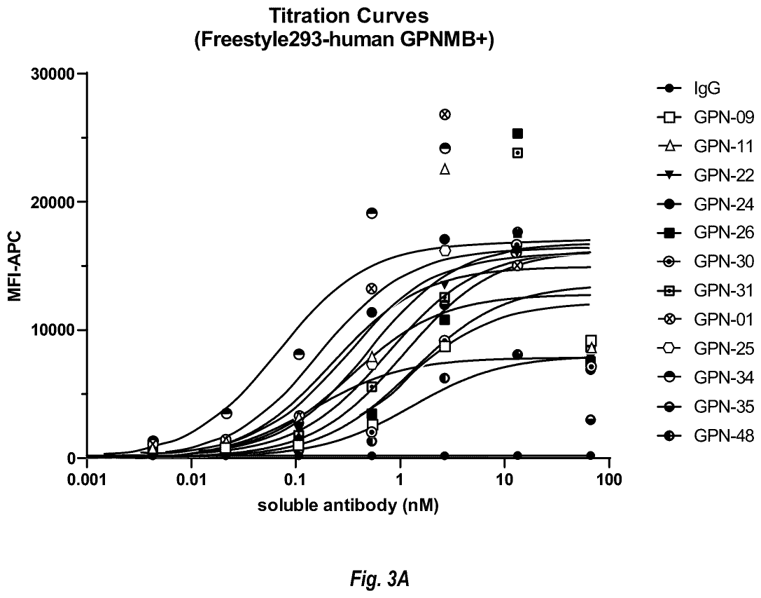

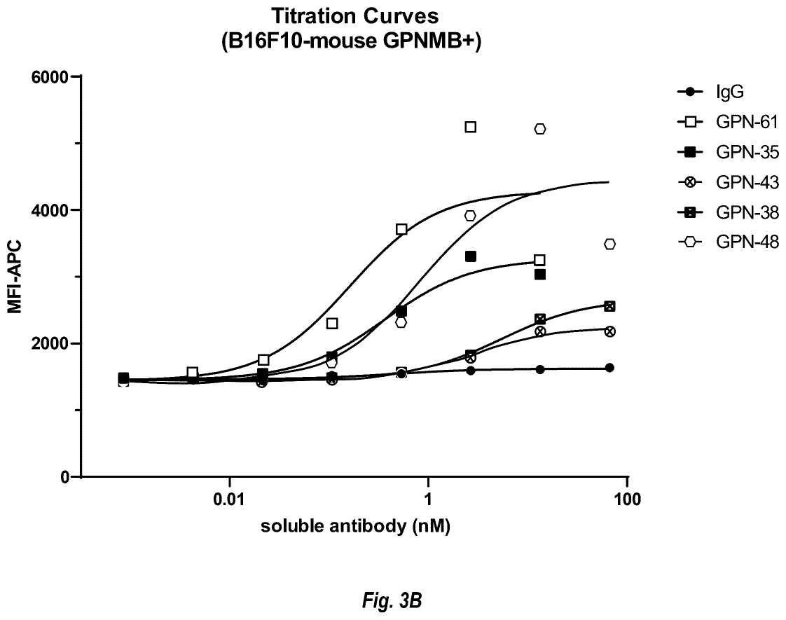

Cell-based binding measurements were also performed to measure the apparent affinities of anti-GPNMB antibodies of the present disclosure to cell-surface expressed GPNMB. Serial dilutions of the monoclonal anti-GPNMB antibodies were added to 100,000 Freestyle293-huGPNMB+ or B16F10 cells and allowed to achieve binding equilibrium at 4° C. After addition of fluorescently labeled secondary antibody and brief washing steps, MFI values as a function of titrated antibody concentration was recorded via FACS analysis. Curves were fit using nonlinear regression analysis with Graphpad Prism 6 software. (FIG. 3A and FIG. 3B) EC50 values obtained from these cell-based titration experiments are provided in Table 5 below.

TABLE 5

Cell Binding (EC50, nM)

| Antibody | huGPNMB | moGPNMB |
|---|---|---|
| GPN-01 | 0.17 | 0 |
| GPN-25 | 0.50 | 0 |
| GPN-24 | 0.22 | 0 |
| GPN-11 | 0.30 | 0 |
| GPN-31 | 0.76 | 0 |
| GPN-22 | 0.33 | 0 |
| GPN-30 | 1.12 | 0 |
| GPN-09 | 1.31 | 0 |
| GPN-07 | 18.36 | 0 |
| GPN-34 | 0.41 | 0 |
| GPN-08 | 0.87 | 0 |
| GPN-03 | 3.38 | 0 |
| GPN-06 | 1.61 | 0 |
| GPN-52 | 1.27 | 1.60 |
| GPN-61 | 7.25 | 0 |
| GPN-26 | 1.09 | 0 |
| GPN-33 | 0 | 17.84 |
| GPN-35 | 1.33 | 2.29 |
| GPN-37 | 0.75 | 0 |
| GPN-38 | 0.99 | 5.38 |
| GPN-41 | 3.88 | 2.62 |
| GPN-42 | 0 | 76.51 |
| GPN-43 | 0 | 36.19 |
| GPN-48 | 18.84 | 0 |
| GPN-65 | 2.60 | 3.23 |

As shown in FIG. 3A, FIG. 3B and in Table 5, this cell-based assay showed that certain anti-GPNMB antibodies of the present disclosure showed binding to both human and mouse GPNMB on cells expressing GPNMB (GPN-52, GPN-35, GPN-38, GPN-41, and GPN-65); other anti-GPNMB antibodies of the present disclosure showed binding to human GPNMB but not to mouse GPNMB (GPN-01, GPN-25, GPN-24, GPN-11, GPN-31, GPN-22, GPN-30, GPN-09, GPN-07, GPN-34, GPN-08, GPN-03, GPN-06, GPN-61, GPN-26, GPN-37, and GPN-48); and other anti-GPNMB antibodies of the present disclosure showed binding to mouse GPNMB but not to human GPNMB (GPN-33, GPN-42, and GPN-43).

Additionally, these results showed that certain anti-GPNMB antibodies of the present disclosure showed a range of EC50 values derived from this cell-based assay of approximately 0.17 nM to approximately 19 nM for human GPNMB, while other anti-GPNMB antibodies of the present disclosure showed a range of EC50 values derived from this cell-based assay of approximately 1.6 nM to approximately 77 nM for mouse GPNMB.

### Example 7: Ligand-Blocking and Non-Blocking GPNMB Antibodies

The extracellular domain of GPNMB protein contains putative protein- or carbohydrate-binding domains capable of recognizing various ligands on the cell surface. Though specific ligands for GPNMB have not been verified, previous studies reported that soluble GPNMB can bind to multiple cell lines, which supports the view of the receptor possessing promiscuous binding activity. Soluble mouse GPNMB-Fc polypeptide, consisting of the extracellular domain of mouse GPNMB and an Fc region of human IgG1 or mouse IgG2A (R&D System) (moGPNMB or mGPNMB) was used to confirm the binding activity of GPNMB towards different cell lines. Binding properties of soluble moGPNMB-Fc were examined by flow cytometry. Different cell lines (mouse SVEC4-10 cells, normal human lung fibroblasts (NHLF), mouse astrocytes, and human astrocytes were nonenzymatically harvested, plated at 100,000 cells/well, and incubated with increasing concentrations moGPNMB-Fc or control human IgG1 or control mouse IgG2A, in binding buffer (Hanks' balanced salt solution containing 1.3 mM CaCl₂, 0.8 mM MgSO₄, 3% fetal calf serum, and 10 mM HEPES) for 30 min on ice.

After washing, the cells were labeled with 1 μg/ml PE-conjugated anti-human IgG Fc or anti-mouse IgG Fc for 30 min. Binding of soluble moGPNMB-Fc to cells was examined by FACSCanto (Becton Dickinson, San Jose, CA). In some experiments, the binding was performed in the presence of known GPNMB binding inhibitors (heparin, dextran sulfate, all purchased from Sigma).

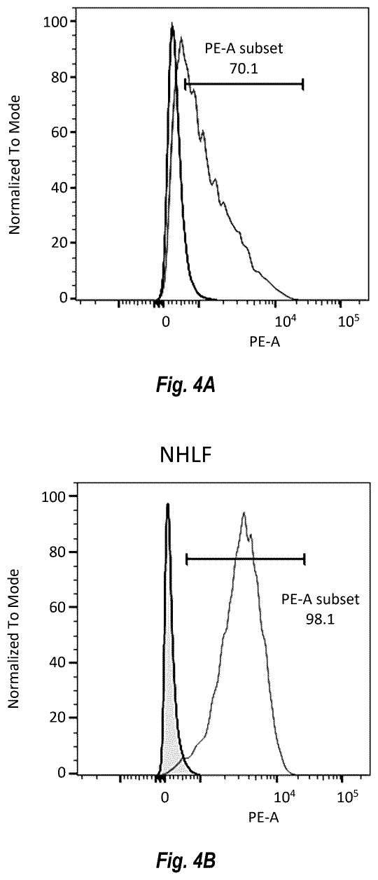

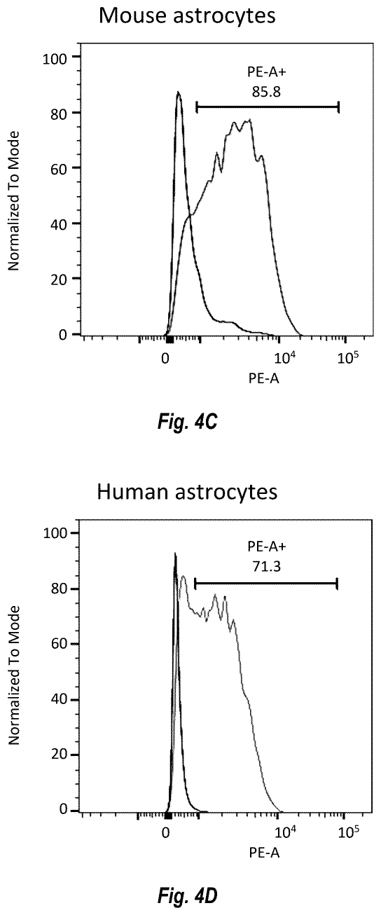

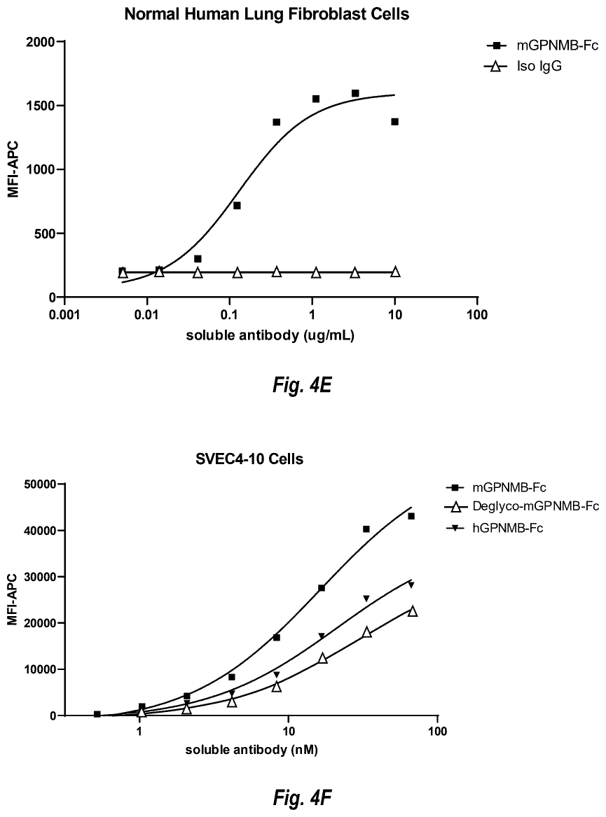

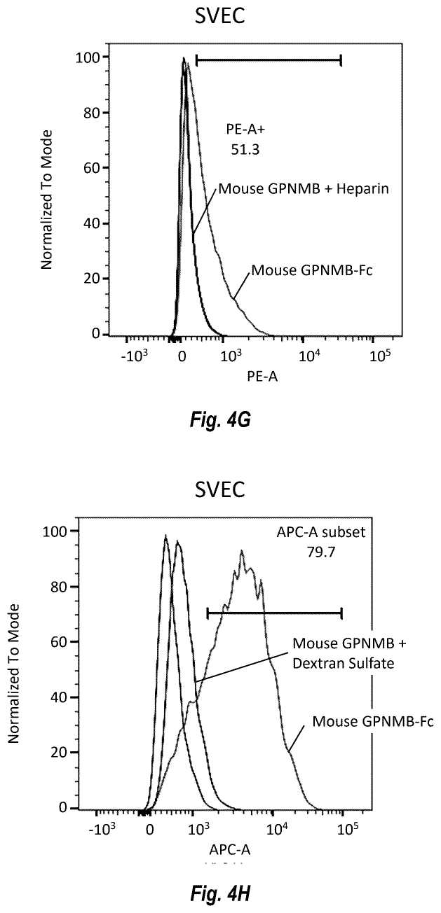

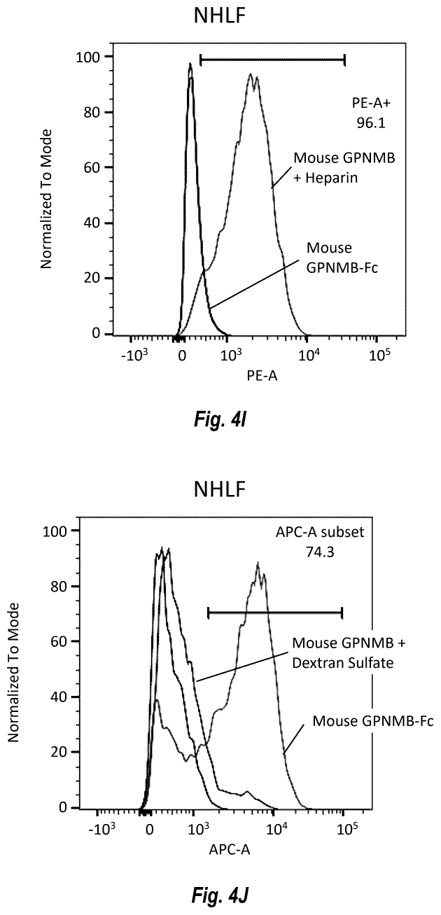

The moGPNMB-Fc polypeptide showed significant binding to mouse SVEC4-10 cells (mouse endothelial cell line, ATCC), NHLF (Lonza), primary mouse astrocytes (Lonza), and human astrocytes (FIG. 4A-FIG. 4D), further demonstrating that soluble GPNMB retained ability to bind to different cell types. FIG. 4E and FIG. 4F show that moGPNMB-Fc binds to NHLF and SVEC, respectively, in a dose-dependent manner. Additionally, huGPNMB-Fc binds SVEC cells in dose-dependent manner (FIG. 4F). In these studies, the moGPNMB-Fc polypeptide was deglycosylated to remove carbohydrate/sugar molecules from moGPNMB; FIG. 4F shows that deglycosylated moGPNMB-Fc displayed diminished binding to SVEC4-10 cells, implicating the role of carbohydrates in GPNMB binding to cells. Furthermore, inhibition studies showed that soluble moGPNMB-Fc polypeptide binding to SVEC4-10 cells and NHLF cells was reduced significantly by adding heparin or dextran sulfate to the binding buffer (FIG. 4G-FIG. 4J). These results were consistent with previously reported observations and suggested that GPNMB interacts with heavily glycosylated proteins on the cell surface (Shikano et al., 2001, J Biol Chem, 275).

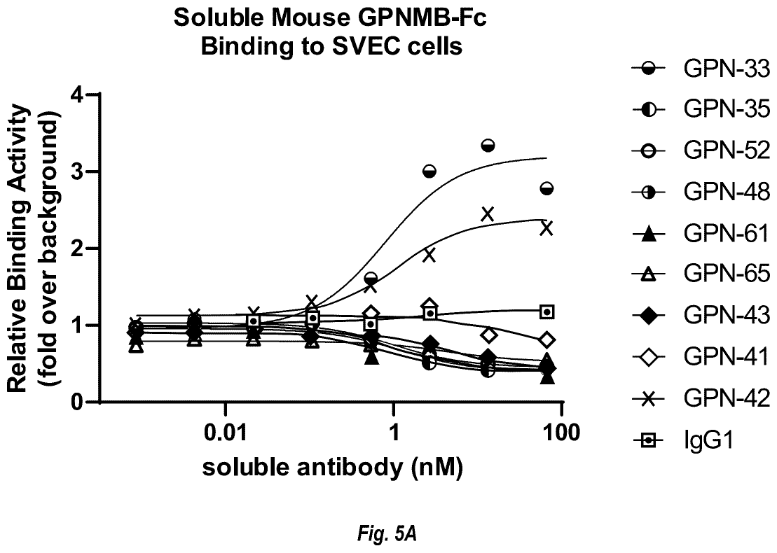

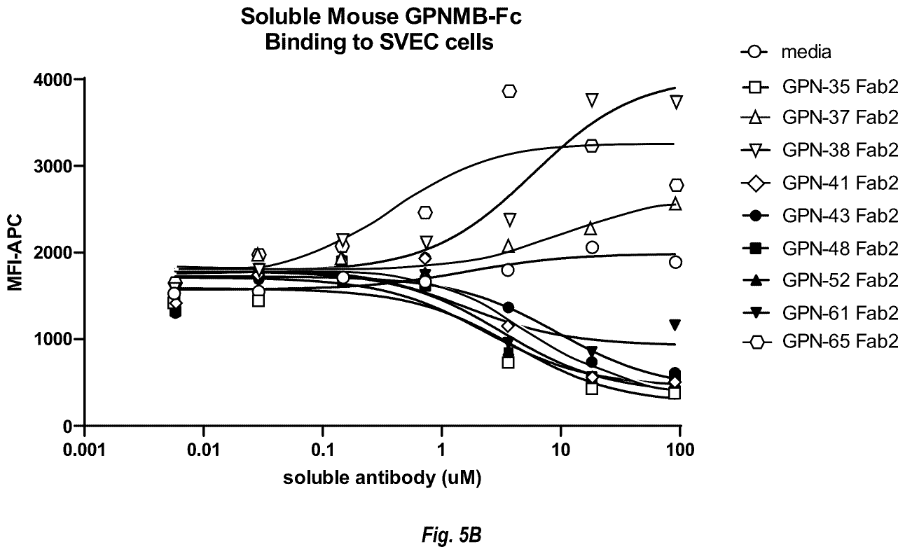

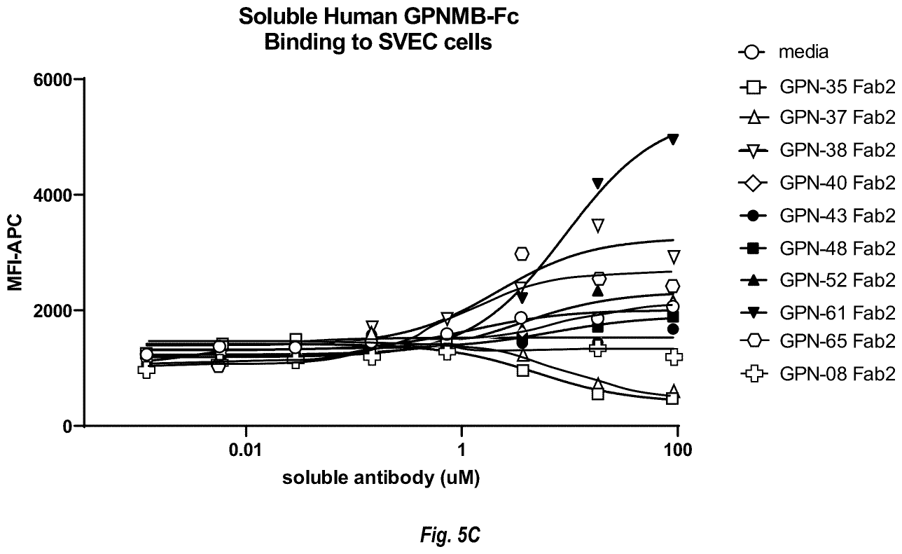

Purified full-length anti-GPNMB antibodies of the present disclosure were evaluated for their ability to block binding of moGPNMB-Fc to SVEC4-10 cells to determine the ligand blocking potency of each anti-GPNMB monoclonal antibody. Briefly, 2 μg/mL moGPNMB-Fc (mouse IgG2A Fc) diluted in binding buffer was pre-complexed with increasing concentrations of anti-GPNMB antibody of the present disclosure or human IgG1 isotype control antibody in 96-well plates. Subsequently, 100,000 SVEC4-10 cells were added per well and incubated on ice for 30 minutes. Soluble moGPNMB-Fc bound to cell surface was detected with fluorescent anti-mouse IgG2A secondary antibody and measured on FACSCanto. (FIG. 5A and FIG. 5B) IC50 values (for antibodies that block moGPNMB-Fc cell binding) and EC50 values (for antibodies that enhance moGPNMB-Fc cell binding) based on changes in MFI values were calculated (average+standard error) for each anti-GPNMB antibody and listed in Table 6. In Table 6, NA indicates no activity. FIG. 5C shows soluble huGPNMB-Fc binding to SVEC cells. In all cases, certain anti-GPNMB antibodies of the present disclosure blocked soluble GPNMB binding to cells, while other anti-GPNMB antibodies of the present disclosure enhanced soluble GPNMB binding to cells.

TABLE 6

Ligand Binding
moGPNMB-Fc

| Antibody | Status | EC/IC50 (nM) |
|---|---|---|
| GPN-01 | NA | 0 |
| GPN-25 | NA | 0 |
| GPN-24 | NA | 0 |
| GPN-11 | NA | 0 |
| GPN-31 | NA | 0 |
| GPN-22 | NA | 0 |
| GPN-30 | NA | 0 |
| GPN-09 | NA | 0 |
| GPN-07 | NA | 0 |
| GPN-34 | NA | 0 |
| GPN-08 | NA | 0 |
| GPN-03 | NA | 0 |
| GPN-06 | NA | 0 |
| GPN-52 | Blocker | 1.00 |
| GPN-61 | Blocker | 0.77 |
| GPN-26 | NA | 0 |
| GPN-33 | Enhancer | 0.79 |
| GPN-35 | Blocker | 1.17 |
| GPN-37 | NA | 0 |
| GPN-38 | NA | 0 |
| GPN-41 | NA | 0 |
| GPN-42 | Enhancer | 1.19 |
| GPN-43 | Blocker | 5.37 |
| GPN-48 | Blocker | 1.2 |
| GPN-65 | Enhancer | 4.27 |

The results showed that several anti-GPNMB antibodies of the present disclosure, including anti-GPNMB antibodies GPN-52, GPN-61, GPN-35, GPN-43, and GPN-48, blocked soluble moGPNMB-Fc binding to SVEC4-10 cells in a dose-dependent manner. Other anti-GPNMB antibodies of the present disclosure, including anti-GPNMB antibodies GPN-33, GPN-42, and GPN-65 enhanced soluble moGPNMB-Fc binding to SVEC4-10 cells in a dose-dependent manner.

Purified recombinant Fab2 fragments of select anti-GPNMB antibodies of the present disclosure were produced through transient transfection and purified. Briefly, 2 ug/mL soluble mouse or human GPNMB-Fc diluted in binding buffer was pre-complexed with increasing concentrations of anti-GPNMB Fab2 fragments in 96-well plates. Subsequently, 100,000 SVEC4-10 cells were added per well and incubated on ice for 30 minutes. Soluble moGPNMB-Fc or huGPNMB-Fc bound to cell surface was detected with fluorescent anti-human IgG1 secondary antibody and measured on FACSCanto. IC50 and EC50 values based on changes in MFI values were calculated for each antibody. The results of these studies are provided in Table 7.

TABLE 7

| | |
|---|---|
| Ligand Binding: | Ligand Binding: |
| moGPNMB-Fc | huGPNMB-Fc |

| | | EC/IC50 | | EC/IC50 |
|---|---|---|---|---|
| Antibody | Status | (nM) | Status | (nM) |
| GPN-08 | ND | | NA | 0 |
| GPN-52 | Blocker | 3.19 | NA | 0 |
| GPN-61 | Blocker | 1.56 | Enhancer | 9.05 |
| GPN-35 | Blocker | 4.05 | Blocker | 5.08 |
| GPN-37 | NA | 0 | Blocker | 9.49 |
| GPN-38 | Enhancer | 5.83 | Enhancer | 2.15 |
| GPN-43 | Blocker | 9.42 | NA | 0 |
| GPN-48 | Blocker | 2.33 | NA | 0 |
| GPN-65 | Enhancer | 0.38 | Enhancer | 0.83 |

Consistent with that observed with full-length anti-GPNMB antibodies, recombinant Fab2 fragments of anti-GPNMB antibodies GPN-35, GPN-43, GPN-61, GPN-41, GPN-52, and GPN-48 blocked moGPNMB-Fc binding to SVEC4-10 cells. In contrast, anti-GPNMB antibodies GPN-38 and GPN-65 enhanced moGPNMB-Fc binding to SVEC4-10 cells. Consistent with full-length antibodies, recombinant Fab2 fragments of anti-GPNMB antibodies GPN-37 and GPN-35 blocked huGPNMB-Fc binding to SVEC4-10 cells, while anti-GPNMB antibodies GPN-61 and GPN-38 enhanced human GPNMB-Fc binding to SVEC4-10 cells.

### Example 8: GPNMB Antibodies Increase Cell Surface Expression of PDL1 and Other Activation Markers in Human Macrophages

GPNMB has been shown to modulate inflammation and promote macrophage M2 polarization under certain in vitro conditions. Anti-GPNMB antibodies of the present disclosure were evaluated for the ability to modulate expression of activation surface markers on human macrophages. Human macrophages were generated from CD14+ monocytes enriched from whole blood of healthy volunteers. Briefly, monocytes from peripheral human blood samples were isolated using the RosetteSep™ monocyte isolation antibody cocktail (StemCell Technologies) and differentiated into macrophages with 50 ng/mL human M-CSF (PeproTech) and cultured for 7 days. Cells were plated on culture dishes in RPMI 1640 medium (Invitrogen) containing 10% fetal calf serum (Hyclone) and 20 mM HEPES and cultured at 37° C. in 5% CO₂. On day 7, the human macrophages were harvested and seeded at 100,000 cells per well in 96-well plates. Cells were treated with 20 ng/mL M-CSF and incubated with anti-GPNMB antibodies of the present disclosure or isotype control antibody for 48 hours. Macrophages were stained for PDL1 (Biolegend, clone MIH3), CD40 (Biolegend, clone 5C3), and CD80 (Biolegend, clone 2D10) in 100 L FACS buffer (PBS supplemented with 2% FBS, 2 mM EDTA) containing FcR blocking agents (eBioscience) for 30 minutes on ice. Cells were washed twice in cold FACS buffer and acquired on a BD FACS Canto. Data analysis and calculation of mean fluorescence intensity (MFI) values was performed with FlowJo (TreeStar) software version 10.0.8. In some experiments, macrophages were treated with indicated antibodies in the presence of Jak Inhibitor I, a potent pharmacological inhibitor of Jak1, Jak2, and Jak3 (StemCell Technologies).

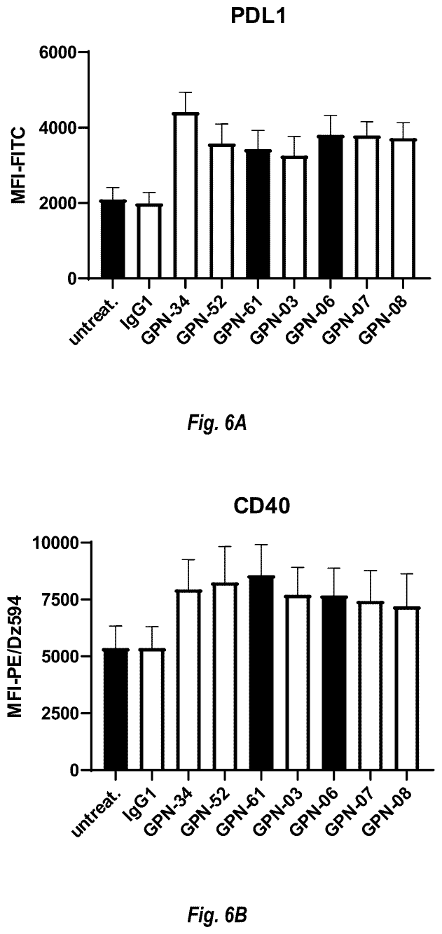

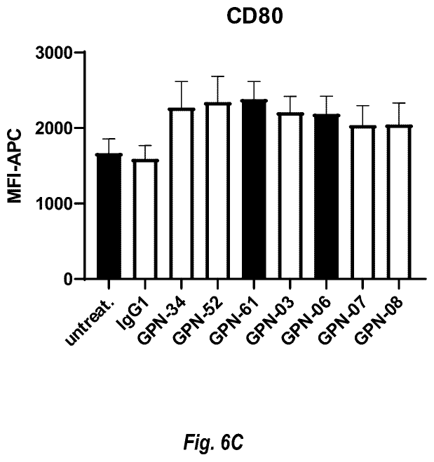

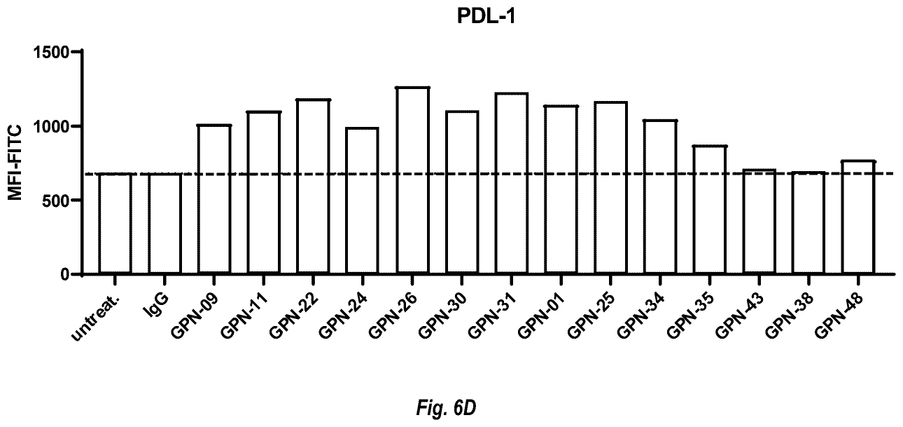

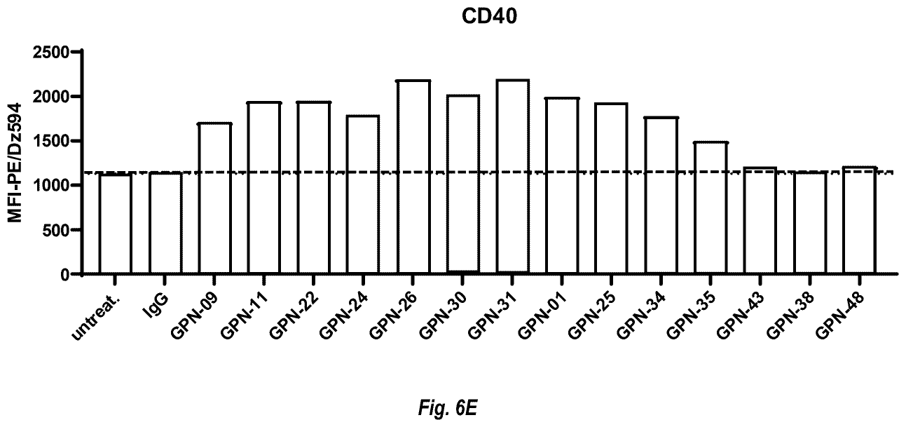

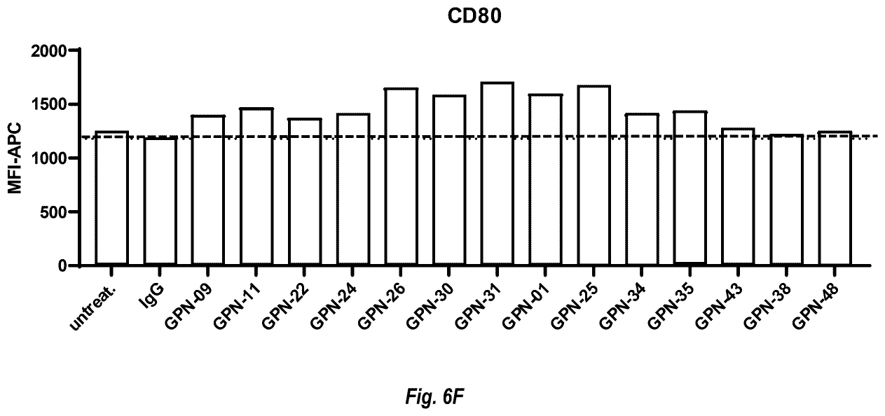

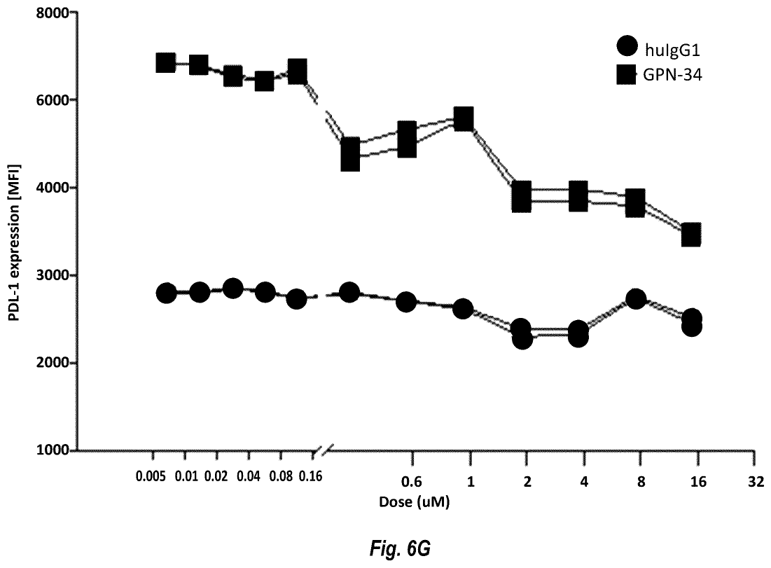

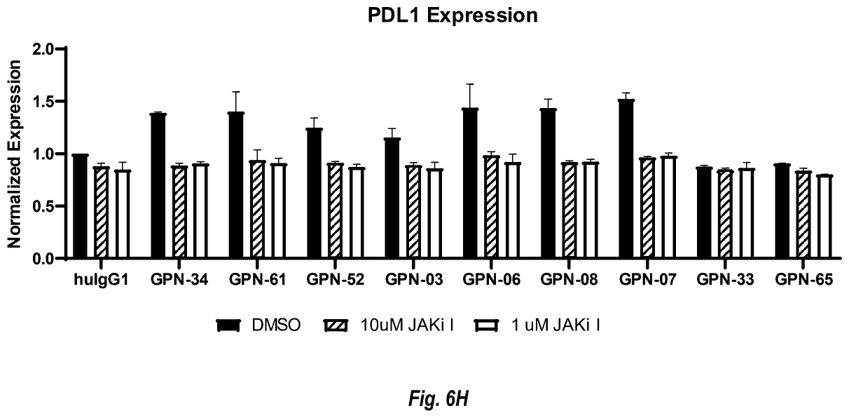

FIG. 6A-FIG. 6F show the results of these studies analyzing the effect of anti-GPNMB antibodies of the present disclosure on expression levels of PDL1, CD40, and CD80. As shown in FIG. 6A-FIG. 6C, several anti-GPNMB antibodies of the present disclosure, including anti-GPNMB antibodies GPN-34, GPN-52, GPN-61, GPN-03, GPN-06, GPN-07, and GPN-08, increased expression of PDL1, CD40, and CD80 in human macrophages. As shown in FIG. 6D-FIG. 6F, additional anti-GPNMB antibodies increased expression of PDL1, CD40, and CD80, including GPN-GPN-09, GPN-11, GPN-22, GPN-24, GPN-26, GPN-30, GPN-31, GPN-01, GPN-34, and GPN-35. These results are further presented in Table 8 below (Example 9).

Certain macrophage activation markers and PDL1 have been reported to be induced by interferon signaling pathways regulated by Jak/Stat. To verify that PDL1 induction by anti-GPNMB antibodies of the present disclosure required Jak/Stat signaling, human macrophages were treated with anti-GPNMB antibody GPN-34 and increasing concentrations of Jak inhibitor I. As shown in FIG. 6G, exemplary anti-GPNMB antibody GPN-34 induced robust expression of PDL1 in the absence of Jak inhibitor treatment. However, addition of the Jak inhibitor reduced the anti-GPNMB antibody-mediated PDL1 induction in human macrophages in a dose-dependent manner. Furthermore, Jak/Stat signaling was required for PDL1 induction for other anti-GPNMB antibodies tested (FIG. 6H). These results were consistent with anti-GPNMB antibodies of the present disclosure antagonizing GPNMB function and promoting activation of human macrophages in vitro.

### Example 9: Anti-GPNMB Antibodies Increase Glucocerebrosidase Activity in Human Macrophages

GPNMB localizes to the plasma membrane and intracellular compartments, including the lysosome. The cytoplasmic domain of GPNMB contains a di-leucine motif, which functions as a sorting signal to target the receptor to the endo-lysosomal pathway. Anti-GPNMB antibodies of the present disclosure were evaluated for their ability to modulate lysosome function in human macrophages, as measured by changes in glucocerebrosidase (GCase) activity, as follows. Human macrophages were generated from CD14+ monocytes enriched from whole blood of healthy volunteers. Briefly, monocytes from peripheral human blood samples were isolated using the RosetteSep™ monocyte isolation antibody cocktail (StemCell Technologies) and differentiated into macrophages with 50 ng/mL human M-CSF (PeproTech) and cultured for 7 days. Cells were plated on culture dishes in RPMI 1640 medium (Invitrogen) containing 10% fetal calf serum (Hyclone) and 20 mM HEPES and cultured at 37° C. in 5% CO₂. On day 7, human macrophages were harvested and seeded at 100,000 cells per well in 96-well plates. Cells were treated with 20 ng/mL MCSF and indicated anti-GPNMB antibodies or isotype control for 48 hours. Macrophages were pelleted and resuspended in fresh RPMI 1640 media+10% FBS with 100 μM FDGlu or PFB-FDGlu (ThermoFisher) for 1 hour at 37° C. Cells were washed twice in cold FACS buffer and acquired on a BD FACS Canto. Data analysis and calculation of median fluorescence intensity (MFI) values was performed with FlowJo (TreeStar) software version 10.0.8. In some experiments, macrophages were treated with conduritol-3-epoxide (CBE), a potent pharmacological inhibitor of glucocerebrosidase, or recombinant progranulin, a positive regulator of glucocerebrosidase (Adipogene).

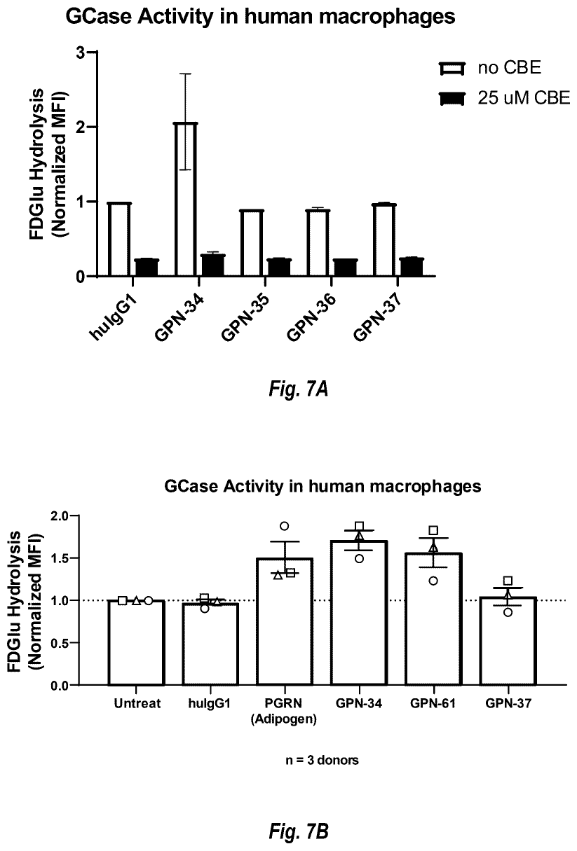

FDGlu is a fluoregenic glucose-based probe that functions as a substrate for the GBA. Though non-fluorescent in its native form, FDGlu is internalized by cells and hydrolyzed by glucocerebrosidase to produce a fluorescein dye byproduct detectable by flow cytometry. As shown in FIG. 7A, human macrophages demonstrated robust glucocerebrosidase (GCase) activity as determined by fluorescence of added FDGlu reagent. In the presence of CBE, the fluorescence signal is abolished, confirming the specificity of the probe to GCase activity.

Additionally, treating macrophages with recombinant progranulin significantly increased fluorescence, consistent with progranulin augmenting lysosome function in cells. Treating human macrophages with anti-GPNMB antibodies of the present disclosure showed that anti-GPNMB antibodies, including anti-GPNMB antibodies GPN-34 and GPN-61, were capable of increasing GCase activity. Anti-GPNMB antibodies of the present disclosure were as potent as recombinant progranulin in augmenting GCase activity in human macrophages (FIG. 78). Importantly, anti-GPNMB antibodies of the present disclosure that enhanced GCase activity also increased PDL1 levels and certain activation markers on macrophages, suggesting that both cellular functions are linked through the inhibition of GPNMB.

Table 8 below shows quantitative data from these GCase studies, as well as changes in PDL1, CD40, and CD80 as described above in Example 8.

TABLE 8

Functional Assay (FoB)

| Antibody | PDL1 | CD40 | GCase | CD80 |
|---|---|---|---|---|
| GPN-01 | 1.70 | 1.74 | 1.05 | 1.36 |
| GPN-25 | 1.73 | 1.68 | 1.24 | 1.41 |
| GPN-24 | 1.48 | 1.56 | 1.22 | 1.20 |
| GPN-11 | 1.64 | 1.70 | 1.24 | 1.25 |
| GPN-31 | 1.82 | 1.90 | 1.22 | 1.44 |
| GPN-22 | 1.76 | 1.70 | 1.21 | 1.16 |
| GPN-30 | 1.64 | 1.75 | 1.27 | 1.34 |
| GPN-09 | 1.50 | 1.49 | 1.26 | 1.19 |
| GPN-07 | 1.67 | 1.39 | 2.13 | 1.29 |
| GPN-34 | 1.56 | 1.52 | 1.76 | 1.36 |
| GPN-08 | 1.59 | 1.35 | 1.18 | 1.29 |
| GPN-03 | 1.32 | 1.45 | 1.47 | 1.40 |
| GPN-06 | 1.61 | 1.44 | 1.58 | 1.38 |
| GPN-52 | 1.44 | 1.53 | ND | 1.46 |
| GPN-61 | 1.52 | 1.61 | 1.46 | 1.50 |
| GPN-26 | 1.88 | 1.91 | ND | 1.40 |
| GPN-33 | NA | NA | NA | NA |
| GPN-35 | 1.30 | 1.31 | 0.90 | 1.22 |
| GPN-37 | NA | NA | 1.01 | NA |
| GPN-38 | 1.03 | 1.01 | ND | 1.03 |
| GPN-41 | NA | NA | NA | NA |
| GPN-42 | NA | NA | NA | NA |
| GPN-43 | 1.05 | 1.06 | 1.01 | 1.08 |
| GPN-48 | 1.14 | 1.06 | 1.02 | 1.06 |
| GPN-65 | NA | NA | NA | NA |

### Example 10: GPNMB Antibodies Decrease Cell Surface Expression of GPNMB in Human Macrophages

The ability of anti-GPNMB antibodies of the present disclosure to reduce cell surface levels of GPNMB on human macrophages was evaluated as follows. Human monocytes were isolated from peripheral blood of healthy donors and differentiated into macrophages in vitro. Following differentiation, 100,000 human macrophages were harvested and seeded onto 96-well tissue culture plates with increasing concentrations of isotype control antibody or soluble anti-GPNMB antibodies of the present disclosure. Cells were analyzed by flow cytometry for GPNMB surface expression following overnight incubation at 37 C (to induce receptor internalization) or incubation at 4° C. for 1 hour (to determine antibody competition with FACS detection antibody). GPNMB expression was detected using a commercial anti-human GPNMB (clone HOST5DS, ThermoFisher) belonging to a separate epitope bin than the test antibody.

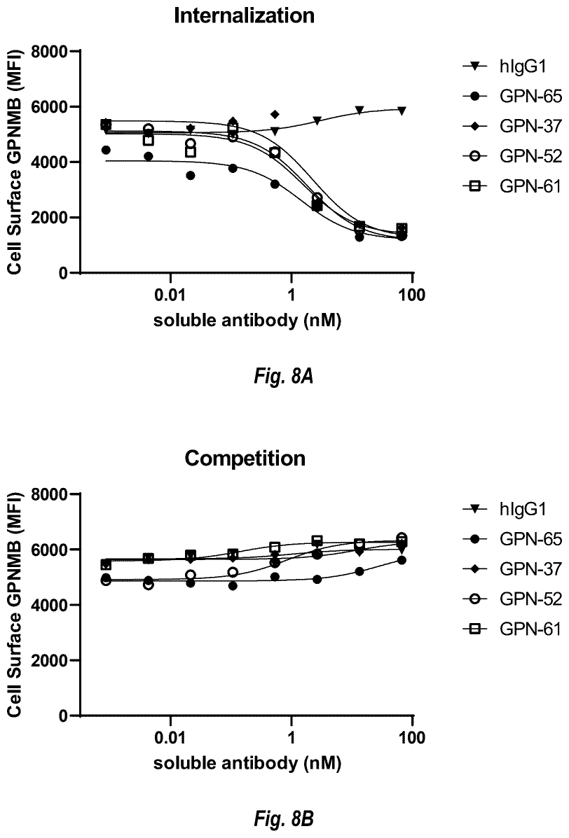

As shown in FIG. 8A, anti-human GPNMB antibodies of the present disclosure significantly reduced cell surface expression of GPNMB by approximately 70% relative to that observed in isotype control antibody-treated macrophages in this internalization assay. FACS analysis revealed that receptor down-regulation occurred within hours following anti-GPNMB antibody addition and was sustained through overnight treatment (data not shown). Results of a competition-based assay performed at 4° C. confirmed that the test antibodies do not interfere with the FACS antibody for GPNMB detection (FIG. 8B). Consequently, the reduction of GPNMB fluorescence signal observed in this internalization assay was likely the result of antibody-mediated downregulation of surface receptor on human macrophages. By reducing GPNMB receptor expression, such anti-GPNMB antibodies of the present disclosure may be effective at antagonizing GPNMB activity independent of ligand blocking activity.

### Example 11: Binding Kinetics of Anti-GPNMB Antibodies

Binding kinetics of human anti-GPNMB IgG1 antibodies of the present disclosure to human, cynomolgus, and mouse GPNMB were evaluated using a Carterra LSA instrument (Carterra, Salt Lake City, UT). Briefly, anti-GPNMB antibodies were prepared by diluting to 10 μg/ml in 10 mM Acetate, pH 4.25 (Carterra), at 300 μl/well. A HC200M sensor chip (Carterra) was activated using the single channel flow cell with a 7-minute injection of a 1:1:1 mixture of 100 mM MES pH 5.5, 100 mM sulfo-NHS, 400 mM EDC (all reconstituted in MES pH 5.5; 100 μl of each mixed in vial immediately before running assay). After switching to the multi-channel array flow cell, the antibodies were injected over the activated chip in a 96-spot array for 15 minutes. The remaining unconjugated active groups on the chip were then blocked by injecting 1M Ethanolamine pH 8.5 (Carterra) for 7 minutes using the single channel flow cell.

After priming with running buffer (HBS-EP+, Teknova) with 0.5 mg/ml BSA (Sigma), the immobilized anti-GPNMB antibodies were tested for their ability to bind to several forms of recombinant GPNMB extracellular domain, including human, cynomolgus, and mouse orthologs as described above. Estimates of affinity were generated by injecting each analyte over the entire antibody array using the single channel flow cell. GPNMB analytes were diluted to 2.5, 7.4, 22, 67, 200, and 600 nM in running buffer, and injected in serial from lowest to highest concentration without regeneration. Two buffer blanks were run between each series (one species per series). Data were processed and analyzed using Kinetics high-throughput kinetics analysis software (Carterra).

The equilibrium dissociation constants (K_{D}) were then calculated from the fitted association and dissociation rate constants (k-on and k-off) for anti-GPNMB antibodies of the present disclosure. The analyses showed that anti-GPNMB antibodies of the present disclosure showed a range of affinities to GPNMB and species binding specificity. In particular, affinity of anti-GPNMB antibodies of the present disclosure for binding to human GPNMB ranged from about 0.4 nm to 120 nM (Table 9); affinity of anti-GPNMB antibodies of the present disclosure for binding to cynomolgus GPNMB ranged from 0.4 nm to 104 nM (Table 10); and affinity of anti-GPNMB antibodies of the present disclosure for binding to mouse GPNMB ranged from 0.3 nM to 5 nM (Table 11).

TABLE 9

| | Human GPNMB | | |
|---|---|---|---|
| Antibody | kₐ (M⁻¹ s⁻¹) | k_{d} (s⁻¹) | K_{D} (nM) |
| GPN-01 | 9.1E+04 | 3.8E−03 | 42.5 |
| GPN-03 | 1.0E+05 | 1.9E−03 | 29.58 |
| GPN-06 | 5.4E+04 | 3.2E−03 | 73.9 |
| GPN-07 | 1.9E+04 | 2.9E−04 | 16.6 |
| GPN-08 | 4.0E+04 | 1.4E−03 | 36 |
| GPN-09 | 1.2E+04 | 1.1E−04 | 9 |
| GPN-11 | 6.9E+04 | 9.5E−04 | 14.4 |
| GPN-22 | 2.8E+04 | 3.4E−03 | 119.6 |
| GPN-24 | 6.9E+04 | 5.7E−04 | 8.8 |
| GPN-25 | 2.6E+04 | 2.8E−04 | 11.4 |
| GPN-26 | 2.7E+04 | 2.7E−04 | 11.1 |
| GPN-30 | 2.6E+04 | 1.2E−04 | 5 |
| GPN-31 | 2.6E+04 | 2.8E−04 | 11.7 |
| GPN-34 | 1.9E+05 | 6.3E−05 | 0.4 |
| GPN-35 | 7.1E+04 | 1.5E−03 | 21# |
| GPN-37 | 1.1E+05 | 1.1E−04 | 1.0 |
| GPN-38 | 1.1E+05 | 5.3E−04 | 4.92 |
| GPN-41 | 2.0E+05 | 8.2E−05 | 0.441 |
| GPN-42 | 1.4E+04 | 8.3E−05 | 6.1 |
| GPN-43 | 1.8E+04 | 2.0E−03 | 111 |
| GPN-48 | 3.3E+05 | 3.7E−04 | 1.1 |
| GPN-52 | 8.6E+04 | 3.2E−03 | 37.9 |
| GPN-61 | 7.1E+04 | 3.5E−03 | 50.1 |
| GPN-65 | 2.1E+05 | 1.0E−04 | 0.49 |

TABLE 10

| | Cynomolgus GPNMB | | |
|---|---|---|---|
| Antibody | kₐ (M⁻¹ s⁻¹) | k_{d} (s⁻¹) | K_{D} (nM) |
| GPN-01 | 1.1E+05 | 3.4E−03 | 31 |
| GPN-03 | 5.4E+04 | 2.1E−03 | 38.7 |
| GPN-06 | 2.1E+04 | 2.1E−03 | 97.9 |
| GPN-07 | 2.2E+04 | 2.5E−04 | 11.3 |
| GPN-08 | 4.8E+04 | 1.5E−03 | 32.8 |
| GPN-09 | 1.4E+04 | 1.2E−04 | 8.4 |
| GPN-11 | 8.3E+04 | 9.5E−04 | 12.1 |
| GPN-22 | 3.2E+04 | 3.3E−03 | 103.9 |
| GPN-24 | 7.7E+04 | 6.1E−04 | 8.3 |
| GPN-25 | 3.0E+04 | 2.0E−04 | 7.4 |
| GPN-26 | 3.1E+04 | 2.7E−04 | 8.9 |
| GPN-30 | 2.6E+04 | 1.3E−04 | 5.1 |
| GPN-31 | 3.2E+04 | 3.6E−04 | 11.2 |
| GPN-34 | 2.5E+05 | 1.1E−04 | 0.4 |
| GPN-52 | 9.0E+04 | 3.6E−03 | 40.1 |
| GPN-61 | 8.6E+04 | 4.1E−03 | 47.9 |

TABLE 11

| | Mouse GPNMB | | |
|---|---|---|---|
| Antibody | kₐ (M⁻¹ s⁻¹) | k_{d} (s⁻¹) | K_{D} (nM) |
| GPN-33 | 4.0E+04 | <0.00001 | <0.3 |
| GPN-35 | 5.6E+04 | 1.2E−04 | 2.1 |
| GPN-38 | 2.3E+04 | 7.8E−05 | 3.45 |
| GPN-41 | 9.7E+04 | 3.7E−05 | 0.4 |
| GPN-52 | 1.9E+05 | 8.2E−04 | 4.6 |
| GPN-61 | 1.3E+05 | 2.9E−04 | 2.1 |
| GPN-65 | 1.0E+05 | 5.1E−05 | 0.5 |

### Example 12: Cross-Reactivity of Anti-GPNMB Antibodies to Human, Cynomolgus, and Mouse GPNMB

Species cross-reactivity of anti-GPNMB antibodies of the present disclosure was determined from the binding kinetic analysis data described above. The results of species binding cross-reactivity analysis are shown below in Table 12.

TABLE 12

| Antibody | Cell binding | SPR binding |
|---|---|---|
| GPN-01 | Hu | Hu, Cy |
| GPN-03 | Hu | Hu, Cy |
| GPN-06 | Hu | Hu, Cy |
| GPN-07 | Hu | Hu, Cy |
| GPN-08 | Hu | Hu, Cy |
| GPN-09 | Hu | Hu, Cy |
| GPN-11 | Hu | Hu, Cy |
| GPN-22 | Hu | Hu, Cy |
| GPN-24 | Hu | Hu, Cy |
| GPN-25 | Hu | Hu, Cy |
| GPN-26 | Hu | Hu, Cy |
| GPN-30 | Hu | Hu, Cy |
| GPN-31 | Hu | Hu, Cy |
| GPN-33 | Mo | Mo |
| GPN-34 | Hu | Hu, Cy |
| GPN-35 | Hu, Mo | Hu, Mo |
| GPN-37 | Hu | Hu |
| GPN-38 | Hu, Mo | Hu, Mo, Cy |
| GPN-41 | Hu, Mo | Hu, Mo, Cy |
| GPN-42 | Hu, Mo | Hu, Mo, Cy |
| GPN-43 | Hu, Mo | Hu, Mo, Cy |
| GPN-48 | Hu, Mo | Hu, Mo, Cy |
| GPN-52 | Hu, Mo | Hu, Mo, Cy |
| GPN-61 | Hu, Mo | Hu, Mo, Cy |
| GPN-65 | Hu, Mo | Hu, Mo, Cy |

These binding experiments showed that the majority of the anti-GPNMB antibodies of the present disclosure tested showed binding cross-reactivity to both human and cynomolgus GPNMB (GPN-01, GPN-03, GPN-06, GPN-07, GPN-08, GPN-09, GPN-11, GPN-22, GPN-24, GPN-25, GPN-26, GPN-30, GPN-31, and GPN-34). Certain anti-GPNMB antibodies of the present disclosure cross-reacted with human, mouse, and cynomolgus (GPN-38, GPN-41, GPN-42, GPN-43, GPN-48, GPN-52, GPN-61, and GPN-65).

### Example 13: Epitope Binning Analysis of Anti-GPNMB Antibodies

Epitope binning analysis was performed on the anti-GPNMB antibodies of the present disclosure by performing a tandem injection approach using a Carterra LSA instrument (Carterra, Salt Lake City, UT). Briefly, purified anti-GPNMB antibodies of the present disclosure and anti-his IgG were diluted to 10 μg/ml in 10 mM Acetate, pH 4.25 (Carterra), at 300 μl/well. A second set of samples was prepared by 2-fold dilution of the antibodies to 5 μg/ml in the same buffer. A HC200M sensor chip (Carterra) was activated using the single channel flow cell with a 7-minute injection of a 1:1:1 mixture of 100 mM MES pH 5.5, 100 mM sulfo-NHS, 400 mM EDC (as described above). After switching to the multi-channel array flow cell, the antibodies were injected over the activated chip in a 96-spot array for 15 minutes. A second array was printed by repeating the injection in another position on the chip. The remaining unconjugated active groups on the chip were then blocked by injecting 1M Ethanolamine pH 8.5 (Carterra) over the entire chip surface for 7 minutes using the single channel flow cell.

After priming with running buffer (HBS-EP+, Teknova) containing 0.5 mg/ml BSA (Sigma), the immobilized antibodies were tested for their ability to bind to recombinant human GPNMB extracellular domain. Bound GPNMB was removed by two 30-second injections of 10 mM Glycine pH2.0 (Carterra). For each cycle, GPNMB was injected over the chip, followed by a test antibody diluted to 30 μg/ml in running buffer. Data were processed and analyzed using Epitope high-throughput binning analysis software (Carterra). Antibodies which were able to bind antigen captured by an immobilized antibody were designated as “sandwich” or “pairing” antibodies, and these antibodies were assigned into a different epitope bin from that of the immobilized antibody. A matrix of pairing and non-pairing antibodies was constructed from the binding results of these experiments, which allowed for an epitope bin landscape of the anti-GPNMB antibodies to be generated.

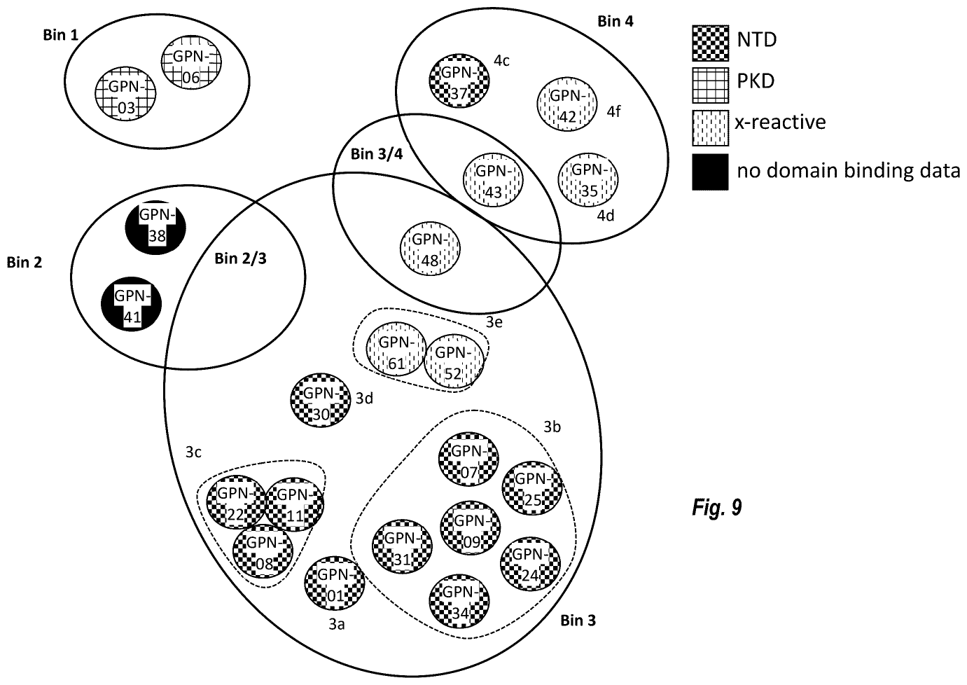

The epitope bins identified from these studies for the anti-GPNMB antibodies are summarized in in Table 13 and FIG. 9 (epitope binning clusters).

TABLE 13

Epitope Bin Assignment

| Antibody | huGPNMB | moGPNMB |
|---|---|---|
| GPN-03 | 1 | |
| GPN-06 | 1 | |
| GPN-38 | 2 | |
| GPN-41 | 2 | |
| GPN-01 | 3a | |
| GPN-07 | 3b | |
| GPN-09 | 3b | |
| GPN-24 | 3b | |
| GPN-25 | 3b | |
| GPN-31 | 3b | |
| GPN-34 | 3b | |
| GPN-08 | 3c | |
| GPN-11 | 3c | |
| GPN-22 | 3c | |
| GPN-30 | 3d | |
| GPN-52 | 3e | 5 |
| GPN-61 | 3e | 5 |
| GPN-43 | 3/4 | 7 |
| GPN-48 | 3/4 | 6 |
| GPN-37 | 4c | |
| GPN-35 | 4d | 9 |
| GPN-42 | 4f | 9 |
| GPN-33 | NB | 10 |
| GPN-26 | ND | |
| GPN-65 | ND | |

### Example 14: Domain Binding Analysis of Anti-GPNMB Antibodies

The following studies were performed to analyze the binding sites of various anti-GPNMB antibodies of the present disclosure on human GPNMB. Human GPNMB extracellular domain can be divided into the following domains, the amino acid sequences of which are shown below:

The resulting chimeric proteins were recombinantly expressed in Expi293 cells and binding of various anti-GPNMB antibodies of the present disclosure were then analyzed for their ability to bind the human/mouse domain-swapped chimera proteins. DNA encoding the domain-swapped chimeras and deletion mutants were prepared by gene synthesis and cloned into the expression vector pcDNA3.4-TOPO (ThermoFisher). Expi293 cells were transfected with 20 μg of plasmids and ExpiFectamine following recommended procedures (ThermoFisher). Transfected cells were grown in 20 mL cultures with shaking at 37° C. and 5% CO₂ for four days. Cells were pelleted by centrifugation and the supernatants were filtered through 0.2 μM filters by vacuum.

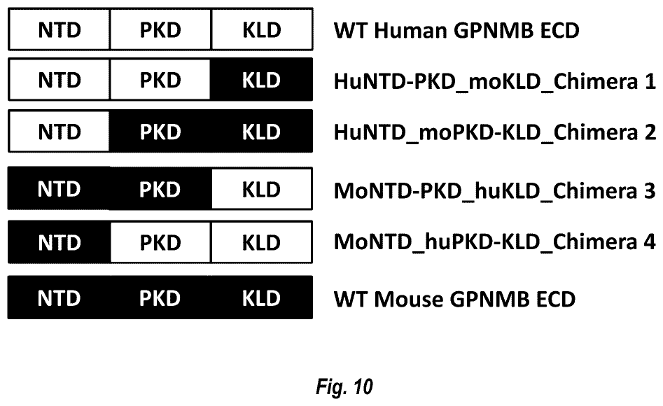

FIG. 10 shows the configurations of the various human/mouse GPNMB domain-swapped chimeras that were generated for use in these studies. In FIG. 10, an H (huGPNMB) or an M (moGPNMB) indicates from which species that particular domain was included (i.e., swapped) in the corresponding chimeric protein construct; NTD (N-terminal domain), PKD (polycystic kidney disease), and KLD (kringle-like domain). Table 7 defines the amino acid sequences of each region of GPNMB used to construct chimeric proteins.

Binding of anti-GPNMB antibodies of the present disclosure to these domain-swapped chimera or deletion mutants (prepared as described above) was tested by Surface Plasmon Resonance (SPR) using Carterra LSA. Purified anti-GPNMB antibodies were immobilized in duplicate on a HC30M chip (Carterra) by amine coupling, following the manufacturer's instructions (described previously). Supernatants containing the constructs were diluted 1:1 with running buffer containing 0.5 mg/mL BSA (HBS-EP+, Teknova) and injected over the immobilized antibodies. The surface was regenerated with 10 mM Glycine pH2.0 after each construct injection. Sensorgrams were analyzed using Carterra Kinetics software to identify patterns of construct binding. Binding to huGPNMB constructs with domains deleted or swapped in from moGPNMB was evaluated. Data from these studies were used to construct a domain binding map for anti-GPNMB antibodies and their binding to various domains of human GPNMB. The results of these binding studies are shown in Table 14.

TABLE 14

Binding

| | Domain | Chimera |
|---|---|---|
| Antibody | Binding | Binding |
| GPN-03 | PKD (hu) | 1, 2 |
| GPN-06 | PKD (hu) | 1, 2 |
| GPN-38 | nd | nd |
| GPN-41 | nd | nd |
| GPN-01 | NTD (hu) | 1, 2 |
| GPN-07 | NTD (hu) | 1, 2 |
| GPN-09 | NTD (hu) | 1, 2 |
| GPN-24 | NTD (hu) | 1, 2 |
| GPN-25 | NTD (hu) | 1, 2 |
| GPN-31 | NTD (hu) | 1, 2 |
| GPN-34 | NTD (hu) | 1, 2 |
| GPN-08 | NTD (hu) | 1, 2 |
| GPN-11 | NTD (hu) | 1, 2 |
| GPN-22 | NTD (hu) | 1, 2 |
| GPN-30 | NTD (hu) | 1, 2 |
| GPN-52 | x-reactive | x-reactive |
| GPN-61 | x-reactive | x-reactive |
| GPN-43 | x-reactive | x-reactive |
| GPN-48 | x-reactive | x-reactive |
| GPN-37 | NTD (hu) | 1, 2 |
| GPN-35 | x-reactive | x-reactive |
| GPN-42 | x-reactive | x-reactive |
| GPN-33 | NTD (mo) | 3, 4 (mo) |
| GPN-26 | ND | |
| GPN-65 | ND | |

### Example 15: Transcript Profiling in Macrophages Derived from GPNMB Knockout Mice or Macrophages Treated with Anti-GPNMB Antibodies

To determine how genetic depletion of GPNMB affects gene expression programs in polarized macrophages, the following studies were performed. Bone marrow derived mouse macrophages were obtained as follows. Femur and tibia bones from mice aged 6-8 weeks were harvested from female wild-type or GPNMB homozygous (−/−) GPNMB knockout (KO) animals. The bones were cut open, and a 21G needle was used to flush out marrow with ice cold PBS+2% FBS. Cells were passed through a 70 μm cell strainer to remove cell clumps, bone, hair, and other cells/tissues. Cells were centrifuged at 400×g for 5 min at 4° C. and resuspended at 1 million cells/ml of BMDM growth media (DMEM with 10% FBS and 20 ng/ml of M-CSF). Cells were left to differentiate for 7 days (M0 macrophages), and on day 7 polarization media was added to the cells as follows: for M1 polarization, the cells were treated with 20 ng/ml M-CSF, 20 ng/ml of IFNγ, and 10 ng/ml of LPS; for M2 polarization, the cells were treated with 20 ng/ml of M-CSF, 20 ng/ml of IL-4, and 20 ng/ml of IL-13. In both cases, the cells were incubated under these polarization conditions for 48 hours before harvesting for RNA isolation. RNA was isolated using Qiagen RNeasy Kits as per manufacturer's protocol.

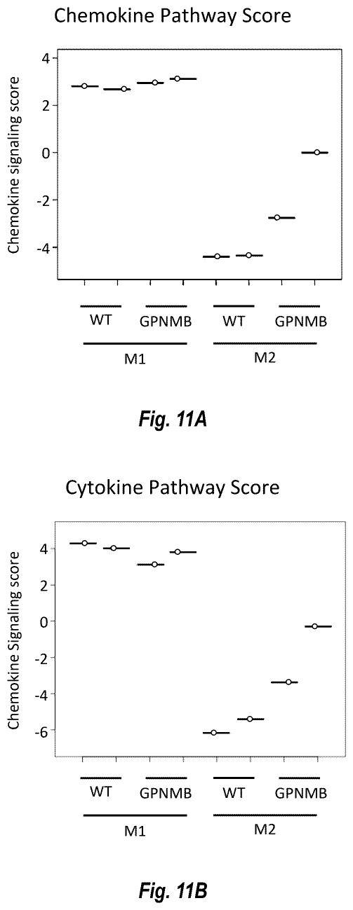

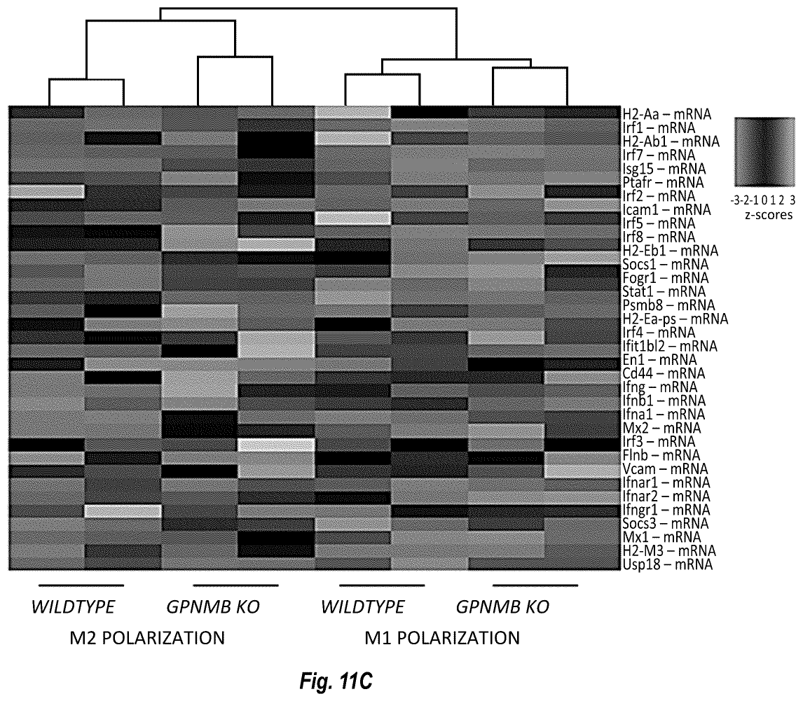

Nanostring analysis of RNA obtained from the mouse macrophages was performed using Murine Myeloid Innate Immunity V2 code set with 125 ng of total RNA, as per the manufacturer's protocol, with a 16-hour overnight hybridization step. nCounter Advanced Analysis 2.0 was used for data analysis and figure generation using the built-in normalization methods, the Gene Set Analysis Module, and Pathway Scoring Module. Nanostring analysis of RNA obtained from the human macrophages was performed using Human Myeloid Innate Immunity V2 code set with 125 ng of total RNA, as per the manufacturer's protocol. A heatmap was generated using ROSALIND software following the nCounter Advanced Analysis protocol. Type I Interferon-associated transcripts that showed a 1.2-fold increase or greater are shown in the heatmap (FIG. 11C). Pathway analysis was performed using ROSALIND. Hypergeometric distribution was used to analyze the enrichment of pathways. Bioplanet Pathway annotations were used. Enrichment was calculated relative to a set of background genes relevant for the experiment.

Analysis of polarized mouse macrophages obtained as described above showed that M2 polarized mouse macrophages robustly express GPNMB mRNA (data not shown). Consistent with this observation, mouse macrophages obtained from GPNMB KO mice showed the most significant effect on the transcriptome in M2 polarized macrophages when compared to that observed in M0-polarized or M1-polarized macrophages. Specifically, the Nanostring Platform compared a subset of transcripts in macrophage obtained from wild-type and GPNMB KO mice that had been polarized to M0, M1, or M2 polarization states. The results showed that genetic loss of GPNMB had the largest effect on differential gene expression in M2 cells, compared to that observed in M0 or M1 cells (data not shown). Based on these results, the remainder of the analysis focused on M2-polarized and M1-polarized mouse macrophages. Under M2 polarizing conditions, loss of GPNMB resulted in gene-expression pathway scores that trended to a more M1, proinflammatory state, as shown in the chemokine and cytokine pathway analysis (FIG. 11A and FIG. 11B). These results indicated that M2 macrophages genetically-depleted of GPNMB were more pro-inflammatory and ‘M1-like’ relative to wild-type control cells. Further examination of transcripts implicated in interferon signaling revealed that in M2 macrophages, loss of GPNMB resulted in an elevated interferon response signature (FIG. 11C).

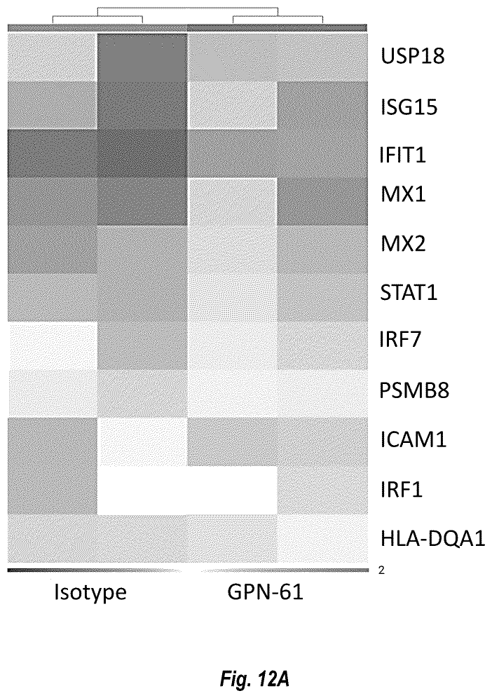

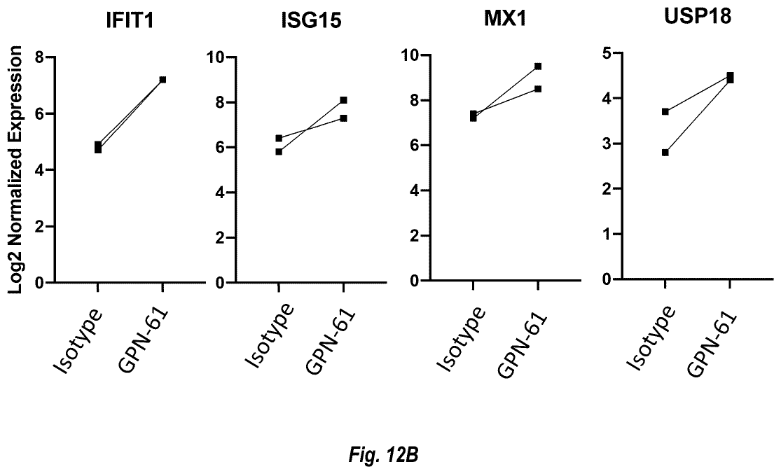

To determine how anti-GPNMB antibodies of the present disclosure might affect gene expression profiles, the following studies were performed with human macrophages derived as follows. 50 million monocytes (obtained from blood of multiple donors) were resuspended in 50 ml differentiation media (40 ml RPMI+10% FBS, 4 ml human serum, 1 mM HEPES, 50 ng/ml of M-CSF (Peprotech), and 10 ng/ml IL-10. Cells were differentiated for 7 days to obtain macrophages. The human monocyte-derived macrophages were then cultured with anti-GPNMB antibody GPN-61 for 48 hours. RNA transcripts from these cells were analyzed using the Nanostring Platform. Despite the anticipated donor-to-donor differences in overall gene expression profiles, treatment of these cells with anti-GPNMB antibody GPN-61 identified 15 mRNA transcripts, 11 of which are associated with the interferon pathway, that showed differential expression compared to that observed in isotype control antibody treated human macrophages. In these analyses, differential expression refers to at least a ±1.5 fold change in gene expression (with a p-value of 0.05 or less). In particular, anti-GPNMB antibody GPN-61 addition to these cells resulted in elevated expression of mRNA-encoding transcripts associated with interferon signaling (p-Adj—0.01183 BioPlanet Interferon Pathway Annotations) (FIG. 12A). A subset of these differentially expressed transcripts is also shown (FIG. 12B). These results showed consistent trends in differentially expressed transcripts between multiple donors.

Taken together, these results showed that in human macrophages, genetic depletion of GPNMB or treatment with anti-GPNMB antibody GPN-61 resulted in an increased inflammatory state, as evidenced by changes in gene expression patterns, including changes in gene expression patterns associated with the interferon pathway. These results further suggested that anti-GPNMB antibodies of the present disclosure are capable of phenocopying differential gene expression in macrophages as observed in cells having a GPNMB genetic depletion.

### Example 16: GPNMB and Tumor Growth in Mouse Model

In order to examine the role of GPNMB on tumor growth, the following experiments were performed. Female wild-type mice, GPNMB−/−homozygous mice, and GPNMB+/−heterozygous mice aged 6-8 weeks were anesthetized with isoflurane and 400,000 MC38 tumor cells were injected subcutaneously in the right flank at a volume of 0.1 ml/animal. Tumor volumes were measured three times per week, and tumors were harvested 16 days post injection.

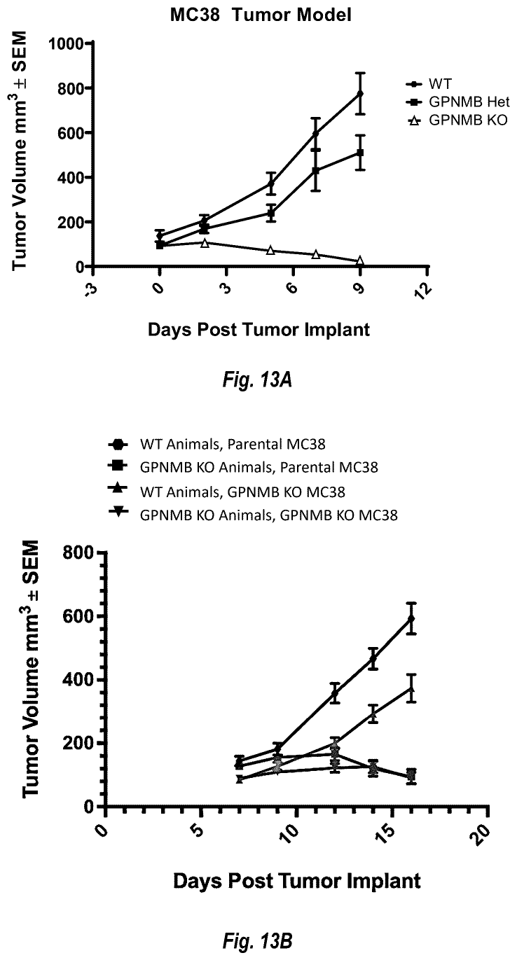

Tumor growth was compared between wild-type mice (WT), GPNMB heterozygote mice (GPNMB Het), and GPNMB homozygous knockout mice (GPNMB KO). Wild-type mice, heterozygous mice (Het), and homozygous mice had tumors with a mean size of 774.3 mm³±92.29, 509 mm³±77.56, and 25.20 mm³±6.654, respectively. (FIG. 13A) These results showed that loss of GPNMB expression or reduced GPNMB expression in mice resulted in lower tumor volume, indicating that lower or no GPNMB expression was protective against tumor growth in the animals.

To confirm that the observed reduction in tumor growth in these mice was not a result of immune rejection of the implanted tumor cells in the GPNMB knockout animals, MC38 cells lacking GPNMB (generated using CRISPR/CAS9 technology by Synthego Corporation) were injected into either wildtype mice or GPNMB KO mice. Consistent with the results obtained above in the tumor growth experiments using mice lacking or with reduced expression of GPNMB, MC38 tumor cells lacking GPNMB grew as poorly as that observed in MC38 parental cells in the GPNMB knockout animals, with average tumor volumes 19 days post inoculation of 135.6 mm³±42.23 and 77.31 mm³±36.76, respectively (FIG. 13B).

Tumors were harvested in PBS on ice, cut into small pieces, and transferred to Miltenyi Gentle MACS C Tubes with 5 ml of RPMI. The tubes were put on GentleMACs and m-tumor program was run 2×. Tumor homogenate was transferred to a 15 ml falcon tube by straining the homogenate over a 70 μM cell strainer (washed 2× with PBS and strained a second time prior to resuspension). Single cell suspensions were either harvested for RNA using Qiagen RNeasy Kits (per manufacturer's protocol) or resuspended in FACs buffer containing 5 mM EDTA. Mouse FcR block was added at a 1:50 dilution, incubated on ice for 15 minutes, and washed in the FACs buffer prior to staining

Cells were stained with anti-CD45 (APC-Cy7, BD Bioscience, Clone 30-F11))+L/D marker (Sytox Blue). Viable CD45+ cells were collected for RNA analysis as follows. RNA was isolated from single-cell suspensions obtained from these harvested tumors using Qiagen RNeasy Kits. Nanostring analysis was performed using the Mouse PanCancer IO 36 codeset and data analysis was performed using ROSALIND as described above.

scRNA-seq sequencing and read alignment was performed as follows. CD45+ cells obtained from the tumors as described above were prepared via 10× Genomics v3 3′ Gene Expression Kit and sequenced on NovaSeq flow cells to achieve a read depth of 25,000 reads per cell and approximately 5,000 cells per sample. Demultiplexed FASTQs were aligned to the mouse genome (mm10 build) using 10× Genomics' CellRanger version 6.0.1 with the command “cellranger count” with default parameters. Mouse-level matrices were concatenated to produce the raw input UMI?count matrix. Cells were filtered out to remove low viability and poorly sequenced cells. Cells that had greater than 10% reads coming from mitochondrial genes were also removed from these analyses. 67,644 CD45+ cells remained after this filtering with a median 4,834 UMIs per mouse and average 5,637 cells per mouse. The resulting UMI counts were normalized, scaled, and centered using the mouse UMI matrix.

Normalization, Dimensional Reduction, and Clustering analysis was performed as follows. The Seurat R package (Butler et al., 2018) was used for all scRNA-seq analysis and processing. All steps were performed using default parameters unless otherwise noted. Log normalization was performed on the UMI count matrix. The number of UMIs per cell and percentage of mitochondrial reads per cell were treated as undesired covariates and regressed out of the normalized expression values. The normalized expression values were scaled and centered for input into subsequent dimensional reductions of principal component analysis (PCA) and uniform manifold approximation projection (UMAP). PCA was performed using the top 2,000 variable genes identified using variance-stabilizing transformation (VST). Cells were segmented based on their neighbors (FindNeighbors Seurat function) using the top 20 principal components (PCs) from PCA and using only the top 2,000 variable genes. The number of PCs used was based on the screen plot (i.e., a plot of the PC eigenvalues in decreasing order). Other parameters of FindNeighbors were k.param of 8. To find clusters (FindClusters Seurat function), a resolution of 0.3 was used. UMAP was computed using the top 20 PCs and cells were plotted onto the first 2 UMAP dimensions.

Cell types were classified based unsupervised learning of each cell's transcriptome and compared to well-known cell-type gene markers. Cluster 2 was defined as monocytes, but we note that a portion of this cluster additionally exhibited high myeloid derived suppressor cell marker genes (e.g., Nos2, Vegfa). Proliferating cells had high expression of G2-, M-, and S-phase genes (e.g., Top2a, Mki67, Mcm5).

Differential expression analysis was performed using functions in the Seurat R package, namely FindAllMarkers and FindMarkers for cluster-defining gene markers and specific cluster comparisons, respectively. To calculate p-values and average log fold-changes, the Wilcoxon rank-sum test was used in conjunction with FDR correction to adjust p-values due to multiple hypothesis testing. To identify cluster-defining gene markers, we compared one cluster to all other clusters. Genes that were expressed in 25% of the cells of a cluster and had greater than absolute value of 0.25 log fold-change were defined to be differentially expressed genes (DEGs) for that group. Cluster defining genes were defined using this differential expression.

Due to varying levels of the number of cells in each experimental group (i.e., both animal and genotype groups), a correction was implemented to adjust for the number of cells in each group when comparing group compositions within clusters. Group-level proportions within clusters were adjusted to account for the raw number of observations of each category in the group. For each group (e.g., cluster), we divided each category's (e.g. mouse #3) observation size by fraction of cells in that category amongst all single-cells, thus giving a value representing the ‘n-corrected observation size’. The n-corrected observation sizes were divided by the n-corrected observation sizes summed across all categories (known and referred to as the n-corrected category percentage).

The t-test function in R was used with the format (data$CellTypeFraction˜data$Genotype) and p-values were taken to denote significant differences in proportion of cell types from GPNMB wildtype and GPNMB heterozygous mice. Analyses were done using CellRanger 6.0.1, R version 4.0.5, Seurat version 4.1.0.

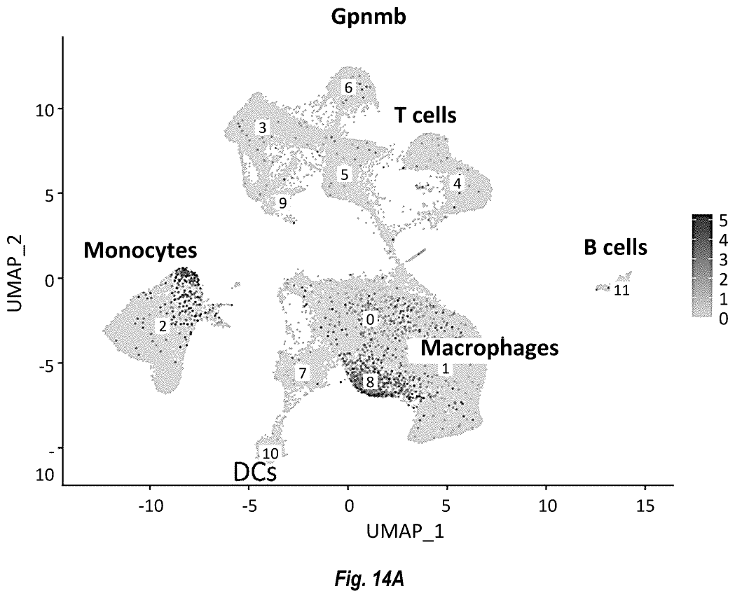

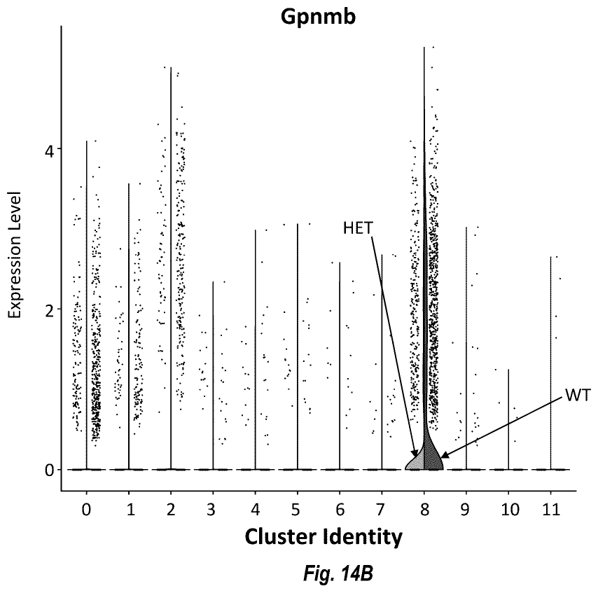

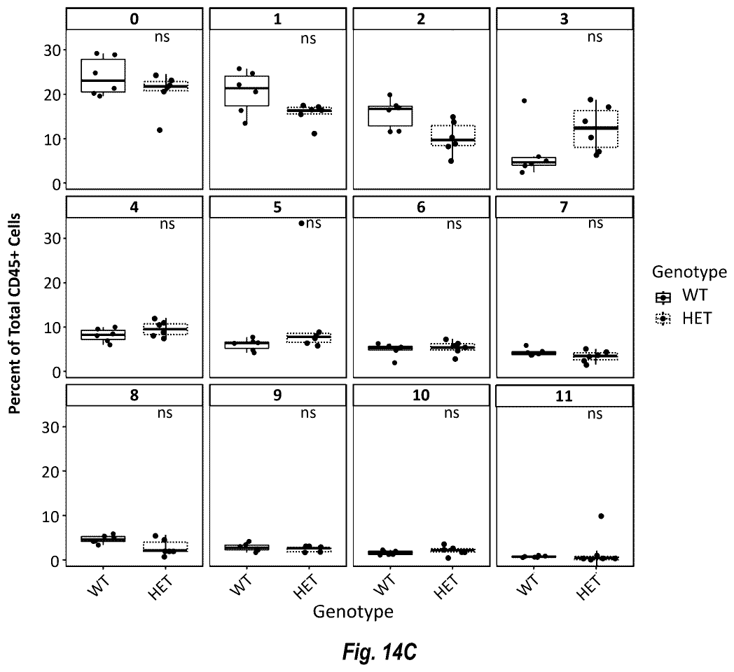

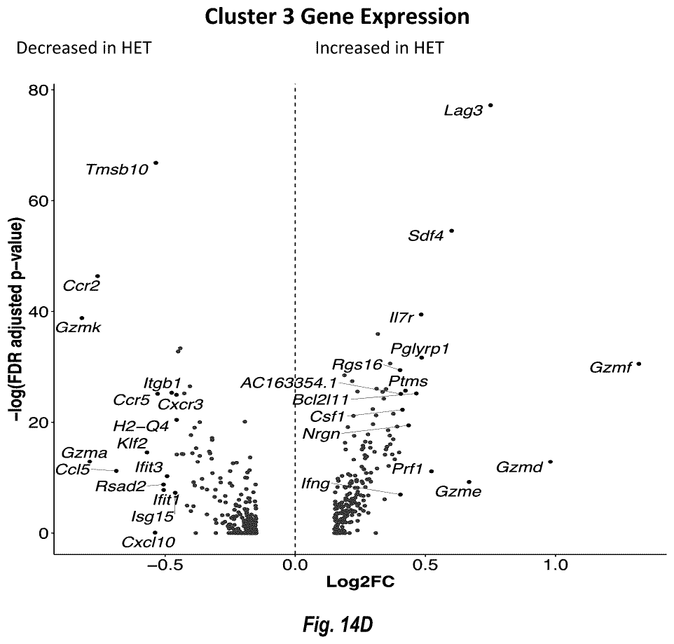

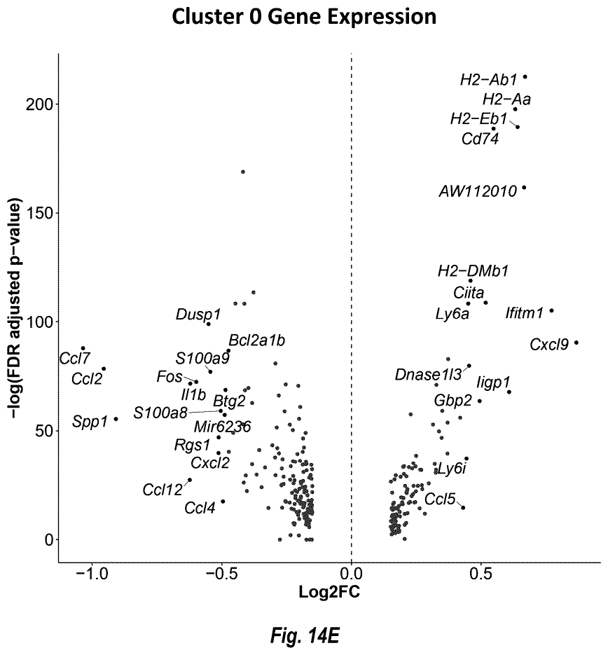

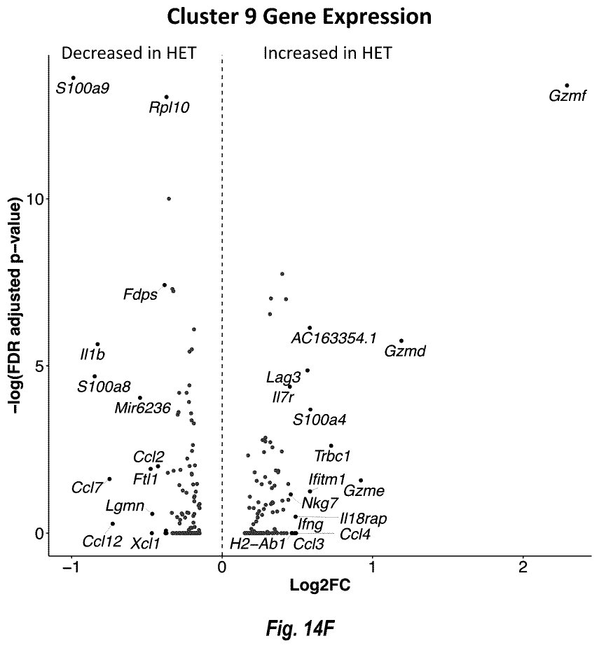

To gain insight into how GPNMB affected tumor growth, CD45+ cells were isolated from wild-type and GPNMB−/+ heterozygote mice and analyzed using scRNA-seq. Unsupervised clustering of the CD45+ cell fraction identified 12 distinct cell populations, representing all cell populations expected to be present in MC38 tumors. Clusters were labeled based on expression of established cell type markers. Of these, GPNMB is most robustly detected in Cluster 8 (macrophages) and a subset of Cluster 2 (monocytes) (FIG. 14A and FIG. 14B).

Cell type fraction statistical analysis was also performed, and the proportion of cells was compared between GPNMB wildtype and GPNMB heterozygote mice. As a proportion of total CD45+ cells captured, tumors obtained from GPNMB heterozygote mice had elevated levels of CD8+ T-cells (Cluster 3) and a reduction of monocytes (Cluster 2) (FIG. 14C). A subset of Cluster 2 monocytes scored high for genes characterizing myeloid-derived suppressive cells. These results suggested that GPNMB heterozygosity resulted in reduction in the number of suppressive-like monocytes from Cluster 2.

Differential expression statistical analysis was performed on each cluster of cells identified in these experiments. Examination of Cluster 3 (CD8+ T-cells) and Cluster 9 (proliferating T-cells) not only showed an increase in their relative abundance, but also showed altered expression of transcripts encoding granzymes and other pro-inflammatory/anti-tumor proteins (FIG. 14D and FIG. 14E). Differential expression in Cluster 0 (C1QC+ macrophages) showed an upregulation in MHC class II and antigen presentation molecules associated with GPNMB heterozygote mice (FIG. 14F).

Taken together, these results indicated that loss of GPNMB resulted in both increased inflammatory states of multiple cell populations, and changes in the overall tumor microenvironment. Such increased inflammatory state changes to the tumor microenvironment are important because tumors contain suppressive myeloid cells and typically create an anti-inflammatory environment, which prevents any necessary signaling that enables recruitment of cytotoxic T cells.

### Example 17: Effect of Anti-GPNMB Antibodies in MC38 Mouse Tumor Models

To examine the effect of anti-GPNMB antibodies of the present disclosure in an MC38 mouse tumor model, the following experiments were performed. Female wild-type mice, GPNMB homozygous mice, and GPNMB heterozygous mice aged 6-8 weeks were anesthetized with isoflurane and 400,000 MC38 tumor cells were injected subcutaneously in the right flank at a volume of 0.1 ml/animal. At day 9 following inoculation, animals were randomized based on tumor volume and were administered various antibodies as follows: 1) isotype control antibody 40 mg/kg; 2) anti-PDL-1 antibody at 3 mg/kg+isotype control antibody at 40 mg/kg; 3) anti-GPNMB antibody GRN-61 at 40 mg/kg+isotype control antibody at 3 mg/kg; or 4) anti-GPNMB antibody GRN-61 at 40 mg/kg+anti-PDL-1 antibody at 3 mg/kg.

Tumor volumes were measured 3×/week and the study was terminated 26 Days following inoculation, when animals had been dosed six times. Statistical Analysis was performed using an Ordinary one-way ANOVA with multiple comparisons, and Dunnett's multiple comparisons test to compare all groups to the Isotype alone group.

Wild-type animals inoculated with MC38 cancer cells were randomized and treated with drug starting 9 days following inoculation, receiving a total of 6 doses of anti-GPNMB antibody treatment. Treatment groups were: isotype control antibody alone; isotype control antibody+anti-PDL-1 antibody; isotype control antibody+anti-GPNMB antibody GPN-61; and anti-GPNMB antibody GPN-61+anti-PDL1 antibody.

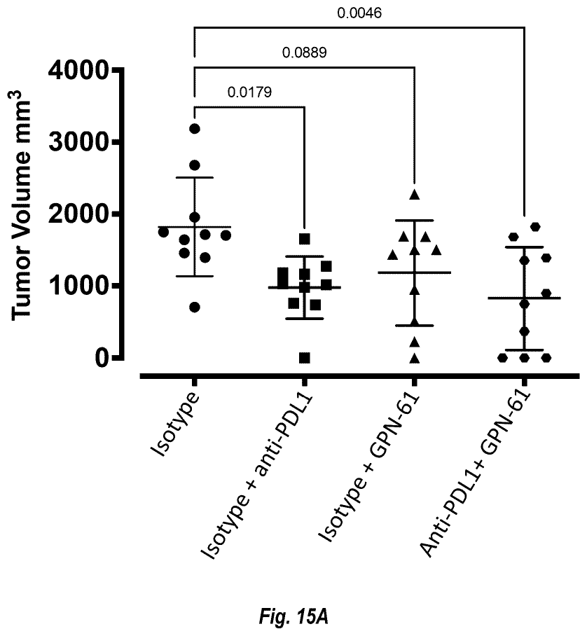

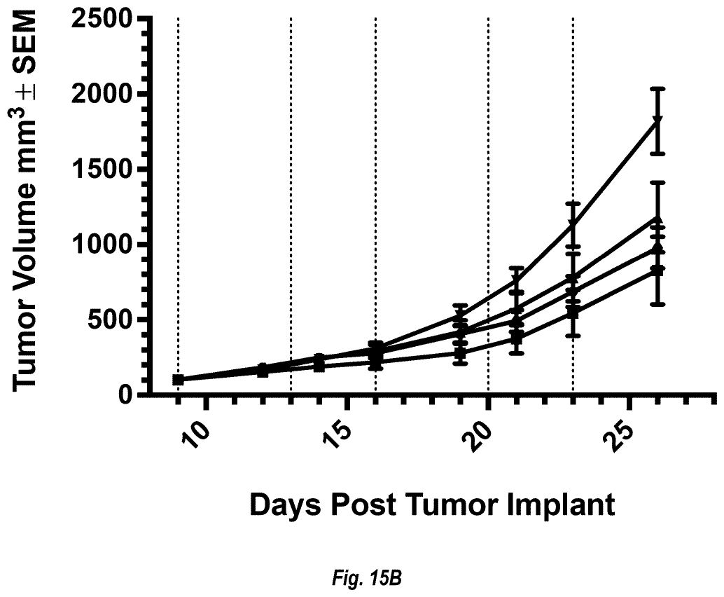

Average tumor volumes 26 days following inoculation were: 1,818 mm³±216.9 (isotype control antibody alone); 977.16 mm³±136.8 (isotype control antibody+anti-PDL-1 antibody); 1,180 mm³±231.1 (isotype control antibody+anti-GPNMB antibody GPN-61); and 825.9 6 mm³±225.8 (anti-GPNMB antibody GPN-61+anti-PDL1 antibody) (FIG. 15A and FIG. 15B). Compared to that observed in mice administered isotype control antibody alone, anti-GPNMB antibody GPN-61 was as efficacious as anti-PDL1 antibody in reducing tumor volume and rate of tumor growth. Combination treatment with anti-PDL1 and anti-GPNMB antibody GPN-61 resulted in the most significant decrease in tumor volume and rate of tumor growth. These results demonstrated in vivo efficacy of anti-GPNMB antibody GPN-61 in a mouse tumor model.

Taken together, these results showed that anti-GPNMB antibodies of the present disclosure were effective at reducing tumor volume and at reducing rate of tumor growth both as a monotherapy and in combination with anti-PDL1 antibody treatment.

### Example 18: Anti-GPNMB Antibodies Increase Glucocerebrosidase Activity In Vivo

Progranulin knockout mice (GRN−/−) have previously been shown to have reduced GCase activity and elevated levels of GPNMB mRNA in multiple tissues and cell types. The effect of anti-GPNMB antibodies on the activity of glucocerebrosidase activity in vivo in wildtype mice and in GRN−/− mice was examined as follows. Wild-type mice and progranulin knockout (GRN−/−) male mice of approximately 9 months of age were dosed twice per week for 2 weeks with anti-GPNMB antibody GPN-61 or an isotype control antibody (both at 10 mg/kg dose by i.p. injection). 24 hours following the 4^{th} and final dose, animals were euthanized, and whole blood and serum were harvested. Serum was analyzed for changes in expression levels of 31 cytokines and chemokines (Eve Technologies). Peripheral blood mononuclear cells were analyzed for changes in GCase activity as follows. 1 ml of ACK Lysis buffer was added to each well of a 24-well plate, to which 500 μl of whole blood was added; the samples were then incubated for 5 minutes at room temperature. Cells were spun at 300×G for 5 minutes and supernatant was discarded. Cell pellets were washed 1× with PBS and resuspended in 500 μl of RPMI-G media. C12-FDGlu was added to each well to a final concentration of 10 μM, mixed thoroughly, and incubated at 37° C. for 1 hour. Plates were transferred to ice, and 300 μL of cold FACs buffer was added to each well to stop the enzymatic reaction. Cells were pelleted, re-suspended in 50 μL FACs buffer+FcR block, incubated on ice for 10 minutes, and then stained with a FACs antibody panel consisting of: CD45 (APC-Cy7, BD Bioscience, Clone 30-F11), CD11b (BV605, Biolegend, Clone M1/70), Ly6C (PE-Cy7, Biolegend, Clone HK1.4), Ly6G (BV785, Clone), and CD115 (BV421).

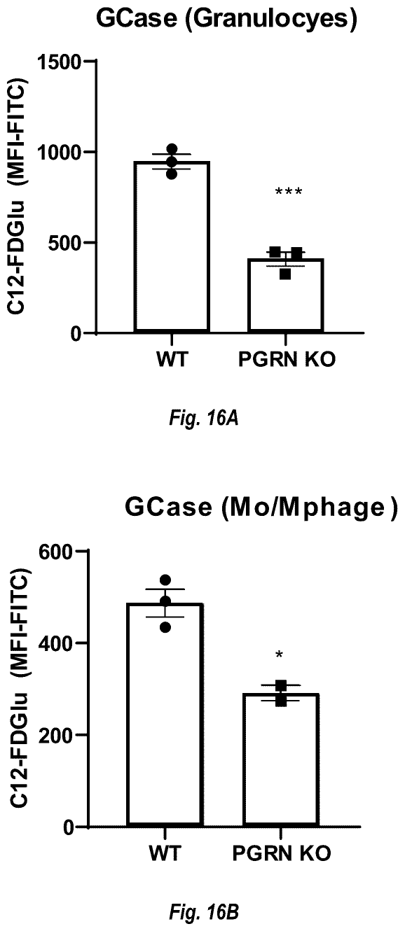

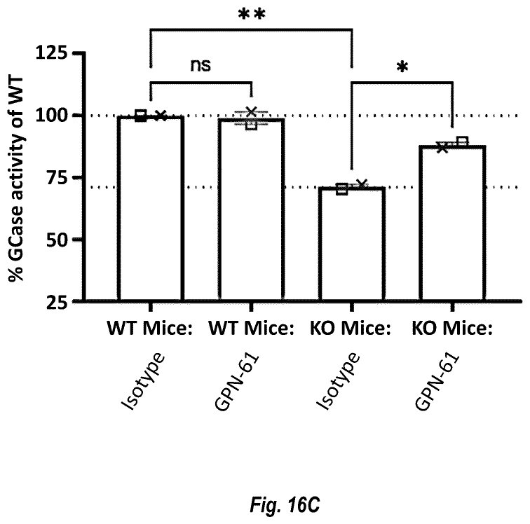

As shown in (FIG. 16A and FIG. 16B), a significant decrease in GCase activity in granulocytes and monocytes/macrophages (Mo/MΦ), respectively, was observed in GRN−/− (PGRN KO) mice compared to that observed in wildtype (WT) mice, confirming prior reports. Treatment of wildtype mice with anti-GPNMB antibody GPN-61 showed no change in GCase activity in monocytes (FIG. 16C). However, treatment of GRN−/− mice (KO Mice) with anti-GPNMB antibody GPN-61 resulted in higher GCase activity in monocytes compared to that observed in GRN−/− mice treated with isotype control antibody (FIG. 16C) Taken together, these results showed that anti-GPNMB antibodies of the present disclosure are effective at overcoming a decrease in GCase activity associated with reduced progranulin levels. Additionally, these results were consistent with the desired properties of an anti-GPNMB antibody therapeutic for use in treating disorders associated with lysosomal deficits.

### Example 19: Anti-GPNMB Antibodies Alter Serum Cytokine Levels In Vivo

The effect of anti-GPNMB antibodies on serum cytokine/chemokine levels in vivo in wildtype mice and in GRN−/− mice was examined as follows. Cytokine/Chemokine Analysis was performed by Eve Technologies, using their Discovery Assay Platform (MD31 array).

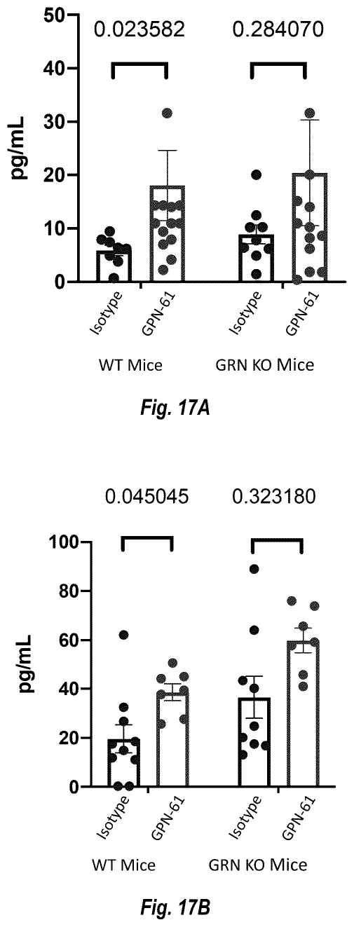

As shown in FIG. 17A (IL-12p40) and FIG. 17B (CCL5), treatment of either wildtype mice or GRN−/− (GRN KO Mice) with anti-GPNMB antibody GPN-61 resulted in increased levels of IL-12p40 and CCL5 in serum compared to that observed in isotype control antibody treated animals. These results indicated that anti-GPNMB antibody GPN-61 was efficacious in vivo and was able to affect the levels of multiple cytokines in serum.

### Example 20: Differential Gene Expression Analyses with Loss of GPNMB

A series of studies were performed to examine whether loss of GPNMB (in the context of CBE-induced neuroinflammation, used as a model to recapitulate neuroinflammation observed in Parkinson's disease) reduces a harmful pro-inflammatory response consistent with brain tissue of humans with Parkinson's disease.

Mice used in these studies were obtained as described above in Example 9. Mice were perfused with ice cold PBS at PsychoGenics and the hippocampus and cortex were immediately dissected and placed into 1.5 ml Eppendorf tubes filled with ice cold Hibernate A Low Fluorescence medium and kept at 4° C. Samples were transferred to an ice-cold petri dish, minced into smaller pieces with a fresh chilled razor blade, and then placed into a 2 ml Eppendorf tube with 1.6 ml of fresh ice cold hibernate. The tissue was manually triturated 7 times, each time passing the sample through a pre-wetted 70 μm filter. The resulting suspension was centrifuged at 400G for 5 minutes at 4° C. and the cell pellet was resuspended in 1 ml of hibernate. The cell suspension was loaded onto a discontinuous Percoll gradient having three different densities, centrifuged at 430G for 4 minutes at 4° C., followed by removal of the top 2 ml. The remaining cells were pelleted by at 550G centrifugation for 4 minutes. The supernatant was discarded, and the cell pellet was resuspended in 1 ml of ice cold Hibernate A for staining.

Microglia immunostaining, FACS, and RNA isolation was performed as follows. The cell suspension was kept in the cold room at 4° C. on a rotator for 20 minutes after addition of APC-conjugated anti-CD11b (561690, BD Biosciences) at 1:250. Cells were then pelleted by centrifugation at 400G for 5 minutes at 4° C., resuspended in 400 μl of Hibernate A, and 1 μl of DAPI (1 mg/ml stock) was added. The sample was passed one final time through a 40 μm filter into the FACs sorting tube before being ready to sort. Cells of appropriate size were first gated based on FSC-A vs SSC-A and then further identified as microglia based on DAPI-ve signal as a live/dead cell stain (DAPI-ve being live) and on a positive CD11b antibody signal. Microglia were collected into LoBind Eppendorf tubes that had 350 μl of RLT+BME media. Samples were stored at −80° C. until RNA isolation. RNA isolation was done on a later day and extracted from the suspension using the Qiagen RNeasy Plus Micro kit in a final volume of 16 ul of nuclease free water. RNA was sent to the service provider Lexogen located in Austria where RNA quality was quantified using a bioanalyzer and Illumina compatible libraries were created using the Lexogen QuantSeq 3′ mRNA-Seq FWD Library kit then sequenced on an Illumina NextSeq.

RNA sequencing was performed as follows. Sequencing libraries were prepared as per the instructions for the Lexogen Quant Seq 3′ end FWD library prep kit. Libraries were checked for quality and correct DNA banding then sequenced using an Illumina MiSeq v3 kit. Raw data was processed with the following steps: reads trimming, alignment to reference genome, sequence and alignment quality control, and differential expression analysis. TrimGalore tool was used to remove low-quality ends from reads (with quality score Phred<28) and reads shorter than 25 bp. STAR aligner (version 2.7.2b) was used to align reads to the mouse genome (mm10). Sequence and alignment quality were analyzed with FastQC and MultiQC tools respectively. All samples were of good quality with sufficient number of mapped reads (>2.5 million), and only 1 outlier were detected once viewing samples on Principal Component dimensions. This outlier was removed from further analysis.

Differentially expressed gene (DEG) analysis was performed as follows. Reads were normalized to the total number of reads per sample by dividing read counts by the total number of reads within a sample. Differential gene expression analysis was conducted using DESeq2, comparing gene expression profile changes under CBE and PBS (control) treatments within GPNMB WT and GPNMB KO animals. Mouse sex was regressed out to find DEGs robust to sex associated changes. p-value cutoff at less than 5%, and absolute value of log 2 fold change (FC) above 0.5. Applying Benjamini-Hochberg False Discovery Rate (FDR) gives significant DEGs.

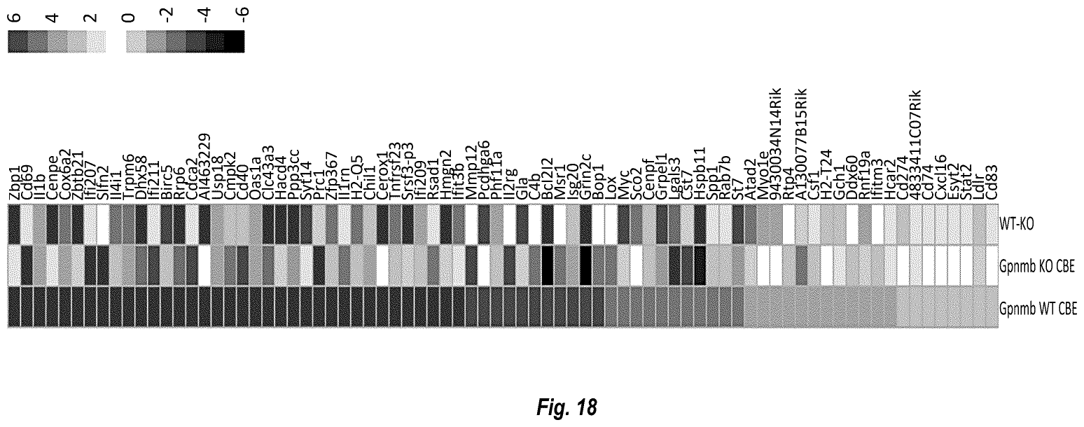

FIG. 18 shows a heatmap comparing DEGs from GPNMB knockout mice (WT-KO), GPNMB knockout mice treated with CBE (Gpnmb KO CBE), and wildtype mice treated with CBE (Gpnmb WT CBE). Data presented in FIG. 18 includes DEGs showing a greater fold-change (FC) in the GPNMB wildtype mice compared to that observed in GPNMB KO mice (defined as DEGs with a fold-change in wildtype mice at least 1.5× that seen in the GPNMB KO group). Using DAVID Functional Annotation Tool to identify enriched pathways, a selective enrichment of inflammatory genes related to antiviral response was revealed.

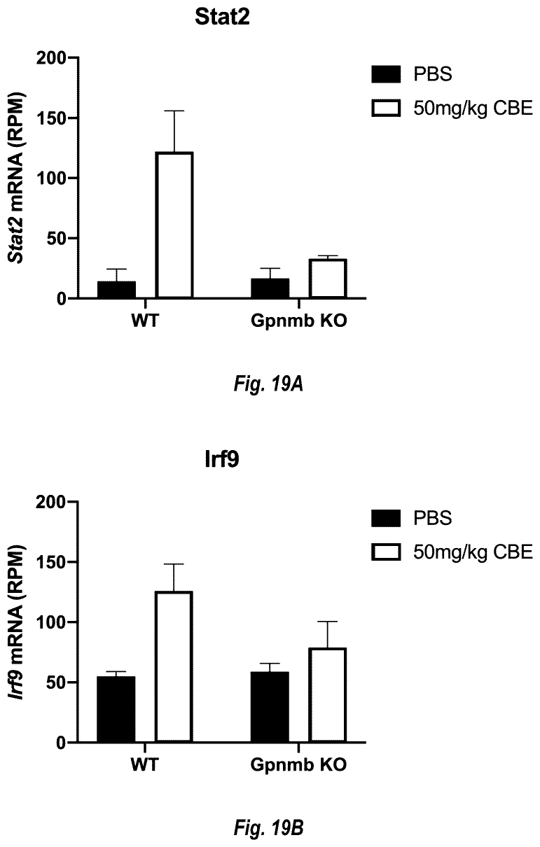

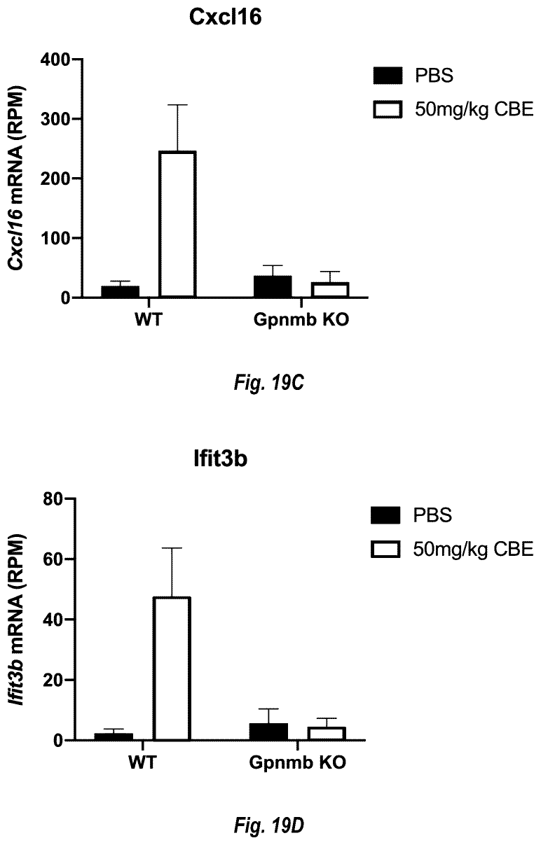

FIG. 19 shows a comparison of a select few of these antiviral, interferon-related genes showing differential expression (Stat2, Irf9, Cxcl16, and Ifit3b). These results showed a significant elevation in expression of these genes in WT animals in response to CBE that was either blunted or absent in GPNMB KO animals. RNA-seq readout showed that in microglia, there was a strong decrease in interferon response genes in GPNMB KO vs wildtype mice (both treated with CBE and normalized to PBS control).

### Example 21: Humanization of Anti-GPNMB Antibodies

Murine anti-GPNMB antibodies identified as described above were humanized as follows. One method of humanizing non-human antibodies is to transplant the CDRs from a non-human (e.g., murine) antibody onto a human antibody acceptor framework. Such CDR transplantation may result in attenuation or complete loss of affinity of the humanized antibody to its target due to perturbation in its framework. As a result, certain amino acid residues in the human framework may need to be replaced by amino acid residues from the corresponding positions of the murine antibody framework (back mutations) to restore attenuated or lost affinity. Therefore, the amino acid residues to be replaced in the context of the selected human antibody germline acceptor framework must be determined so that the humanized antibody substantially retains functions and paratopes. In addition, retained or improved thermal stability and solubility are desired for good manufacturability and downstream development.

Structure-based antibody modeling was applied in the process of humanizing mouse anti-GPNMB monoclonal antibodies GPN-08 and GPN-52 utilizing the BioMOE module of MOE (Molecular Operating Environment, Chemical Computing Group, Montreal, Canada). Briefly, Variable heavy chain (VH) and variable light chain (VL) amino acid sequences of the mouse anti-GPNMB monoclonal antibodies GPN-08 and GPN-52 were compared to human VL, VH, LJ, HJ functional germline amino acid sequences taken from IMGT (www.imgt.org/). Pseudo-genes and ORFs were excluded from analysis.

Per one mouse monoclonal antibody (query), one or two of the most similar VH and one or two of the most similar VL germline amino acid sequences were selected and combined with the most similar VJ and HJ genes, producing two to four humanized amino acid sequences. The CDRs to be transplanted onto the human framework were defined according to the AbM definition (www.bioinf.org.uk/abs/#cdrdef).

The query and the humanized amino acid sequences were used to create variable domain (Fv) homology models using BioMOE module or the Antibody Modeler module of MOE. AMBER10:EHT force field analysis was used for energy minimization through the entire antibody homology modeling process. Based on the Fv homology models obtained, molecular descriptors such as interaction energy between VL and VH, coordinate-based isoelectric point (3D pI), hydrophobic patch, and charged surface area were calculated, analyzed, and sorted by scoring metrics provided by MOE. These molecular descriptors were utilized to prioritize the humanized monoclonal antibodies for downstream experimental procedures, including protein expression, purification, binding affinity studies, and functional assays.

The BioMOE module of MOE provides a tool, Mutation Site Properties, to visualize and classify potential residues for back-mutation. In this context, back-mutation was defined as amino acid substitution which was reverted to the original query amino acid sequence replacing the humanized amino acid sequence. Using this tool, the original query (reference) was compared individually to the selected humanized variants for both the primary amino acid sequence and the 3D structure of the 3D Fv homology model.

Changes between the reference (i.e., parental) antibody and the humanized variant were classified based on amino acid type difference, interaction potential with CDR residues, impact potential for VL/VH pairing, and potential change in hydrophobic and charged surface area in and near the CDRs.

Mutations near the CDRs or the VL/VH interface having a significant charge difference or containing strong H-bond interactions were individually evaluated and the significantly disrupting mutations were reverted to the original query amino acid residues.

Twenty-six (26) different humanized variants of anti-GPNMB antibody GPN-08 and nine (9) different humanized variants of anti-GPNMB antibody GPN-52 were expressed and purified from Expi293 cells and screened for binding by SPR and functional activity as described below. Based on desirable parameters of the anti-GPNMB antibodies, including affinity, biological activity, expression yield, and developability, anti-GPNMB antibody GPN-81 and anti-GPNMB antibody GPN-92 were selected for affinity maturation.

Affinity maturation of humanized anti-GPNMB antibody GPN-81 and of humanized anti-GPNMB antibody GPN-92 were performed. Briefly, certain amino acid residues in the heavy chain or light chain were selectively mutagenized and mutants that improved binding were selected through additional rounds of screening. This process simultaneously improved specificity, species cross-reactivity, and developability profiles of the anti-GPNMB antibodies variants. These anti-GPNMB antibodies were characterized for affinity measurements by SPR and in functional assays on human macrophages as described below.

Amino acid sequences of the humanized and affinity matured variable heavy chains and variable light chains of anti-GPNMB antibodies of the present disclosure identified herein are provided below in Table 15.

TABLE 15

| | | SEQ | | SEQ |
|---|---|---|---|---|
| | | ID | | ID |
| Antibody | Heavy Chain Variable | NO: | Light Chain Variable | NO: |
| GPN-81 | QVQLQESGPGLVKPSETLSLTC | 234 | DIVMTQSPDSLAVSLGERATINC | 241 |
| | TVSGISITTGNYRWSWIRQPPG | | KSSESVDYYGIGFLNWYQQKPGQ | |
| | KGLEWIGYIYYSGTITYNPSLK | | PPKLLIYAASTQGSGVPDRFSGS | |
| | SRVTITRDTSKNQFSLELSSVT | | GSGTDETLTISSLQAEDVAVYYC | |
| | AADTAVYYCARDLTHWYFDVWG | | QQSKEVPPTFGGGTKVEIK | |
| | QGTLVTVSS | | | |
| GPN-82 | QVQLQESGPGLVKPSETLSLTC | 235 | DIVMTQSPDSLAVSLGERATINC | 242 |
| | TVSGISITTGNYRWSWIRQPPG | | KSSRSVDYYGIGELNWYQQKPGQ | |
| | KGLEWIGYIYYSGTIIYNPSLK | | PPKLLIYATSTQGSGVPDRFSGS | |
| | SRVTITRDTSKNQFSLELSSVT | | GSGTDFTLTISSLQAEDVAVYYC | |
| | AADTAVYYCARDLTHWYFDVWG | | QQQKEVPPTFGGGTKVEIK | |
| | QGTLVTVSS | | | |
| GPN-83 | QVQLQESGPGLVKPSETLSLTC | 236 | DIVMTQSPDSLAVSLGERATINC | 243 |
| | TVSGISITTGNYRWSWIRQPPG | | KSSRSVDYYGIGFVNWYQQKPGQ | |
| | KGLEWIGYIDYSGTITYNPSLK | | PPKLLIYATSIQGSGVPDRFSGS | |
| | SRVTITRDTSKNQFSLELSSVT | | GSGTDETLTISSLQAEDVAVYYC | |
| | AADTAVYYCARDLTHWYFDVWG | | QQQKEVPPTFGGGTKVEIK | |
| | QGTLVTVSS | | | |
| GPN-84 | QVQLQESGPGLVKPSETLSLTC | 237 | DIVMTQSPDSLAVSLGERATINC | 243 |
| | TVSGISITTGNYRWSWIRQPPG | | KSSRSVDYYGIGFVNWYQQKPGQ | |
| | KGLEWIGYIDYSGTITYNPSLK | | PPKLLIYATSIQGSGVPDRFSGS | |
| | SRVTITRDTSKNQFSLELSSVT | | GSGTDFTLTISSLQAEDVAVYYC | |
| | AADTAVYYCARDLTHWYYDVWG | | QQQKEVPPTFGGGTKVEIK | |
| | QGTLVTVSS | | | |
| GPN-85 | QVQLQESGPGLVKPSETLSLTC | 238 | DIVMTQSPDSLAVSLGERATINC | 244 |
| | TVSGISITTGNYRWSWIRQPPG | | KSSRSVDYFGIGELNWYQQKPGQ | |
| | KGLEWIGYIDYSGTITYNPSLK | | PPKLLIYATSHQGSGVPDRFSGS | |
| | QRVTITRDTSKNQFSLELSSVT | | GSGTDFTLTISSLQAEDVAVYYC | |
| | AADTAVYYCARDLTHWYFDVWG | | QQQKEVPPTFGGGTKVEIK | |
| | QGTLVTVSS | | | |
| GPN-86 | QVQLQESGPGLVKPSETLSLTC | 236 | DIVMTQSPDSLAVSLGERATINC | 245 |
| | TVSGISITTGNYRWSWIRQPPG | | KSSRSVDYYGIGFLNWYQQKPGQ | |
| | KGLEWIGYIDYSGTITYNPSLK | | PPKLLIYATSKQGSGVPDRFSGS | |
| | SRVTITRDTSKNQFSLELSSVT | | GSGTDFTLTISSLQAEDVAVYYC | |
| | AADTAVYYCARDLTHWYFDVWG | | QQQKEVPPTFGGGTKVEIK | |
| | QGTLVTVSS | | | |
| GPN-87 | QVQLQESGPGLVKPSETLSLTC | 237 | DIVMTQSPDSLAVSLGERATINC | 245 |
| | TVSGISITTGNYRWSWIRQPPG | | KSSRSVDYYGIGFLNWYQQKPGQ | |
| | KGLEWIGYIDYSGTITYNPSLK | | PPKLLIYATSKQGSGVPDRFSGS | |
| | SRVTITRDTSKNQFSLELSSVT | | GSGTDFTLTISSLQAEDVAVYYC | |
| | AADTAVYYCARDLTHWYYDVWG | | QQQKEVPPTFGGGTKVEIK | |
| | QGTLVTVSS | | | |
| GPN-88 | QVQLQESGPGLVKPSETLSLTC | 236 | DIVMTQSPDSLAVSLGERATINC | 246 |
| | TVSGISITTGNYRWSWIRQPPG | | KSSRSVDYYGIGFLNWYQQKPGQ | |
| | KGLEWIGYIDYSGTITYNPSLK | | PPKLLIYATSHQGSGVPDRFSGS | |
| | SRVTITRDTSKNQFSLELSSVT | | GSGTDETLTISSLQAEDVAVYYC | |
| | AADTAVYYCARDLTHWYFDVWG | | QQQKHVPPTFGGGTKVEIK | |
| | QGTLVTVSS | | | |
| GPN-89 | QVQLQESGPGLVKPSETLSLTC | 237 | DIVMTQSPDSLAVSLGERATINC | 247 |
| | TVSGISITTGNYRWSWIRQPPG | | KSSRSVDYYGIGFLNWYQQKPGQ | |
| | KGLEWIGYIDYSGTITYNPSLK | | PPKLLIYATSHQGSGVPDRFSGS | |
| | SRVTITRDTSKNQFSLELSSVT | | GSGTDFTLTISSLQAEDVAVYYC | |
| | AADTAVYYCARDLTHWYYDVWG | | QQQKEVPPTFGGGTKVEIK | |
| | QGTLVTVSS | | | |
| GPN-90 | QVQLQESGPGLVKPSETLSLTC | 238 | DIVMTQSPDSLAVSLGERATINC | 247 |
| | TVSGISITTGNYRWSWIRQPPG | | KSSRSVDYYGIGFLNWYQQKPGQ | |
| | KGLEWIGYIDYSGTITYNPSLK | | PPKLLIYATSHQGSGVPDRFSGS | |
| | QRVTITRDTSKNQFSLELSSVT | | GSGTDFTLTISSLQAEDVAVYYC | |
| | AADTAVYYCARDLTHWYFDVWG | | QQQKEVPPTFGGGTKVEIK | |
| | QGTLVTVSS | | | |
| GPN-91 | QVQLQESGPGLVKPSETLSLTC | 236 | DIVMTQSPDSLAVSLGERATINC | 248 |
| | TVSGISITTGNYRWSWIRQPPG | | KSSRSVDYYGIGFLNWYQQKPGQ | |
| | KGLEWIGYIDYSGTITYNPSLK | | PPKLLIYATSHQGSGVPDRFSGS | |
| | SRVTITRDTSKNQFSLELSSVT | | GSGTDFTLTISSLQAEDVAVYYC | |
| | AADTAVYYCARDLTHWYFDVWG | | QQQKERPPTFGGGTKVEIK | |
| | QGTLVTVSS | | | |
| GPN-92 | QVQLVQSGAEVKKPGASVKVSC | 239 | DIVMTQSPDSLAVSLGERATINC | 249 |
| | KASGYTFTSYWMHWVRQAPGQG | | KASQDVSPSLAWYQQKPGQPPKL | |
| | LEWMGEINPSDGRTNYAQKFQG | | LIYSASYRYTGVPDRFSGSGSGT | |
| | RVTMTVDKSTSTVYMELSSLRS | | DFTLTISSLQAEDVAVYYCQQHY | |
| | EDTAVYYCARSGGNYAWFAYWG | | SIPLTFGGGTKVEIK | |
| | QGTLVTVSS | | | |
| GPN-93 | QVQLVQSGAEVKKPGASVKVSC | 240 | DIVMTQSPDSLAVSLGERATINC | 250 |
| | KASGYTFTSYWMHWVRQAPGQG | | KTSQDVSPSLAWYQQKPGQPPKL | |
| | LEWMGEINPYDQRTNYAQKFQG | | LIYSASKRYTGVPDRFSGSGSGT | |
| | RVTMTVDKSTSTVYMELSSLRS | | DFTLTISSLQAEDVAVYYCQQHY | |
| | EDTAVYYCARSTGNYAWFAYWG | | SIPLQFGGGTKVEIK | |
| | QGTLVTVSS | | | |
| GPN-94 | QVQLVQSGAEVKKPGASVKVSC | 240 | DIVMTQSPDSLAVSLGERATINC | 251 |
| | KASGYTFTSYWMHWVRQAPGQG | | KTSQDVSPSLAWYQQKPGQPPKL | |
| | LEWMGEINPYDQRTNYAQKFQG | | LIYSASKRYTGVPDRFSGSGSGT | |
| | RVTMTVDKSTSTVYMELSSLRS | | DETLTISSLQAEDVAVYYCQQHY | |
| | EDTAVYYCARSTGNYAWFAYWG | | SIPLTFGGGTKVEIK | |
| | QGTLVTVSS | | | |
| GPN-95 | QVQLVQSGAEVKKPGASVKVSC | 240 | DIVMTQSPDSLAVSLGERATINC | 252 |
| | KASGYTFTSYWMHWVRQAPGQG | | KTSQDVSPSLAWYQQKPGQPPKL | |
| | LEWMGEINPYDQRTNYAQKFQG | | LIYSARYRYTGVPDRFSGSGSGT | |
| | RVTMTVDKSTSTVYMELSSLRS | | DFTLTISSLQAEDVAVYYCQQHY | |
| | EDTAVYYCARSTGNYAWFAYWG | | SIPLQFGGGTKVEIK | |
| | QGTLVTVSS | | | |
| GPN-96 | QVQLVQSGAEVKKPGASVKVSC | 240 | DIVMTQSPDSLAVSLGERATINC | 253 |
| | KASGYTFTSYWMHWVRQAPGQG | | KASQDVSPSLAWYQQKPGQPPKL | |
| | LEWMGEINPYDQRTNYAQKFQG | | LIYRASYRYTGVPDRFSGSGSGT | |
| | RVTMTVDKSTSTVYMELSSLRS | | DFTLTISSLQAEDVAVYYCQQHY | |
| | EDTAVYYCARSTGNYAWFAYWG | | SIPLTFGGGTKVEIK | |
| | QGTLVTVSS | | | |
| GPN-97 | QVQLVQSGAEVKKPGASVKVSC | 240 | DIVMTQSPDSLAVSLGERATINC | 254 |
| | KASGYTFTSYWMHWVRQAPGQG | | KTSQDVSPSLAWYQQKPGQPPKL | |
| | LEWMGEINPYDQRTNYAQKFQG | | LIYSGSYRYTGVPDRFSGSGSGT | |
| | RVTMTVDKSTSTVYMELSSLRS | | DFTLTISSLQAEDVAVYYCQQHY | |
| | EDTAVYYCARSTGNYAWFAYWG | | SIPLTFGGGTKVEIK | |
| | QGTLVTVSS | | | |
| GPN-98 | QVQLVQSGAEVKKPGASVKVSC | 240 | DIVMTQSPDSLAVSLGERATINC | 249 |
| | KASGYTFTSYWMHWVRQAPGQG | | KASQDVSPSLAWYQQKPGQPPKL | |
| | LEWMGEINPYDQRTNYAQKFQG | | LIYSASYRYTGVPDRFSGSGSGT | |
| | RVTMTVDKSTSTVYMELSSLRS | | DETLTISSLQAEDVAVYYCQQHY | |
| | EDTAVYYCARSTGNYAWFAYWG | | SIPLTFGGGTKVEIK | |
| | QGTLVTVSS | | | |

The CDR sequences according to Kabat for the anti-GPNMB-antibodies of the present disclosure of Table 15 above are provided below in Table 16 (heavy chain) and Table 17 (light chain).

TABLE 16

| | | SEQ | | SEQ | | SEQ |
|---|---|---|---|---|---|---|
| Antibody | HVR-H1 | ID NO: | HVR-H2 | ID NO: | HVR-H3 | ID NO: |
| GPN-81 | TGNYRWS | 71 | YIYYSGTITYN | 255 | DLTHWYFDV | 73 |
| | | | PSLKS | | | |
| GPN-82 | TGNYRWS | 71 | YIYYSGTIIYN | 256 | DLTHWYFDV | 73 |
| | | | PSLKS | | | |
| GPN-83 | TGNYRWS | 71 | YIDYSGTITYN | 257 | DLTHWYFDV | 73 |
| | | | PSLKS | | | |
| GPN-84 | TGNYRWS | 71 | YIDYSGTITYN | 257 | DLTHWYYDV | 261 |
| | | | PSLKS | | | |
| GPN-85 | TGNYRWS | 71 | YIDYSGTITYN | 258 | DLTHWYFDV | 73 |
| | | | PSLKQ | | | |
| GPN-86 | TGNYRWS | 71 | YIDYSGTITYN | 257 | DLTHWYFDV | 73 |
| | | | PSLKS | | | |
| GPN-87 | TGNYRWS | 71 | YIDYSGTITYN | 257 | DLTHWYYDV | 261 |
| | | | PSLKS | | | |
| GPN-88 | TGNYRWS | 71 | YIDYSGTITYN | 257 | DLTHWYFDV | 73 |
| | | | PSLKS | | | |
| GPN-89 | TGNYRWS | 71 | YIDYSGTITYN | 257 | DLTHWYYDV | 261 |
| | | | PSLKS | | | |
| GPN-90 | TGNYRWS | 71 | YIDYSGTITYN | 258 | DLTHWYFDV | 73 |
| | | | PSLKQ | | | |
| GPN-91 | TGNYRWS | 71 | YIDYSGTITYN | 257 | DLTHWYFDV | 73 |
| | | | PSLKS | | | |
| GPN-92 | SYWMH | 23 | EINPSDGRTNY | 259 | SGGNYAWFAY | 19 |
| | | | AQKFQG | | | |
| GPN-93 | SYWMH | 23 | EINPYDQRTNY | 260 | STGNYAWFAY | 262 |
| | | | AQKFQG | | | |
| GPN-94 | SYWMH | 23 | EINPYDQRTNY | 260 | STGNYAWFAY | 262 |
| | | | AQKFQG | | | |
| GPN-95 | SYWMH | 23 | EINPYDQRTNY | 260 | STGNYAWFAY | 262 |
| | | | AQKFQG | | | |
| GPN-96 | SYWMH | 23 | EINPYDQRTNY | 260 | STGNYAWFAY | 262 |
| | | | AQKFQG | | | |
| GPN-97 | SYWMH | 23 | EINPYDQRTNY | 260 | STGNYAWFAY | 262 |
| | | | AQKFQG | | | |
| GPN-98 | SYWMH | 23 | EINPYDQRTNY | 260 | STGNYAWFAY | 262 |
| | | | AQKFQG | | | |

TABLE 17

| | | SEQ | | SEQ | | SEQ |
|---|---|---|---|---|---|---|
| Antibody | HVR-L1 | ID NO: | HVR-L2 | ID NO: | HVR-L3 | ID NO: |
| GPN-81 | KSSESVDY | 263 | AASTQGS | 269 | QQSKEVPPT | 151 |
| | YGIGFLN | | | | | |
| GPN-82 | KSSRSVDY | 264 | ATSTQGS | 270 | QQQKEVPPT | 278 |
| | YGIGFLN | | | | | |
| GPN-83 | KSSRSVDY | 265 | ATSIQGS | 271 | QQQKEVPPT | 278 |
| | YGIGFVN | | | | | |
| GPN-84 | KSSRSVDY | 265 | ATSIQGS | 271 | QQQKEVPPT | 278 |
| | YGIGFVN | | | | | |
| GPN-85 | KSSRSVDY | 266 | ATSHQGS | 272 | QQQKEVPPT | 278 |
| | FGIGFLN | | | | | |
| GPN-86 | KSSRSVDY | 264 | ATSKQGS | 273 | QQQKEVPPT | 278 |
| | YGIGFLN | | | | | |
| GPN-87 | KSSRSVDY | 264 | ATSKQGS | 273 | QQQKEVPPT | 278 |
| | YGIGELN | | | | | |
| GPN-88 | KSSRSVDY | 264 | ATSHQGS | 272 | QQQKHVPPT | 279 |
| | YGIGFLN | | | | | |
| GPN-89 | KSSRSVDY | 264 | ATSHQGS | 272 | QQQKEVPPT | 278 |
| | YGIGFLN | | | | | |
| GPN-90 | KSSRSVDY | 264 | ATSHQGS | 272 | QQQKEVPPT | 278 |
| | YGIGFLN | | | | | |
| GPN-91 | KSSRSVDY | 264 | ATSHQGS | 272 | QQQKERPPT | 280 |
| | YGIGFLN | | | | | |
| GPN-92 | KASQDVSP | 267 | SASYRYT | 96 | QQHYSIPLT | 97 |
| | SLA | | | | | |
| GPN-93 | KTSQDVSP | 268 | SASKRYT | 274 | QQHYSIPLQ | 281 |
| | SLA | | | | | |
| GPN-94 | KTSQDVSP | 268 | SASKRYT | 274 | QQHYSIPLT | 97 |
| | SLA | | | | | |
| GPN-95 | KTSQDVSP | 268 | SARYRYT | 275 | QQHYSIPLQ | 281 |
| | SLA | | | | | |
| GPN-96 | KASQDVSP | 267 | RASYRYT | 276 | QQHYSIPLT | 97 |
| | SLA | | | | | |
| GPN-97 | KTSQDVSP | 268 | SGSYRYT | 277 | QQHYSIPLT | 97 |
| | SLA | | | | | |
| GPN-98 | KASQDVSP | 267 | SASYRYT | 96 | QQHYSIPLT | 97 |
| | SLA | | | | | |

### Example 22: Binding Kinetics of Humanized and Affinity Matured Anti-GPNMB Antibodies

Binding kinetics of the humanized and affinity matured anti-GPNMB antibodies described above were evaluated using the Biacore T200 (Cytiva). Briefly, IgGs were diluted to 10 μg/mL and captured using a Protein A/G (ThermioFisher, #21186) surface on a CM5 chip that was prepared by amine coupling according to the instrument manufacturer's recommendations. The captured antibodies were tested for their ability to bind human, cynomolgus monkey, and mouse GPNMB recombinant proteins as follows.

Human, cynomolgus, and mouse GPNMB analytes were diluted in running buffer (HBS-EP+, Teknova, #8022, with 0.5 mg/ml BSA, MP Biomedicals LLC, #820451), to a concentration of 300 nM, then diluted 3-fold serially to 100 nM, 33 nM, and 11 nM. Each sample was injected for 2 minutes to allow association, followed by dissociation in buffer alone for 10 minutes. Each sample injection was followed by the regeneration of the chip with three 30-second injections of 10 mM glycine, pH 1.7. Fresh antibody was captured at the beginning of each cycle.

Data were analyzed using Biacore evaluation software to generate kinetic constants. The equilibrium dissociation constants (KD) were calculated from the fitted association and dissociation rate constants (k-on and k-off) for each of the anti-GPNMB antibodies.

Additionally, one of the anti-GPNMB antibodies of the present disclosure was evaluated using a Carterra LSA instrument (Carterra, Salt Lake City, UT). Briefly, the anti-GPNMB antibody was immobilized in duplicate spots onto an HC30M sensor chip (Carterra) by amine coupling according to the instrument manufacturer's recommended protocol. After priming with running buffer (see above) the immobilized antibody was tested for its ability to bind to recombinant human, cynomolgus, and mouse GPNMB. Analytes were diluted to 600 nM in running buffer, then further diluted 3-fold serially to 200 nM, 67 nM, 22.2 nM, 7.4 nM, and 2.5 nM. Analyte at each concentration was injected for five minutes over the entire chip using the single channel flow cell. Dissociation was measured for 5 min, followed by regeneration of the chip with two 30-second injections of 10 mM glycine, pH 2.0. The data were analyzed using the NextGenKIT high-throughput kinetics analysis software from Carterra. The KD values from both methods are summarized in Table 18 below.

As shown in Table 18, anti-GPNMB antibody GPN-81 and its engineered variants (anti-GPNMB antibodies GPN-82, GPN-83, GPN-84, GPN-85, GPN-86, GPN-87, GPN-88, GPN-89, GPN-90, and GPN-91) bound to all three species of GPNMB. The binding affinities of these engineered anti-GPNMB antibody variants ranged from approximately 1 nM to 14 nM for human and cynomolgus GPNMB, and 0.5 nM to 3 nM for mouse GPNMB, compared to a binding affinity of over 130 nM to human GPNMB and 12 nM to mouse GPNMB for the parental anti-GPNMB antibody GPN-81.

Anti-GPNMB antibody GPN-92 and its engineered variants (anti-GPNMB antibodies GPN-93, GPN-94, PGN-95, GPN-96, GPN-97, and GPN-98) bound to human and cynomolgus GPNMB, but were not-cross-reactive to mouse GPNMB. The binding affinities of these engineered anti-GPNMB antibody variants ranged from approximately 3 nM to 15 nM for human and cynomolgus GPNMB, compared to a binding affinity of over 60 nM to human GPNMB for the parental anti-GPNMB antibody GPN-92. Table 18 below provides kinetic rate constant values for engineered anti-GPNMB antibodies of the present disclosure binding to human, cynomolgus, or mouse GPNMB.

TABLE 18

| | Human GPNMB | | | Cynomolgus GPNMB | | | Mouse GPNMB | | |
|---|---|---|---|---|---|---|---|---|---|
| | ka | kd | KD | ka | kd | KD | ka | kd | KD |
| Ab ID | (1/Ms) | (1/s) | (nM) | (1/Ms) | (1/s) | (nM) | (1/Ms) | (1/s) | (nM) |
| GPN-81* | 7.6E+04 | 9.8E−03 | 135 | 9.7E+04 | 1.0E−02 | 107 | 1.8E+05 | 1.9E−03 | 12 |
| GPN-82 | 2.0E+05 | 2.4E−03 | 11.9 | 1.6E+05 | 2.2E−03 | 13.5 | 1.5E+05 | 3.9E−04 | 2.6 |
| GPN-83 | 2.1E+05 | 1.6E−03 | 7.5 | 1.7E+05 | 1.4E−03 | 8.1 | 1.6E+05 | 2.4E−04 | 1.5 |
| GPN-84 | 2.2E+05 | 2.1E−03 | 9.6 | 1.8E+05 | 1.9E−03 | 10.3 | 1.6E+05 | 3.4E−04 | 2.1 |
| GPN-85 | 1.7E+05 | 1.8E−04 | 1.0 | 1.5E+05 | 2.1E−04 | 1.4 | 1.4E+05 | 7.4E−05 | 0.5 |
| GPN-86 | 2.6E+05 | 1.2E−03 | 4.5 | 1.9E+05 | 1.1E−03 | 5.5 | 1.9E+05 | 2.3E−04 | 1.2 |
| GPN-87 | 2.5E+05 | 1.3E−03 | 5.4 | 1.9E+05 | 1.2E−03 | 6.2 | 1.9E+05 | 2.8E−04 | 1.5 |
| GPN-88 | 2.0E+05 | 6.3E−04 | 3.1 | 1.6E+05 | 6.1E−04 | 3.7 | 1.5E+05 | 1.7E−04 | 1.1 |
| GPN-89 | 2.0E+05 | 5.7E−04 | 2.9 | 1.7E+05 | 5.5E−04 | 3.3 | 1.6E+05 | 1.2E−04 | 0.8 |
| GPN-90 | 1.9E+05 | 5.3E−04 | 2.8 | 1.6E+05 | 5.1E−04 | 3.2 | 1.6E+05 | 1.1E−04 | 0.7 |
| GPN-91 | 1.8E+05 | 5.3E−04 | 3.0 | 1.5E+05 | 5.2E−04 | 3.6 | 1.4E+05 | 1.4E−04 | 1.1 |
| GPN-92 | 7.5E+04 | 5.0E−03 | 66.5 | 8.3E+04 | 5.7E−03 | 67.8 | nb | nb | nb |
| GPN-93 | 6.8E+04 | 5.9E−04 | 8.7 | 6.7E+04 | 7.1E−04 | 10.6 | nb | nb | nb |
| GPN-94 | 7.8E+04 | 4.2E−04 | 5.4 | 7.8E+04 | 5.1E−04 | 6.5 | nb | nb | nb |
| GPN-95 | 7.3E+04 | 5.3E−04 | 7.3 | 7.1E+04 | 6.0E−04 | 8.3 | nb | nb | nb |
| GPN-96 | 7.8E+04 | 2.0E−04 | 2.5 | 7.7E+04 | 2.5E−04 | 3.2 | nb | nb | nb |
| GPN-97 | 8.1E+04 | 5.4E−04 | 6.7 | 7.9E+04 | 6.5E−04 | 8.2 | nb | nb | nb |
| GPN-98 | 7.6E+04 | 9.1E−04 | 11.9 | 7.4E+04 | 1.1E−03 | 14.6 | nb | nb | nb |

*Kinetics measured on the Carterra SPR instrument; all others measured on Biacore T200
nb = no binding observed

### Example 23: GPNMB Exacerbates Neuroinflammation an Animal Model of Gaucher's Disease

Beginning at 8-12 weeks of age, mice were dosed with PBS or 50 mg/kg Conduritol B-epoxide (CBE) daily via IP injection for 28 days. Dosing and takedown/tissue harvest of mice was performed by Psychogenics (Paramus, NJ). Mice (gender mixed) were purchased from Jackson Laboratory [Stock 400748, DBA/2J-Gpnmb+/SjJ (Gpnmb wildtype) and Stock #000671, DBA/2J (Gpnmb knockout)].

At takedown, a terminal bleed was performed to obtain blood for serum cytokine analysis and blood cell GCase enzyme activity assay. Mice were perfused with PBS and the brains were extracted and micro-dissected to obtain cortex, hippocampus, midbrain, and cerebellum tissues. For FACS sorting of microglia, cortex/hippocampus tissue was shipped overnight from Psychogenics in Hibernate solution at 4° C. For protein Western blot analysis and GCase assay experiments, various brain regions were flash frozen in liquid nitrogen. For some mice, one hemisphere was drop-fixed in 4% PFA and embedded in OCT, followed by slicing and mounting of the tissue on slides for immunohistochemistry (IHC) analysis.

Frozen cortex samples were lysed in N-PER lysis reagent+protease inhibitor cocktail. Protein concentrations were measured using BCA reagent assay and 50 μg of protein per sample was loaded and run on SDS-PAGE gel. Total protein was measured via imaging StainFree gel on Biorad imager, and then samples were transferred to PDVF membrane and blocked in either 4% BSA or dry milk in TBST (depending on primary antibody specifications) for 1 hour at room temperature. Membranes were then incubated with indicated antibodies in blocking solution overnight at 4° C. Membranes were washed 3× in TBST and then incubated in appropriate HRP-conjugated secondary antibody in blocking solution for 1 hour. Membranes were again washed 3× and then incubated with SuperSignal West Pico Plus chemiluminescent substrate for 5 minutes and imaged on BioRad imager. Protein bands were analyzed in BioRad Image Lab software and intensities were normalized to loading control (GAPDH, β-Actin, or total protein from StainFree Gel image).

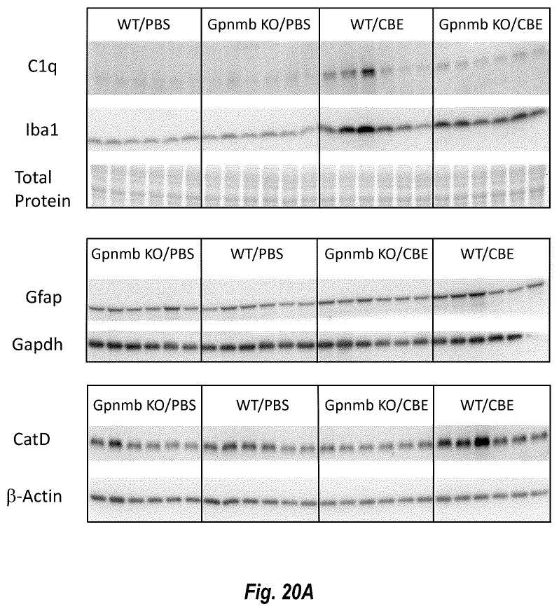

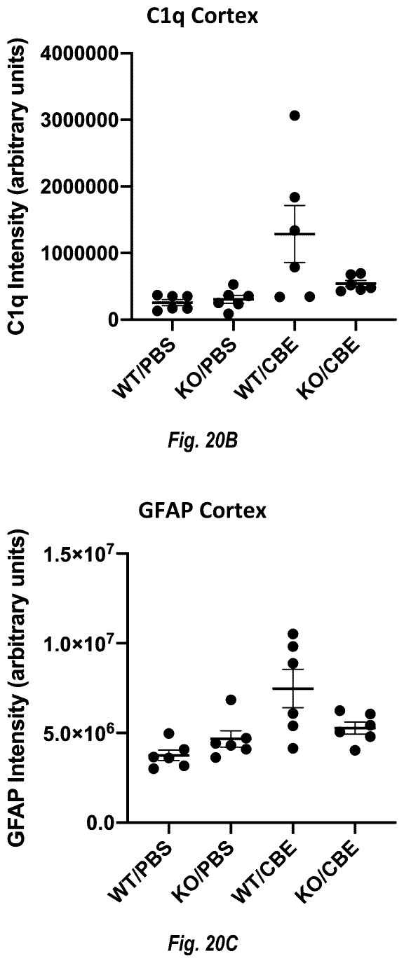

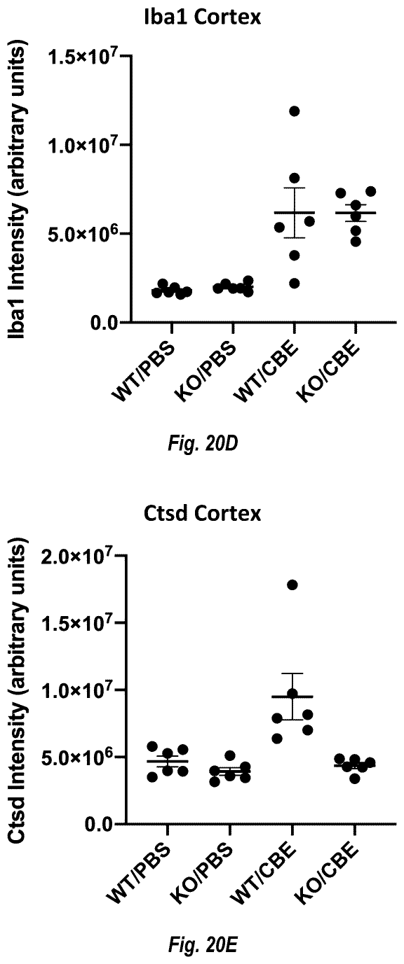

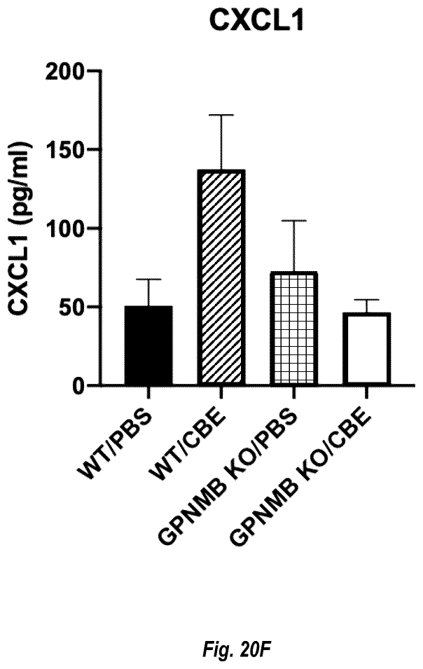

As shown in FIG. 20A, CBE treatment induced a significant upregulation of C1q (complement component 1q), GFAP (glial fibrillary acidic protein), IBA1 (ionized calcium-binding adaptor molecule 1), and CTSD (cathepsin D, CatD) in the cortex of wildtype (WT) animals. The upregulation observed were blunted in GPNMB knockout animals for C1q, GFAP, and CTSD. The increase in IBA1 staining observed in these studies was equivalent in wildtype and GPNMB knockout animals, suggesting the microgliosis induced by CBE is GPNMB-independent, but perhaps the resulting neuroinflammation is reduced. FIG. 20B-FIG. 20E show the relative intensities of the indicated bands detected by western blots and demonstrates significant upregulation of neuroinflammatory markers in CBE-treated wildtype mice compared to GPNMB knockout mice.

Terminal blood samples were incubated at room temperature to allow coagulation, and samples were spun down to pellet platelets; serum was collected from the supernatant. Serum samples were submitted to Eve Technologies (Calgary, AB, Canada) for measurement of cytokines/chemokines using a bead-based multiplex assay.

The results of this assay (FIG. 20F) revealed a significant increase in serum CXCL1 (CXC motif chemokine ligand 1) levels in wildtype mice treated with CBE compared to control PBS treated wildtype mice. GPNMB knockout mice did not show any significant differences in CXCL1 levels when treated with CBE. These results suggested a role for GPNMB in regulating induction of peripheral inflammation following CBE treatment in mice.

### Example 24: Inhibiting Inflammasome Activation with Anti-GPNMB Antibodies

Inflammation has been implicated as a key contributor to the pathogenesis of neurodegeneration, and recent studies have identified inflammasomes as critical mediators of the inflammatory response in the brain. Inflammasomes are multi-protein complexes that play an important role in innate immunity and inflammation. Activation of inflammasomes leads to the maturation and secretion of pro-inflammatory cytokines, such as interleukin-1β (IL-1β), and the induction of pyroptosis, a form of programmed cell death. Therefore, methods for inhibiting inflammasomes are of great interest for the treatment of neurodegenerative disorders.

In the following series of experiments, a method for inhibiting inflammasome activation on human macrophages with anti-GPNMB antibodies of the present disclosure is described. Human macrophages were generated from CD14+ monocytes enriched from whole blood of healthy volunteers. Briefly, monocytes from peripheral human blood samples were isolated using the RosetteSep™ monocyte isolation antibody cocktail (StemCell Technologies) and differentiated into macrophages with 100 ng/ml human M-CSF (StemCell Technologies) and cultured for 7 days. Cells were plated on culture dishes in RPMI 1640 medium (Invitrogen) containing 10% fetal calf serum (Hyclone) and 20 mM HEPES and cultured at 37° C. in 5% CO₂. On day 7, human macrophages were harvested and seeded at 100,000 cells per well in 96-well plates. Cells were treated with 20 ng/ml MCSF and indicated for 48 hours with anti-GPNMB antibodies of the present disclosure or isotype control antibodies. In some experiments, macrophages were also treated with 100 μM conduritol B epoxide (CBE), a chemical inhibitor of GBA enzyme, to exacerbate inflammasome activation. On the second day of incubation, macrophages were primed with 625 ng/mL of lipopolysaccharide (LPS; Invivogen) for 6 hours at 37° C. Subsequently, macrophages were activated with 2-3 μM nigericin (Adipogen) for 1 hour at 37° C. Cells were then pelleted and the supernatant fraction collected to measure IL-1β concentration by ELISA following manufacturer's instructions (R&D Systems).

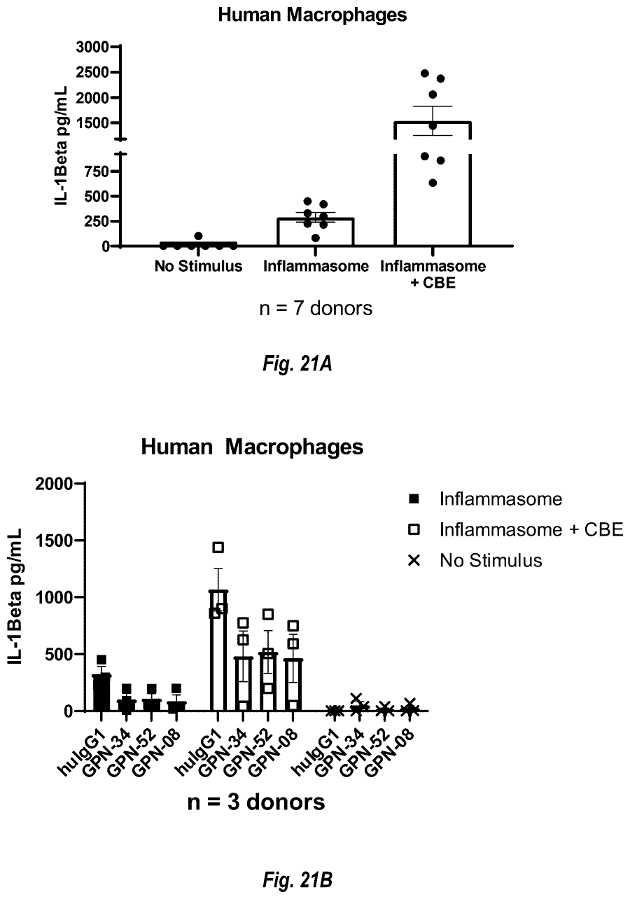

As shown in FIG. 21A, human macrophages stimulated with LPS+nigericin to activate inflammasomes induced a significant increase in IL-1β release relative to cells treated with vehicle control (average value of 290 μg/ml vs 16 μg/ml, respectively). Additionally, in the presence of the GCase inhibitor, CBE, LPS+nigericin stimulation elicited a significant increase in IL-1β release in macrophages relative to cells stimulated in the absence of CBE (average value of 1,535 μg/ml vs 290 μg/ml, respectively). These results are consistent with previous reports implicating lysosome function, and GCase activity, in modulating inflammasome activation. Human macrophages pre-treated with 1 μg/ml anti-GPNMB antibodies were stimulated with LPS+nigericin to activate inflammasomes. Results shown in FIG. 21B demonstrate that anti-GPNMB antibodies GPN-34, GPN-52, and GPN-08 inhibited inflammasome activation in macrophages as measured by IL-1β release. Furthermore, anti-GPNMB antibodies of the present disclosure also inhibited inflammasome activation in macrophages in the presence of CBE treatment. Taken together, these results suggested that GPNMB plays a role in inflammasome activation, likely through regulating lysosome function, and blocking GPNMB activity (for example, by using anti-GPNMB antibodies of the present disclosure) may suppress inflammasome-mediated inflammation and improve lysosome function.

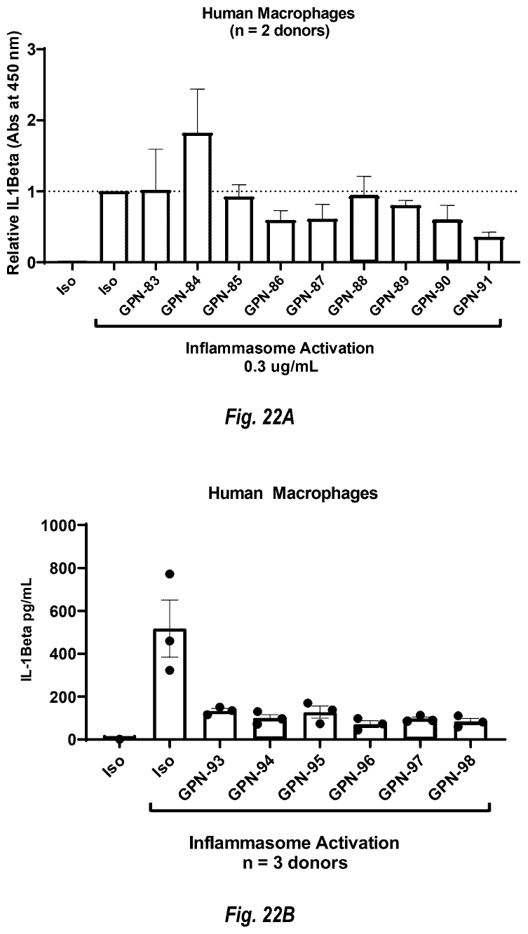

Affinity matured anti-GPNMB antibody variants were expressed on a human IgG1 wildtype backbone and screened for inhibition of IL-1β release from human macrophages. Human macrophages were pre-treated with 0.3 μg/ml or 1 μg/ml anti-GPNMB antibodies for 48 hours and then stimulated with LPS+nigericin to activate inflammasomes. As shown in FIG. 22A, human macrophages pre-treated with 0.3 μg/ml of anti-GPNMB antibody variants from the anti-GPNMB antibody GPN-52 lineage identified several anti-GPNMB antibodies capable of inhibiting IL-1β release in this assay. Similarly, FIG. 22B shows that antibodies from the anti-GPNMB antibody GPN-08 lineage reduced IL-1β release when added to human macrophages at 1 μg/ml. Data from these studies comparing the inflammasome inhibition by anti-GPNMB antibodies as measured by IL-1β levels in human macrophages are also provide below in Table 19.

TABLE 19

| | Inflammasome |
|---|---|
| | Fold Decrease |
| | IL-1β (max response |
| Antibody | at 0.3 μg/ml) |
| GPN-82 | 0.378 |
| GPN-83 | 0.472 |
| GPN-85 | 0.601 |
| GPN-86 | 0.404 |
| GPN-87 | 0.428 |
| GPN-89 | 0.501 |
| GPN-90 | 0.564 |
| GPN-93 | 0.594 |
| GPN-94 | 0.532 |
| GPN-95 | 0.450 |
| GPN-96 | 0.525 |
| GPN-97 | 0.476 |
| GPN-98 | 0.664 |

Anti-GPNMB antibodies of the present disclosure were further characterized on different human IgG Fc backbones to determine the contribution of Fc receptors on antibody-mediated inhibition of inflammasome activation. Anti-GPNMB antibodies of the present disclosure were engineered with the following Fc domains: human IgG1 wildtype Fc; human IgG1 N325S/L328F (NSLF) Fc; human IgG1 G236A/S239D/A330L/I332E (GASDALIE) Fc; human IgG1 L234A/L235A/P331S (LALAPS) Fc; and human IgG4 S228P. NSLF Fc mutations abrogate human IgG1 binding to CD16 to remove ADCC effector function; LALAPS Fc mutations abrogate human IgG1 binding to all Fc receptors to eliminate Fe-mediated effector functions; and GASDALIE Fe mutations enhance human IgG1 binding to activating Fe receptors (e.g., CD64, CD32A, and CD16) to promote cellular activation. Amino acid numbering of human Fe regions used herein is according to EU numbering.

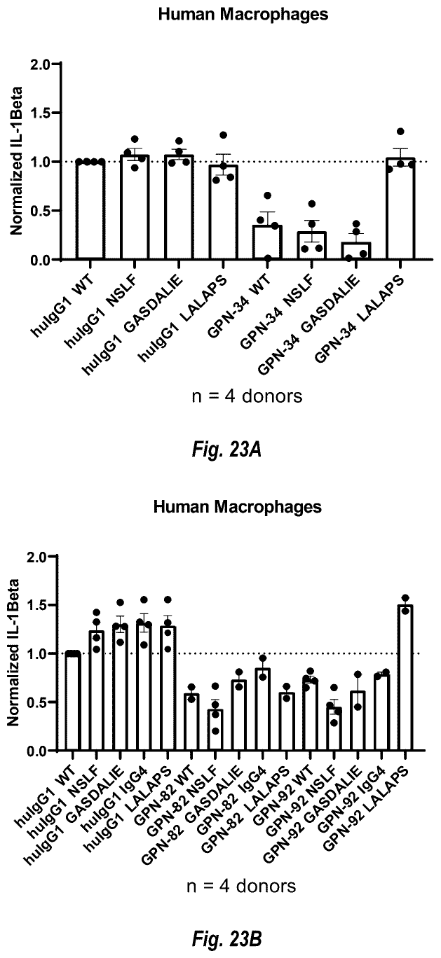

As shown in FIG. 23A, Fe variants of anti-GPNMB antibody GPN-34 that engage Fe receptors (huIgG1 WT, NSLF, GASDALIE) retained the ability to inhibit inflammasome activation on human macrophages. However, abrogating Fe receptor engagement by introducing the LALAPS Fe mutations to anti-GPNMB antibody GPN-34 abolished the ability to inhibit IL-1β release from stimulated macrophages. Similarly, human IgG1 and IgG4 Fe variants of anti-GPNMB antibody GPN-08 that engage Fe receptors reduced IL-1β release in human macrophages, whereas the LALAPS Fe variant displayed no activity in this assay (FIG. 23B). In contrast, all human IgG1 Fe variants of anti-GPNMB antibody GPN-52 demonstrated IL-1β inhibition on activated macrophages with human IgG1 wildtype and LALAPS Fe variants showing equivalent activity. These results further confirmed that anti-GPNMB antibodies of the present disclosure inhibited inflammasome activation on human macrophages. Additionally, these results showed that certain anti-GPNMB antibodies of the present disclosure (e.g., GPN-34 and GPN-08) depended, at least in part, on Fe receptor for reducing IL-1β expression and inhibition of inflammasome activation, whereas other anti-GPNMB antibodies (e.g., GPN-52) appear to function independently of Fe receptor engagement for reducing IL-IP expression and inhibition of inflammasome activation.

### Example 25: Anti-GPNMB Antibodies Rescue Rapamycin-Induced Lysosome Stress

Lysosomes play a critical role in cellular degradation and recycling. Rapamycin is a widely used drug that inhibits the mammalian target of rapamycin (mTOR) pathway and has been shown to induce autophagy and lysosome biogenesis, as well as increase lysosome volume. While autophagy is generally a beneficial process that helps to maintain cellular homeostasis and prevent the accumulation of toxic aggregates, excessive autophagy can be detrimental, particularly in the context of neurodegeneration. In neurodegenerative diseases such as Alzheimer's disease, Parkinson's disease, and Huntington's disease, there is evidence showing that autophagy is dysregulated, leading to the accumulation of protein aggregates and the death of neurons. In some cases, rapamycin-induced autophagy can exacerbate these effects by promoting excessive degradation of essential cellular components, including proteins and organelles that are necessary for neuronal survival.

In the following experiments, a method for inhibiting rapamycin-induced lysosomal stress on human macrophages with anti-GPNMB antibodies of the present disclosure is described. Human macrophages were generated from CD14+ monocytes enriched from whole blood of healthy volunteers. Briefly, monocytes from peripheral human blood samples were isolated using the RosetteSep™ monocyte isolation antibody cocktail (StemCell Technologies) and differentiated into macrophages with 100 ng/mL human M-CSF (StemCell Technologies) and cultured for 7 days. Cells were plated on culture dishes in RPMI 1640 medium (Invitrogen) containing 10% fetal calf serum (Hyclone) and 20 mM HEPES and cultured at 37° C. in 5% CO₂. On day 7, human macrophages were harvested and seeded at 100,000 cells per well in 96-well plates. Cells were treated with 100 nM rapamycin and indicated anti-GPNMB antibodies or isotype control antibody for 48 hours. As a negative control, cells were treated with vehicle alone and isotype control antibody. Following treatment, cells were pelleted and stained with 200 nM Lysotracker Red fluorescent dye for 15 minutes at 37° C. Cells were washed twice with FACS buffer (PBS supplemented with 2% FBS, 2 mM EDTA) and resuspended in FACS buffer supplemented with Sytox Green Live/Dead cell stain (Thermo Fisher) at 1:1000 dilution. Samples were acquired on a BD FACS Canto and Lysotracker fluorescence recorded on live cells. Data analysis and calculation of mean fluorescence intensity (MFI) values was performed with FlowJo (TreeStar) software version 10.0.8.

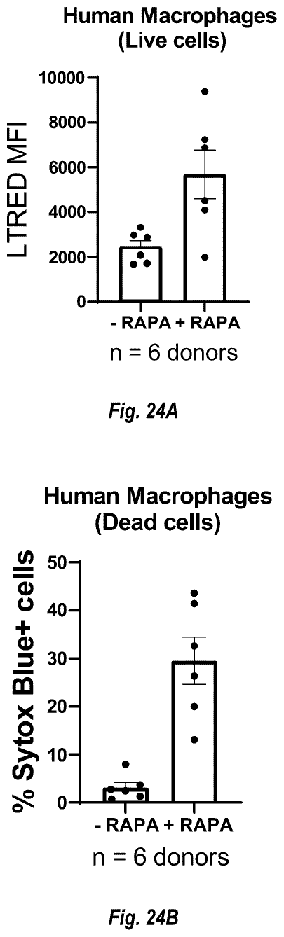

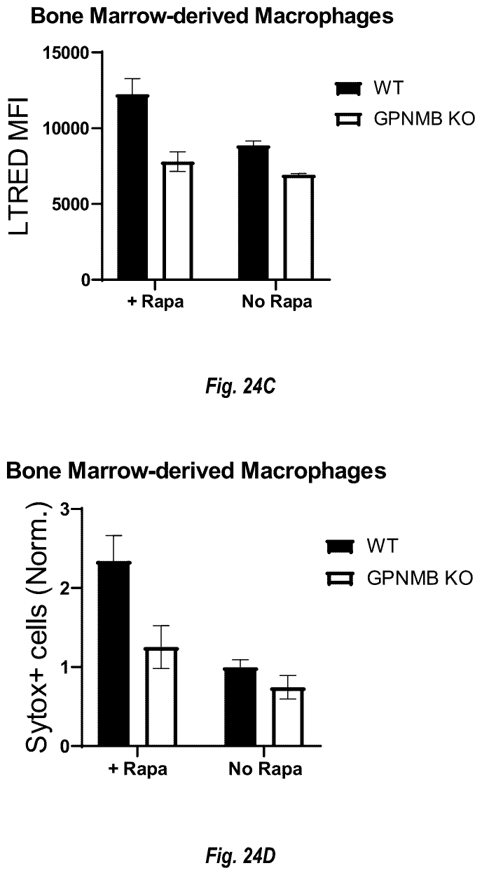

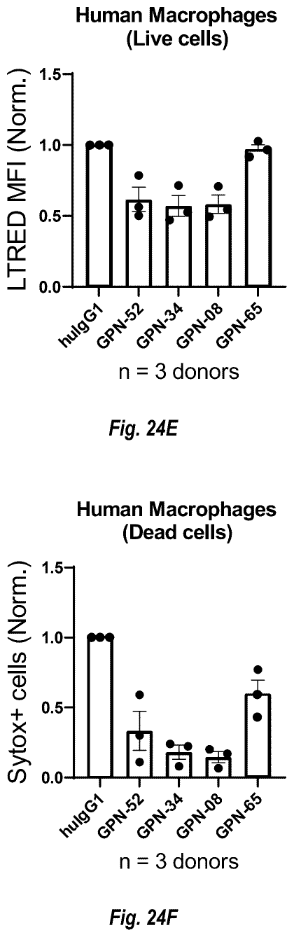

As shown in FIG. 24A and FIG. 24B, human macrophages stimulated with 100 nM rapamycin for 48 hours demonstrated a greater than 2-fold increase in Lysotracker fluorescence staining, indicative of increased lysosome volume in these stressed cells, and an increased percentage of Sytox+ dead cells. Together, these results showed that treatment of human macrophages with rapamycin resulted in autophagic cell death.

To ascertain if GPNMB contributes to the lysosomal stress response of macrophages following rapamycin treatment, bone marrow-derived macrophages (BMDM) from wildtype and GPNMB knock-out mice were tested using this assay. Femur and tibia bones from mice aged 6-8 weeks were harvested from female wildtype or GPNMB homozygous (−/−) GPNMB knockout (KO) animals. The bones were cut open, and a 21G needle was used to flush out marrow with ice cold PBS+2% FBS. Cells were passed through a 70 μm cell strainer to remove cell clumps, bone, hair, and other cells/tissues. Cells were centrifuged at 400×g for 5 min at 4° C. and resuspended at 1 million cells/ml of BMDM growth media (DMEM with 10% FBS and 20 ng/ml of M-CSF). Cells were left to differentiate for 7 days. BMDM were harvested, seeded at 100,000 cells per well in 96-well plates, and treated with 100 nM rapamycin or vehicle control for 48 hours. Following treatment, cells were pelleted and stained with 200 nM Lysotracker Red fluorescent dye for 15 minutes at 37° C. Cells were washed twice with FACS buffer (PBS supplemented with 2% FBS, 2 mM EDTA) and resuspended in FACS buffer supplemented with Sytox Green Live/Dead cell stain (Thermo Fisher) at 1:1000 dilution. Samples were acquired on a BD FACS Canto and Lysotracker fluorescence recorded on live cells. Data analysis and calculation of mean fluorescence intensity (MFI) values was performed with FlowJo (TreeStar) software version 10.0.8.

As shown in FIG. 24C and FIG. 24D, BMDM from wildtype animals treated with rapamycin demonstrated ˜40% increase in Lysotracker staining relative to vehicle control-treated cells. However, BMDM from GPNMB knock-out animals treated with rapamycin showed a modest 10% increase in Lysotracker staining, considerably less than that observed in BMDM obtained from wildtype animals. Similarly, rapamycin treatment significantly increased cell death in wildtype BMDM relative to that observed in GPNMB knockout BMDM. These results suggested that GPNMB participates in a lysosomal stress response in cells, and blocking GPNMB (for example, with an anti-GPNMB antibody of the present disclosure) protected cells from lysosomal stress conditions.

Anti-GPNMB antibodies of the present disclosure were screened on human macrophages to determine their ability to rescue cells from rapamycin-induced lysosomal stress. Human macrophages were plated with 1 μg/ml anti-GPNMB antibodies or isotype control antibody with or without rapamycin for 48 hours. FIG. 24D and FIG. 24E show that human macrophages treated with anti-GPNMB antibodies GPN-52, GPN-34, and GPN-08 in the presence of rapamycin decreased Lysotracker staining ˜50% relative to isotype control-treated cells. Similarly, anti-GPNMB antibodies GPN-52, GPN-34, and GPN-08 protected macrophages from rapamycin-induced cell death by 70-85% percent in the donors tested. Anti-GPNMB antibody GPN-65 did not reduce Lysotracker staining on macrophages and only partially decreased rapamycin-induced cell death by ˜40% relative to isotype control-treated cells.

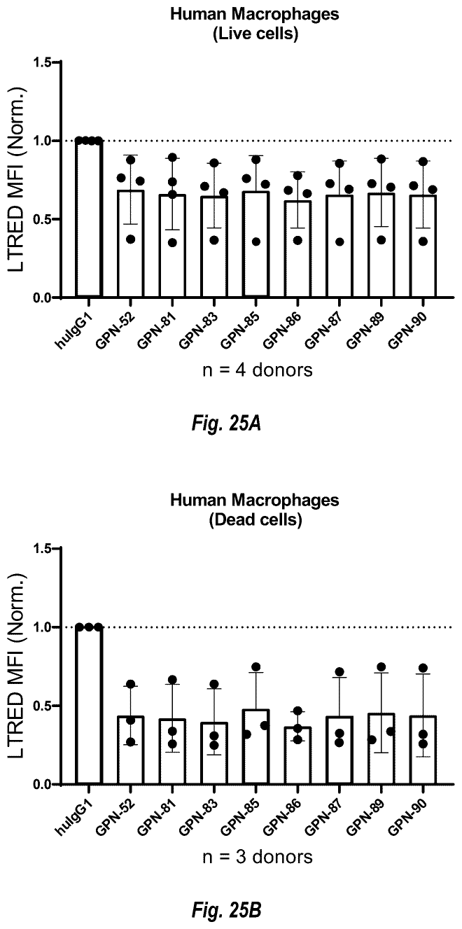

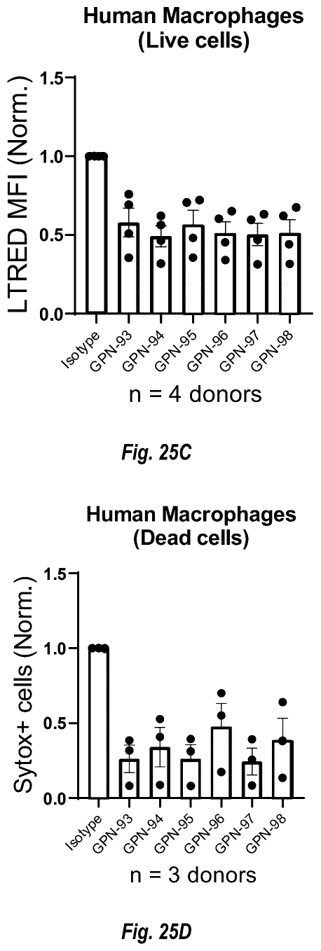

Additional experiments were performed with affinity matured variants of anti-GPNMB antibodies derived from anti-GPNMB antibodies GPN-08 and GPN-52 expressed on a human IgG1 wildtype backbone. Human macrophages were treated 1 μg/ml anti-GPNMB antibody or isotype control antibody for 48 hours in the presence of rapamycin. As shown in FIG. 25A-FIG. 25D, affinity matured anti-GPNMB antibodies from both lineages retained the ability to reduce Lysostracker and Sytox staining in rapamycin-treated human macrophages. Table 20 below provides additional data showing the effect of anti-GPNMB antibodies of the present disclosure on increases in GCase activity and reduce lysosomal dysfunction.

Taken together, these results suggest that anti-GPNMB antibodies of the present disclosure mimic the GPNMB knockout phenotype by preventing lysosome biogenesis and cell death induced by exposure to rapamycin.

TABLE 20

| | Lysosome Assays | | | |
|---|---|---|---|---|
| | Fold Increase | | Fold Decrease | EC50 |
| | GCase | EC50 | Lyso Tracker | Lyso |
| | (max response | GCase | (max response | Tracker |
| Antibody | at 1 μg/ml) | (nM) | at 1 μg/ml) | (nM) |
| GPN-82 | 1.54 | 0.365 | 0.569 | 0.6401 |
| GPN-83 | 1.32 | 0.834 | 0.755 | 1.172 |
| GPN-85 | 1.31 | 2.202 | 0.755 | 1.387 |
| GPN-86 | 1.34 | 0.166 | 0.731 | 1.198 |
| GPN-87 | 1.36 | 0.767 | 0.716 | 1.063 |
| GPN-89 | 1.35 | 0.898 | 0.757 | 1.183 |
| GPN-90 | 1.37 | 0.685 | 0.761 | 1.583 |
| GPN-92 | 1.25 | 0.27 | | |
| GPN-93 | 1.45 | 0.50 | | |
| GPN-94 | 1.40 | 0.54 | | |
| GPN-95 | 1.33 | 0.23 | | |
| GPN-96 | 1.31 | 0.23 | | |
| GPN-97 | 1.36 | 0.22 | | |
| GPN-98 | 1.39 | 0.25 | | |

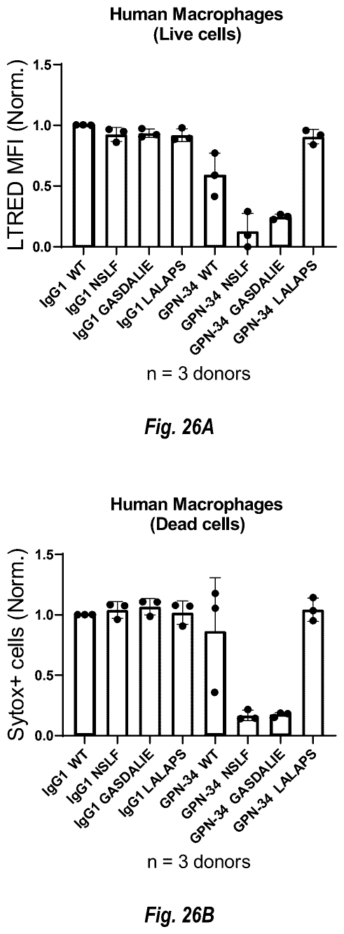

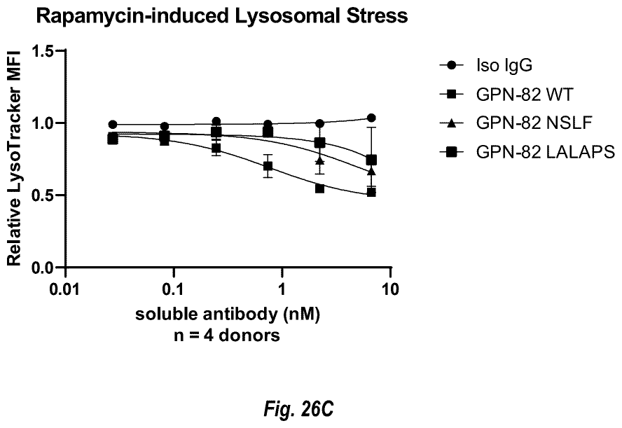

Anti-GPNMB antibodies of the present disclosure were further characterized on different human IgG Fc backbones to determine the contribution of Fc receptors on antibody-mediated inhibition of rapamycin stress. As shown in FIG. 26A and FIG. 26 B, Fc variants of anti-GPNMB antibody GPN-34 that engage Fc receptors (huIgG1 WT, NSLF, GASDALIE) retained the ability to decrease Lysotracker and/or Sytox dead cell staining on rapamycin-treated human macrophages. However, abrogating Fc receptor engagement by introducing the LALAPS Fc mutations to anti-GPNMB antibody GPN-34 abolished the ability to reduce Lysotracker and/or Sytox dead cell staining in macrophages. Similar observations were seen with Fc variants of anti-GPNMB antibody GPN-08. In contrast, Fc variants of anti-GPNMB antibody GPN-82 rescued rapamycin-induced lysosomal biogenesis on macrophages independent of FcR engagement (FIG. 26C). These results further confirmed that anti-GPNMB antibodies of the present disclosure protect human macrophages from rapamycin-induced lysosomal stress. These results also showed that certain anti-GPNMB antibodies (e.g., GPN-34 and GPN-08) depended on Fc receptor for certain activity, whereas other anti-GPNMB antibodies (e.g., GPN-52) showed this activity independent of Fc receptor engagement.

### Example 26: Characterization of Affinity Matured Anti-GPNMB Antibodies Binding to Cells and Ligand Blockade

Affinity matured anti-GPNMB antibodies of the present disclosure were examined to determine their ability to bind human and mouse GPNMB expressed on FS293-huGPNMB+ or B16F10 cells, respectively. Cell-based affinity measurements were performed to ascertain the apparent affinities of anti-GPNMB antibodies to cell-surface expressed GPNMB. Serial dilutions of anti-GPNMB antibodies of the present disclosure were added to 100,000 FS293-huGPNMB+ or B16F10 cells and allowed to achieve binding equilibrium at 4° C. After addition of fluorescently labeled secondary antibody and brief washing steps, MFI values as a function of titrated antibody concentration were recorded via FACS analysis. Equilibrium binding curves were fit using nonlinear regression analysis with Graphpad Prism 6 software. These cell-based titration experiments yielded EC50 values<1.0 nM (Table 21).

TABLE 21

| | Human GPNMB | Mouse GPNMB | mGPNMB-Fc |
|---|---|---|---|
| Antibody | (nM) | (nM) | Blockade (nM) |
| GPN-83 | 0.2029 | 0.2929 | 1.49 |
| GPN-84 | 0.2415 | 0.3298 | 1.439 |
| GPN-85 | 0.3848 | 0.3166 | 1.513 |
| GPN-86 | 0.2034 | 0.1776 | 1.26 |
| GPN-87 | 0.317 | 0.37 | 0.9803 |
| GPN-88 | 0.1919 | 0.2617 | 0.625 |
| GPN-89 | 0.1668 | 0.4432 | 1.181 |
| GPN-90 | 0.1478 | 0.4159 | 1.425 |
| GPN-91 | 0.2397 | 0.273 | 0.6402 |
| GPN-93 | 0.5679 | n.d. | n.d. |
| GPN-94 | 0.1859 | n.d. | n.d. |
| GPN-95 | 0.1815 | n.d. | n.d. |
| GPN-96 | 0.6521 | n.d. | n.d. |
| GPN-97 | 0.5464 | n.d. | n.d. |
| GPN-98 | 0.4071 | n.d. | n.d. |

Anti-GPNMB antibodies of the present disclosure demonstrated binding to FS293-huGPNMB+ or B16F10 cells as indicated by positive MFI values recorded with antibody staining. The negative isotype control did not bind to cells. Anti-GPNMB antibodies did not bind to parental Freestyle293 cells, which lack antigen expression (data not shown). Importantly, several anti-GPNMB antibodies of the present disclosure also bound to primary human macrophages, the principal target cell population for in vivo efficacy.

Mouse GPNMB-Fc showed significant binding to several cell lines, including SVEC4-10. Purified full-length affinity matured, anti-GPNMB antibodies were evaluated for their ability to block binding of mouse GPNMB-Fc to SVEC cells to calculate the ligand blocking potency of each clone. Briefly, 2 μg/ml soluble mGPNMB-Fc (mouse IgG2A Fc) diluted in binding buffer was pre-complexed with increasing concentrations of anti-GPNMB antibody or human IgG1 isotype control in 96-well plates. Subsequently, 100,000 SVEC cells were added per well and incubated on ice for 30 minutes. Soluble GPNMB-Fc bound to cell surface was detected with fluorescent anti-mouse IgG2A secondary antibody and measured on FACSCanto. Curves were fit using nonlinear regression analysis with Graphpad Prism 6 software. Cell-based titration experiments yielded IC50 values<2.5 nM.

### Example 27: Affinity Matured Anti-GPNMB Antibodies Increase Cell Surface Expression of PDL1 and Other Activation Markers in Human Macrophages

Anti-GPNMB antibodies of the present disclosure were evaluated for their ability to modulate expression of activation surface markers on human macrophages. Human macrophages were generated from CD14+ monocytes enriched from whole blood of healthy volunteers. Briefly, monocytes from peripheral human blood samples were isolated using the RosetteSep™ monocyte isolation antibody cocktail (StemCell Technologies) and differentiated into macrophages with 50-100 ng/mL human M-CSF (Stem Cell Technology) and cultured for 7 days. Cells were plated on culture dishes in RPMI 1640 medium (Invitrogen) containing (% fetal calf serum (Hyclone) and 20 mM HEPES and cultured at 37C in 5% CO₂. On day 7, human macrophages were harvested and seeded at 100,000 cells per well in 96-well plates. Cells were treated with 20 ng/ml MCSF and indicated anti-GPNMB antibodies or isotype control antibody for 24 hours. Macrophages were stained for PDL1 (Biolegend, clone MIH3) and CD40 (Biolegend, clone 5C3) in 100 μL FACS buffer (PBS supplemented with 2% FBS, 2 mM EDTA) containing FcR blocking agents (eBioscience) for 30 minutes on ice. Cells were washed twice in cold FACS buffer and acquired on a BD FACS Canto. Data analysis and calculation of mean fluorescence intensity (MFR) values was performed with FlowJo (TreeStar) software version 10.0.8. In some experiments, macrophages were treated with increasing concentrations of antibodies. Curves were fit using nonlinear regression analysis with Graphpad Prism 6 software. Cell-based titration experiments yielded EC50 values for PDL1 increase are shown below in Table 22 (far-right column).

TABLE 22

| | Macrophage Marker | | |
|---|---|---|---|
| | Fold Increase | Fold Increase | |
| | PDL1 | CD40 | |
| | (max response | (max response | EC50 |
| Antibody | at 1 μg/ml) | at 1 μg/ml) | PDL1 (nM) |
| GPN-82 | 4.602 | 2.29 | 1.94 |
| GPN-83 | 4.307 | 2.10 | 0.91 |
| GPN-85 | 4.222 | 2.10 | 0.97 |
| GPN-86 | 4.571 | 2.15 | 1.37 |
| GPN-87 | 4.399 | 2.15 | 1.36 |
| GPN-89 | 4.22 | 2.12 | 1.00 |
| GPN-90 | 4.281 | 2.12 | 0.68 |
| GPN-92 | 1.991 | 1.59 | 1.36 |
| GPN-93 | 2.182 | 1.87 | * |
| GPN-94 | 1.875 | 1.72 | 1.14 |
| GPN-95 | 1.967 | 1.74 | 0.70 |
| GPN-96 | 1.904 | 1.72 | 0.58 |
| GPN-97 | 2.053 | 1.73 | 0.75 |
| GPN-98 | 1.973 | 1.71 | 1.24 |

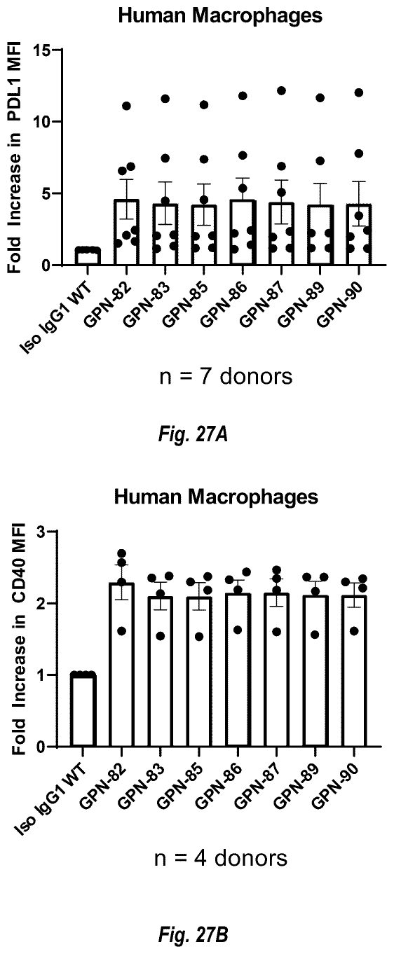

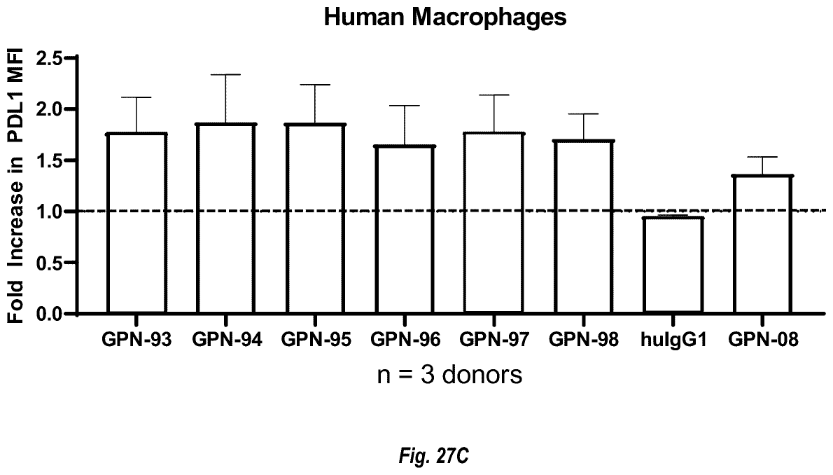

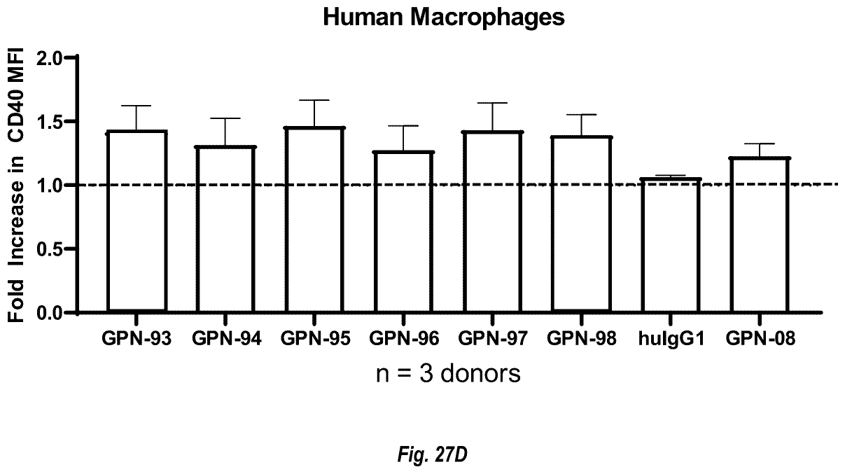

As shown in FIG. 27A and FIG. 27B, affinity matured anti-GPNMB antibodies derived from the anti-GPNMB antibody GPN-52 lineage, for example anti-GPNMB antibody GPN-82, GPN-83, GPN-85, GPN-86, GPN-87, GPN-89, and GPN-90, increased expression of PDL1 and CD40 relative to that observed in isotype control treated cells. The fold-increase in PDL1 and CD40 expression relative to isotype control-treated human macrophages following anti-GPNMB antibody incubation is shown above in Table 22.

Similarly, affinity matured anti-GPNMB antibodies derived from the anti-GPNMB antibody GPN-08 lineage, for example anti-GPNMB antibodies GPN-93, GPN-94, GPN-95, GPN-96, GPN-97, and GPN-98, increased expression of PDL1 and CD40 relative to isotype control treated cells (FIG. 27C and FIG. 27D). Importantly, affinity matured anti-GPNMB antibody variants increased PDL1 and CD40 expression on human macrophages to a greater extent than their parental antibodies (i.e., GPN-52 and GPN-08). These results were consistent with affinity matured anti-GPNMB antibodies retaining their ability to antagonize GPNMB function and promote activation of human macrophages in vitro.

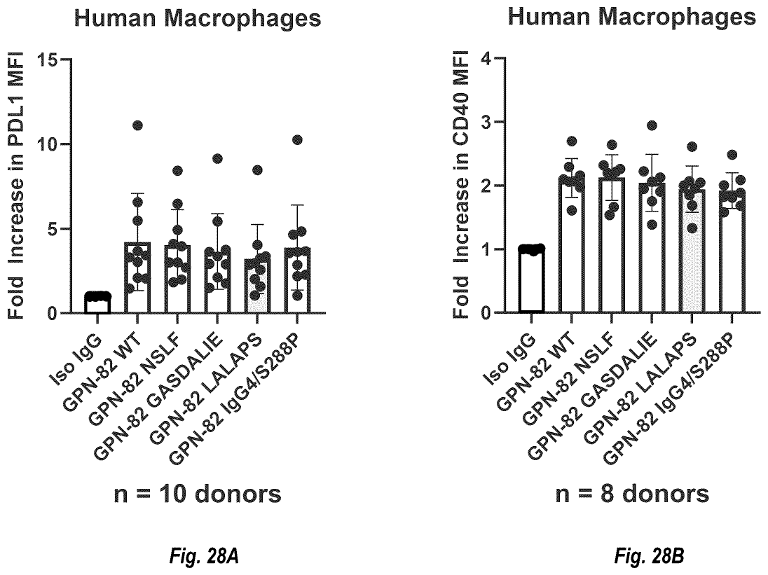

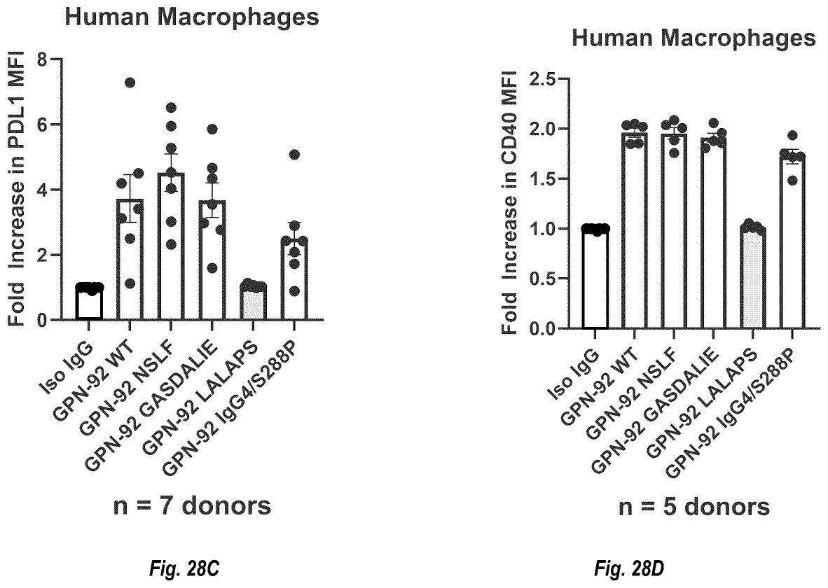

Affinity matured anti-GPNMB antibodies of the present disclosure were further characterized on different human IgG Fc backbones to determine the contribution of Fc receptors on antibody-mediated increase in PDL1 and CD40 expression. As shown in FIG. 28A and FIG. 28B, all Fc variants of anti-GPNMB antibody GPN-82, including Fc-silent mutations (LALAPS), retained the ability to induce PDL1 and CD40 expression on human macrophages. Fc variants of anti-GPNMB antibody GPN-92 that engage Fc receptors (e.g., huIgG1 WT, NSLF, GASDALIE) retained the ability to induce PDL1 and CD40 expression on human macrophages. However, Fc variants that abrogate Fc receptor engagement (e.g., LALAPS) abolished the ability of anti-GPNMB antibody GPN-92 to increase PDL1 and CD40 expression. Of note is that anti-GPNMB antibody GPN-92 with an IgG4 Fc backbone showed partial activity of increasing PDL1 and CD40 expression, reflecting the attenuated Fc effector function of this variant Fc. Taken together, these results were consistent with results from previous assays showing that anti-GPNMB antibodies of the present disclosure induced PDL1 and CD40 expression in human macrophages. Additionally, these results showed that certain anti-GPNMB antibody variants (derived from anti-GPNMB antibody GPN-08 or GPN-92) were dependent on Fc receptor for this activity, whereas other anti-GPNMB antibody variants (derived from anti-GPNMB antibody GPN-52 or GPN-82) functioned independent of Fc receptor engagement for this activity.

### Example 28: Affinity Matured Anti-GPNMB Antibodies Increase Glucocerebrosidase Activity in Human Macrophages

Affinity matured anti-GPNMB antibodies of the present disclosure on different human IgG Fc backbones were evaluated to determine the contribution of Fc receptors on antibody-mediated increases in GCase activity in human macrophages. GCase activity was measured by flow cytometry on human monocyte-derived macrophages by measuring the fluorescence intensity of FDGlu substrate added to cells, as described above.

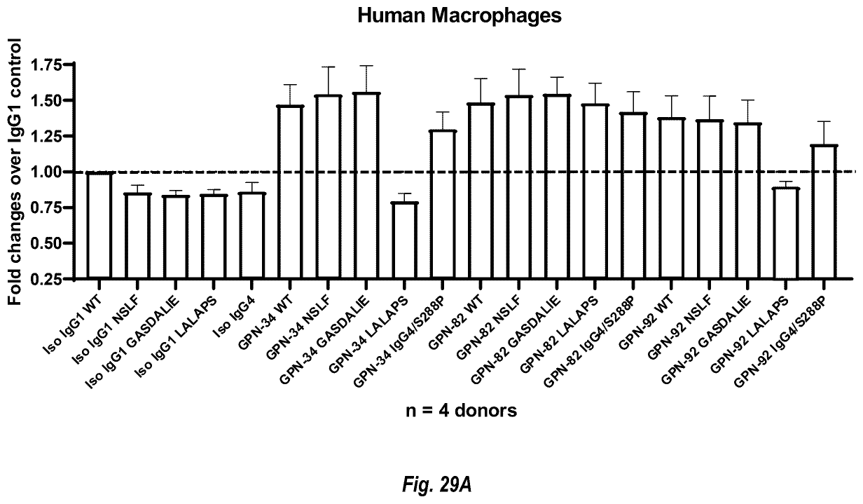

FIG. 29A shows that Fc variants of anti-GPNMB antibodies GPN-34 and GPN-92 that engage Fc receptors (e.g., huIgG1 WT, NSLF, GASDALIE) retained the ability to increase GCase activity on human macrophages. However, anti-GPNMB antibodies GPN-34 and GPN-92 on human Fc variants that abrogate Fc receptor engagement (e.g., LALAPS) did not show an increase in GCase activity on human macrophages. Both anti-GPNMB antibodies GPN-34 and GPN-92 showed partial activity on a human IgG4 Fc backbone, which is consistent with the attenuated Fc effector function of this Fc variant. In contrast, all Fc variants of anti-GPNMB antibody GPN-82, including Fc-silent mutations (e.g., LALAPS), retained the ability to increase GCase activity on human macrophages. Selected Fc variants of GPN-82 and GPN-92 were titrated on human macrophages to yield an EC50 curve for GCase activity.

As shown in FIG. 29B and FIG. 29C, anti-GPNMB antibody GPN-82 on a human IgG1 wildtype Fc backbone showed better activity compared to that observed with anti-GPNMB antibody GPN-82 on an NSLF or LALAPS Fc backbone. These results indicated that although anti-GPNMB antibody GPN-82 did not require Fc receptor binding for activity, FcRs may augment the activity of anti-GPNMB antibodies from the GPN-52/GPN-82 lineage. The human IgG1 wildtype and NSLF versions of anti-GPNMB antibody GPN-92 showed overlapping EC50 curves when titrated on human macrophages. These results were consistent with anti-GPNMB antibody GPN-92 requiring FcRs for full functional activity in the GCase assay. These results were consistent with those obtained in previous assays, showing that anti-GPNMB antibodies of the present disclosure induced GCase activity in human macrophages; and that certain anti-GPNMB antibody lineages (e.g., GPN-08 and GPN-92) were dependent on Fc receptor for this activity, whereas other anti-GPNMB antibody lineages (e.g., GPN-52 and GPN-82) functioned independent of Fc receptor engagement for this activity.

### Example 29: GPNMB Antibodies Reduce GPNMB Expression in Human Macrophages

Previous studies demonstrated that anti-GPNMB antibodies reduced cell surface expression of GPNMB on human macrophages. Anti-GPNMB antibodies of the present disclosure were further evaluated for their ability to reduce GPNMB expression when added to human macrophages. Human monocytes were isolated from peripheral blood of healthy donors and differentiated into macrophages in vitro. Following differentiation, 2 million cells were seeded onto 6-well tissue culture plates with 1-5 μg/ml of isotype control or anti-GPNMB antibodies. Cells were incubated overnight at 37° C. Cells were harvested and washed with cold PBS and lysed in cold lysis buffer (n-dodecyl-β-maltoside 1%, 50 mM Tris-HCl [pH 8.0], 150 mM NaCl, 1 mM EDTA, 1.5 mM MgCl₂, and 10% glycerol plus protease and phosphatase inhibitors). Whole-cell lysates were recovered by pelleting cellular debris and collecting the supernatant fraction. Proteins were resolved by SDS-PAGE, transferred to a polyvinylidene difluoride membrane, and probed with rabbit anti-GPNMB Ab (Cell Signaling Technology, clone E4D7P) for chemiluminescent western blots or anti-human GPNMB APC (clone HOST5DS, ThermoFisher) for fluorescent western blots.

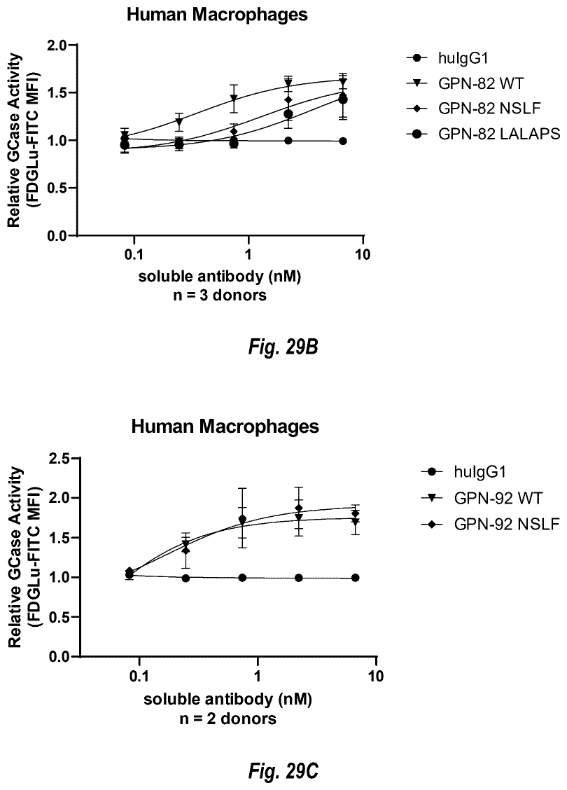

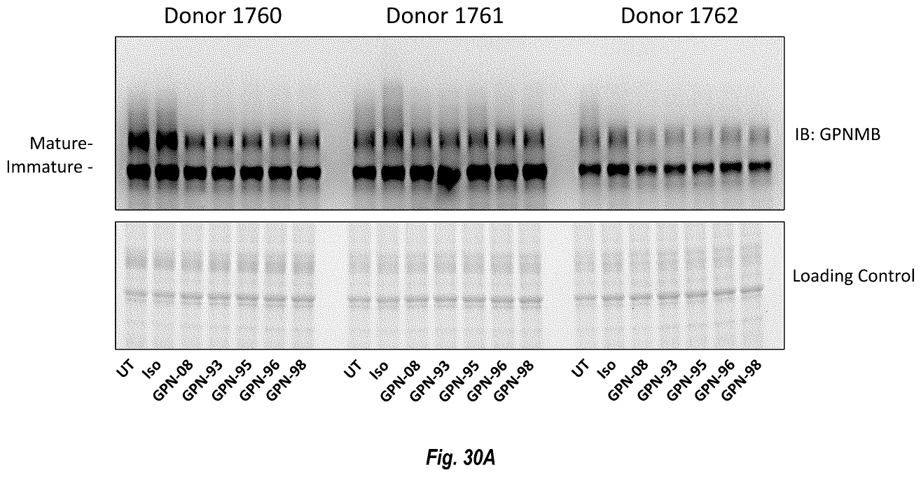

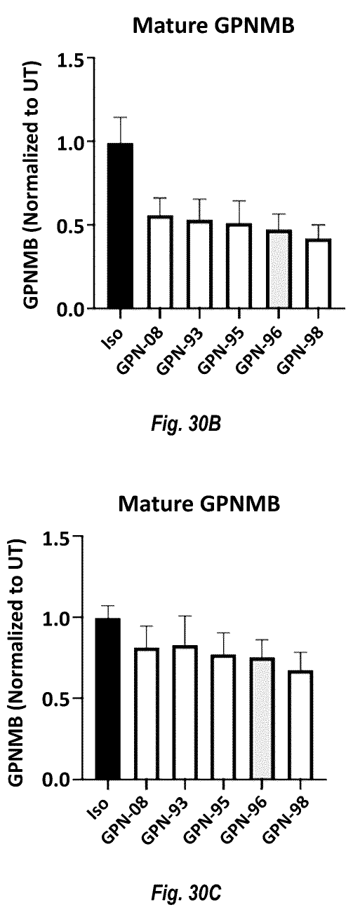

GPNMB resolves as two distinct bands on western blots; the higher molecular weight band is the mature, highly glycosylated form of GPNMB, while the lower molecular weight band is the immature, less glycosylated form of GPNMB. As shown in FIG. 30A, human macrophages treated with anti-GPNMB antibody GPN-08 and variants derived from this lineage (e.g., GPN-93, GPN-95, GPN-96, and GPN-98), reduced expression of mature GPNMB when compared to untreated or isotype control antibody treated human macrophages. In contrast, expression of the low molecular weight or immature form of GPNMB did not decrease compared to that observed in controls.

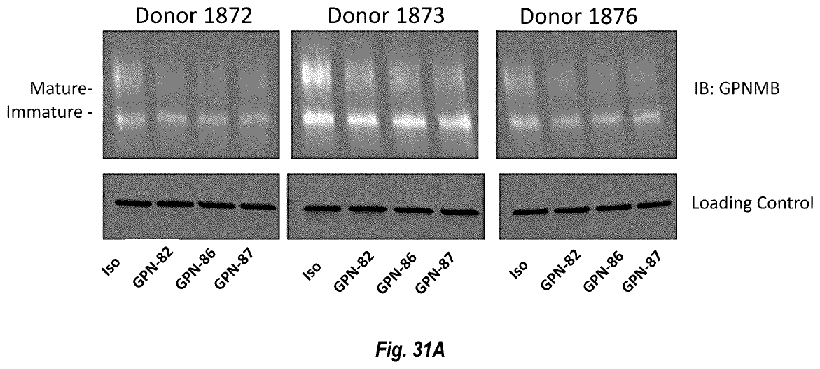

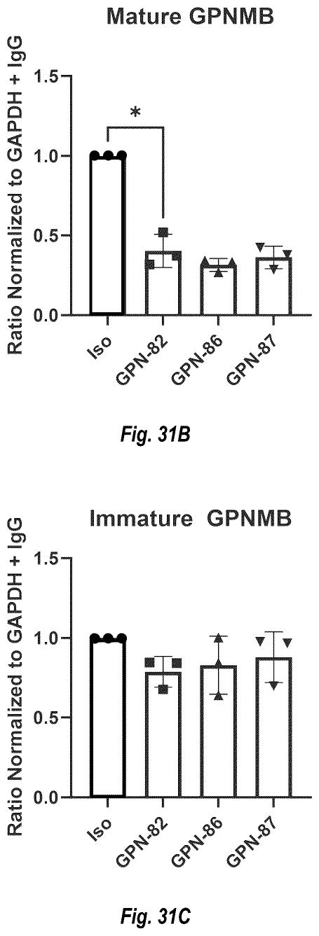

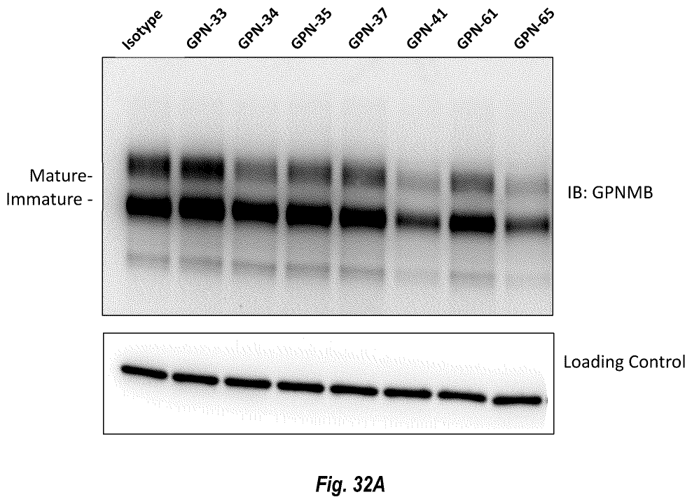

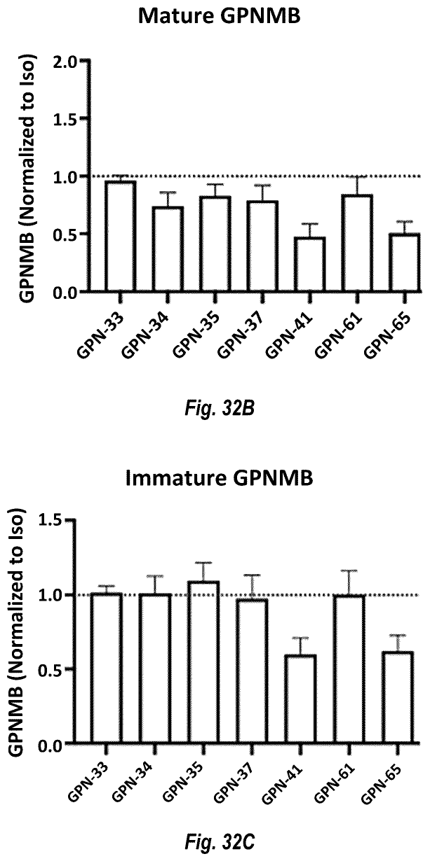

FIG. 30B and FIG. 30C show the relative intensities of the mature and immature forms of GPNMB detected by western blot and is consistent with anti-GPNMB antibodies preferentially reducing expression of the mature form of GPNMB over that of the immature form of GPNMB. Similarly, anti-GPNMB antibodies GPN-82, GPN-86, and GPN-87 also preferentially reduced expression of the mature form of GPNMB when added to human macrophages (FIG. 31A-FIG. 31C). FIG. 32A, however, shows that anti-GPNMB antibodies GPN-41 and GPN-65 reduced the expression of both mature and immature forms of GPNMB when added to human macrophages. Relative intensities shown in FIG. 32B and FIG. 32C indicated that anti-GPNMB antibodies GPN-41 and GPN-61 reduced both forms of GPNMB by ˜50% compared to that observed in control antibody treated cells. Anti-GPNMB antibodies of the present disclosure may antagonize GPNMB function through a distinct mechanism relating to the degradation of the mature and immature forms of GPNMB.

### Example 30: Reduction of LAMP-2 Expression Levels in Peripheral Immune Cells

The lysosomal protein Lysosome-associated membrane protein 2 (LAMP-2, also known as CD107b and Mac-3) is involved in lipid metabolism and autophagy. Lysosomal dysfunctions present in neurodegenerative diseases are often associated with lipid metabolism defects, resulting in increased lysosomal marker levels. Anti-GPNMB antibodies of the present disclosure reduced rapamycin-induced increase in lysosomal size in human monocyte-derived macrophages (shown above). To determine whether this pathway is regulated in vivo, LAMP-2 levels were determined in mouse primary immune cells following 24 hour treatment with anti-GPNMB antibodies of the present disclosure.

Wildtype or human GPNMB BAC transgenic mice were injected intravenously with various anti-GPNMB antibodies of the present disclosure at a dose of 50 mg/kg of body weight. Twenty-four hours later, tissues were collected and single-cell suspensions prepared using standard technique. One to two million peripheral leukocytes or splenocytes were stained with cell-type specific fluorescent antibodies and anti-LAMP-2 fluorescent antibody (listed in Table 23 below). Briefly, cells were incubated with an amine-reactive live cell marker for 15 min on ice. After quenching the live cell marker with Stain buffer (BD) supplemented with 2 mM EDTA, Fc receptors were blocked (FcX Plus, Biolegend) for 15 min on ice. Cells were then incubated with anti-surface protein antibodies for 30 min on ice. After washing, cells were fixed and permeabilized (BD Cytofix/Cytoperm) for intracellular LAMP-2 staining. Intracellular Fc receptors were blocked for 15 min on ice, then a fluorescent-conjugated LAMP-2 antibody or its corresponding isotype control was added for 30 min on ice. Cells were then washed and acquired on a LSR Fortessa flow cytometer. LAMP-2 expression was determined by comparing anti-LAMP-2 fluorescent signal to isotype control signal. The gating strategy used in these experiments lead to the identification of blood leukocyte cell populations, splenocyte subpopulations, and primary microglia.

TABLE 23

| Target | Manufacturer | Panel |
|---|---|---|
| Live/Dead Aqua | Invitrogen | Spleen/Blood |
| CD45 | BD | Spleen/Blood |
| CD11b | Biolegend | Spleen/Blood/Microglia |
| Ly6C | Biolegend | Spleen/Blood |
| Ly6G | Biolegend | Spleen/Blood |
| CD115 | Biolegend | Blood |
| F4/80 | Biolegend | Spleen |
| CD3 (lineage) | Biolegend | Spleen/Blood |
| CD19 (lineage) | Biolegend | Spleen/Blood |
| CD49b (lineage) | Biolegend | Spleen/Blood |
| B220 (lineage) | Biolegend | Spleen/Blood |
| LAMP-2 | Biolegend | Spleen/Blood |
| (intracellular) | | |

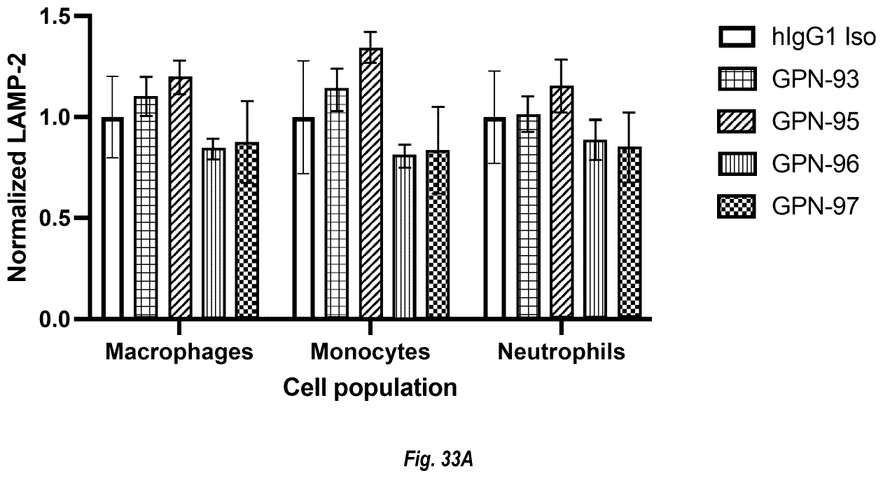

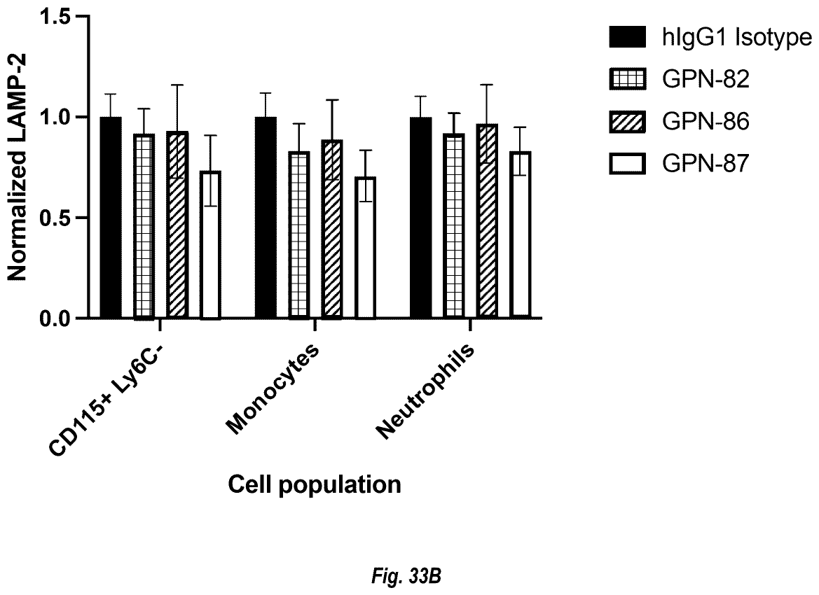

As shown in FIG. 33A, treatment of various immune cells with anti-GPNMB antibodies GPN-96 and GPN-97 resulted in a reduction in LAMP-2 signal in splenocytes of up to a 15% reduction in macrophages, 19% reduction in monocytes, and 15% reduction in neutrophils, compared to that observed in controls. FIG. 33B shows that treatment with anti-GPNMB antibody GPN-87 resulted in a reduction in LAMP-2 signal in blood monocytes (29% reduction), CD115+Ly6C− cells (27% reduction), and neutrophils (17% reduction) compared to that observed in controls. These results suggested that anti-GPNMB antibodies of the present disclosure are capable of regulating lysosomal size in vivo in the absence of lysosomal stress induction. Taken together, these results indicated that anti-GPNMB antibodies of the present disclosure reduced LAMP-2 levels in different peripheral immune cell populations, including for example macrophages, monocytes, and neutrophils.

### Example 31: Anti-GPNMB Antibodies Increase GCase Activity in Mouse Microglia and Peripheral Immune Cells

As disclosed above, anti-GPNMB antibodies of the present disclosure increased the activity of the lysosomal enzyme GCase in human monocyte-derived macrophages (in vitro). To determine whether GCase activity can be modulated in vivo, GCase activity was measured in mouse primary immune cells following 24 h treatment with various anti-GPNMB antibodies of the present disclosure.

Wildtype or human GPNMB BAC transgenic mice were injected intravenously with anti-GPNMB antibodies of the present disclosure at a dose of 10 mg/kg or 50 mg/kg of body weight. Twenty-four or forty-eight hours later, PBS-perfused brain and spleen were collected and single-cell suspensions prepared using standard techniques. GCase activity was measured in cells by the activation of a fluorescent substrate (PFB-FDGlu, Invitrogen). Briefly, enriched microglia cells were incubated for 30 min at 37° C. with PFB-FDGlu in HBSS (Gibco) with 1% BSA and 1 mM EDTA (Invitrogen). The reaction was stopped by adding cold media and placing the cells on ice. The cells were then stained with an anti-CD11b fluorescent antibody and dead cells were labeled using DAPI. GCase activity was determined by measuring the fluorescence intensity of PFB-FDGlu in live CD11b+ cells by flow cytometry (BD FACSCanto II). The gating strategy used in these experiments lead to the identification of blood leukocyte cell populations, splenocyte subpopulations, and primary microglia.

Splenocytes were prepared in RPMI 1640 (Corning) supplemented with 10% Fetal Bovine Serum (Hyclone) and 1% Penicillin/Streptomycin (Gibco). Approximately 2 million cells were incubated with PFB-FDGlu for 1 hour at 37° C. The reaction was stopped by adding cold media and placing the cells on ice. The splenocytes were then stained with population-specific markers (Table MC_1). Briefly, cells were incubated with an amine-reactive live cell marker for 15 min on ice. After quenching the live cell marker with Stain buffer (BD) supplemented with 2 mM EDTA, Fc receptors were blocked (FcX Plus, Biolegend) for 15 min on ice. Cells were then incubated with anti-surface protein antibodies for 30 min on ice. Cells were then washed and GCase activity was determined by measuring the fluorescence intensity of PFB-FDGlu in splenic subpopulations by flow cytometry (BD LSR Fortessa).

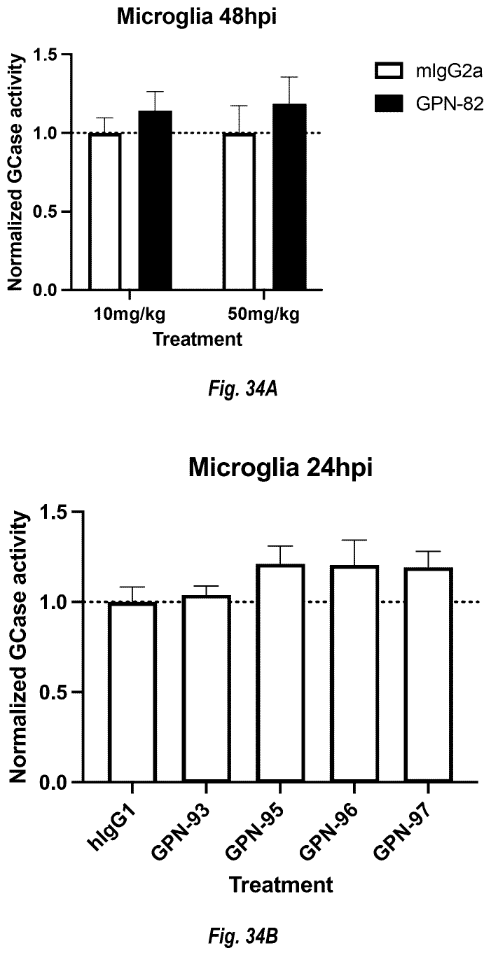

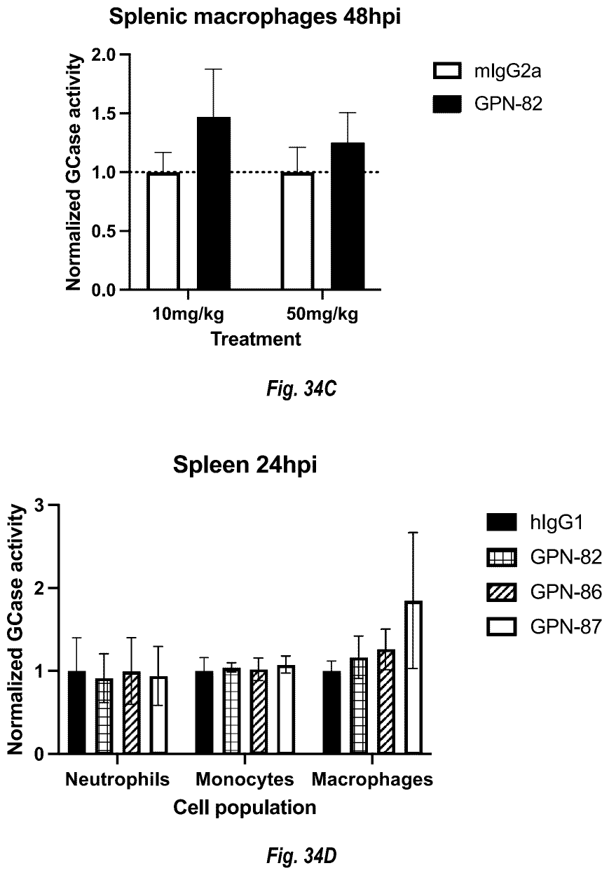

As shown in FIG. 34A, administration of anti-GPNMB antibody GPN-82 increased GCase activity measured in primary microglia 48 hours post injection by 14% at 10 mg/kg, and by 18% at 50 mg/kg. FIG. 34B shows that administration of anti-GPNMB antibodies GPN-95, GPN-96, and GPN-97 increased GCase activity 21%, 20%, and 19%, respectively, in primary mouse microglia 24 hours post injection with 50 mg/kg anti-GPNMB antibody. FIG. 34C shows that administration of anti-GPNMB antibody GPN-82 increased GCase activity measured in splenic macrophages 48 hours post injection by 47% at 10 mg/kg and by 25% at 50 mg/kg, which is consistent with saturation effects observed in vitro. FIG. 34D shows that administration of anti-GPNMB antibody GPN-87 at 50 mg/kg increased GCase activity in splenic macrophages by 84%. Anti-GPNMB antibodies GPN-86 and GPN-87 showed a more moderate increase in GCase activity in splenic macrophages of 16% and 25%, respectively. These results indicated that intravenous injection of anti-GPNMB antibodies of the present disclosure increased the activity of GCase in vivo both in the periphery (splenocyte subpopulations) and in the brain (microglia), consistent with enhanced lysosomal function. Enhancing lysosomal activity of glial cells with anti-GPNMB antibodies of the present disclosure may increase their ability to degrade pathological protein aggregates and lipids.

Provided below are various heavy chain IgG Fc amino acid sequences for use in the instant disclosure.

huIgG1-WT with C-terminal lysine
(SEQ ID NO: 213)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPGK
huIgG1-WT without C-terminal lysine
(SEQ ID NO: 214)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPG
huIgG1-K322A with C-terminal lysine
(SEQ ID NO: 215)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCAVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPGK
huIgG1-K322A without C-terminal lysine
(SEQ ID NO: 216)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCAVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPG
huIgG1-LALAPS (L234A; L235A, P331S) with C-terminal lysine
(SEQ ID NO: 217)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPASIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPGK
huIgG1-LALAPS (L234A; L235A, P331S) without C-terminal lysine
(SEQ ID NO: 218)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPASIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPG
huIgG1-PS (P331S) with C-terminal lysine
(SEQ ID NO: 219)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPASIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPGK
huIgG1-PS (P331S) without C-terminal lysine
(SEQ ID NO: 220)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPASIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPG
huIgG1-PSEG (P331S; E430G) with C-terminal lysine
(SEQ ID NO: 221)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPASIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHGALHNHYTQK
SLSLSPGK
huIgG1-PSEG (P331S; E430G) without C-terminal lysine
(SEQ ID NO: 222)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPASIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHGALHNHYTQK
SLSLSPG
huIgG1-NSLF (N325S; L328F) with C-terminal lysine
(SEQ ID NO: 223)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSSKAFPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPGK
huIgG1-NSLF (N325S; L328F) without C-terminal lysine
(SEQ ID NO: 224)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSSKAFPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPG
huIgG1-SELF (S267E; L328F) with C-terminal lysine
(SEQ ID NO: 225)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVEHEDPEVKFNWYVDGVEVHNAKTKPREEQYNSTYRVVSVLTVLHQ
DWLNGKEYKCKVSNKAFPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPGK
huIgG1-SELF (S267E; L328F) without C-terminal lysine
(SEQ ID NO: 226)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPK
DTLMISRTPEVTCVVVDVEHEDPEVKFNWYVDGVEVHNAKTKPREEQYNSTYRVVSVLTVLHQ
DWLNGKEYKCKVSNKAFPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPG
huIgG1-LS with C-terminal lysine
(SEQ ID NO: 227)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPASIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVLHEALHSHYTQKS
LSLSPGK
huIgG1-LS without C-terminal lysine
(SEQ ID NO: 228)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPASIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVLHEALHSHYTQKS
LSLSPG
huIgG2-WT with C-terminal lysine
(SEQ ID NO: 229)
ASTKGPSVFPLAPCSRSTSESTAALGCLVKDYFPEPVTVSWNSGALTSGVHTFPAVLQSSGLYSLS
SVVTVPSSNFGTQTYTCNVDHKPSNTKVDKTVERKCCVECPPCPAPPVAGPSVFLFPPKPKDTLM
ISRTPEVTCVVVDVSHEDPEVQFNWYVDGVEVHNAKTKPREEQFNSTFRVVSVLTVVHQDWLN
GKEYKCKVSNKGLPAPIEKTISKTKGQPREPQVYTLPPSREEMTKNQVSLTCLVKGFYPSDIAVE
WESNGQPENNYKTTPPMLDSDGSFFLYSKLTVDKSRWQQGNVFSCSVMHEALHNHYTQKSLSL
SPGK
huIgG2-WT without C-terminal lysine
(SEQ ID NO: 230)
ASTKGPSVFPLAPCSRSTSESTAALGCLVKDYFPEPVTVSWNSGALTSGVHTFPAVLQSSGLYSLS
SVVTVPSSNFGTQTYTCNVDHKPSNTKVDKTVERKCCVECPPCPAPPVAGPSVFLFPPKPKDTLM
ISRTPEVTCVVVDVSHEDPEVQFNWYVDGVEVHNAKTKPREEQFNSTFRVVSVLTVVHQDWLN
GKEYKCKVSNKGLPAPIEKTISKTKGQPREPQVYTLPPSREEMTKNQVSLTCLVKGFYPSDIAVE
WESNGQPENNYKTTPPMLDSDGSFFLYSKLTVDKSRWQQGNVFSCSVMHEALHNHYTQKSLSL
SPG
huIgG4-WT with C-terminal lysine
(SEQ ID NO: 231)
ASTKGPSVFPLAPCSRSTSESTAALGCLVKDYFPEPVTVSWNSGALTSGVHTFPAVLQSSGLYSLS
SVVTVPSSSLGTKTYTCNVDHKPSNTKVDKRVESKYGPPCPSCPAPEFLGGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYTQKSLSL
SLGK
huIgG4-WT without C-terminal lysine
(SEQ ID NO: 232)
ASTKGPSVFPLAPCSRSTSESTAALGCLVKDYFPEPVTVSWNSGALTSGVHTFPAVLQSSGLYSLS
SVVTVPSSSLGTKTYTCNVDHKPSNTKVDKRVESKYGPPCPSCPAPEFLGGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYTQKSLSL
SLG
huIgG1-LALAPS (L234A; L235A, P329S) with C-terminal lysine
(SEQ ID NO: 282)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALSAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPGK
(SEQ ID NO: 283)
huIgG1-LALAPS (L234A; L235A, P329S) without C-terminal lysine
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALSAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQK
SLSLSPG
huIgG1-LALAPS (L234A; L235A; P331G) with C-terminal lysine
(SEQ ID NO: 284)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAGIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQ
KSLSLSPGK
huIgG1-LALAPS (L234A; L235A; P331G) without C-terminal lysine
(SEQ ID NO: 285)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAGIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQ
KSLSLSPG
huIgG1-LALAPG (L234A; L235A, P329G) with C-terminal lysine
(SEQ ID NO: 286)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALGAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQ
KSLSLSPGK
huIgG1-LALAPG (L234A; L235A, P329G) without C-terminal lysine
(SEQ ID NO: 287)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALGAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQ
KSLSLSPG
huIgG4 (S228P)-with C-terminal lysine
(SEQ ID NO: 288)
ASTKGPSVFPLAPCSRSTSESTAALGCLVKDYFPEPVTVSWNSGALTSGVHTFPAVLQSSGLYSLS
SVVTVPSSSLGTKTYTCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVXYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSL
SLSLGK
huIgG4 (S228P)-without C-terminal lysine
(SEQ ID NO: 289)
ASTKGPSVFPLAPCSRSTSESTAALGCLVKDYFPEPVTVSWNSGALTSGVHTFPAVLQSSGLYSLS
SVVTVPSSSLGTKTYTCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYTQKSLSL
SLG
Human IgG light chain constant region amino acid sequence
(SEQ ID NO: 233)
RTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC

Provided below are amino acid sequences of full-length heavy chain anti-GPNMB antibodies of the present disclosure comprising different Fc variants and amino acid sequences of full-length light chain anti-GPNMB antibodies of the present disclosure.

GPN-81 huIgG1 - WT with C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 290)
GPN-81 huIgG1 - WT without C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 291)
GPN-81 LALAPS (L234A; L235A; P331S) - with C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 292)
GPN-81 huIgG1 - LALAPS (L234A; L235A; P331S) - without C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 293)
GPN-81 huIgG1 - LALAPS (L234A; L235A; P329S) - with C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 294)
GPN-81 huIgG1 - LALAPS (L234A; L235A; P329S) - without C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 295)
GPN-81 huIgG1- LALAPG (L234A; L235A; P331G) - with C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 296)
GPN-81huIgG1- LALAPG (L234A; L235A; P331G) - without C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 297)
GPN-81 huIgG1 - LALAPG (L234A; L235A; P329G) - with C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 298)
GPN-81 huIgG1 - LALAPG (L234A; L235A; P329G) - without C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 299)
GPN-81 huIgG1 - PSEG (P331S; E430G) - with C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPGK
(SEQ ID NO: 300)
GPN-81 huIgG1 - PSEG (P331S; E430G) - without C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPG
(SEQ ID NO: 301)
GPN-81 huIgG1 - NSLF (N325S: L328F) - with C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 302)
GPN-81 huIgG1 - NSLF (N325S: L328F) - without C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 303)
GPN-81 huIgG4 - WT with C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 304)
GPN-81 huIgG4 - WT without C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 305)
GPN-81 huIgG4 (S228P) - with C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 306)
GPN-81 huIgG4 (S228P) - without C-terminal lysine
QVQLQESGPGLVKPSETLSLTCTVSGISITTGNYRWSWIRQPPGKGLEWIGYIYYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 307)
GPN-81 Light Chain
DIVMTQSPDSLAVSLGERATINCKSSESVDYYGIGFLNWYQQKPGQPPKLLIYAASTQGSGVPDR
FSGSGSGTDFTLTISSLQAEDVAVYYCQQSKEVPPTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKV
YACEVTHQGLSSPVTKSFNRGEC (SEQ ID NO: 416)
GPN-82 huIgG1 - WT with C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 308)
GPN-82 huIgG1 - WT without C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 309)
GPN-82 huIgG1 - LALAPS (L234A; L235A; P331S) - with C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 310)
GPN-82 huIgG1 - LALAPS (L234A; L235A; P331S) - without C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 311)
GPN-82 huIgG1 - LALAPS (L234A; L235A; P329S) - with C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 312)
GPN-82 huIgG1 - LALAPS (L234A; L235A; P329S) - without C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 313)
GPN-82 huIgG1- LALAPG (L234A; L235A; P331G) - with C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 314)
GPN-82 huIgG1- LALAPG (L234A; L235A; P331G) - without C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 315)
GPN-82 huIgG1 - LALAPG (L234A; L235A; P329G) - with C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 316)
GPN-82 huIgG1 - LALAPG (L234A; L235A; P329G) - without C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 317)
GPN-82 huIgG1 - PSEG (P331S; E430G) - with C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPGK
(SEQ ID NO: 318)
GPN-82 huIgG1 - PSEG (P331S; E430G) - without C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPG
(SEQ ID NO: 319)
GPN-82 huIgG1 - NSLF (N325S: L328F) - with C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 320)
GPN-82 huIgG1 - NSLF (N325S: L328F) - without C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 321)
GPN-82 huIgG4 - WT with C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 322)
GPN-82 huIgG4 - WT without C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 323)
GPN-82 huIgG4 (S228P) - with C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 324)
GPN-82 huIgG4 (S228P) - without C-terminal lysine
QVQLQESGPGLVKPSETLSLTCTVSGISITTGNYRWSWIRQPPGKGLEWIGYIYYSGTIIYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 325)
GPN-82 Light Chain
DIVMTQSPDSLAVSLGERATINCKSSRSVDYYGIGFLNWYQQKPGQPPKLLIYATSTQGSGVPDR
FSGSGSGTDFTLTISSLQAEDVAVYYCQQQKEVPPTFGGGTKVEIKRTVAAPSVFIFPPSDEQLKS
GTASVVCLLNNFYPREAKVQWKVDNALQSGNSQESVTEQDSKDSTYSLSSTLTLSKADYEKHK
VYACEVTHQGLSSPVTKSFNRGEC (SEQ ID NO: 417)
GPN-83 huIgG1 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 326)
GPN-83 huIgG1 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 327)
GPN-83 huIgG1 - LALAPS (L234A; L235A; P331S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 328)
GPN-83 huIgG1 - LALAPS (L234A; L235A; P331S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 329)
GPN-83 huIgG1 - LALAPS (L234A; L235A; P329S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 330)
GPN-83 huIgG1 - LALAPS (L234A; L235A; P329S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 331)
GPN-83 huIgG1- LALAPG (L234A; L235A; P331G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 332)
GPN-83 huIgG1- LALAPG (L234A; L235A; P331G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 333)
GPN-83 huIgG1 - LALAPG (L234A; L235A; P329G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 334)
GPN-83 huIgG1 - LALAPG (L234A; L235A; P329G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 335)
GPN-83 huIgG1 - PSEG (P331S; E430G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPGK
(SEQ ID NO: 336)
GPN-83 huIgG1 - PSEG (P331S; E430G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPG
(SEQ ID NO: 337)
GPN-83 huIgG1 - NSLF (N325S: L328F) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 338)
GPN-83 huIgG1 - NSLF (N325S: L328F) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 339)
GPN-83 huIgG4 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 340)
GPN-83 huIgG4 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 341)
GPN-83 huIgG4 (S228P) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 342)
GPN-83 huIgG4 (S228P) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 343)
GPN-83 Light Chain
DIVMTQSPDSLAVSLGERATINCKSSRSVDYYGIGFVNWYQQKPGQPPKLLIYATSIQGSGVPDRF
SGSGSGTDFTLTISSLQAEDVAVYYCQQQKEVPPTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKV
YACEVTHQGLSSPVTKSFNRGEC (SEQ ID NO: 418)
GPN-84 huIgG1 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 344)
GPN-84 huIgG1 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 345)
GPN-84 huIgG1 - LALAPS (L234A; L235A; P331S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 346)
GPN-84 huIgG1 - LALAPS (L234A; L235A; P331S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 347)
GPN-84 huIgG1 - LALAPS (L234A; L235A; P329S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 348)
GPN-84 huIgG1 - LALAPS (L234A; L235A; P329S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 349)
GPN-84 huIgG1- LALAPG (L234A; L235A; P331G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 350)
GPN-84 huIgG1- LALAPG (L234A; L235A; P331G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 351)
GPN-84 huIgG1 - LALAPG (L234A; L235A; P329G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 352)
GPN-84 huIgG1 - LALAPG (L234A; L235A; P329G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 353)
GPN-84 huIgG1 - PSEG (P331S; E430G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPGK
(SEQ ID NO: 354)
GPN-84 huIgG1 - PSEG (P331S; E430G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPG
(SEQ ID NO: 355)
GPN-84 huIgG1 - NSLF (N325S: L328F) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 356)
GPN-84 huIgG1 - NSLF (N325S: L328F) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 357)
GPN-84 huIgG4 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 358)
GPN-84 huIgG4 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 359)
GPN-84 huIgG4 (S228P) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 360)
GPN-84 huIgG4 (S228P) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 361)
GPN-84 Light Chain
DIVMTQSPDSLAVSLGERATINCKSSRSVDYYGIGFVNWYQQKPGQPPKLLIYATSIQGSGVPDRF
SGSGSGTDFTLTISSLQAEDVAVYYCQQQKEVPPTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKV
YACEVTHQGLSSPVTKSFNRGEC (SEQ ID NO: 419)
GPN-85 huIgG1 - WT with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPGK
(SEQ ID NO: 362)
GPN-85 huIgG1 - WT without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPG
(SEQ ID NO: 363)
GPN-85 huIgG1 - LALAPS (L234A; L235A; P331S) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALPASIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGK
(SEQ ID NO: 364)
GPN-85 huIgG1 - LALAPS (L234A; L235A; P331S) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALPASIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
(SEQ ID NO: 365)
GPN-85 huIgG1 - LALAPS (L234A; L235A; P329S) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALSAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGK
(SEQ ID NO: 366)
GPN-85 huIgG1 - LALAPS (L234A; L235A; P329S) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALSAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
(SEQ ID NO: 367)
GPN-85 huIgG1- LALAPG (L234A; L235A; P331G) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALPAGIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGK
(SEQ ID NO: 368)
GPN-85 huIgG1- LALAPG (L234A; L235A; P331G) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALPAGIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
(SEQ ID NO: 369)
GPN-85 huIgG1 - LALAPG (L234A; L235A; P329G) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALGAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGK
(SEQ ID NO: 370)
GPN-85 huIgG1 - LALAPG (L234A; L235A; P329G) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALGAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
(SEQ ID NO: 371)
GPN-85 huIgG1 - PSEG (P331S; E430G) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPASIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHGALHNHYTQKSLSLSPGK
(SEQ ID NO: 372)
GPN-85 huIgG1 - PSEG (P331S; E430G) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPASIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHGALHNHYTQKSLSLSPG
(SEQ ID NO: 373)
GPN-85 huIgG1 - NSLF (N325S: L328F) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSSKAFPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 374)
GPN-85 huIgG1 - NSLF (N325S: L328F) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSSKAFPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 375)
GPN-85 huIgG4 - WT with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 376)
GPN-85 huIgG4 - WT without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 377)
GPN-85 huIgG4 (S228P) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLGK
(SEQ ID NO: 378)
GPN-85 huIgG4 (S228P) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLG
(SEQ ID NO: 379)
GPN-85 Light Chain
DIVMTQSPDSLAVSLGERATINCKSSRSVDYFGIGFLNWYQQKPGQPPKLLIYATSHQGSGVPDR
FSGSGSGTDFTLTISSLQAEDVAVYYCQQQKEVPPTFGGGTKVEIKRTVAAPSVFIFPPSDEQLKS
GTASVVCLLNNFYPREAKVQWKVDNALQSGNSQESVTEQDSKDSTYSLSSTLTLSKADYEKHK
VYACEVTHQGLSSPVTKSFNRGEC (SEQ ID NO: 420)
GPN-86 huIgG1 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 326)
GPN-86 huIgG1 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 327)
GPN-86 huIgG1 - LALAPS (L234A; L235A; P331S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 328)
GPN-86 huIgG1 - LALAPS (L234A; L235A; P331S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 329)
GPN-86 huIgG1 - LALAPS (L234A; L235A; P329S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 330)
GPN-86 huIgG1 - LALAPS (L234A; L235A; P329S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 331)
GPN-86 huIgG1- LALAPG (L234A; L235A; P331G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 332)
GPN-86 huIgG1- LALAPG (L234A; L235A; P331G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 333)
GPN-86 huIgG1 - LALAPG (L234A; L235A; P329G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 334)
GPN-86 huIgG1 - LALAPG (L234A; L235A; P329G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 335)
GPN-86 huIgG1 - PSEG (P331S; E430G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPGK
(SEQ ID NO: 336)
GPN-86 huIgG1 - PSEG (P331S; E430G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPG
(SEQ ID NO: 337)
GPN-86 huIgG1 - NSLF (N325S: L328F) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 338)
GPN-86 huIgG1 - NSLF (N325S: L328F) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 339)
GPN-86 huIgG4 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 340)
GPN-86 huIgG4 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 341)
GPN-86 huIgG4 (S228P) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 342)
GPN-86 huIgG4 (S228P) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 343)
GPN-86 Light Chain
DIVMTQSPDSLAVSLGERATINCKSSRSVDYYGIGFLNWYQQKPGQPPKLLIYATSKQGSGVPDR
FSGSGSGTDFTLTISSLQAEDVAVYYCQQQKEVPPTFGGGTKVEIKRTVAAPSVFIFPPSDEQLKS
GTASVVCLLNNFYPREAKVQWKVDNALQSGNSQESVTEQDSKDSTYSLSSTLTLSKADYEKHK
VYACEVTHQGLSSPVTKSFNRGEC (SEQ ID NO: 421)
GPN-87 huIgG1 - WT with C-terminal lysine
(SEQ ID NO: 344)
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
GPN-87 huIgG1 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 345)
GPN-87 huIgG1 - LALAPS (L234A; L235A; P331S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 346)
GPN-87 huIgG1 - LALAPS (L234A; L235A; P331S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 347)
GPN-87 huIgG1 - LALAPS (L234A; L235A; P329S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 348)
GPN-87 huIgG1 - LALAPS (L234A; L235A; P329S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 349)
GPN-87 huIgG1- LALAPG (L234A; L235A; P331G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 350)
GPN-87 huIgG1- LALAPG (L234A; L235A; P331G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 351)
GPN-87 huIgG1 - LALAPG (L234A; L235A; P329G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 352)
GPN-87 huIgG1 - LALAPG (L234A; L235A; P329G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 353)
GPN-87 huIgG1 - PSEG (P331S; E430G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPGK
(SEQ ID NO: 354)
GPN-87 huIgG1 - PSEG (P331S; E430G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPG
(SEQ ID NO: 355)
GPN-87 huIgG1 - NSLF (N325S: L328F) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 356)
GPN-87 huIgG1 - NSLF (N325S: L328F) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 357)
GPN-87 huIgG4 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 358)
GPN-87 huIgG4 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 359)
GPN-87 huIgG4 (S228P) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 360)
GPN-87 huIgG4 (S228P) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 361)
GPN-87 Light Chain
DIVMTQSPDSLAVSLGERATINCKSSRSVDYYGIGFLNWYQQKPGQPPKLLIYATSKQGSGVPDR
FSGSGSGTDFTLTISSLQAEDVAVYYCQQQKEVPPTFGGGTKVEIKRTVAAPSVFIFPPSDEQLKS
GTASVVCLLNNFYPREAKVQWKVDNALQSGNSQESVTEQDSKDSTYSLSSTLTLSKADYEKHK
VYACEVTHQGLSSPVTKSFNRGEC (SEQ ID NO: 422)
GPN-88 huIgG1 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 326)
GPN-88 huIgG1 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 327)
GPN-88 huIgG1 - LALAPS (L234A; L235A; P331S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 328)
GPN-88 huIgG1 - LALAPS (L234A; L235A; P331S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 329)
GPN-88 huIgG1 - LALAPS (L234A; L235A; P329S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 330)
GPN-88 huIgG1 - LALAPS (L234A; L235A; P329S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 331)
GPN-88 huIgG1- LALAPG (L234A; L235A; P331G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 332)
GPN-88 huIgG1- LALAPG (L234A; L235A; P331G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 333)
GPN-88 huIgG1 - LALAPG (L234A; L235A; P329G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 334)
GPN-88 huIgG1 - LALAPG (L234A; L235A; P329G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 335)
GPN-88 huIgG1 - PSEG (P331S; E430G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPGK
(SEQ ID NO: 336)
GPN-88 huIgG1 - PSEG (P331S; E430G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPG
(SEQ ID NO: 337)
GPN-88 huIgG1 - NSLF (N325S: L328F) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 338)
GPN-88 huIgG1 - NSLF (N325S: L328F) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 339)
GPN-88 huIgG4 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 340)
GPN-88 huIgG4 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 341)
GPN-88 huIgG4 (S228P) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 342)
GPN-88 huIgG4 (S228P) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 343)
GPN-88 Light Chain
DIVMTQSPDSLAVSLGERATINCKSSRSVDYYGIGFLNWYQQKPGQPPKLLIYATSHQGSGVPDR
FSGSGSGTDFTLTISSLQAEDVAVYYCQQQKHVPPTFGGGTKVEIKRTVAAPSVFIFPPSDEQLKS
GTASVVCLLNNFYPREAKVQWKVDNALQSGNSQESVTEQDSKDSTYSLSSTLTLSKADYEKHK
VYACEVTHQGLSSPVTKSFNRGEC (SEQ ID NO: 423)
GPN-89 huIgG1 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 344)
GPN-89 huIgG1 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 345)
GPN-89 huIgG1 - LALAPS (L234A; L235A; P331S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 346)
GPN-89 huIgG1 - LALAPS (L234A; L235A; P331S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 347)
GPN-89 huIgG1 - LALAPS (L234A; L235A; P329S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 348)
GPN-89 huIgG1 - LALAPS (L234A; L235A; P329S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 349)
GPN-89 huIgG1- LALAPG (L234A; L235A; P331G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 350)
GPN-89 huIgG1- LALAPG (L234A; L235A; P331G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 351)
GPN-89 huIgG1 - LALAPG (L234A; L235A; P329G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 352)
GPN-89 huIgG1 - LALAPG (L234A; L235A; P329G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 353)
GPN-89 huIgG1 - PSEG (P331S; E430G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPGK
(SEQ ID NO: 354)
GPN-89 huIgG1 - PSEG (P331S; E430G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPG
(SEQ ID NO: 355)
GPN-89 huIgG1 - NSLF (N325S: L328F) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 356)
GPN-89 huIgG1 - NSLF (N325S: L328F) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 357)
GPN-89 huIgG4 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 358)
GPN-89 huIgG4 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 359)
GPN-89 huIgG4 (S228P) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 360)
GPN-89 huIgG4 (S228P) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYYDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 361)
GPN-89 Light Chain
DIVMTQSPDSLAVSLGERATINCKSSRSVDYYGIGFLNWYQQKPGQPPKLLIYATSHQGSGVPDR
FSGSGSGTDFTLTISSLQAEDVAVYYCQQQKEVPPTFGGGTKVEIKRTVAAPSVFIFPPSDEQLKS
GTASVVCLLNNFYPREAKVQWKVDNALQSGNSQESVTEQDSKDSTYSLSSTLTLSKADYEKHK
VYACEVTHQGLSSPVTKSFNRGEC (SEQ ID NO: 424)
GPN-90 huIgG1 - WT with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPGK
(SEQ ID NO: 362)
GPN-90 huIgG1 - WT without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPG
(SEQ ID NO: 363)
GPN-90 huIgG1 - LALAPS (L234A; L235A; P331S) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALPASIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGK
(SEQ ID NO: 364)
GPN-90 huIgG1 - LALAPS (L234A; L235A; P331S) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALPASIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
(SEQ ID NO: 365)
GPN-90 huIgG1 - LALAPS (L234A; L235A; P329S) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALSAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGK
(SEQ ID NO: 366)
GPN-90 huIgG1 - LALAPS (L234A; L235A; P329S) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALSAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
(SEQ ID NO: 367)
GPN-90 huIgG1- LALAPG (L234A; L235A; P331G) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALPAGIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGK
(SEQ ID NO: 368)
GPN-90 huIgG1- LALAPG (L234A; L235A; P331G) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALPAGIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
(SEQ ID NO: 369)
GPN-90 huIgG1 - LALAPG (L234A; L235A; P329G) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALGAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGK
(SEQ ID NO: 370)
GPN-90 huIgG1 - LALAPG (L234A; L235A; P329G) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEY
KCKVSNKALGAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
(SEQ ID NO: 371)
GPN-90 huIgG1 - PSEG (P331S; E430G) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPASIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHGALHNHYTQKSLSLSPGK
(SEQ ID NO: 372)
GPN-90 huIgG1 - PSEG (P331S; E430G) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPASIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHGALHNHYTQKSLSLSPG
(SEQ ID NO: 373)
GPN-90 huIgG1 - NSLF (N325S: L328F) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSSKAFPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 374)
GPN-90 huIgG1 - NSLF (N325S: L328F) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSSKAFPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 375)
GPN-90 huIgG4 - WT with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 376)
GPN-90 huIgG4 - WT without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 377)
GPN-90 huIgG4 (S228P) - with C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLGK
(SEQ ID NO: 378)
GPN-90 huIgG4 (S228P) - without C-terminal lysine
QVQLQESGPGLVKPSETLSLTCTVSGISITTGNYRWSWIRQPPGKGLEWIGYIDYSGTITYNPSLK
QRVTITRDTSKNQFSLELSSVTAADTAVYYCARDLTHWYFDVWGQGTLVTVSSASTKGPSVFPL
APCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLG
(SEQ ID NO: 379)
GPN-90 Light Chain
DIVMTQSPDSLAVSLGERATINCKSSRSVDYYGIGFLNWYQQKPGQPPKLLIYATSHQGSGVPDR
FSGSGSGTDFTLTISSLQAEDVAVYYCQQQKEVPPTFGGGTKVEIKRTVAAPSVFIFPPSDEQLKS
GTASVVCLLNNFYPREAKVQWKVDNALQSGNSQESVTEQDSKDSTYSLSSTLTLSKADYEKHK
VYACEVTHQGLSSPVTKSFNRGEC (SEQ ID NO: 425)
GPN-91 huIgG1 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 326)
GPN-91 huIgG1 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 327)
GPN-91 huIgG1 - LALAPS (L234A; L235A; P331S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 328)
GPN-91 huIgG1 - LALAPS (L234A; L235A; P331S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPASIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 329)
GPN-91 huIgG1 - LALAPS (L234A; L235A; P329S) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGK
(SEQ ID NO: 330)
GPN-91 huIgG1 - LALAPS (L234A; L235A; P329S) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALSAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPG
(SEQ ID NO: 331)
GPN-91 huIgG1- LALAPG (L234A; L235A; P331G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 332)
GPN-91 huIgG1- LALAPG (L234A; L235A; P331G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALPAGIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 333)
GPN-91 huIgG1 - LALAPG (L234A; L235A; P329G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK
(SEQ ID NO: 334)
GPN-91 huIgG1 - LALAPG (L234A; L235A; P329G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
(SEQ ID NO: 335)
GPN-91 huIgG1 - PSEG (P331S; E430G) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPGK
(SEQ ID NO: 336)
GPN-91 huIgG1 - PSEG (P331S; E430G) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPASIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHGALHNHYTQKSLSLSPG
(SEQ ID NO: 337)
GPN-91 huIgG1 - NSLF (N325S: L328F) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
(SEQ ID NO: 338)
GPN-91 huIgG1 - NSLF (N325S: L328F) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSSKAFPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
(SEQ ID NO: 339)
GPN-91 huIgG4 - WT with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 340)
GPN-91 huIgG4 - WT without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 341)
GPN-91 huIgG4 (S228P) - with C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 342)
GPN-91 huIgG4 (S228P) - without C-terminal lysine
QVQLQESGPGLVKPSETLSLTCTVSGISITTGNYRWSWIRQPPGKGLEWIGYIDYSGTITYNPSLKS
RVTITRDTSKNQFSLELSSVTAADTAVYYCARDLTHWYFDVWGQGTLVTVSSASTKGPSVFPLA
PCSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
(SEQ ID NO: 343)
GPN-91 Light Chain
DIVMTQSPDSLAVSLGERATINCKSSRSVDYYGIGFLNWYQQKPGQPPKLLIYATSHQGSGVPDR
FSGSGSGTDFTLTISSLQAEDVAVYYCQQQKERPPTFGGGTKVEIKRTVAAPSVFIFPPSDEQLKS
GTASVVCLLNNFYPREAKVQWKVDNALQSGNSQESVTEQDSKDSTYSLSSTLTLSKADYEKHK
VYACEVTHQGLSSPVTKSFNRGEC (SEQ ID NO: 426)
GPN-92 huIgG1 - WT with C-terminal lysine
(SEQ ID NO: 380)
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK
GPN-92 huIgG1 - WT without C-terminal lysine
(SEQ ID NO: 381)
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG
GPN-92 huIgG1 - LALAPS (L234A; L235A; P331S) - with C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 382)
GPN-92 huIgG1 - LALAPS (L234A; L235A; P331S) - without C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 383)
GPN-92 huIgG1 - LALAPS (L234A; L235A; P329S) - with C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 384)
GPN-92 huIgG1 - LALAPS (L234A; L235A; P329S) - without C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 385)
GPN-92 huIgG1- LALAPG (L234A; L235A; P331G) - with C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 386)
GPN-92 huIgG1- LALAPG (L234A; L235A; P331G) - without C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 387)
GPN-92 huIgG1 - LALAPG (L234A; L235A; P329G) - with C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 388)
GPN-92 huIgG1 - LALAPG (L234A; L235A; P329G) - without C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 389)
GPN-92 huIgG1 - PSEG (P331S; E430G) - with C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPGK (SEQ ID NO: 390)
GPN-92 huIgG1 - PSEG (P331S; E430G) - without C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPG (SEQ ID NO: 391)
GPN-92 huIgG1 - NSLF (N325S: L328F) - with C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 392)
GPN-92 huIgG1 - NSLF (N325S: L328F) - without C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 393)
GPN-92 huIgG4 - WT with C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 394)
GPN-92 huIgG4 - WT without C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 395)
GPN-92 huIgG4 (S228P) - with C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 396)
GPN-92 huIgG4 (S228P) - without C-terminal lysine
QVQLVQSGAEVKKPGASVKVSCKASGYTFTSYWMHWVRQAPGQGLEWMGEINPSDGRTNYA
QKFQGRVTMTVDKSTSTVYMELSSLRSEDTAVYYCARSGGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 397)
GPN-92 Light Chain
DIVMTQSPDSLAVSLGERATINCKASQDVSPSLAWYQQKPGQPPKLLIYSASYRYTGVPDRFSGS
GSGTDFTLTISSLQAEDVAVYYCQQHYSIPLTFGGGTKVEIKRTVAAPSVFIFPPSDEQLKSGTASV
VCLLNNFYPREAKVQWKVDNALQSGNSQESVTEQDSKDSTYSLSSTLTLSKADYEKHKVYACE
VTHQGLSSPVTKSFNRGEC (SEQ ID NO: 427)
GPN-93 huIgG1 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 398)
GPN-93 huIgG1 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 399)
GPN-93 huIgG1 - LALAPS (L234A; L235A; P331S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 400)
GPN-93 huIgG1 - LALAPS (L234A; L235A; P331S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 401)
GPN-93 huIgG1 - LALAPS (L234A; L235A; P329S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 402)
GPN-93 huIgG1 - LALAPS (L234A; L235A; P329S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 403)
GPN-93 huIgG1- LALAPG (L234A; L235A; P331G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 404)
GPN-93 huIgG1- LALAPG (L234A; L235A; P331G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 405)
GPN-93 huIgG1 - LALAPG (L234A; L235A; P329G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 406)
GPN-93 huIgG1 - LALAPG (L234A; L235A; P329G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 407)
GPN-93 huIgG1 - PSEG (P331S; E430G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPGK (SEQ ID NO: 408)
GPN-93 huIgG1 - PSEG (P331S; E430G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPG (SEQ ID NO: 409)
GPN-93 huIgG1 - NSLF (N325S: L328F) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 410)
GPN-93 huIgG1 - NSLF (N325S: L328F) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 411)
GPN-93 huIgG4 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 412)
GPN-93 huIgG4 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 413)
GPN-93 huIgG4 (S228P) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 414)
GPN-93 huIgG4 (S228P) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 415)
GPN-93 Light Chain
DIVMTQSPDSLAVSLGERATINCKTSQDVSPSLAWYQQKPGQPPKLLIYSASKRYTGVPDRFSGS
GSGTDFTLTISSLQAEDVAVYYCQQHYSIPLQFGGGTKVEIKRTVAAPSVFIFPPSDEQLKSGTAS
VVCLLNNFYPREAKVQWKVDNALQSGNSQESVTEQDSKDSTYSLSSTLTLSKADYEKHKVYAC
EVTHQGLSSPVTKSFNRGEC (SEQ ID NO: 428)
GPN-94 huIgG1 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 398)
GPN-94 huIgG1 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 399)
GPN-94 huIgG1 - LALAPS (L234A; L235A; P331S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 400)
GPN-94 huIgG1 - LALAPS (L234A; L235A; P331S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 401)
GPN-94 huIgG1 - LALAPS (L234A; L235A; P329S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 402)
GPN-94 huIgG1 - LALAPS (L234A; L235A; P329S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 403)
GPN-94 huIgG1- LALAPG (L234A; L235A; P331G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 404)
GPN-94 huIgG1- LALAPG (L234A; L235A; P331G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 405)
GPN-94 huIgG1 - LALAPG (L234A; L235A; P329G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 406)
GPN-94 huIgG1 - LALAPG (L234A; L235A; P329G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 407)
GPN-94 huIgG1 - PSEG (P331S; E430G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPGK (SEQ ID NO: 408)
GPN-94 huIgG1 - PSEG (P331S; E430G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPG (SEQ ID NO: 409)
GPN-94 huIgG1 - NSLF (N325S: L328F) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 410)
GPN-94 huIgG1 - NSLF (N325S: L328F) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 411)
GPN-94 huIgG4 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 412)
GPN-94 huIgG4 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 413)
GPN-94 huIgG4 (S228P) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 414)
GPN-94 huIgG4 (S228P) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 415)
GPN-94 Light Chain
DIVMTQSPDSLAVSLGERATINCKTSQDVSPSLAWYQQKPGQPPKLLIYSASKRYTGVPDRFSGS
GSGTDFTLTISSLQAEDVAVYYCQQHYSIPLTFGGGTKVEIKRTVAAPSVFIFPPSDEQLKSGTASV
VCLLNNFYPREAKVQWKVDNALQSGNSQESVTEQDSKDSTYSLSSTLTLSKADYEKHKVYACE
VTHQGLSSPVTKSFNRGEC (SEQ ID NO: 429)
GPN-95 huIgG1 - WT with C-terminal lysine
(SEQ ID NO: 398)
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK
GPN-95 huIgG1 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 399)
GPN-95 huIgG1 - LALAPS (L234A; L235A; P331S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 400)
GPN-95 huIgG1 - LALAPS (L234A; L235A; P331S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 401)
GPN-95 huIgG1 - LALAPS (L234A; L235A; P329S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 402)
GPN-95 huIgG1 - LALAPS (L234A; L235A; P329S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 403)
GPN-95 huIgG1- LALAPG (L234A; L235A; P331G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 404)
GPN-95 huIgG1- LALAPG (L234A; L235A; P331G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 405)
GPN-95 huIgG1 - LALAPG (L234A; L235A; P329G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 406)
GPN-95 huIgG1 - LALAPG (L234A; L235A; P329G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 407)
GPN-95 huIgG1 - PSEG (P331S; E430G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPGK (SEQ ID NO: 408)
GPN-95 huIgG1 - PSEG (P331S; E430G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPG (SEQ ID NO: 409)
GPN-95 huIgG1 - NSLF (N325S: L328F) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 410)
GPN-95 huIgG1 - NSLF (N325S: L328F) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 411)
GPN-95 huIgG4 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 412)
GPN-95 huIgG4 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 413)
GPN-95 huIgG4 (S228P) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 414)
GPN-95 huIgG4 (S228P) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 415)
GPN-95 Light Chain
DIVMTQSPDSLAVSLGERATINCKTSQDVSPSLAWYQQKPGQPPKLLIYSARYRYTGVPDRFSGS
GSGTDFTLTISSLQAEDVAVYYCQQHYSIPLQFGGGTKVEIKRTVAAPSVFIFPPSDEQLKSGTAS
VVCLLNNFYPREAKVQWKVDNALQSGNSQESVTEQDSKDSTYSLSSTLTLSKADYEKHKVYAC
EVTHQGLSSPVTKSFNRGEC (SEQ ID NO: 430)
GPN-96 huIgG1 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 398)
GPN-96 huIgG1 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 399)
GPN-96 huIgG1 - LALAPS (L234A; L235A; P331S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 400)
GPN-96 huIgG1 - LALAPS (L234A; L235A; P331S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 401)
GPN-96 huIgG1 - LALAPS (L234A; L235A; P329S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 402)
GPN-96 huIgG1 - LALAPS (L234A; L235A; P329S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 403)
GPN-96 huIgG1- LALAPG (L234A; L235A; P331G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 404)
GPN-96 huIgG1- LALAPG (L234A; L235A; P331G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 405)
GPN-96 huIgG1 - LALAPG (L234A; L235A; P329G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 406)
GPN-96 huIgG1 - LALAPG (L234A; L235A; P329G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 407)
GPN-96 huIgG1 - PSEG (P331S; E430G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPGK (SEQ ID NO: 408)
GPN-96 huIgG1 - PSEG (P331S; E430G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPG (SEQ ID NO: 409)
GPN-96 huIgG1 - NSLF (N325S: L328F) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 410)
GPN-96 huIgG1 - NSLF (N325S: L328F) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 411)
GPN-96 huIgG4 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 412)
GPN-96 huIgG4 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 413)
GPN-96 huIgG4 (S228P) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 414)
GPN-96 huIgG4 (S228P) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 415)
GPN-96 Light Chain
DIVMTQSPDSLAVSLGERATINCKASQDVSPSLAWYQQKPGQPPKLLIYRASYRYTGVPDRFSGS
GSGTDFTLTISSLQAEDVAVYYCQQHYSIPLTFGGGTKVEIKRTVAAPSVFIFPPSDEQLKSGTASV
VCLLNNFYPREAKVQWKVDNALQSGNSQESVTEQDSKDSTYSLSSTLTLSKADYEKHKVYACE
VTHQGLSSPVTKSFNRGEC (SEQ ID NO: 431)
GPN-97 huIgG1 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 398)
GPN-97 huIgG1 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 399)
GPN-97 huIgG1 - LALAPS (L234A; L235A; P331S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 400)
GPN-97 huIgG1 - LALAPS (L234A; L235A; P331S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 401)
GPN-97 huIgG1 - LALAPS (L234A; L235A; P329S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 402)
GPN-97 huIgG1 - LALAPS (L234A; L235A; P329S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 403)
GPN-97 huIgG1- LALAPG (L234A; L235A; P331G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 404)
GPN-97 huIgG1- LALAPG (L234A; L235A; P331G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 405)
GPN-97 huIgG1 - LALAPG (L234A; L235A; P329G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 406)
GPN-97 huIgG1 - LALAPG (L234A; L235A; P329G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 407)
GPN-97 huIgG1 - PSEG (P331S; E430G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPGK (SEQ ID NO: 408)
GPN-97 huIgG1 - PSEG (P331S; E430G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPG (SEQ ID NO: 409)
GPN-97 huIgG1 - NSLF (N325S: L328F) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 410)
GPN-97 huIgG1 - NSLF (N325S: L328F) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 411)
GPN-97 huIgG4 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 412)
GPN-97 huIgG4 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 413)
GPN-97 huIgG4 (S228P) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 414)
GPN-97 huIgG4 (S228P) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 415)
GPN-97 Light Chain
DIVMTQSPDSLAVSLGERATINCKTSQDVSPSLAWYQQKPGQPPKLLIYSGSYRYTGVPDRFSGS
GSGTDFTLTISSLQAEDVAVYYCQQHYSIPLTFGGGTKVEIKRTVAAPSVFIFPPSDEQLKSGTASV
VCLLNNFYPREAKVQWKVDNALQSGNSQESVTEQDSKDSTYSLSSTLTLSKADYEKHKVYACE
VTHQGLSSPVTKSFNRGEC (SEQ ID NO: 432)
GPN-98 huIgG1 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 398)
GPN-98 huIgG1 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 399)
GPN-98 huIgG1 - LALAPS (L234A; L235A; P331S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 400)
GPN-98 huIgG1 - LALAPS (L234A; L235A; P331S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 401)
GPN-98 huIgG1 - LALAPS (L234A; L235A; P329S) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 402)
GPN-98 huIgG1 - LALAPS (L234A; L235A; P329S) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALSAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 403)
GPN-98 huIgG1- LALAPG (L234A; L235A; P331G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 404)
GPN-98 huIgG1- LALAPG (L234A; L235A; P331G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAGIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 405)
GPN-98 huIgG1 - LALAPG (L234A; L235A; P329G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPGK (SEQ ID NO: 406)
GPN-98 huIgG1 - LALAPG (L234A; L235A; P329G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSL
SLSPG (SEQ ID NO: 407)
GPN-98 huIgG1 - PSEG (P331S; E430G) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPGK (SEQ ID NO: 408)
GPN-98 huIgG1 - PSEG (P331S; E430G) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHGALHNHYTQKSLSL
SPG (SEQ ID NO: 409)
GPN-98 huIgG1 - NSLF (N325S: L328F) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPGK (SEQ ID NO: 410)
GPN-98 huIgG1 - NSLF (N325S: L328F) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSSKAFPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSL
SPG (SEQ ID NO: 411)
GPN-98 huIgG4 - WT with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 412)
GPN-98 huIgG4 - WT without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 413)
GPN-98 huIgG4 (S228P) - with C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 414)
GPN-98 huIgG4 (S228P) - without C-terminal lysine
QVQLVQSGAEVKKPGASVKVSCKASGYTFTSYWMHWVRQAPGQGLEWMGEINPYDQRTNYA
QKFQGRVTMTVDKSTSTVYMELSSLRSEDTAVYYCARSTGNYAWFAYWGQGTLVTVSSASTK
GPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG
(SEQ ID NO: 415)
GPN-98 Light Chain
DIVMTQSPDSLAVSLGERATINCKASQDVSPSLAWYQQKPGQPPKLLIYSASYRYTGVPDRFSGS
GSGTDFTLTISSLQAEDVAVYYCQQHYSIPLTFGGGTKVEIKRTVAAPSVFIFPPSDEQLKSGTASV
VCLLNNFYPREAKVQWKVDNALQSGNSQESVTEQDSKDSTYSLSSTLTLSKADYEKHKVYACE
VTHQGLSSPVTKSFNRGEC (SEQ ID NO: 433)
Full-Length Cynomolgus GPNMB Amino Acid Sequence (SEQ ID NO: 434):
MECLYYFLGFLLLAARLPLDAAKRFHDVLGNERPSAYMREHNQLNGWSSDENDWNEKLYP
VWKRGDMRWKNSWKGGRVQAVLTSDSPALVGSNITFVVNLVFPRCQKEDANGNIVYEKNC
RNEAGLSADPYVYNWTAWSEDSDGENGTGQSHHNVFPDGKPFPHHPGWRRWNFVYVFHTL
GQYFQKLGRCSVRVSVNTANVTLGPQLMEVTVYRRHGRAYVPIAQVKDVYVVTDQIPVFV
TMSQKNDRNSSDETFLKDLPITFDVLIHDPSHFLNYSTINYKWSFGDNTGLFVSINHTVN
HTYVLNGTFSLNLTVKAAAPGPCPPPPPPPPRPPKPTPSLATTLKSCYSNTPGPAGDNPL
GLSEIPDENCQINRYGHFQATITIVEGILEVNIIQTTDVLMLVPGPDSSLIDFVVTCQGS
IPTEVCTIISDPTCEITQNAVCSPVDVDEMCLLTVRRAFNGSGTYCVNLTLGDDTSLALT
STLISVPDRDPASSLRMANTALISVGCLAIFVTVISLLVYKKHKKYKPIENSPANVVGSK
GLSAFLNRAKAVFFPGNQEKDPLLKNQEFKGVS
Full-Length Mouse GPNMB Amino Acid Sequence (SEQ ID NO: 435):
MECLYYFLGFLLLAARLPLDAAKRFHDVLGNERPSAYMREHNQLNGWSSDENDWNEKLYP
VWKRGDMRWKNSWKGGRVQAVLTSDSPALVGSNITFVVNLVFPRCQKEDANGNIVYEKNC
RNEAGLSADPYVYNWTAWSEDSDGENGTGQSHHNVFPDGKPFPHHPGWRRWNFVYVFHTL
GQYFQKLGRCSVRVSVNTANVTLGPQLMEVTVYRRHGRAYVPIAQVKDVYVVTDQIPVFV
TMSQKNDRNSSDETFLKDLPITFDVLIHDPSHFLNYSTINYKWSFGDNTGLFVSINHTVN
HTYVLNGTFSLNLTVKAAAPGPCPPPPPPPPRPPKPTPSLATTLKSCYSNTPGPAGDNPL
GLSEIPDENCQINRYGHFQATITIVEGILEVNIIQTTDVLMLVPGPDSSLIDFVVTCQGS
IPTEVCTIISDPTCEITQNAVCSPVDVDEMCLLTVRRAFNGSGTYCVNLTLGDDTSLALT
STLISVPDRDPASSLRMANTALISVGCLAIFVTVISLLVYKKHKKYKPIENSPANVVGSK
GLSAFLNRAKAVFFPGNQEKDPLLKNQEFKGVS

1. An isolated anti-Glycoprotein nonmetastatic melanoma protein B (GPNMB) antibody, wherein the anti-GPNMB antibody antagonizes GPNMB activity.

2-13. (canceled)

14. The anti-GPNMB antibody of claim 1, wherein the anti-GPNMB antibody is a GPNMB ligand blocking antibody.

15. The anti-GPNMB antibody of claim 1, wherein the anti-GPNMB antibody is a non-blocking antibody.

16. The anti-GPNMB antibody of claim 1, wherein the anti-GPNMB antibody antagonizes GPNMB activity independent of ligand blocking activity.

17-39. (canceled)

40. An isolated anti-Glycoprotein nonmetastatic melanoma protein B (GPNMB) antibody, wherein the anti-GPNMB antibody comprises:
a. a VH comprising an HVR-H1 comprising the amino acid sequence of SEQ ID NO: 5, an HVR-H2 comprising the amino acid sequence of SEQ ID NO: 6, and an HVR-H3 comprising the amino acid sequence of SEQ ID NO: 7; and a VL comprising an HVR-L1 comprising the amino acid sequence of SEQ ID NO: 83, an HVR-L2 comprising the amino acid sequence of SEQ ID NO: 84, and an HVR-L3 comprising the amino acid sequence of SEQ ID NO: 85;
b. a VH comprising an HVR-H1 comprising the amino acid sequence of SEQ ID NO: 8, an HVR-H2 comprising the amino acid sequence of SEQ ID NO: 9, and an HVR-H3 comprising the amino acid sequence of SEQ ID NO: 10; and a VL comprising an HVR-L1 comprising the amino acid sequence of SEQ ID NO: 86, an HVR-L2 comprising the amino acid sequence of SEQ ID NO: 87, and an HVR-L3 comprising the amino acid sequence of SEQ ID NO: 88;
c. a VH comprising an HVR-H1 comprising the amino acid sequence of SEQ ID NO: 11, an HVR-H2 comprising the amino acid sequence of SEQ ID NO: 12, and an HVR-H3 comprising the amino acid sequence of SEQ ID NO: 13; and a VL comprising an HVR-L1 comprising the amino acid sequence of SEQ ID NO: 89, an HVR-L2 comprising the amino acid sequence of SEQ ID NO: 90, and an HVR-L3 comprising the amino acid sequence of SEQ ID NO: 91
d. a VH comprising an HVR-H1 comprising the amino acid sequence of SEQ ID NO: 14, an HVR-H2 comprising the amino acid sequence of SEQ ID NO: 15, and an HVR-H3 comprising the amino acid sequence of SEQ ID NO: 16; and a VL comprising an HVR-L1 comprising the amino acid sequence of SEQ ID NO: 92, an HVR-L2 comprising the amino acid sequence of SEQ ID NO: 93, and an HVR-L3 comprising the amino acid sequence of SEQ ID NO: 94;
e. a VH comprising an HVR-H1 comprising the amino acid sequence of SEQ ID NO: 17, an HVR-H2 comprising the amino acid sequence of SEQ ID NO: 18, and an HVR-H3 comprising the amino acid sequence of SEQ ID NO: 19; and a VL comprising an HVR-L1 comprising the amino acid sequence of SEQ ID NO: 95, an HVR-L2 comprising the amino acid sequence of SEQ ID NO: 96, and an HVR-L3 comprising the amino acid sequence of SEQ ID NO: 97;
f. a VH comprising an HVR-H1 comprising the amino acid sequence of SEQ ID NO: 20, an HVR-H2 comprising the amino acid sequence of SEQ ID NO: 21, and an HVR-H3 comprising the amino acid sequence of SEQ ID NO: 22; and a VL comprising an HVR-L1 comprising the amino acid sequence of SEQ ID NO: 98, an HVR-L2 comprising the amino acid sequence of SEQ ID NO: 99, and an HVR-L3 comprising the amino acid sequence of SEQ ID NO: 100;
g. a VH comprising an HVR-H1 comprising the amino acid sequence of SEQ ID NO: 23, an HVR-H2 comprising the amino acid sequence of SEQ ID NO: 24, and an HVR-H3 comprising the amino acid sequence of SEQ ID NO: 25; and a VL comprising an HVR-L1 comprising the amino acid sequence of SEQ ID NO: 101, an HVR-L2 comprising the amino acid sequence of SEQ ID NO: 102, and an HVR-L3 comprising the amino acid sequence of SEQ ID NO: 103;
h. a VH comprising an HVR-H1 comprising the amino acid sequence of SEQ ID NO: 26, an HVR-H2 comprising the amino acid sequence of SEQ ID NO: 27, and an HVR-H3 comprising the amino acid sequence of SEQ ID NO: 28; and a VL comprising an HVR-L1 comprising the amino acid sequence of SEQ ID NO: 104, an HVR-L2 comprising the amino acid sequence of SEQ ID NO: 105, and an HVR-L3 comprising the amino acid sequence of SEQ ID NO: 106;
i. a VH comprising an HVR-H1 comprising the amino acid sequence of SEQ ID NO: 29, an HVR-H2 comprising the amino acid sequence of SEQ ID NO: 30, and an HVR-H3 comprising the amino acid sequence of SEQ ID NO: 31; and a VL comprising an HVR-L1 comprising the amino acid sequence of SEQ ID NO: 107, an HVR-L2 comprising the amino acid sequence of SEQ ID NO: 108, and an HVR-L3 comprising the amino acid sequence of SEQ ID NO: 109;
j. a VH comprising an HVR-H1 comprising the amino acid sequence of SEQ ID NO: 32, an HVR-H2 comprising the amino acid sequence of SEQ ID NO: 33, and an HVR-H3 comprising the amino acid sequence of SEQ ID NO: 34; and a VL comprising an HVR-L1 comprising the amino acid sequence of SEQ ID NO: 110, an HVR-L2 comprising the amino acid sequence of SEQ ID NO: 111, and an HVR-L3 comprising the amino acid sequence of SEQ ID NO: 112;
k. a VH comprising an HVR-H1 comprising the amino acid sequence of SEQ ID NO: 35, an HVR-H2 comprising the amino acid sequence of SEQ ID NO: 36, and an HVR-H3 comprising the amino acid sequence of SEQ ID NO: 37; and a VL comprising an HVR-L1 comprising the amino acid sequence of SEQ ID NO: 113, an HVR-L2 comprising the amino acid sequence of SEQ ID NO: 114, and an HVR-L3 comprising the amino acid sequence of SEQ ID NO: 115;
l. a VH comprising an HVR-H1 comprising the amino acid sequence of SEQ ID NO: 38, an HVR-H2 comprising the amino acid sequence of SEQ ID NO: 39, and an HVR-H3 comprising the amino acid sequence of SEQ ID NO: 40; and a VL comprising an HVR-L1 comprising the amino acid sequence of SEQ ID NO: 116, an HVR-L2 comprising the amino acid sequence of SEQ ID NO: 117, and an HVR-L3 comprising the amino acid sequence of SEQ ID NO: 118;
m. a VH comprising an HVR-H1 comprising the amino acid sequence of SEQ ID NO: 41, an HVR-H2 comprising the amino acid sequence of SEQ ID NO: 42, and an HVR-H3 comprising the amino acid sequence of SEQ ID NO: 43; and a VL comprising an HVR-L1 comprising the amino acid sequence of SEQ ID NO: 119, an HVR-L2 comprising the amino acid sequence of SEQ ID NO: 120, and an HVR-L3 comprising the amino acid sequence of SEQ ID NO: 121;
n. a VH comprising an HVR-H1 comprising the amino acid sequence of SEQ ID NO: 44, an HVR-H2 comprising the amino acid sequence of SEQ ID NO: 45, and an HVR-H3 comprising the amino acid sequence of SEQ ID NO: 46; and a VL comprising an HVR-L1 comprising the amino acid sequence of SEQ ID NO: 122, an HVR-L2 comprising the amino acid sequence of SEQ ID NO: 123, and an HVR-L3 comprising the amino acid sequence of SEQ ID NO: 124;
o. a VH comprising an HVR-H1 comprising the amino acid sequence of SEQ ID NO: 47, an HVR-H2 comprising the amino acid sequence of SEQ ID NO: 48, and an HVR-H3 comprising the amino acid sequence of SEQ ID NO: 49; and a VL comprising an HVR-L1 comprising the amino acid sequence of SEQ ID NO: 125, an HVR-L2 comprising the amino acid sequence of SEQ ID NO: 126, and an HVR-L3 comprising the amino acid sequence of SEQ ID NO: 127;
p. a VH comprising an HVR-H1 comprising the amino acid sequence of SEQ ID NO: 50, an HVR-H2 comprising the amino acid sequence of SEQ ID NO: 51, and an HVR-H3 comprising the amino acid sequence of SEQ ID NO: 52; and a VL comprising an HVR-L1 comprising the amino acid sequence of SEQ ID NO: 128, an HVR-L2 comprising the amino acid sequence of SEQ ID NO: 129, and an HVR-L3 comprising the amino acid sequence of SEQ ID NO: 130;
q. a VH comprising an HVR-H1 comprising the amino acid sequence of SEQ ID NO: 53, an HVR-H2 comprising the amino acid sequence of SEQ ID NO: 54, and an HVR-H3 comprising the amino acid sequence of SEQ ID NO: 55; and a VL comprising an HVR-L1 comprising the amino acid sequence of SEQ ID NO: 131, an HVR-L2 comprising the amino acid sequence of SEQ ID NO: 132, and an HVR-L3 comprising the amino acid sequence of SEQ ID NO: 133;
r. a VH comprising an HVR-H1 comprising the amino acid sequence of SEQ ID NO: 56, an HVR-H2 comprising the amino acid sequence of SEQ ID NO: 57, and an HVR-H3 comprising the amino acid sequence of SEQ ID NO: 58; and a VL comprising an HVR-L1 comprising the amino acid sequence of SEQ ID NO: 134, an HVR-L2 comprising the amino acid sequence of SEQ ID NO: 135, and an HVR-L3 comprising the amino acid sequence of SEQ ID NO: 136;
s. a VH comprising an HVR-H1 comprising the amino acid sequence of SEQ ID NO: 59, an HVR-H2 comprising the amino acid sequence of SEQ ID NO: 60, and an HVR-H3 comprising the amino acid sequence of SEQ ID NO: 61; and a VL comprising an HVR-L1 comprising the amino acid sequence of SEQ ID NO: 137, an HVR-L2 comprising the amino acid sequence of SEQ ID NO: 138, and an HVR-L3 comprising the amino acid sequence of SEQ ID NO: 139;
t. a VH comprising an HVR-H1 comprising the amino acid sequence of SEQ ID NO: 62, an HVR-H2 comprising the amino acid sequence of SEQ ID NO: 63, and an HVR-H3 comprising the amino acid sequence of SEQ ID NO: 64; and a VL comprising an HVR-L1 comprising the amino acid sequence of SEQ ID NO: 140, an HVR-L2 comprising the amino acid sequence of SEQ ID NO: 141, and an HVR-L3 comprising the amino acid sequence of SEQ ID NO: 142;
u. a VH comprising an HVR-H1 comprising the amino acid sequence of SEQ ID NO: 65, an HVR-H2 comprising the amino acid sequence of SEQ ID NO: 66, and an HVR-H3 comprising the amino acid sequence of SEQ ID NO: 67; and a VL comprising an HVR-L1 comprising the amino acid sequence of SEQ ID NO: 143, an HVR-L2 comprising the amino acid sequence of SEQ ID NO: 144, and an HVR-L3 comprising the amino acid sequence of SEQ ID NO: 145;
v. a VH comprising an HVR-H1 comprising the amino acid sequence of SEQ ID NO: 68, an HVR-H2 comprising the amino acid sequence of SEQ ID NO: 69, and an HVR-H3 comprising the amino acid sequence of SEQ ID NO: 70; and a VL comprising an HVR-L1 comprising the amino acid sequence of SEQ ID NO: 146, an HVR-L2 comprising the amino acid sequence of SEQ ID NO: 147, and an HVR-L3 comprising the amino acid sequence of SEQ ID NO: 148;
w. a VH comprising an HVR-H1 comprising the amino acid sequence of SEQ ID NO: 71, an HVR-H2 comprising the amino acid sequence of SEQ ID NO: 72, and an HVR-H3 comprising the amino acid sequence of SEQ ID NO: 73; and a VL comprising an HVR-L1 comprising the amino acid sequence of SEQ ID NO: 149, an HVR-L2 comprising the amino acid sequence of SEQ ID NO: 150, and an HVR-L3 comprising the amino acid sequence of SEQ ID NO: 151;
x. a VH comprising an HVR-H1 comprising the amino acid sequence of SEQ ID NO: 74, an HVR-H2 comprising the amino acid sequence of SEQ ID NO: 75, and an HVR-H3 comprising the amino acid sequence of SEQ ID NO: 76; and a VL comprising an HVR-L1 comprising the amino acid sequence of SEQ ID NO: 152, an HVR-L2 comprising the amino acid sequence of SEQ ID NO: 153, and an HVR-L3 comprising the amino acid sequence of SEQ ID NO: 154;
y. a VH comprising an HVR-H1 comprising the amino acid sequence of SEQ ID NO: 77, an HVR-H2 comprising the amino acid sequence of SEQ ID NO: 78, and an HVR-H3 comprising the amino acid sequence of SEQ ID NO: 79; and a VL comprising an HVR-L1 comprising the amino acid sequence of SEQ ID NO: 155, an HVR-L2 comprising the amino acid sequence of SEQ ID NO: 156, and an HVR-L3 comprising the amino acid sequence of SEQ ID NO: 157;
z. a VH comprising an HVR-H1 comprising the amino acid sequence of SEQ ID NO:71, an HVR-H2 comprising the amino acid sequence of SEQ ID NO:255, an HVR-H3 comprising the amino acid sequence of SEQ ID NO:73, a VL comprising an HVR-L1 comprising the amino acid sequence of SEQ ID NO:263, an HVR-L2 comprising the amino acid sequence of SEQ ID NO:269, and an HVR-L3 comprising the amino acid sequence of SEQ ID NO:151;
aa. a VH comprising an HVR-H1 comprising amino acid sequence of SEQ ID NO:71, an HVR-H2 comprising the amino acid sequence of SEQ ID NO:256, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:270, and an HVR-L3 comprising the amino acid sequence of SEQ ID NO:278;
bb. a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:73, a VL comprising an HVR-L1 comprising the amino acid sequence of SEQ ID NO:265, an HVR-L2 comprising the amino acid sequence of SEQ ID NO:271, and an HVR-L3 comprising the amino acid sequence of SEQ ID NO:278;
cc. a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:261, a VL comprising an HVR-L1 comprising the amino acid sequence of SEQ ID NO:265, an HVR-L2 comprising the amino acid sequence of SEQ ID NO:271, and an HVR-L3 comprising the amino acid sequence of SEQ ID NO:278;
dd. a VH comprising an HVR-H1 comprising the amino acid sequence of SEQ ID NO:71, an HVR-H2 comprising the amino acid sequence of SEQ ID NO:258, an HVR-H3 comprising the amino acid sequence of SEQ ID NO:73, a VL comprising an HVR-L1 comprising the amino acid sequence of SEQ ID NO:266, an HVR-L2 comprising the amino acid sequence of SEQ ID NO:272, and an HVR-L3 comprising the amino acid sequence of SEQ ID NO:278;
ee. a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:273, and an HVR-L3 comprising the amino acid sequence of SEQ ID NO:278;
ff. a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:261, a VL comprising an HVR-L1 comprising the amino acid sequence of SEQ ID NO:264, an HVR-L2 comprising the amino acid sequence of SEQ ID NO:273, and an HVR-L3 comprising the amino acid sequence of SEQ ID NO:278;
gg. a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:272, and an HVR-L3 comprising the amino acid sequence of SEQ ID NO:279;
hh. a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:261, a VL comprising an HVR-L1 comprising the amino acid sequence of SEQ ID NO:264, an HVR-L2 comprising the amino acid sequence of SEQ ID NO:272, and an HVR-L3 comprising the amino acid sequence of SEQ ID NO:278;
ii. a VH comprising an HVR-H1 comprising the amino acid sequence of SEQ ID NO:71, an HVR-H2 comprising the amino acid sequence of SEQ ID NO:258, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:272, and an HVR-L3 comprising the amino acid sequence of SEQ ID NO:278;
jj. a VH comprising an HVR-H1 comprising the amino acid sequence of SEQ ID NO:71, an HVR-H2 comprising the amino acid sequence of SEQ ID NO:257, an HVR-H3 comprising the amino acid sequence of SEQ ID NO:73, a VL comprising an HVR-L1 comprising the amino acid sequence of SEQ ID NO:264, an HVR-L2 comprising the amino acid sequence of SEQ ID NO:272, and an HVR-L3 comprising the amino acid sequence of SEQ ID NO:280;
kk. a VH comprising an HVR-H1 comprising the amino acid sequence of SEQ ID NO:23, an HVR-H2 comprising the amino acid sequence of SEQ ID NO:259, an HVR-H3 comprising the amino acid sequence of SEQ ID NO:19, a VL comprising an HVR-L1 comprising the amino acid sequence of SEQ ID NO:267, an HVR-L2 comprising the amino acid sequence of SEQ ID NO:96, and an HVR-L3 comprising the amino acid sequence of SEQ ID NO:97;
ll. a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:268, an HVR-L2 comprising the amino acid sequence of SEQ ID NO:274, and an HVR-L3 comprising the amino acid sequence of SEQ ID NO:281;
mm. a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:268, an HVR-L2 comprising the amino acid sequence of SEQ ID NO:274, and an HVR-L3 comprising the amino acid sequence of SEQ ID NO:97;
nn. a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:268, an HVR-L2 comprising the amino acid sequence of SEQ ID NO:275, and an HVR-L3 comprising the amino acid sequence of SEQ ID NO:281;
oo. a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:267, an HVR-L2 comprising the amino acid sequence of SEQ ID NO:276, and an HVR-L3 comprising the amino acid sequence of SEQ ID NO:97;
pp. a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:268, an HVR-L2 comprising the amino acid sequence of SEQ ID NO:277, and an HVR-L3 comprising the amino acid sequence of SEQ ID NO:97; or
qq. a VH comprising an HVR-H1 comprising the amino acid sequence of SEQ ID NO:23, an HVR-H2 comprising the amino acid sequence of SEQ ID NO:260, an HVR-H3 comprising the amino acid sequence of SEQ ID NO:262, a VL comprising an HVR-L1 comprising the amino acid sequence of SEQ ID NO:267, an HVR-L2 comprising the amino acid sequence of SEQ ID NO:96, and an HVR-L3 comprising the amino acid sequence of SEQ ID NO:97.

41-44. (canceled)

45. The antibody of claim 40, wherein the antibody comprises
a. a VH comprising the amino acid sequence of SEQ ID NO: 161 and a VL comprising the amino acid sequence of SEQ ID NO: 162;
b. a VH comprising the amino acid sequence of SEQ ID NO: 163 and a VL comprising the amino acid sequence of SEQ ID NO: 164;
c. a VH comprising the amino acid sequence of SEQ ID NO: 165 and a VL comprising the amino acid sequence of SEQ ID NO: 166;
d. a VH comprising the amino acid sequence of SEQ ID NO: 167 and a VL comprising the amino acid sequence of SEQ ID NO: 168;
e. a VH comprising the amino acid sequence of SEQ ID NO: 169 and a VL comprising the amino acid sequence of SEQ ID NO: 170;
f. a VH comprising the amino acid sequence of SEQ ID NO: 171 and a VL comprising the amino acid sequence of SEQ ID NO: 172;
g. a VH comprising the amino acid sequence of SEQ ID NO: 173 and a VL comprising the amino acid sequence of SEQ ID NO: 174;
h. a VH comprising the amino acid sequence of SEQ ID NO: 175 and a VL comprising the amino acid sequence of SEQ ID NO: 176;
i. a VH comprising the amino acid sequence of SEQ ID NO: 177 and a VL comprising the amino acid sequence of SEQ ID NO: 178;
j. a VH comprising the amino acid sequence of SEQ ID NO: 179 and a VL comprising the amino acid sequence of SEQ ID NO: 180;
k. a VH comprising the amino acid sequence of SEQ ID NO: 181 and a VL comprising the amino acid sequence of SEQ ID NO: 182;
l. a VH comprising the amino acid sequence of SEQ ID NO: 183 and a VL comprising the amino acid sequence of SEQ ID NO: 184;
m. a VH comprising the amino acid sequence of SEQ ID NO: 185 and a VL comprising the amino acid sequence of SEQ ID NO: 186;
n. a VH comprising the amino acid sequence of SEQ ID NO: 187 and a VL comprising the amino acid sequence of SEQ ID NO: 188;
o. a VH comprising the amino acid sequence of SEQ ID NO: 189 and a VL comprising the amino acid sequence of SEQ ID NO: 190;
p. a VH comprising the amino acid sequence of SEQ ID NO: 191 and a VL comprising the amino acid sequence of SEQ ID NO: 192;
q. a VH comprising the amino acid sequence of SEQ ID NO: 193 and a VL comprising the amino acid sequence of SEQ ID NO: 194;
r. a VH comprising the amino acid sequence of SEQ ID NO: 195 and a VL comprising the amino acid sequence of SEQ ID NO: 196;
s. a VH comprising the amino acid sequence of SEQ ID NO: 197 and a VL comprising the amino acid sequence of SEQ ID NO: 198;
t. a VH comprising the amino acid sequence of SEQ ID NO: 199 and a VL comprising the amino acid sequence of SEQ ID NO: 200;
u. a VH comprising the amino acid sequence of SEQ ID NO: 201 and a VL comprising the amino acid sequence of SEQ ID NO: 202;
v. a VH comprising the amino acid sequence of SEQ ID NO: 203 and a VL comprising the amino acid sequence of SEQ ID NO: 204;
w. a VH comprising the amino acid sequence of SEQ ID NO: 205 and a VL comprising the amino acid sequence of SEQ ID NO: 206;
x. a VH comprising the amino acid sequence of SEQ ID NO: 207 and a VL comprising the amino acid sequence of SEQ ID NO: 208;
y. a VH comprising the amino acid sequence of SEQ ID NO: 209 and a VL comprising the amino acid sequence of SEQ ID NO: 210;
z. a VH comprising the amino acid sequence of SEQ ID NO: 234 and a VL comprising the amino acid sequence of SEQ ID NO: 241;
aa. a VH comprising the amino acid sequence of SEQ ID NO: 235 and a VL comprising the amino acid sequence of SEQ ID NO: 242;
bb. a VH comprising the amino acid sequence of SEQ ID NO: 236 and a VL comprising the amino acid sequence of SEQ ID NO: 243;
cc. a VH comprising the amino acid sequence of SEQ ID NO: 237 and a VL comprising the amino acid sequence of SEQ ID NO: 243;
dd. a VH comprising the amino acid sequence of SEQ ID NO: 238 and a VL comprising the amino acid sequence of SEQ ID NO: 244;
ee. a VH comprising the amino acid sequence of SEQ ID NO: 236 and a VL comprising the amino acid sequence of SEQ ID NO: 245;
ff. a VH comprising the amino acid sequence of SEQ ID NO: 237 and a VL comprising the amino acid sequence of SEQ ID NO: 245;
gg. a VH comprising the amino acid sequence of SEQ ID NO: 236 and a VL comprising the amino acid sequence of SEQ ID NO: 246;
hh. a VH comprising the amino acid sequence of SEQ ID NO: 237 and a VL comprising the amino acid sequence of SEQ ID NO: 247;
ii. a VH comprising the amino acid sequence of SEQ ID NO: 238 and a VL comprising the amino acid sequence of SEQ ID NO: 247;
jj. a VH comprising the amino acid sequence of SEQ ID NO: 236 and a VL comprising the amino acid sequence of SEQ ID NO: 248;
kk. a VH comprising the amino acid sequence of SEQ ID NO: 239 and a VL comprising the amino acid sequence of SEQ ID NO: 249;
ll. a VH comprising the amino acid sequence of SEQ ID NO: 240 and a VL comprising the amino acid sequence of SEQ ID NO: 250;
mm. a VH comprising the amino acid sequence of SEQ ID NO: 240 and a VL comprising the amino acid sequence of SEQ ID NO: 251;
nn. a VH comprising the amino acid sequence of SEQ ID NO: 240 and a VL comprising the amino acid sequence of SEQ ID NO: 252;
oo. a VH comprising the amino acid sequence of SEQ ID NO: 240 and a VL comprising the amino acid sequence of SEQ ID NO: 253;
pp. a VH comprising the amino acid sequence of SEQ ID NO: 240 and a VL comprising the amino acid sequence of SEQ ID NO: 254; or
qq. a VH comprising the amino acid sequence of SEQ ID NO: 240 and a VL comprising the amino acid sequence of SEQ ID NO: 249.

46-48. (canceled)

49. An isolated anti-Glycoprotein nonmetastatic melanoma protein B (GPNMB) antibody, wherein the anti-GPNMB antibody comprises:
a. a heavy chain comprising the amino acid sequence of SEQ ID NO:290 or SEQ ID NO:291 and a light chain comprising the amino acid sequence of SEQ ID NO:416;
b. a heavy chain comprising the amino acid sequence of SEQ ID NO:292 or SEQ ID NO:293 and a light chain comprising the amino acid sequence of SEQ ID NO:416;
c. a heavy chain comprising the amino acid sequence of SEQ ID NO:294 or SEQ ID NO:295 and a light chain comprising the amino acid sequence of SEQ ID NO:416;
d. a heavy chain comprising the amino acid sequence of SEQ ID NO:296 or SEQ ID NO:297 and a light chain comprising the amino acid sequence of SEQ ID NO:416;
e. a heavy chain comprising the amino acid sequence of SEQ ID NO:298 or SEQ ID NO:299 and a light chain comprising the amino acid sequence of SEQ ID NO:416;
f. a heavy chain comprising the amino acid sequence of SEQ ID NO:300 SEQ ID NO:301 and a light chain comprising the amino acid sequence of SEQ ID NO:416;
g. a heavy chain comprising the amino acid sequence of SEQ ID NO:302 or SEQ ID NO:303 and a light chain comprising the amino acid sequence of SEQ ID NO:416;
h. a heavy chain comprising the amino acid sequence of SEQ ID NO:304 SEQ ID NO:305 and a light chain comprising the amino acid sequence of SEQ ID NO:416;
i. a heavy chain comprising the amino acid sequence of SEQ ID NO:306 or SEQ ID NO:307 and a light chain comprising the amino acid sequence of SEQ ID NO:416;
j. a heavy chain comprising the amino acid sequence of SEQ ID NO:308 or SEQ ID NO:309 and a light chain comprising the amino acid sequence of SEQ ID NO:417;
k. a heavy chain comprising the amino acid sequence of SEQ ID NO:310 or SEQ ID NO:311 and a light chain comprising the amino acid sequence of SEQ ID NO:417;
l. a heavy chain comprising the amino acid sequence of SEQ ID NO:312 or SEQ ID NO:313 and a light chain comprising the amino acid sequence of SEQ ID NO:417;
m. a heavy chain comprising the amino acid sequence of SEQ ID NO:314 or SEQ ID NO:315 and a light chain comprising the amino acid sequence of SEQ ID NO:417;
n. a heavy chain comprising the amino acid sequence of SEQ ID NO:316 or SEQ ID NO:317 and a light chain comprising the amino acid sequence of SEQ ID NO:417;
o. a heavy chain comprising the amino acid sequence of SEQ ID NO:318 or SEQ ID NO:319 and a light chain comprising the amino acid sequence of SEQ ID NO:417;
p. a heavy chain comprising the amino acid sequence of SEQ ID NO:320 or SEQ ID NO:321 and a light chain comprising the amino acid sequence of SEQ ID NO:417;
q. a heavy chain comprising the amino acid sequence of SEQ ID NO:322 or SEQ ID NO:323 and a light chain comprising the amino acid sequence of SEQ ID NO:417;
r. a heavy chain comprising the amino acid sequence of SEQ ID NO:324 or SEQ ID NO:325 and a light chain comprising the amino acid sequence of SEQ ID NO:417;
s. a heavy chain comprising the amino acid sequence of SEQ ID NO:326 or SEQ ID NO:327 and a light chain comprising the amino acid sequence of SEQ ID NO:418;
t. a heavy chain comprising the amino acid sequence of SEQ ID NO:328 or SEQ ID NO:329 and a light chain comprising the amino acid sequence of SEQ ID NO:418;
u. a heavy chain comprising the amino acid sequence of SEQ ID NO:330 or SEQ ID NO:331 and a light chain comprising the amino acid sequence of SEQ ID NO:418;
v. a heavy chain comprising the amino acid sequence of SEQ ID NO:332 or SEQ ID NO:333 and a light chain comprising the amino acid sequence of SEQ ID NO:418;
w. a heavy chain comprising the amino acid sequence of SEQ ID NO:334 or SEQ ID NO:335 and a light chain comprising the amino acid sequence of SEQ ID NO:418;
x. a heavy chain comprising the amino acid sequence of SEQ ID NO:336 or SEQ ID NO:337 and a light chain comprising the amino acid sequence of SEQ ID NO:418;
y. a heavy chain comprising the amino acid sequence of SEQ ID NO:338 or SEQ ID NO:339 and a light chain comprising the amino acid sequence of SEQ ID NO:418;
z. a heavy chain comprising the amino acid sequence of SEQ ID NO:340 or SEQ ID NO:341 and a light chain comprising the amino acid sequence of SEQ ID NO:418;
aa. a heavy chain comprising the amino acid sequence of SEQ ID NO:342 or SEQ ID NO:343 and a light chain comprising the amino acid sequence of SEQ ID NO:418;
bb. a heavy chain comprising the amino acid sequence of SEQ ID NO:344 or SEQ ID NO:345 and a light chain comprising the amino acid sequence of SEQ ID NO:419;
cc. a heavy chain comprising the amino acid sequence of SEQ ID NO:346 or SEQ ID NO:347 and a light chain comprising the amino acid sequence of SEQ ID NO:419;
dd. a heavy chain comprising the amino acid sequence of SEQ ID NO:348 or SEQ ID NO:349 and a light chain comprising the amino acid sequence of SEQ ID NO:419;
ee. a heavy chain comprising the amino acid sequence of SEQ ID NO:350 or SEQ ID NO:351 and a light chain comprising the amino acid sequence of SEQ ID NO:419;
ff. a heavy chain comprising amino acid sequence of SEQ ID NO:352 or SEQ ID NO:353 and a light chain comprising the amino acid sequence of SEQ ID NO:419;
gg. a heavy chain comprising the amino acid sequence of SEQ ID NO:354 or SEQ ID NO:355 and a light chain comprising the amino acid sequence of SEQ ID NO:419;
hh. a heavy chain comprising the amino acid sequence of SEQ ID NO:356 or SEQ ID NO:357 and a light chain comprising the amino acid sequence of SEQ ID NO:419;
ii. a heavy chain comprising the amino acid sequence of SEQ ID NO:358 or SEQ ID NO:359 and a light chain comprising the amino acid sequence of SEQ ID NO:419;
jj. a heavy chain comprising the amino acid sequence of SEQ ID NO:360 or SEQ ID NO:361 and a light chain comprising the amino acid sequence of SEQ ID NO:419;
kk. a heavy chain comprising the amino acid sequence of SEQ ID NO:362 or SEQ ID NO:363 and a light chain comprising the amino acid sequence of SEQ ID NO:420;
ll. a heavy chain comprising the amino acid sequence of SEQ ID NO:364 or SEQ ID NO:365 and a light chain comprising the amino acid sequence of SEQ ID NO:420;
mm. a heavy chain comprising the amino acid sequence of SEQ ID NO:366 or SEQ ID NO:367 and a light chain comprising the amino acid sequence of SEQ ID NO:420;
nn. a heavy chain comprising the amino acid sequence of SEQ ID NO:368 or SEQ ID NO:369 and a light chain comprising the amino acid sequence of SEQ ID NO:420;
oo. a heavy chain comprising the amino acid sequence of SEQ ID NO:370 or SEQ ID NO:371 and a light chain comprising the amino acid sequence of SEQ ID NO:420;
pp. a heavy chain comprising the amino acid sequence of SEQ ID NO:372 or SEQ ID NO:373 and a light chain comprising the amino acid sequence of SEQ ID NO:420;
qq. a heavy chain comprising the amino acid sequence of SEQ ID NO:374 or SEQ ID NO:375 and a light chain comprising the amino acid sequence of SEQ ID NO:420;
rr. a heavy chain comprising the amino acid sequence of SEQ ID NO:376 or SEQ ID NO:377 and a light chain comprising the amino acid sequence of SEQ ID NO:420;
ss. a heavy chain comprising the amino acid sequence of SEQ ID NO:378 or SEQ ID NO:379 and a light chain comprising the amino acid sequence of SEQ ID NO:420;
tt. a heavy chain comprising the amino acid sequence of SEQ ID NO:326 or SEQ ID NO:327 and a light chain comprising the amino acid sequence of SEQ ID NO:421;
uu. a heavy chain comprising the amino acid sequence of SEQ ID NO:328 or SEQ ID NO:329 and a light chain comprising the amino acid sequence of SEQ ID NO:421;
vv. a heavy chain comprising the amino acid sequence of SEQ ID NO:330 or SEQ ID NO:331 and a light chain comprising the amino acid sequence of SEQ ID NO:421;
ww. a heavy chain comprising the amino acid sequence of SEQ ID NO:332 or SEQ ID NO:333 and a light chain comprising the amino acid sequence of SEQ ID NO:421;
xx. a heavy chain comprising the amino acid sequence of SEQ ID NO:334 or SEQ ID NO:335 and a light chain comprising the amino acid sequence of SEQ ID NO:421;
yy. a heavy chain comprising the amino acid sequence of SEQ ID NO:336 or SEQ ID NO:337 and a light chain comprising the amino acid sequence of SEQ ID NO:421;
zz. a heavy chain comprising the amino acid sequence of SEQ ID NO:338 or SEQ ID NO:339 and a light chain comprising the amino acid sequence of SEQ ID NO:421;
aaa. a heavy chain comprising the amino acid sequence of SEQ ID NO:340 or SEQ ID NO:341 and a light chain comprising the amino acid sequence of SEQ ID NO:421;
bbb. a heavy chain comprising the amino acid sequence of SEQ ID NO:342 or SEQ ID NO:343 and a light chain comprising the amino acid sequence of SEQ ID NO:421;
ccc. a heavy chain comprising the amino acid sequence of SEQ ID NO:344 or SEQ ID NO:345 and a light chain comprising the amino acid sequence of SEQ ID NO:422;
ddd. a heavy chain comprising the amino acid sequence of SEQ ID NO:346 or SEQ ID NO:347 and a light chain comprising the amino acid sequence of SEQ ID NO:422;
eee. a heavy chain comprising the amino acid sequence of SEQ ID NO:348 or SEQ ID NO:349 and a light chain comprising the amino acid sequence of SEQ ID NO:422;
fff. a heavy chain comprising the amino acid sequence of SEQ ID NO:350 or SEQ ID NO:351 and a light chain comprising the amino acid sequence of SEQ ID NO:422;
ggg. a heavy chain comprising the amino acid sequence of SEQ ID NO:352 or SEQ ID NO:353 and a light chain comprising the amino acid sequence of SEQ ID NO:422;
hhh. a heavy chain comprising the amino acid sequence of SEQ ID NO:354 or SEQ ID NO:355 and a light chain comprising the amino acid sequence of SEQ ID NO:422;
iii. a heavy chain comprising the amino acid sequence of SEQ ID NO:356 or SEQ ID NO:357 and a light chain comprising the amino acid sequence of SEQ ID NO:422;
jjj. a heavy chain comprising the amino acid sequence of SEQ ID NO:358 or SEQ ID NO:359 and a light chain comprising the amino acid sequence of SEQ ID NO:422;
kkk. a heavy chain comprising the amino acid sequence of SEQ ID NO:360 or SEQ ID NO:361 and a light chain comprising the amino acid sequence of SEQ ID NO:422;
lll. a heavy chain comprising the amino acid sequence of SEQ ID NO:326 or SEQ ID NO:327 and a light chain comprising the amino acid sequence of SEQ ID NO:423;
mmm. a heavy chain comprising the amino acid sequence of SEQ ID NO:328 or SEQ ID NO:329 and a light chain comprising the amino acid sequence of SEQ ID NO:423;
nnn. a heavy chain comprising the amino acid sequence of SEQ ID NO:330 or SEQ ID NO:331 and a light chain comprising the amino acid sequence of SEQ ID NO:423;
ooo. a heavy chain comprising the amino acid sequence of SEQ ID NO:332 or SEQ ID NO:333 and a light chain comprising the amino acid sequence of SEQ ID NO:423;
ppp. a heavy chain comprising the amino acid sequence of SEQ ID NO:334 or SEQ ID NO:335 and a light chain comprising the amino acid sequence of SEQ ID NO:423;
qqq. a heavy chain comprising the amino acid sequence of SEQ ID NO:336 or SEQ ID NO:337 and a light chain comprising the amino acid sequence of SEQ ID NO:423;
rrr. a heavy chain comprising the amino acid sequence of SEQ ID NO:338 or SEQ ID NO:339 and a light chain comprising the amino acid sequence of SEQ ID NO:423;
sss. a heavy chain comprising the amino acid sequence of SEQ ID NO:340 or SEQ ID NO:341 and a light chain comprising the amino acid sequence of SEQ ID NO:423;
ttt. a heavy chain comprising the amino acid sequence of SEQ ID NO:342 or SEQ ID NO:343 and a light chain comprising the amino acid sequence of SEQ ID NO:423;
uuu. a heavy chain comprising the amino acid sequence of SEQ ID NO:344 or SEQ ID NO:345 and a light chain comprising the amino acid sequence of SEQ ID NO:424;
vvv. a heavy chain comprising the amino acid sequence of SEQ ID NO:346 or SEQ ID NO:347 and a light chain comprising the amino acid sequence of SEQ ID NO:424;
www. a heavy chain comprising the amino acid sequence of SEQ ID NO:348 or SEQ ID NO:349 and a light chain comprising the amino acid sequence of SEQ ID NO:419;
xxx. a heavy chain comprising the amino acid sequence of SEQ ID NO:350 or SEQ ID NO:351 and a light chain comprising the amino acid sequence of SEQ ID NO:424;
yyy. a heavy chain comprising amino acid sequence of SEQ ID NO:352 or SEQ ID NO:353 and a light chain comprising the amino acid sequence of SEQ ID NO:424;
zzz. a heavy chain comprising the amino acid sequence of SEQ ID NO:354 or SEQ ID NO:355 and a light chain comprising the amino acid sequence of SEQ ID NO:424;
aaaa. a heavy chain comprising the amino acid sequence of SEQ ID NO:356 or SEQ ID NO:357 and a light chain comprising the amino acid sequence of SEQ ID NO:424;
bbbb. a heavy chain comprising the amino acid sequence of SEQ ID NO:358 or SEQ ID NO:359 and a light chain comprising the amino acid sequence of SEQ ID NO:424;
cccc. a heavy chain comprising the amino acid sequence of SEQ ID NO:360 or SEQ ID NO:361 and a light chain comprising the amino acid sequence of SEQ ID NO:424;
dddd. a heavy chain comprising the amino acid sequence of SEQ ID NO:362 or SEQ ID NO:363 and a light chain comprising the amino acid sequence of SEQ ID NO:425;
eeee. a heavy chain comprising the amino acid sequence of SEQ ID NO:364 or SEQ ID NO:365 and a light chain comprising the amino acid sequence of SEQ ID NO:425;
ffff. a heavy chain comprising the amino acid sequence of SEQ ID NO:366 or SEQ ID NO:367 and a light chain comprising the amino acid sequence of SEQ ID NO:425;
gggg. a heavy chain comprising the amino acid sequence of SEQ ID NO:368 or SEQ ID NO:369 and a light chain comprising the amino acid sequence of SEQ ID NO:425;
hhhh. a heavy chain comprising the amino acid sequence of SEQ ID NO:370 or SEQ ID NO:371 and a light chain comprising the amino acid sequence of SEQ ID NO:425;
iiii. a heavy chain comprising the amino acid sequence of SEQ ID NO:372 or SEQ ID NO:373 and a light chain comprising the amino acid sequence of SEQ ID NO:425;
jjjj. a heavy chain comprising the amino acid sequence of SEQ ID NO:374 or SEQ ID NO:375 and a light chain comprising the amino acid sequence of SEQ ID NO:425;
kkkk. a heavy chain comprising the amino acid sequence of SEQ ID NO:376 or SEQ ID NO:377 and a light chain comprising the amino acid sequence of SEQ ID NO:425;
llll. a heavy chain comprising the amino acid sequence of SEQ ID NO:378 or SEQ ID NO:379 and a light chain comprising the amino acid sequence of SEQ ID NO:425;
mmmm. a heavy chain comprising the amino acid sequence of SEQ ID NO:326 or SEQ ID NO:327 and a light chain comprising the amino acid sequence of SEQ ID NO:426;
nnnn. a heavy chain comprising the amino acid sequence of SEQ ID NO:328 or SEQ ID NO:329 and a light chain comprising the amino acid sequence of SEQ ID NO:426;
oooo. a heavy chain comprising the amino acid sequence of SEQ ID NO:330 or SEQ ID NO:331 and a light chain comprising the amino acid sequence of SEQ ID NO:426;
pppp. a heavy chain comprising the amino acid sequence of SEQ ID NO:332 or SEQ ID NO:333 and a light chain comprising the amino acid sequence of SEQ ID NO:426;
qqqq. a heavy chain comprising the amino acid sequence of SEQ ID NO:334 or SEQ ID NO:335 and a light chain comprising the amino acid sequence of SEQ ID NO:426;
rrrr. a heavy chain comprising the amino acid sequence of SEQ ID NO:336 or SEQ ID NO:337 and a light chain comprising the amino acid sequence of SEQ ID NO:426;
ssss. a heavy chain comprising the amino acid sequence of SEQ ID NO:338 or SEQ ID NO:339 and a light chain comprising the amino acid sequence of SEQ ID NO:426;
tttt. a heavy chain comprising the amino acid sequence of SEQ ID NO:340 or SEQ ID NO:341 and a light chain comprising the amino acid sequence of SEQ ID NO:426;
uuuu. a heavy chain comprising the amino acid sequence of SEQ ID NO:342 or SEQ ID NO:343 and a light chain comprising the amino acid sequence of SEQ ID NO:426;
vvvv. a heavy chain comprising the amino acid sequence of SEQ ID NO:380 or SEQ ID NO:381 and a light chain comprising the amino acid sequence of SEQ ID NO:427;
wwww. a heavy chain comprising the amino acid sequence of SEQ ID NO:382 or SEQ ID NO:383 and a light chain comprising the amino acid sequence of SEQ ID NO:427;
xxxx. a heavy chain comprising the amino acid sequence of SEQ ID NO:384 or SEQ ID NO:385 and a light chain comprising the amino acid sequence of SEQ ID NO:427;
yyyy. a heavy chain comprising the amino acid sequence of SEQ ID NO:386 or SEQ ID NO:387 and a light chain comprising the amino acid sequence of SEQ ID NO:427;
zzzz. a heavy chain comprising the amino acid sequence of SEQ ID NO:388 or SEQ ID NO:389 and a light chain comprising the amino acid sequence of SEQ ID NO:427;
aaaaa. a heavy chain comprising the amino acid sequence of SEQ ID NO:390 or SEQ ID NO:391 and a light chain comprising the amino acid sequence of SEQ ID NO:427;
bbbbb. a heavy chain comprising the amino acid sequence of SEQ ID NO:392 or SEQ ID NO:393 and a light chain comprising the amino acid sequence of SEQ ID NO:427;
ccccc. a heavy chain comprising the amino acid sequence of SEQ ID NO:394 or SEQ ID NO:395 and a light chain comprising the amino acid sequence of SEQ ID NO:427;
ddddd. a heavy chain comprising the amino acid sequence of SEQ ID NO:396 or SEQ ID NO:397 and a light chain comprising the amino acid sequence of SEQ ID NO:427;
eeeee. a heavy chain comprising the amino acid sequence of SEQ ID NO:398 or SEQ ID NO:399 and a light chain comprising the amino acid sequence of SEQ ID NO:428;
fffff. a heavy chain comprising the amino acid sequence of SEQ ID NO:400 or SEQ ID NO:401 and a light chain comprising the amino acid sequence of SEQ ID NO:428;
ggggg. a heavy chain comprising the amino acid sequence of SEQ ID NO:402 or SEQ ID NO:403 and a light chain comprising the amino acid sequence of SEQ ID NO:428;
hhhhh. a heavy chain comprising the amino acid sequence of SEQ ID NO:404 or SEQ ID NO:405 and a light chain comprising the amino acid sequence of SEQ ID NO:428;
iiiii. a heavy chain comprising the amino acid sequence of SEQ ID NO:406 or SEQ ID NO:407 and a light chain comprising the amino acid sequence of SEQ ID NO:428;
jjjjj. a heavy chain comprising the amino acid sequence of SEQ ID NO:408 or SEQ ID NO:409 and a light chain comprising the amino acid sequence of SEQ ID NO:428;
kkkkk. a heavy chain comprising the amino acid sequence of SEQ ID NO:410 or SEQ ID NO:411 and a light chain comprising the amino acid sequence of SEQ ID NO:428;
lllll. a heavy chain comprising the amino acid sequence of SEQ ID NO:412 or SEQ ID NO:413 and a light chain comprising the amino acid sequence of SEQ ID NO:428;
mmmmm. a heavy chain comprising the amino acid sequence of SEQ ID NO:414 or SEQ ID NO:415 and a light chain comprising the amino acid sequence of SEQ ID NO:428;
nnnnn. a heavy chain comprising the amino acid sequence of SEQ ID NO:398 or SEQ ID NO:399 and a light chain comprising the amino acid sequence of SEQ ID NO:429;
ooooo. a heavy chain comprising the amino acid sequence of SEQ ID NO:400 or SEQ ID NO:401 and a light chain comprising the amino acid sequence of SEQ ID NO:429;
ppppp. a heavy chain comprising the amino acid sequence of SEQ ID NO:402 or SEQ ID NO:403 and a light chain comprising the amino acid sequence of SEQ ID NO:429;
qqqqq. a heavy chain comprising the amino acid sequence of SEQ ID NO:404 or SEQ ID NO:405 and a light chain comprising the amino acid sequence of SEQ ID NO:429;
rrrrr. a heavy chain comprising the amino acid sequence of SEQ ID NO:406 or SEQ ID NO:407 and a light chain comprising the amino acid sequence of SEQ ID NO:429;
sssss. a heavy chain comprising the amino acid sequence of SEQ ID NO:408 or SEQ ID NO:409 and a light chain comprising the amino acid sequence of SEQ ID NO:429;
ttttt. a heavy chain comprising the amino acid sequence of SEQ ID NO:410 or SEQ ID NO:411 and a light chain comprising the amino acid sequence of SEQ ID NO:429;
uuuuu. a heavy chain comprising the amino acid sequence of SEQ ID NO:412 or SEQ ID NO:413 and a light chain comprising the amino acid sequence of SEQ ID NO:429;
vvvvv. a heavy chain comprising the amino acid sequence of SEQ ID NO:414 or SEQ ID NO:415 and a light chain comprising the amino acid sequence of SEQ ID NO:429;
wwwww. a heavy chain comprising the amino acid sequence of SEQ ID NO:398 or SEQ ID NO:399 and a light chain comprising the amino acid sequence of SEQ ID NO:430;
xxxxx. a heavy chain comprising the amino acid sequence of SEQ ID NO:400 or SEQ ID NO:401 and a light chain comprising the amino acid sequence of SEQ ID NO:430;
yyyyy. a heavy chain comprising the amino acid sequence of SEQ ID NO:402 or SEQ ID NO:403 and a light chain comprising the amino acid sequence of SEQ ID NO:430;
zzzzz. a heavy chain comprising the amino acid sequence of SEQ ID NO:404 or SEQ ID NO:405 and a light chain comprising the amino acid sequence of SEQ ID NO:430;
aaaaaa. a heavy chain comprising the amino acid sequence of SEQ ID NO:406 or SEQ ID NO:407 and a light chain comprising the amino acid sequence of SEQ ID NO:430;
bbbbbb. a heavy chain comprising the amino acid sequence of SEQ ID NO:408 or SEQ ID NO:409 and a light chain comprising the amino acid sequence of SEQ ID NO:430;
cccccc. a heavy chain comprising the amino acid sequence of SEQ ID NO:410 or SEQ ID NO:411 and a light chain comprising the amino acid sequence of SEQ ID NO:430;
dddddd. a heavy chain comprising the amino acid sequence of SEQ ID NO:412 or SEQ ID NO:413 and a light chain comprising the amino acid sequence of SEQ ID NO:430;
eeeeee. a heavy chain comprising the amino acid sequence of SEQ ID NO:414 or SEQ ID NO:415 and a light chain comprising the amino acid sequence of SEQ ID NO:430;
ffffff. a heavy chain comprising the amino acid sequence of SEQ ID NO:398 or SEQ ID NO:399 and a light chain comprising the amino acid sequence of SEQ ID NO:431;
gggggg. a heavy chain comprising the amino acid sequence of SEQ ID NO:400 or SEQ ID NO:401 and a light chain comprising the amino acid sequence of SEQ ID NO:431;
hhhhhh. a heavy chain comprising the amino acid sequence of SEQ ID NO:402 or SEQ ID NO:403 and a light chain comprising the amino acid sequence of SEQ ID NO:431;
iiiiii. a heavy chain comprising the amino acid sequence of SEQ ID NO:404 or SEQ ID NO:405 and a light chain comprising the amino acid sequence of SEQ ID NO:431;
jjjjjj. a heavy chain comprising the amino acid sequence of SEQ ID NO:406 or SEQ ID NO:407 and a light chain comprising the amino acid sequence of SEQ ID NO:431;
kkkkkk. a heavy chain comprising the amino acid sequence of SEQ ID NO:408 or SEQ ID NO:409 and a light chain comprising the amino acid sequence of SEQ ID NO:431;
llllll. a heavy chain comprising the amino acid sequence of SEQ ID NO:410 or SEQ ID NO:411 and a light chain comprising the amino acid sequence of SEQ ID NO:431;
mmmmmm. a heavy chain comprising the amino acid sequence of SEQ ID NO:412 or SEQ ID NO:413 and a light chain comprising the amino acid sequence of SEQ ID NO:431;
nnnnnn. a heavy chain comprising the amino acid sequence of SEQ ID NO:414 or SEQ ID NO:415 and a light chain comprising the amino acid sequence of SEQ ID NO:431;
oooooo. a heavy chain comprising the amino acid sequence of SEQ ID NO:398 or SEQ ID NO:399 and a light chain comprising the amino acid sequence of SEQ ID NO 432;
pppppp. a heavy chain comprising the amino acid sequence of SEQ ID NO:400 or SEQ ID NO:401 and a light chain comprising the amino acid sequence of SEQ ID NO 432;
qqqqqq. a heavy chain comprising the amino acid sequence of SEQ ID NO:402 or SEQ ID NO:403 and a light chain comprising the amino acid sequence of SEQ ID NO 432;
rrrrrr. a heavy chain comprising the amino acid sequence of SEQ ID NO:404 or SEQ ID NO:405 and a light chain comprising the amino acid sequence of SEQ ID NO 432;
ssssss. a heavy chain comprising the amino acid sequence of SEQ ID NO:406 or SEQ ID NO:407 and a light chain comprising the amino acid sequence of SEQ ID NO 432;
tttttt. a heavy chain comprising the amino acid sequence of SEQ ID NO:408 or SEQ ID NO:409 and a light chain comprising the amino acid sequence of SEQ ID NO:432;
uuuuuu. a heavy chain comprising the amino acid sequence of SEQ ID NO:410 or SEQ ID NO:411 and a light chain comprising the amino acid sequence of SEQ ID NO:432;
vvvvvv. a heavy chain comprising the amino acid sequence of SEQ ID NO:412 or SEQ ID NO:413 and a light chain comprising the amino acid sequence of SEQ ID NO:432;
wwwwww. a heavy chain comprising the amino acid sequence of SEQ ID NO:414 or SEQ ID NO:415 and a light chain comprising the amino acid sequence of SEQ ID NO:432;
xxxxxx. a heavy chain comprising the amino acid sequence of SEQ ID NO:398 or SEQ ID NO:399 and a light chain comprising the amino acid sequence of SEQ ID NO:433;
yyyyyy. a heavy chain comprising the amino acid sequence of SEQ ID NO:400 or SEQ ID NO:401 and a light chain comprising the amino acid sequence of SEQ ID NO:433;
zzzzzz. a heavy chain comprising the amino acid sequence of SEQ ID NO:402 or SEQ ID NO:403 and a light chain comprising the amino acid sequence of SEQ ID NO:433;
aaaaaaa. a heavy chain comprising the amino acid sequence of SEQ ID NO:404 or SEQ ID NO:405 and a light chain comprising the amino acid sequence of SEQ ID NO:433;
bbbbbbb. a heavy chain comprising the amino acid sequence of SEQ ID NO:406 or SEQ ID NO:407 and a light chain comprising the amino acid sequence of SEQ ID NO:433;
ccccccc. a heavy chain comprising the amino acid sequence of SEQ ID NO:408 or SEQ ID NO:409 and a light chain comprising the amino acid sequence of SEQ ID NO:433;
ddddddd. a heavy chain comprising the amino acid sequence of SEQ ID NO:410 or SEQ ID NO:411 and a light chain comprising the amino acid sequence of SEQ ID NO:433;
eeeeeee. a heavy chain comprising the amino acid sequence of SEQ ID NO:412 or SEQ ID NO:413 and a light chain comprising the amino acid sequence of SEQ ID NO:433; or
fffffff. a heavy chain comprising the amino acid sequence of SEQ ID NO:414 or SEQ ID NO:415 and a light chain comprising the amino acid sequence of SEQ ID NO:433.

50-54. (canceled)

55. The antibody of claim 40, wherein the antibody has one or more of the following properties:
a. the antibody modulates expression of activation surface markers on myeloid cells;
b. the antibody increases cell surface expression of PD-L1 in human macrophages;
c. the antibody increases cell surface expression of CD40 in human macrophages;
d. the antibody increases cell surface expression of CD80 in human macrophages;
e. the antibody modulates lysosome function in myeloid cells;
f. the antibody increases glucocerebrosidase activity in human macrophages;
g. the antibody decreases cell surface expression of GPNMB on human macrophages;
h. the antibody changes interferon pathway gene expression patterns in human macrophages;
i. the antibody reduces tumor volume in a murine tumor model, such as an MC38 tumor model;
j. the antibody reduces tumor growth rate in a murine model, such as an MC38 tumor model;
k. the antibody increases levels of IL-12p40 in serum;
l. the antibody increases levels of CCL5 in serum;
m. the antibody is a GPNMB ligand blocking antibody;
n. the antibody is a non-blocking antibody;
o. the antibody antagonizes GPNMB activity independent of ligand blocking activity;
p. the antibody antagonizes GPNMB activity in vivo;
q. the antibody binds to GPNMB expressed on a cell surface;
r. the antibody promotes macrophage activation;
s. the antibody overcomes a decrease in glucocerebrosidase activity associated with reduced progranulin levels;
t. the antibody decreases GPNMB expression levels in cells, optionally in macrophages;
u. the antibody decreases LAMP2 expression levels in cells, optionally in macrophages, monocytes, or neutrophils;
v. the antibody inhibits or reduces inflammasome activation;
w. the antibody inhibits IL-1β expression or release;
x. the antibody reduces neural inflammation;
y. the antibody reduces expression of C1q, GFAP, IBA1, and CTSD associated with neural inflammation; and
z. the antibody reduces lysosomal stress.

56-58. (canceled)

59. The antibody of claim 40, wherein the antibody is a humanized antibody.

60. (canceled)

61. The antibody of claim 40, wherein the antibody is a bispecific or multispecific antibody.

62. The antibody of claim 40, wherein the antibody is of the IgG class, the IgM class, or the IgA class.

63-70. (canceled)

71. The antibody of claim 40, wherein the antibody further comprises an IgG Fc amino acid sequence selected from the group consisting of SEQ ID NOs: 213-233.

72. A pharmaceutical composition comprising the anti-GPNMB antibody of claim 40 and a pharmaceutically acceptable carrier.

73. An isolated nucleic acid comprising a nucleic acid sequence encoding the anti-GPNMB antibody claim 40.

74. An isolated vector comprising the nucleic acid of claim 73.

75. An isolated host cell comprising the nucleic acid of claim 73.

76. A method of producing an anti-GPNMB antibody, comprising culturing the cell of claim 75 so that the antibody is produced.

77. (canceled)

78. A method of treating a cancer in a subject in need thereof, the method comprising administering to an individual in need thereof a therapeutically effective amount of an anti-GPNMB antibody of claim 40, thereby treating the cancer.

79-84. (canceled)

85. A method of treating a neurodegenerative disease or disorder in a subject in need thereof, the method comprising administering to an individual in need thereof a therapeutically effective amount of an anti-GPNMB antibody of claim 40, thereby treating the disease or disorder.

86. (canceled)

87. A method of treating a lysosomal storage disease or disorder in a subject in need thereof, the method comprising administering to an individual in need thereof a therapeutically effective amount of an anti-GPNMB antibody of claim 40, thereby treating the disease or disorder.

88. (canceled)

89. A method of detecting the presence of GPNMB in a sample in vitro or an individual, the method comprising an anti-GPNMB antibody of claim 40.

90. (canceled)
